(19) United States
(12) Patent Application Publication
(10) Pub. No.: US 2025/0304686 A1
(43) Pub. Date: Oct. 2, 2025

(54) CONDITIONALLY BISPECIFIC BINDING PROTEINS

(71) Applicant: Takeda Pharmaceutical Company Limited, Osaka-shi, Osaka (JP)

(72) Inventors: Tseng-hui Timothy Chen, Burlingame, CA (US); Patricia A. Culp, Oakland, CA (US)

(73) Assignee: Takeda Pharmaceutical Company Limited, Osaka-shi, Osaka (JP)

(21) Appl. No.: 18/266,848

(22) PCT Filed: Dec. 14, 2021
(86) PCT No.: PCT/IB2021/000868

§ 371 Date: Jun. 13, 2023

Related U.S. Application Data
(60) Provisional application No. 63/125,267, filed on Dec. 14, 2020.

(51) Int. Cl. C07K 16/28; C07K 16/32

(52) U.S. Cl. CPC C07K 16/2809; C07K 16/2818; C07K 16/32; C07K 2317/31; C07K 2317/33; C07K 2317/622; C07K 2317/92

(57) ABSTRACT

The present disclosure, in some aspects, provides Co-stimulatory Conditional Bispecific Redirected Activation constructs, or “co-stim COBRA,” that are administered in an active prodrug format. Upon exposure to tumor proteases, the constructs are cleaved and activated, such that they can bind both tumor target antigens (TTAs) as well and immune cells (e.g., one or more types of immune cells), thus recruiting immune cells to tumor, resulting in treatment.

### RELATED APPLICATIONS

This application is a national stage filing under 35 U.S.C. § 371 of International Patent Application No. PCT/IB2021/000868, filed Dec. 14, 2021, which claims the benefit under 35 U.S.C. § 119 (e) of U.S. provisional application No. 63/125,267 filed Dec. 14, 2020. The contents of these applications are incorporated herein by reference in their entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jun. 8, 2023, is named T083370010US01-SEQ-ZJG and is 412,466 bytes in size.

### BACKGROUND

The selective destruction of an individual cell or a specific cell type is often desirable in a variety of clinical settings. For example, it is a primary goal of cancer therapy to specifically destroy tumor cells, while leaving healthy cells and tissues as intact and undamaged as possible. One such method is by inducing an immune response against the tumor, to make immune effector cells such as natural killer (NK) cells or cytotoxic T lymphocytes (CTLs) attack and destroy tumor cells.

### SUMMARY

The present disclosure, in some aspects, provides methods and compositions for reducing the toxicity and side effects of immune cell engaging bispecific antibodies that bind to cancer and immune cells to stimulate immune cell killing of a target cancer. Many of the proteins provided herein are prodrugs that may be activated by proteases (e.g., proteases found in tumor microenvironments). In some embodiments, the proteins described herein are configured such that, when they are not in a tumor microenvironment, the protein is capable of binding to tumor cells but not immune cells (inactive), and such that when the proteins enter a tumor microenvironment, cleavage of the cleavable linkers in the protein “activates” the protein, resulting in two “active” bi-specific molecules, wherein each can bind to tumor cells and immune cells. In some embodiments, each of the two “active” bi-specific molecules bind a different antigen on immune cells. In some embodiments, the two “active” bi-specific molecules bind two different antigens on the same immune cell. In some embodiments, the two “active” bi-specific molecules bind two different immune cells (e.g., immune cells selected from T-cells, natural killer cells, macrophages, and neutrophils). For example, in some embodiments, the first “active” bi-specific molecule may bind a first target tumor antigen and a first immune cell antigen CD3, while the second “active” bi-specific molecule binds a second target tumor antigen and a second immune cell antigen CD28. The first target tumor antigen and second target tumor antigen may be the same or different. This tumor specific activation decreases potential off-target side effects, and the targeting of two different immune cell antigens enhance the anti-tumor activity of the proteins described herein, e.g., by activating co-stimulating molecules, enhancing T cell recruitment and activity, reducing T-cell exhaustion, enhancing cytotoxicity and IFNγ secretion, stimulating macrophages, and/or enhancing maturation of macrophages.

Some aspects of the present disclosure provide proteins comprising: from N- to C-terminus:
(i) a first single domain antigen binding domain (sdABD) that binds to a first human target tumor antigen (TTA);
(ii) a first domain linker;
(iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first human immune cell antigen, and wherein the first heavy chain variable region and the first light chain variable region are not associated in the first constrained scFv domain and the first constrained scFv domain does not bind to the first human immune cell antigen;
(iv) a first cleavable linker;
(v) a second single domain antigen binding domain (sdABD) that binds to a second human target tumor antigen (TTA);
(vi) a second domain linker;
(vii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second human immune cell antigen, and wherein the first heavy chain variable region and the first light chain variable region are not associated in the second constrained scFv domain, and the second constrained scFv domain does not bind to the second human immune cell antigen;
(viii) a second cleavable linker; and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first immune cell is a T cell, a natural killer (NK) cell, a neutrophil, or a macrophage. In some embodiments, the second immune cell is a T cell, a natural killer (NK) cell, or a macrophage. In some embodiments, the first immune antigen is selected from: CD3, CD28, T cell receptor, programmed cell death protein 1 (PD-1), cytotoxic T-lymphocyte-associated protein 4 (CTLA-4), T-cell immunoglobulin and mucin domain 3 (TIM-3), lymphocyte-activation gene 3 (LAG-3), killer-cell immunoglobulin-like receptor (KIR), CD137, OX40, CD27, GITR (TNFRSF18), TIGIT, inducible T cell costimulatory (ICOS), CD16A, CD226, CD96, CD40L, CD226, CRTAM, LFA-1, CD27, CD96, TIGIT, KIR, NKG2D, CSF1R, CD40, MARCO, VSIG4, and CD163. In some embodiments, the second immune antigen is selected from the group consisting of: CD3, CD28, T cell receptor, programmed cell death protein 1 (PD-1), cytotoxic T-lymphocyte-associated protein 4 (CTLA-4), T cell immunoglobulin and mucin domain 3 (TIM-3), lymphocyte-activation gene 3 (LAG-3), killer-cell immunoglobulin-like receptor (KIR), CD137, OX40, CD27, GITR (TNFRSF18), TIGIT, inducible T cell costimulatory (ICOS), CD16A, CD226, CD96, CD40L, CD226, CRTAM, LFA-1, CD27, CD96, TIGIT, KIR, NKG2D, CSF1R, CD40, MARCO, VSIG4 and CD163.

In some embodiments, the first human immune cell antigen is CD3 and the second immune cell antigen is CD28. In some embodiments, the first human immune cell antigen is CD28 and the second immune cell antigen is CD3.

In some embodiments, the first heavy chain variable region is linked to the N-terminus of the first light chain variable region in the first constrained scFv domain of (iii). In some embodiments, the first heavy chain variable region is linked to the C-terminus of the first light chain variable region in the first constrained scFv domain of (iii). In some embodiments, the second heavy chain variable region is linked to the N-terminus of the second light chain variable region in the second constrained scFv domain of (vii). In some embodiments, the second heavy chain variable region is linked to the C-terminus of the second light chain variable region in the second constrained scFv domain of (vii).

In some embodiments, the first human target tumor antigen is the same as the second human target tumor antigen. In some embodiments, the first sdABD and the second sdABD binds the same epitope. In some embodiments, the first sdABD and the second sdABD binds different epitopes. In some embodiments, the first human target tumor antigen is different from the second human target tumor antigen. In some embodiments, the first human target tumor antigen is selected from EGFR, HER2, Trop2, CA9, LyPD3, FOLR1, EpCAM, and B7H3. In some embodiments, the second human target tumor antigen is selected from EGFR, HER2, Trop2, CA9, LyPD3, FOLR1, EpCAM, and B7H3.

In some embodiments, the first cleavable linker is the same as the second cleavable linker. In some embodiments, the first cleavable linker is different from the second cleavable linker. In some embodiments, the first cleavable linker comprises a cleavage site for a protease that is present in a tumor microenvironment. In some embodiments, the second cleavable linker comprises a cleavage site for a protease that is present in a tumor microenvironment. In some embodiments, the protease is selected from: MMP2, MMP9, Meprin, Cathepsin, granzyme, Matriplase, thrombin, enterokinase, KLK7-6, KLK7-13, KLK7-11, KLK7-10, and uPA.

In some embodiments, the first constrained non-cleavable linker of (iii) and/or the second constrained non-cleavable linker of (vii) is 6-10 amino acids in length, optionally wherein the first constrained non-cleavable linker of (iii) and/or the second constrained non-cleavable linker of (vii) is 8 amino acids in length. In some embodiments, the first domain linker of (ii) and/or the second domain linker of (vi) is a non-cleavable linker.

In some embodiments, the first human target tumor antigen is EGFR and the first sdABD comprises the amino acid sequence of any one of SEQ ID NOs: 4, 5, and 9-11.

In some embodiments, the second human target tumor antigen is EGFR and the second sdABD comprises the amino acid sequence of any one of SEQ ID NOs: 4, 5, and 9-11.

In some embodiments, the second human target tumor antigen is HER2 and the second sdABD comprises the amino acid sequence of any one of SEQ ID NOs: 45, 48-52, 54, 58, 60, 63, 66, 68, 72, 75-78, 82, 85, 89, 95, 96, 99, 103, 104, 108, 112, 116, 117, 121, 299, and 300.

In some embodiments, the first human target tumor antigen is HER2 and the first sdABD comprises the amino acid sequence of any one of SEQ ID NOs: 45, 48-52, 54, 58, 60, 63, 66, 68, 72, 75-78, 82, 85, 89, 95, 96, 99, 103, 104, 108, 112, 116, 117, 121, 299, and 300.

In some embodiments, the second human target tumor antigen is EGFR and the second sdABD comprises the amino acid sequence of any one of SEQ ID NOs: 4, 5, and 9-11.

In some embodiments, the second human target tumor antigen is HER2 and the second sdABD comprises the amino acid sequence of any one of SEQ ID NOs: 45, 48-52, 54, 58, 60, 63, 66, 68, 72, 75-78, 82, 85, 89, 95, 96, 99, 103, 104, 108, 112, 116, 117, 121, 299, and 300.

In some embodiments, the first human target tumor antigen is EGFR and the second human target tumor antigen is EGFR, wherein the first sdABD comprises the amino acid sequence of SEQ ID NO: 5 and the second sdABD comprises the amino acid sequence of SEQ ID NO: 5.

In some embodiments, the first human target tumor antigen is EGFR and the second human target tumor antigen is EGFR, wherein the first sdABD comprises the amino acid sequence of SEQ ID NO: 9 and the second sdABD comprises the amino acid sequence of SEQ ID NO: 9.

In some embodiments, the first human target tumor antigen is HER2 and the second human target tumor antigen is HER2, wherein the first sdABD comprises the amino acid sequence of SEQ ID NO: 96 and the second sdABD comprises the amino acid sequence of SEQ ID NO: 96.

In some embodiments, the first human target tumor antigen is EGFR and the second human target tumor antigen is HER2, wherein the first sdABD comprises the amino acid sequence of SEQ ID NO: 5 or SEQ ID NO: 9 and the second sdABD comprises the amino acid sequence of SEQ ID NO: 96.

In some embodiments, the first human target tumor antigen is HER2 and the second human target tumor antigen is EGFR, wherein the first sdABD comprises the amino acid sequence of SEQ ID NO: 96 and the second sdABD comprises the amino acid sequence of SEQ ID NO: 5 or SEQ ID NO: 9.

In some embodiments, the first human immune cell antigen is CD3, the first heavy chain variable region comprises the amino acid sequence of SEQ ID NO: 205, and the first light chain variable region comprises the amino acid sequence of SEQ ID NO: 206.

In some embodiments, the second human immune cell antigen is CD28, the second heavy chain variable region comprises the amino acid sequence of SEQ ID NO: 213 or SEQ ID NO: 216, and the second light chain variable region comprises the amino acid sequence of SEQ ID NO: 214.

In some embodiments, the first human immune cell antigen is CD28, the first heavy chain variable region comprises the amino acid sequence of SEQ ID NO: 213 or SEQ ID NO: 216, and the first light chain variable region comprises the amino acid sequence of SEQ ID NO: 214.

In some embodiments, the second human immune cell antigen is CD3, the second heavy chain variable region comprises the amino acid sequence of SEQ ID NO: 205, and the second light chain variable region comprises the amino acid sequence of SEQ ID NO: 206.

In some embodiments, the third sdABD comprises the amino acid sequence of SEQ ID NO: 220.

In some embodiments, the protein comprises the amino acid sequence of any one of SEQ ID NOs: 234-249.

Nucleic acid molecules comprising a nucleotide sequence encoding the protein described herein are provided. In some embodiments, the nucleic acid molecule is a vector. In some embodiments, the nucleic acid molecule is an expression vector. Cells comprising the protein or the nucleic acid molecule described herein are also provided.

Other aspects of the present disclosure provide methods of producing a protein comprising culturing the cells described herein under conditions that allow expression of the protein. In some embodiments, the method further comprises isolate the protein.

Compositions comprising the protein described herein are provided.

Other aspects of the present disclosure provide methods of treating cancer, comprising administering the protein or the composition described herein to a subject. In some embodiments, the subject is a human subject.

Further provided herein are compositions comprising:
a first protein and a second protein, each of which comprising, from N- to C-terminus:
(i) a first single domain antigen binding domain (sdABD) that binds to a first human target tumor antigen (TTA);
(ii) a first domain linker;
(iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first human immune cell antigen, and wherein the first heavy chain variable region and the first light chain variable region are not associated in the first constrained scFv domain and the first constrained scFv domain does not bind to the first human immune cell antigen;
(iv) a first cleavable linker;
(v) a second single domain antigen binding domain (sdABD) that binds to a second human target tumor antigen (TTA);
(vi) a second domain linker;
(vii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second human immune cell antigen, and wherein the first heavy chain variable region and the first light chain variable region are not associated in the second constrained scFv domain, and the second constrained scFv domain does not bind to the second human immune cell antigen;
(viii) a second cleavable linker; and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first protein is identical to the second protein.

In some embodiments, upon cleavage of the first cleavable linker of (iv) and the second cleavable linker of (viii) in the first protein and the second protein:
the first heavy chain variable region of the first protein associates with the first light chain variable region of the second protein, forming an active Fv that binds to the first human immune cell antigen;
the first light chain variable region of the first protein associates with the first heavy chain variable region of the second protein, forming an active Fv that binds to the first human immune cell antigen;
the second heavy chain variable region of the first protein associates with the second light chain variable region of the second protein, forming an Fv that binds to the second human immune cell antigen; and
the second light chain variable region of the first protein associates with the second heavy chain variable region of the second protein, forming an Fv that binds to the second human immune cell antigen.

In some embodiments, the cleavage occurs in a tumor microenvironment in a subject upon administration of the composition to the subject.

Yet other aspects of the present disclosure provide compositions comprising:
(a) a first homodimer of a first polypeptide, wherein the first polypeptide comprises:
   (i) a first single domain antigen binding domain (sdABD) that binds to a first human target tumor antigen (TTA);
   (ii) a first domain linker;
   (iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first human immune cell antigen, and wherein the first heavy chain variable region and the first light chain variable region are not associated in the first constrained scFv domain and the first constrained scFv domain does not bind to the first human immune cell antigen;
wherein in the first homodimer, the first VH of one polypeptide associates with the first VL of the other polypeptide, and the first VL of one polypeptide associates with the first VH of the other polypeptide, forming two active variable fragments (Fvs) each capable of binding to the first immune antigen;
(b) a second homodimer of a second polypeptide, wherein the second polypeptide comprises:
   (i) a second single domain antigen binding domain (sdABD) that binds to a second human target tumor antigen (TTA);
   (ii) a second domain linker;
   (iii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second human immune cell antigen, and wherein the second heavy chain variable region and the second light chain variable region are not associated in the second constrained scFv domain, and the second constrained scFv domain does not bind to the second human immune cell antigen;
      wherein in the second homodimer, the second VH of one polypeptide associates with the second VL of the other polypeptide, and the second VL of one polypeptide associates with the second VH of the other polypeptide, forming two active variable fragments (Fvs) each capable of binding to the second immune antigen.

In some embodiments, the first immune cell antigen is different from the second immune cell antigen. In some embodiments, the first immune cell antigen is CD3 and the second immune cell antigen is CD28. In some embodiments, the first immune cell antigen is CD28 and the second immune cell antigen is CD3. In some embodiments, the first human target tumor antigen is EGFR or HER2. In some embodiments, the second human target tumor antigen is EGFR or HER2.

### BRIEF DESCRIPTION OF THE DRAWINGS

FIGS. 1A-ID show an example of a general schematic of the proteins of the disclosure before and after protease cleavage. An inactive (e.g., prior to protease cleavage) protein construct is shown in FIG. 1A. Generally, from N- to C-terminal, the construct contains a first anti-tumor target antigen (anti-TTA) domain, a domain linker, a first anti-CD3 variable domain (as depicted in FIG. 1A, this is the VH domain, although as described herein, these components can be in different orders), a constrained non-cleavable linker (CNCL; in this case a non-cleavable linker that is 8 amino acids long (NCL-8)), a second anti-CD3 variable domain (again, this is the VL domain in this example), a cleavable linker (CL; in this case an MMP9 cleavable linker that is 15 amino acids long), a second α-tumor target antigen (anti-TTA) domain, a domain linker, a first anti-CD28 variable domain (as depicted in FIG. 1A, this is the VL domain, although as described herein, these components can be in different orders), a constrained non-cleavable linker (CNCL; in this case a non-cleavable linker that is 8 amino acids long (NCL-8)), a second anti-CD28 variable domain (again, this is the VH domain in this embodiment), an optional cleavable linker (CL; in this case an MMP9 cleavable linker that is 15 amino acids long), a half-life extension domain (HSA-sdABD), and an optional C-terminal histidine tag (H6). FIG. 1B depicts the cleavage products of the FIG. 1A construct where the second cleavage site is present. FIG. 1C depicts the homodimerization of two anti-CD3 cleavage product components to form active anti-CD3 Fvs, and thus an active bispecific T-cell engaging molecule, that will bind both a tumor cell and either T-cells (as shown in FIGS. 2A-2B) or other CD3 expressing cell types, to activate them. The other homodimer that is formed is the bispecific anti-CD28 binding domains, which can bind both a tumor cell and either T-cells (as shown in FIGS. 2A-2B) or other CD28 expressing cell types, to activate them. FIG. 1D shows the expected domain structure of the protein in each of FIGS. 1A-IC. The predicted intramolecular folded structure of an exemplary protein, where the anti-CD3 VH pairs with the anti-CD28 VL and the anti-CD3 VL pairs with the anti-CD28 VH, such that neither form an active CD3 or CD28 binding domain. The protein is cleaved allowing homodimerization of anti-CD3 and anti-CD28 domains.

FIGS. 2A-2C show a general schematic of the bispecific mode of action of proteins of the present disclosure. In FIG. 2A-2B, the resulting active homodimers, one that binds CD3 and one that binds CD28, can bind to the same T-cell to activate non-specifically the T-cell for cytolysis of the tumor cell. In FIG. 2A, while as shown only one sdABD of each dimer binds to a cancer cell antigen, the other tumor targeting sdAB in the homodimers are capable of binding tumor cells as well. In FIG. 2B both tumor targeting sdABDs of each homodimer bind to a cancer cell antigen. FIG. 2C shows that the active anti-CD28 homodimer can also bind to a cancer specific T cell that has been rendered either anergic or exhausted by the tumor microenvironment. The active anti-CD28 homodimer can break the T-cell's inactive state, reactivate it and induce cytolysis of the tumor cell.

FIGS. 3A-3B are schematics of proteins described herein. FIG. 3A depicts examples of 8 different configurations for the proteins described herein (formats 1-8). The constructs vary in the N-terminal to C-terminal locations of the anti-CD3 constrained Fv VH and VL domains, the anti-CD28 constrained Fv VH and VL domains, and anti-CD3 constrained Fv and the anti-CD28 constrained Fv. FIG. 3B comprises similar reconfigured domains as FIG. 3A, but each comprise a TTA single domain antibody binding domain (TTA-sdABD) that is EGFR binding. Also shown are observed expression levels reached when expressed transiently in HEK293 cells.

FIGS. 4A-4C depict examples of constrained protein constructs, including an uncleaved inactive T-cell engager and co-stimulator construct (FIG. 4A), an active bispecific T cell engager construct upon cleavage (FIG. 4B) and an active bispecific co-stimulator construct upon cleavage (FIG. 4C). The constructs include target tumor antigen (TTA) single domain antigen binding domains (sdABDs; “TTA-sdABDs”).

FIGS. 5A-5B depict examples of active anti-CD28 constrained constructs produced recombinantly.

FIGS. 6A-6C depict in vitro activation of human T cells as measured by proliferation by both anti-CD3 and anti-CD28 active dimers. FIG. 6A shows anti-CD3 active dimer (Pro201) can stimulate T cells similarly to anti-αCD3 antibody when target (EGFR) is immobilized to the plate. In FIG. 6B, T cells are co-stimulated by either anti-CD28 antibody or anti-CD28 active homodimers with suboptimal amounts (200 picomolar (pM)) of Pro201/anti-CD3 active dimer. FIG. 6C shows co-stimulation of T cells by anti-CD28 (Pro938) and anti-CD3 (Pro201) active homodimers with target molecule immobilized to plate vs. beads.

FIGS. 7A-7B depict T cell dependent cellular cytotoxicity of HT29 cells by various anti-CD28 active dimers with suboptimal amounts of anti-CD3/Pro201. In FIG. 7A, target cells were treated with Pro201 first, then mixed with the human T cells, along with increasing concentrations of the anti-CD28 active homodimer. For FIG. 7B, the anti-CD28 active homodimers were incubated with the target cells before mixing with the T cells and Pro201. In both situations, cytotoxicity was observed with anti-EGFR anti-CD28 active homodimers, but not with a cross-linking anti-CD28 antibody.

FIGS. 8A-8C show results of stimulation of exhausted T cells. FIG. 8A shows a schematic of the preparation and stimulation of exhausted T cells-Human primary resting T cells were stimulated extensively with anti-CD3 and IL-10 until they exhibited exhausted phenotype via expression of indicated markers as shown in FIG. 8B. Exhausted T cells were pre-labeled with Cell Trace Violet to track proliferation and viability via FACS then incubated with test molecules in the presence of EGFR-conjugated beads. FIG. 8C shows that exhausted T cells only proliferated by stimulating both CD3 and CD28, not CD3 alone.

FIGS. 9A-9D show that the orientation of the VH and VL domain within the anti-CD3 and anti-CD28 influence the proliferation of exhausted T cells. FIG. 9A is the schematic representation of the anti-CD3 and anti-CD28 active dimer molecules used. Pro938 and Pro1034 have a configuration of sdABD (EGFR)-scFv (CD28 VH/VL)-sdABD (EGFR), and Pro935 and Pro1035 has a configuration of sdABD (EGFR)-scFv (CD28 VL/VH)-sdABD (EGFR). FIG. 9B shows that administering anti-CD28 active dimer molecules Pro935 or Pro938 with Pro201 results in dose-dependent increases in T cell proliferation, with Pro201+Pro935 having a maximum proliferation index of 1.7 and Pro201+Pro938 having a maximum proliferation index of 2.4. FIG. 9C shows that administering anti-CD28 active dimer molecules Pro1034 or Pro1035 with Pro201 results in dose-dependent increases in T cell proliferation, with Pro201+Pro1034 treatment resulting in a maximum proliferation index of 2.1 and Pro201+Pro1035 having a maximum proliferation index of 1.5. FIG. 9D shows that administering any of these proteins alone results in very low levels of proliferation, with maximum proliferation index of less than 1.1.

FIGS. 10A-10E show that the orientation of the VH and VL domain within the anti-CD3 and anti-CD28 influence the proliferation and viability of exhausted T cells. FIG. 10A shows the schematic representation of the active dimer molecules used. Pro1134 has a configuration of sdABD (EGFR)-scFv (CD28 VH/VL) and Pro1135 has a configuration of sdABD (EGFR)-scFv (CD28 VL/VH). FIG. 10B shows that Pro201+Pro1134 is slightly more potent at inducing proliferation than Pro201+Pro1135 with a maximum proliferation index of 3.8 with Pro201+Pro1134 and a maximum proliferation index of 3.6 with Pro201+Pro1135. FIG. 10C shows that treatment with Pro201, Pro1134, or Pro1135 alone is not sufficient to induce proliferation until concentrations are greater than about 100 pM and the maximum proliferation index is less than 1.8. FIG. 10D shows that Pro201+Pro1134 is more potent in increasing the percentage of viable cells than Pro201+Pro1135, with maximum viability of 47% with Pro201+Pro1134 and a maximum viability of 33% with Pro201+Pro1135. FIG. 10E shows that Pro201, Pro1134 or Pro1135 alone increase cell viability, but to a lesser extent than Pro201 with either Pro1134 or Pro1135.

FIGS. 11A-11D show that anti-CD3 and anti-CD28 active dimer molecules are active when both molecules have a single targeting sdABD and the orientation of the VH and VL domain within the anti-CD3 influence T cell activation. FIG. 11A shows the schematic representation of the molecules used. Pro861 is an sdABD (EGFR)-anti-CD3 (VH/VL) construct and Pro863 is an sdABD (EGFR)-anti-CD3 (VL/VH) construct. FIG. 11B shows that exhausted T cells proliferated in the presence of both CD3 and CD28 active dimers, but not when either dimer alone is administered. Results also show that Pro861+Pro1134 had greater potency than Pro863+Pro1134. Results also show that Pro861+Pro1134 had greater potency than Pro863+Pro1134. Additionally, freezing T cells prior to inducing exhaustion had little to no effect on T cell proliferation after treatment with constructs. FIG. 11C shows that even though either anti-CD3 or anti-CD28 active dimers alone can support viability, the combination of both provides the highest potency. Results also show that Pro861+Pro1134 had greater potency than Pro863+Pro1134. FIG. 11D shows the EC50 and maximum values for proliferation and viability with each treatment.

FIG. 12 shows the structure of Pro186, a T cell engager wherein the MMP9 cleavage site is located between a second EGFR binding domain and an inactive VLi domain. Pro186 uses inactive VLi-VHi to block formation of active anti-CD3 dimers.

FIG. 13 shows the structure of Pro646. The MMP9 protease cleavage sites in Pro646 are located between the anti-CD3 VL domain and the second EGFR binding domain, and between the VHi domain and the HSA domain. This molecule has two anti-EGFR sdAbs associated with the anti-CD3 active homodimer versus the four that the Pro186 homoactive dimer has (FIG. 12).

FIG. 14 shows the structure of the Co-Stimulatory COBRA Pro1136: The MMP9 protease cleavage sites are located between the anti-CD3 VL domain and the second EGFR binding domain (identical to Pro646) and between the anti-CD28 VL and the HSA domain. The inactive VLi and VHi are replaced with an anti-CD28 VH-VL to mutually block formation of active anti-CD3 and anti-CD28 active dimers. This molecule has similar proteolytic conditionality as the Pro646 but includes the ability to form both anti-CD3 and anti-CD28 active dimers.

FIGS. 15A-15D show that treatment with anti-CD3 EGFR and anti-CD28 EGFR constructs increased proliferation and viability of exhausted T cells with higher potency. FIG. 15A shows the schematics of the molecules used. FIG. 15B shows that only native and pre-cleaved Pro646 in combination with Pro1134, the anti-CD28 active dimer were able to induce proliferation. FIG. 15C shows that the combination of cleaved Pro646, which forms an active anti-CD3 dimer, and Pro1134, the anti-CD28 active dimer shows the highest potency when inducing viability of exhausted T cells. FIG. 15D shows the EC50 values for proliferation and viability for each treatment.

FIGS. 16A-16E show the effects of different variants of conditionally active proteins described herein (schematics shown in FIG. 3B). FIG. 16A shows that cleaved Pro1136, cleaved Pro1138, cleaved Pro1140, and cleaved Pro1142 induced T cell proliferation of exhausted T cells, whereas uncleaved Pro1136, uncleaved Pro1138, uncleaved Pro1140, and uncleaved Pro1142 did not. FIG. 16B shows that cleaved Pro1136, cleaved Pro1138, cleaved Pro1140, and cleaved Pro1142 induced T cell viability with more potency than uncleaved Pro1136, uncleaved Pro1138, uncleaved Pro1140, and uncleaved Pro1142. FIG. 16C shows EC50 of proliferation and cell viability of cleaved and uncleaved Pro1136, Pro1138, Pro1140, and Pro1142. FIG. 16D compares the in vitro cytotoxicity of the Pro186, Pro646 and Pro1136 COBRA molecules with and without protease cleavage. COBRAs were tested at various concentrations in a standard TDCC assay at a 10:1 (Human T cells:HT29 tumor cell line) ratio. In this series of molecules tested, pre-cleaved Pro186 was the most potent. Pre-cleaved Pro646 was at least 10-fold less active, probably because its anti-CD3 active dimer only has two anti-EGFR sdAbs, versus the four that Pro186 can form. Pro1136 was about 5-fold more active than Pro646. FIG. 16E quantifies the potency of cleaved Pro1136-Pro1143 (schematics shown in FIG. 3B) in T cell-dependent cellular cytotoxicity (TDCC) assay targeting human colorectal adenocarcinoma (HT29) cells. Pro186 is used as a positive control. Results show that Pro1136 and Pro1137 are about 10 fold more potent than Pro1138 and Pro1139 in this assay. Pro1140 and Pro1141 are about 100 fold less potent than Pro1136 and Pro1137 in this assay. Pro1142 and Pro1143 are about 1000 fold less potent than Pro1136 and Pro1137 in this assay.

FIG. 17 shows the effect of an alternative anti-EGFR sdAb on COBRA function. This experiment is similar to that shown in FIG. 16, except the Co-Stimulatory COBRAs tested have targeting domains replaced with an alternative anti-EGFR sdAb hG8. When comparing the cleaved Pro1184 (anti-CD3 VH-VL/anti-CD28 VH-VL) versus cleaved Pro1185 (anti-CD3 VH-VL VLi-VHi), the former is two-fold more potent because it not only contains the usual anti-CD3 active dimer, but the anti-CD28 scFv as well, while the latter only has the anti-CD3 active dimer. Pro1192 (anti-CD28 VH-VL/anti-CD3 VH-VL) was about 60-fold less potent than Pro1184 suggesting that having the anti-CD3 VH-VL N-terminal to the anti-CD28 VH-VL increases potency. As expected, Pro1186 (anti-CD28 VH-VL/VHi-VLi) was thousands of fold less potent than constructs comprising anti-CD3 VH-VL or both the anti-CD3 VH-VL and the anti-CD28 VH-VL.

FIGS. 18A-18B show that anti-CD3 and anti-CD28 active dimer molecules containing sdABDs targeting HER2 or EGFR induce proliferation of exhausted T cells. FIG. 18A is a schematic representation of the active dimer molecules used. Pro1176 is an sdABD (HER2)-anti-CD3 (VH/VL) construct, and Pro1179 is an sdABD (HER2)-anti-CD28 (VH/VL) construct. FIG. 18B shows that Pro1176+Pro1134 treatment results in higher proliferation than with Pro1134 alone, and treatment with Pro861+Pro1179 treatment results in higher proliferation than with Pro861 alone.

FIGS. 19A-19B show conditionally active proteins comprising anti-HER2 sdABDs. FIG. 19A shows different configurations of conditionally active proteins. Different constructs have different N-terminal to C-terminal orientations of the anti-CD3 VH-VL and the anti-CD28 VH-VL, and have two anti-HER2 sdABDs, or one anti-HER2 sdABD and one anti-EGFR sdABD. Constructs having one anti-HER2 sdABD and one anti-EGFR sdABD can have either the anti-HER2 sdABD or anti-EGFR sdABD at the N-terminus, or between the MMP9-15 cleavage site and an NCL. FIG. 19B shows control conditionally active proteins that comprise FLAG-inactivated sdABDs instead of anti-CD28 sdABDs. All control constructs have anti-EGFR sdABDs.

FIG. 20 quantifies the potency of conditionally active multi-specific proteins comprising anti-CD28 VH-VL, anti-CD3 VH-VL and anti-HER2 sdABDs (hu1156) in TDCC assay targeting human B lymphoblast-like cells overexpressing human HER2 (huHER2-RAJI cells). From N-terminal to C-Terminal, Pro1265 comprises the anti-CD28 VH-VL and the anti-CD3 VH-VL. From N-terminal to C-Terminal, Pro1266 comprises the anti-CD3 VH-VL and the anti-CD28 VH-VL. Results show that Pro1265 and Pro1266 have similar TDCC potency regardless of CD3/CD28 location.

FIG. 21 shows that Pro1136 is more potent than Pro186 and Pro1267 when killing Uppsala 87 Malignant Glioma (U87MG) cells. These cells express EGFR but not HER2. Pro1136 is described in FIG. 3B and comprises an anti-EGFR sdABD, an anti-CD28 VH-VL, and an anti-CD3 VH-VL. Pro186 comprises an anti-EGFR sdABD, a FLAG-inactivated VL-VH, and an anti-CD3 VH-VL. Pro1267 comprises an anti-EGFR sdABD, an anti-HER2 sdABD, an anti-CD28 VH-VL, and an anti-CD3 VH-VL. Results show that cleaved Pro1136 is about 3-fold more potent than cleaved Pro1267 and cleaved Pro186. This suggests that having both anti-CD3 VH-VL linked to an anti-EGFR sdABD and an anti-CD28 VH-VL linked to an sdABD that binds the tumor cells increases potency, as Pro1136 has an anti-CD28 VH-VL linked to EGFR that can bind U87-MG cells, when compared to Pro186, which has no anti-CD28 VH-VL and Pro1267, which has an anti-CD28 VH-VL linked to HER2 that cannot bind U87-MG cells.

FIG. 22 shows that Pro1140 is at least 3-fold more potent than Pro1270, Pro1271, and Pro1272 because of additional CD28 active dimer in killing U87MG (only EGFR) cells. Pro1140 is described in FIG. 3B and comprises an anti-CD3 VH-VL linked to an anti-EGFR sdABD and an anti-CD28 VH-VL linked to an anti-EGFR sdABD. Pro1270, Pro1271, and Pro1272 all comprise an anti-CD3 VH-VL linked to an anti-EGFR sdABD, but Pro1270 comprises an anti-CD28 VH-VL linked to an anti-HER2 sdABD, Pro1271 comprises a FLAG-inactivated VL-VH linked to an anti-EGFR sdABD, and Pro1272 comprises a FLAG-inactivated VH-VL linked to an anti-EGFR sdABD. This suggests that having both anti-CD3 VH-VL linked to an anti-EGFR sdABD and an anti-CD28 VH-VL linked to an sdABD that binds the tumor cells increases potency.

### DETAILED DESCRIPTION

Recent developments have shown sufficient benefits of immune cell engaging moieties, that simultaneously bind to important physiological targets such as CD3 on the surface of T-cells and tumor antigens on the surface of cancer cells. An example of this is a “T-cell engager mechanism”, wherein the binding of the bispecific biologic drug to CD3 and the tumor antigen results in the release of cytotoxins by the T-cell, thus killing the tumor cell.

Many antigen binding proteins, such as those used on immune cell engaging moieties, can have significant off-target side effects. Thus there is a need to only activate the proteins in the vicinity of the disease tissue, to avoid off-target interactions. Strategies for activating immune cell engaging moieties within the vicinity of the diseased tissues have been disclosed, e.g., in US20190076524, which is incorporated by reference in its entirety.

The present disclosure, in some aspects, provides methods and compositions for reducing the toxicity and side effects of immune cell engaging bispecific antibodies that bind to cancer and immune cells to stimulate immune cell killing of a target cancer. Many of the proteins provided herein are prodrugs that may be activated by proteases (e.g., proteases found in tumor microenvironments). In some embodiments, the proteins described herein are configured such that, when they are not in a tumor microenvironment, the protein is capable of binding to tumor cells but not immune cells (inactive), and such that when the proteins enter a tumor microenvironment, cleavage of the cleavable linkers in the protein “activates” the protein, resulting in two “active” bi-specific molecules, wherein each can bind to tumor cells and immune cells. In some embodiments, each of the two “active” bi-specific molecules bind a different antigen on immune cells. In some embodiments, the two “active” bi-specific molecules bind two different antigens on the same immune cell. In some embodiments, the two “active” bi-specific molecules bind two different immune cells (e.g., immune cells selected from T-cells, natural killer cells, and macrophages). For example, in some embodiments, the first “active” bi-specific molecule may bind a first target tumor antigen and a first immune cell antigen CD3, while the second “active” bi-specific molecule binds a second target tumor antigen and a second immune cell antigen CD28. The first target tumor antigen and second target tumor antigen may be the same or different. This tumor specific activation decreases potential off-target side effects, and the targeting of two different immune cell antigens enhances the anti-tumor activity of the proteins described herein, e.g., by activating co-stimulating molecules, enhancing T cell recruitment and activity, reducing T cell exhaustion, enhancing cytotoxicity and IFNγ secretion, stimulating macrophages, and/or enhancing maturation of macrophages.

### I. Definitions

In order that the application may be more completely understood, several definitions are set forth below. Such definitions are meant to encompass grammatical equivalents.

By “amino acid” and “amino acid identity” as used herein is meant one of the 20 naturally occurring amino acids or any non-natural analogues that may be present at a specific, defined position. In many embodiments, “amino acid” means one of the 20 naturally occurring amino acids. By “protein” herein is meant at least two covalently attached amino acids, which includes proteins, polypeptides, oligopeptides and peptides.

By “amino acid modification” herein is meant an amino acid substitution, insertion, and/or deletion in a polypeptide sequence or an alteration to a moiety chemically linked to a protein. For example, a modification may be an altered carbohydrate or PEG structure attached to a protein. For clarity, unless otherwise noted, the amino acid modification is always to an amino acid coded for by DNA, e.g., the 20 amino acids that have codons in DNA and RNA. The preferred amino acid modification herein is a substitution.

In some embodiments, the protein specifically binds to immune cell antigens and target tumor antigens (TTAs) such as target cell receptors, as outlined herein. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a Ka for an antigen or epitope of at least 20-, 50-, 100-, 500-, 1000-, 5,000-, 10,000- or more times greater for the epitope relative to a control, where Ka (or KA) refers to an association rate of a particular antibody-antigen interaction. Binding affinity is generally measured using a Biacore assay or Octet as is known in the art.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. A target antigen may be a protein, carbohydrate, lipid, or other chemical compound. A range of suitable exemplary target antigens are described herein, including target tumor antigens.

By “target cell” as used herein is meant a cell that expresses a target antigen. Target cells are either tumor cells that express TTAs or immune cells e.g., T-cells that express an immune cell antigen such as CD3 and/or CD28.

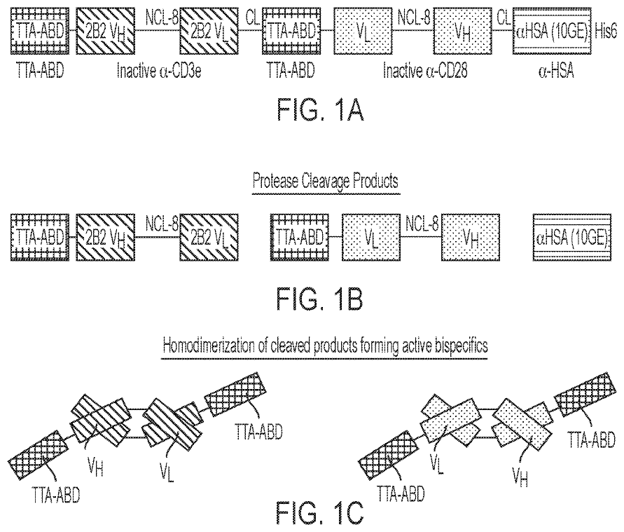

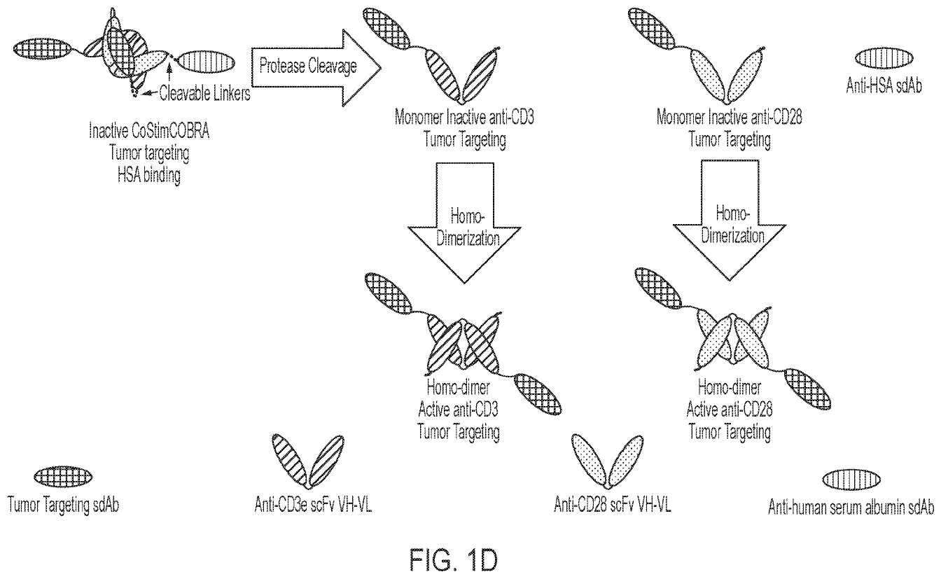

By “single chain variable fragment (scFv),” “Fv,” or “Fv domain” or “Fv region” as used herein is meant a polypeptide that comprises VL and VH domains of an antigen binding domain, generally, but not always, from an antibody. Fv domains usually form “antigen binding domains” or “ABDs” as discussed herein, if they contain VH and VL domains each containing CDRs that will bind to the antigen. An “active Fv” is one that has a variable heavy and a variable light domain each with CDRs that bind the same antigen, e.g., CD3 or CD28. Thus an active Fv can bind its antigen. An “inactive Fv” or a “non-productive Fv” is one that has a variable heavy and a variable light domain but does not bind an antigen. In some cases, the “inactive Fv” is present on a single chain polypeptide with a constrained linker that does not allow for association of the VH and VL within the Fv (also referred to as a “constrained Fv”). In this case, due to the strong preference of the second framework region of the variable heavy and variable light domains to pair, an intramolecular association with another constrained inactive Fv renders two inactive Fvs, e.g., that do not bind antigens. Thus, in this case, an “inactive Fv” or “non-productive Fv” has a VH and a VL that are associated into an Fv, but the Fv is not an antigen binding domain because the respective active VH and VLs are not associated with each other. For example, as illustrated in FIG. 1, the VH and VL in an Fv directed to CD3 are bound to the VL and VH in an Fv directed to CD28, respectively, forming an inactive molecule (e.g., one that does not bind to either CD3 or CD28).

As discussed below, Fv domains can be organized in a number of ways in proteins, and can be “active” or “inactive” (also sometimes referred to herein as “non-productive”), such as in a scFv format, a constrained Fv format, a constrained scFv format, a pseudo Fv format, etc. In addition, as discussed herein, Fv domains containing VH and VL can be/form ABDs, and other ABDs that do not contain VH and VL domains can be formed using sdABDs.

By “variable domain” herein is meant the region of an immunoglobulin that comprises one or more Ig domains substantially encoded by any of the Vκ, Vλ, and/or VH genes that make up the kappa, lambda, and heavy chain immunoglobulin genetic loci respectively. In some cases, a single variable domain, such as a sdFv (also referred to herein as sdABD) can be used.

In embodiments utilizing both variable heavy (VH) and variable light (VL) domains, each VH and VL is composed of three hypervariable regions (“complementary determining regions,” “CDRs”) and four “framework regions”, or “FRs”, arranged from amino-terminus to carboxy-terminus in the following order: FR1-CDR1-FR2-CDR2-FR3-CDR3-FR4. Thus, the VH domain has the structure vhFR1-vhCDR1-vhFR2-vhCDR2-vhFR3-vhCDR3-vhFR4 and the VL domain has the structure vlFR1-vlCDR1-vlFR2-vlCDR2-vlFR3-vlCDR3-vlFR4. As is more fully described herein, the vhFR regions and the vlFR regions self-assemble to form Fv domains. In general, in the prodrug formats of the protein, there are “constrained Fv domains” wherein the VH and VL domains within the same Fv domain cannot self-associate due to the presence of a constrained linker between the VH and VL domains, and “inert Fv domains” for which the CDRs do not form antigen binding domains when self-associated.

The hypervariable regions confer antigen binding specificity and generally encompass amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the proteins are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g., vlCDR1, vlCDR2 and vlCDR3).

A useful comparison of CDR numbering is as below, see Lafranc et al., Dev. Comp. Immunol. 27 (1): 55-77 (2003):

| | Kabat + | | | | | |
|---|---|---|---|---|---|---|
| | Chothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present disclosure provides a number of different CDR sets. In this case, a “full CDR set” in the context of domains that bind to immune cell antigens (e.g., scFvs that bind to CD3 or CD28), means that the component comprises a heavy chain variable region comprising three CDRs (e.g., vhCDR1, vhCDR2 and vhCDR3) and a light chain variable region comprising CDRs (e.g., a vlCDR1, vlCDR2, vlCDR3). As will be appreciated by those in the art, each set of CDRs, the VH and VL CDRs, can bind to antigens, both individually and as a set. For example, in constrained Fv domains, the vhCDRs can bind, for example to CD3 and the vlCDRs can bind to CD3, but in the constrained format they cannot bind to CD3.

By “single domain Fv”, “sdFv” or “sdABD” herein is meant an antigen binding domain that only has three CDRs, generally based on camelid antibody technology. See: Protein Engineering 9 (7): 1129-35 (1994); Rev Mol Biotech 74:277-302 (2001); Ann Rev Biochem 82:775-97 (2013). sdABD generally comprise a single heavy chain variable region, which comprises a set of three CDRs. sdABDs are also sometimes referred to in the art as “VHH” domains or nanobodies. As outlined herein, there are two general types of sdABDs used herein: sdABDs that bind to TTAs, and are annotated as such (sdABD-TTA for the generic term, or, for example, sdABD-EGFR for one that binds to EGFR, sdABD-FOLR1 for one that binds to FOLR1, etc.) and sdABDs that bind to HSA (“sdABD-HSA”).

These CDRs can be part of a larger variable light or variable heavy domain. In addition, as more fully outlined herein, the variable heavy and variable light domains can be on separate polypeptide chains or on a single polypeptide chain in the case of scFv sequences, depending on the format and configuration of the moieties herein.

The CDRs contribute to the formation of the antigen-binding, or more specifically, epitope binding sites. “Epitope” refers to a determinant that interacts with a specific antigen binding site in the variable regions known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specific antigen binding peptide; in other words, the amino acid residue is within the footprint of the specific antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the proteins not only include the enumerated antigen binding domains and antibodies herein, but those that compete for binding with the epitopes bound by the enumerated antigen binding domains.

The term “antigen binding domain” (ABD) characterizes a domain which (specifically) binds to/interacts with/recognizes a given target epitope or a given target site on the target molecules (antigens). ABDs can be scFvs, containing a VH and VL domain, or can be sdABDs as defined herein. In general, ABDs that bind to the target tumor antigens (TTAs) and to human serum albumin (HSA) are sdABDs (“TTA-sdABD” or “HSA-sdABD”), while the ABDs that bind to the immune cell antigens (e.g., CD3 or CD28) are scFvs containing both a VH and VL domain.

By “domain” as used herein is meant a protein sequence with a structure and/or function, as outlined herein. Domains of the proteins described herein include target tumor antigen binding domains (TTA domains), immune cell binding domains, linker domains, and half-life extension domains.

By “domain linker” herein is meant an amino acid sequence that joins two domains as outlined herein. Domain linkers can be cleavable linkers, constrained cleavable linkers, non-cleavable linkers, constrained non-cleavable linkers, scFv linkers, etc.

By “cleavable linker” (“CL”) herein is meant an amino acid sequence that can be cleaved by a protease, preferably a human protease in a disease tissue as outlined herein. Cleavable linkers generally are at least 3 amino acids in length, with from 4, 5, 6, 7, 8, 9, 10, 11, 12, 13, 14, 15 or more amino acids finding use in a protein, depending on the required flexibility. A number of cleavable linker sequences are found in FIG. 13.

By “non cleavable linker” (“NCL”) herein is meant an amino acid sequence that cannot be cleaved by a human protease under normal physiological conditions.

By “constrained non-cleavable linker” (“CNCL”) herein is meant a short polypeptide that that joins two domains as outlined herein in such a manner that the two domains cannot significantly interact with each other, and that is not significantly cleaved by human proteases under physiological conditions.

By “constrained scFv domain” herein is meant an scFv domain that comprises an active variable heavy domain and an active variable light domain, linked covalently with a constrained linker as outlined herein, in such a way that the active heavy and light variable domains cannot interact to form an active Fv that will bind an immune cell antigen such as CD3. Thus, a constrained Fv domain is one that is similar to an scFv but is not able to bind an antigen due to the presence of a constrained linker (although they may assemble intramolecularly with other variable domains (e.g., inert variable domains or variable domains that target a different antigen) to form pseudo Fv domains.

As used herein “protease cleavage site” refers to the amino acid sequence recognized and cleaved by a protease. Suitable protease cleavage sites are outlined below and shown in FIG. 13.

As used herein, “protease cleavage domain” refers to the peptide sequence incorporating the “protease cleavage site” and any linkers between individual protease cleavage sites and between the protease cleavage site(s) and the other functional components of the constructs of the protein (e.g., VH, VL, target antigen binding domain(s), half-life extension domain, etc.). As outlined herein, a protease cleavage domain may also include additional amino acids if necessary, for example to confer flexibility.

### II. Proteins

The present disclosure, in some aspects, provides proteins that are referred to herein as “co-stim COnditional Bispecific Redirected Activation” constructs, or “co-stim “COBRAs”. In some embodiments, a protein described herein comprises two sdABDs each capable of binding to a TTA and two constrained single chain variable fragment (scFv) domains, each constrained scFv domain comprises a VH and a VL that, if associated, are capable of binding to an immune cell antigen. However, the VH and VL within a constrained scFv domain are not able to associate with each other due to the presence of a short peptide linker between the VH and the VL (i.e., “constrained”). Additionally, the two constrained scFv domains associate intramolecularly, forming two inactive Fvs that do not bind to immune cell antigens. In some embodiments, the two sdABDs bind to the same TTA. In some embodiments, the two constrained scFv domains bind to the same immune cell antigen (e.g., same immune cell antigen on the same immune cell or on different immune cells). In some embodiments, the two constrained scFv domains bind to different immune cell antigens (e.g., different immune cell antigens on the same immune cell or on different immune cells). In some embodiments, a protein described herein further comprises one or more cleavable linkers (e.g., linkers cleavable by a protease present in a tumor microenvironment). In some embodiments, a protein described herein further comprises a half-life extension domain (e.g., a sdABD capable of binding to human serum albumin (HSA)).

It is contemplated herein that a protein disclosed herein is inactive when one or more of the cleavable linkers in the protein is uncleaved. In some embodiments, an uncleaved and inactive protein described herein is capable of binding to a TTA but not an immune cell antigen. The protein is cleaved and activated, for example, in a disease-specific microenvironment or in the blood of a subject at internal cleavable linkers that contain protease cleavage sites. Once cleaved, fragments of the cleavage products form two dimers (e.g., homodimers), each of which is a bi-specific molecule capable of binding to both a TTA and an immune cell antigen, thereby stimulating and/or activating one or more immune cells. In some embodiments, the two dimers (e.g., homodimers) bind to different TTAs. In some embodiments, the two dimers (e.g., homodimers) bind to the same TTA but a different epitope in the TTA. In some embodiments, the two dimers (e.g., homodimers) bind to different immune cell antigens (e.g., one binds to CD3 and the other binds to CD28).

In some embodiments, the specificity of the response of T-cells is mediated by the recognition of antigen (displayed in context of a major histocompatibility complex, MHC) by the T-cell receptor complex. As part of the T-cell receptor complex, CD3 is a protein complex that includes a CD3γ (gamma) chain, a CD3δ (delta) chain, two CD3e (epsilon) chains and two CD3ζ (zeta) chains, which are present at the cell surface. CD3 molecules associate with the α (alpha) and β (beta) chains of the T-cell receptor (TCR) to comprise the TCR complex. Clustering of CD3 on T-cells, such as by Fv domains that bind to CD3 leads to T-cell activation similar to the engagement of the T-cell receptor but independent of its clonal-typical specificity.

However, as is known in the art, CD3 activation can cause a number of toxic side effects, and accordingly the present disclosure is directed to providing active CD3 binding of the polypeptides of the disclosure only in the presence of tumor cells, where specific proteases are found, that then cleave the prodrug polypeptides of the proteins to provide an active CD3 binding domain. Thus, binding of an anti-CD3 Fv domain to CD3 is regulated by a protease cleavage domain which restricts binding of the CD3 Fv domain to CD3 only in the microenvironment of a diseased cell or tissue with elevated levels of proteases, for example in a tumor microenvironment as is described herein.

In some embodiments, in addition to a constrained anti-CD3 scFv, a protein described herein may also comprise constrained anti-CD28 scFvs. As is known in the art, CD28 stimulation has been shown to activate various anti-tumor T cells, NK cells, dendritic cells, neutrophils, macrophages, and endothelial cells. There have even been observations that CD28 can suppress Treg cells. The addition of these novel CD28 co-stimulatory moieties into the proteins disclosed herein allows not only killing tumor cells directly via the T cell engaging CD3 bispecific molecules, but also the stimulation of other anti-tumor associated cell types, inhibiting T cell exhaustion, and inducing a long-term, sustained, and systemic immune response.

Proteins of the present disclosure are constructed in a “prodrug (inactive)” form. In some embodiments, such a prodrug protein described herein comprises, from N- to C-terminus:
(i) a first single domain antigen binding domain (sdABD) that binds to a first target tumor antigen (TTA, e.g., a human TTA);
(ii) a first domain linker;
(iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first immune cell antigen (e.g., human immune cell antigen), and wherein the first heavy chain variable region and the first light chain variable region are not associated in the first constrained scFv domain and the first constrained scFv domain does not bind to the first immune cell antigen (e.g., human immune cell antigen);
(iv) a first cleavable linker;
(v) a second single domain antigen binding domain (sdABD) that binds to a second target tumor antigen (TTA, e.g., a human TTA);
(vi) a second domain linker;
(vii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second immune cell antigen (e.g., human immune cell antigen), and wherein the first heavy chain variable region and the first light chain variable region are not associated in the second constrained scFv domain, and the second constrained scFv domain does not bind to the second immune cell antigen (e.g., human immune cell antigen);
(viii) a second cleavable linker; and
(ix) a half-life extension domain (e.g., third sdABD that binds to human serum albumin (HSA));
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

Without being bound by scientific theory, intramolecular association of the first heavy chain variable region of (iii) with the second light chain variable region of (vii) and/or intramolecular association of the second heavy chain variable region of (vii) with the first light chain variable region of (iii) stabilizes (e.g., resulting in stable expression) the prodrug proteins described herein and prevents dimerization of the constrained scFvs intermolecularly between different prodrug proteins prior to activation of the prodrug protein.

Proteins in the prodrug (inactive) form, when administered to a subject in a composition that comprises one or more of such prodrug proteins, can be activated once the first cleavable linker of (iv) is cleaved (e.g., by a protease in a tumor microenvironment). Activation of the prodrug proteins involves cleavage of at least two identical prodrug proteins (a first protein and a second protein that are identical to each other) in the first cleavable linker of (iv). Cleavage of the second cleavable linker of (viii) in the prodrug proteins results in release of the half-life extension domain, which is optionally and is not required for the activation of the prodrug proteins.

Cleavage of each prodrug proteins in the first cleavable linker of (iv) results in a first polypeptide comprising a first sdABD that binds to a first TTA, a first domain linker, and a first constrained scFv domain, and a second polypeptide comprising a second sdABD that binds to a second TTA, a second domain linker, and a second constrained sdFv domain. In some embodiments, the second polypeptide may additionally comprise a half-life extension domain (e.g., a third sdABD that binds to HSA) if the second cleavable linker of (viii) is not cleaved. Conversely, in some embodiments, the second polypeptide may not comprise a half-life extension domain (e.g., a third sdABD that binds to HSA) if the second cleavable linker of (viii) is cleaved.

Upon cleavage of the first cleavable linker of (iv) and optionally the second cleavable linker of (viii) in two prodrug proteins (i.e., a first prodrug protein and a second prodrug protein, which are identical before cleavage), the first heavy chain variable region of the first protein associates with the first light chain variable region of the second protein, forming an active Fv that binds to the first human immune cell antigen; the first light chain variable region of the first protein associates with the first heavy chain variable region of the second protein, forming an active Fv that binds to the first human immune cell antigen; the second heavy chain variable region of the first protein associates with the second light chain variable region of the second protein, forming an Fv that binds to the second human immune cell antigen; and the second light chain variable region of the first protein associates with the second heavy chain variable region of the second protein, forming an Fv that binds to the second human immune cell antigen.

As such, in some embodiments, the cleavage fragments of the prodrug (inactive) proteins assemble into two dimers (a first dimer and a second dimer), each dimer being a bi-specific molecule capable of binding a TTA and an immune cell antigen.

In some embodiments, a first dimer is formed via the dimerization of a first polypeptide (cleavage product of the prodrug protein) comprising:
(i) a first single domain antigen binding domain (sdABD) that binds to a first human target tumor antigen (TTA);
(ii) a first domain linker;
(iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first human immune cell antigen, and wherein the first heavy chain variable region and the first light chain variable region are not associated in the first constrained scFv domain and the first constrained scFv domain does not bind to the first human immune cell antigen;
wherein in the first dimer, the first VH of one polypeptide associates with the first VL of the other polypeptide, and the first VL of one polypeptide associates with the first VH of the other polypeptide, forming two active variable fragments (Fvs) each capable of binding to the first immune antigen.

In some embodiments, a second dimer is formed via the dimerization of a second polypeptide (cleavage product of the prodrug protein) comprising:
(i) a second single domain antigen binding domain (sdABD) that binds to a second human target tumor antigen (TTA);
(ii) a second domain linker;
(iii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second human immune cell antigen, and wherein the second heavy chain variable region and the second light chain variable region are not associated in the second constrained scFv domain, and the second constrained scFv domain does not bind to the second human immune cell antigen, and optionally a half-life extension domain (e.g., a third sdABD that binds to HSA) if the second cleavable linker of (viii) is cleaved;
wherein in the second dimer, the second VH of one polypeptide associates with the second VL of the other polypeptide, and the second VL of one polypeptide associates with the second VH of the other polypeptide, forming two active variable fragments (Fvs) each capable of binding to the second immune antigen.

It is to be understood that the first dimer is a homodimer. The second dimer, in some embodiments, is a homodimer, e.g., when both polypeptides that form the second dimer contain a half-life extension domain (e.g., a third sdABD that binds to HSA), or when both polypeptides that form the second dimer do not contain a half-life extension domain (e.g., a third sdABD that binds to HSA). In some embodiments, the second dimer can be a heterodimer, e.g., when one polypeptide that forms the second dimer contains a half-life extension domain (e.g., a third sdABD that binds to HSA), and the other polypeptide that forms the second dimer does not contain a half-life extension domain (e.g., a third sdABD that binds to HSA).

Non-limiting examples of the components of the proteins described herein, in prodrug form or active form, are provided. In some embodiments, in a protein described herein, the first TTA bound by the first sdABD of (i) is different from the second TTA bound by the second sdABD of (v). In this case, the first sdABD (i) is different from the second sdABD of (v). In some embodiments, in a protein described herein, the first TTA bound by the first sdABD of (i) is the same as the second TTA bound by the second sdABD of (v). It is to be understood that when the first TTA is the same as the second TTA, the first sdABD of (i) may be the same as or different from the second sdABD of (v). For example, the first sdABD of (i) and the second sdABD of (v) may bind to different epitopes of the same TTA. In another example, the first sdABD of (i) and the second sdABD of (v) may bind to the same epitope of the same TTA but have different amino acid sequences. In some embodiments, the first sdABD of (i) and the second sdABD of (v) are the same (i.e., comprising the same amino acid sequences).

In some embodiments, the first TTA and/or the second TTA is selected from: a4-integrin, A33, ACVRL 1/ALK1, ADAM17, ALK, APRIL, B7H3, BCMA, C242, CA9, CA125, Cadherin-19, CAIX, CanAg, Carbonic Anhydrase IX, CCN1, CCR4, CD123, CD133, CD137 (4-1BB), CD138/Syndecanl, CD19, CD2, CD20, CD22, CD30, CD33, CD37, CD38, CD4, CD40, CD44, CD45, CD48, CD5, CD52, CD56, CD59, CD70, CD70b, CD71, CD74, CD79b, CD80, CD86, CD98, CEA, CEACAM, CEACAM1, CK8, c-Kit, Claudin-1 (CLDN1), CLDN18 (including CLDN18.2), CLDN6, c-met/HGFR, c-RET, Cripto, CTLA-4, CXCR4, DKK-1, DLL3, DLL4, TRAIL-R2/DR5, DRS, EGFL7, EGFR, EGFRvIII, endoglin, ENPP3, EpCAM, EphA2, Episialin, FAP, FGFR1, FGFR2, FGFR3, FGFR4, fibronectin extra-domain B, FLT-3, flt4, folate receptor 1, FOLR1, Guanylyl Cyclase C (GCC), GD2, GD3, Glypican-3, Glypicans, GM3, GPNMB, GPR49, GRP78, Her2/Neu, HER3/ERBB3, HLA-DR, ICAM-1, IGF-1R, IGFR, IL-3Ra, Integrin a5b1, Integrin a6b4, Integrin aV, Integrin anb3, Lewis Y, Lewis y/b antigen, LFL2, LIV-1, LRCC15, Ly6E, LYPD3, MCP-1, Mesothelin, MMP-9, MUC1, MUC18, MUC5A, MUC5AC, Myostatin, NaPi2b, Neuropilin 1, NGcGM3, NRP1, P-cadherin, PCLA, PD-1, PDGFRa, PD-L1, PD-L2, Phosphatidylserine, PIVKA-II, PLVAP, PRLR, Progastrin, PSCA, PSMA, RANKL, RG1, Siglec-15, SLAMF6, SLAMF7, SLC44A4, STEAP-1, TACSTD-2, Tenascin C, TPBG, TRAIL-R1/DR4, TROP-2, TWEAKR, TYRP1, VANGL2, VEGF, VEGF-C, VEGFR-2, and VEGF-R2.

In some embodiments, the first TTA is selected from: EGFR, HER2, TROP2, CA9, FOLR1, LyPD3, EpCAM, B7H3, CD19, CD20, CD22, CD38, BCMA, LRRC15 and FAP. In some embodiments, the second TTA is selected from: EGFR, HER2, TROP2, CA9, FOLR1, LyPD3, EpCAM, B7H3, CD19, CD20, CD22, CD38, BCMA, LRRC15 and FAP. In some embodiments, the first TTA is selected from: EGFR, HER2, TROP2, CA9, FOLR1, LyPD3, EpCAM, and B7H3. In some embodiments, the second TTA is selected from: EGFR, HER2, TROP2, CA9, FOLR1, LyPD3, EpCAM, and B7H3. In some embodiments, the first TTA is selected from: EGFR, HER2, TROP2, CA9, FOLR1, LyPD3. In some embodiments, the second TTA is selected from: EGFR, HER2, TROP2, CA9, FOLR1, LyPD3. In some embodiments, any one of the TTAs provided herein is a human TTA.

In some embodiments, the first TTA is selected from: EGFR, HER2, TROP2, CA9, FOLR1, LyPD3, EpCAM, B7H3, CD19, CD20, CD22, CD38, BCMA, LRRC15 and FAP, and the second TTA is selected from: EGFR, HER2, TROP2, CA9, FOLR1, LyPD3, EpCAM, B7H3, CD19, CD20, CD22, CD38, BCMA, LRRC15 and FAP, and wherein the first TTA is the same as the second TTA. In some embodiments, the first TTA is selected from: EGFR, HER2, TROP2, CA9, FOLR1, LyPD3, EpCAM, and B7H3 and the second TTA is selected from: EGFR, HER2, TROP2, CA9, FOLR1, LyPD3, EpCAM, and B7H3, wherein the first TTA is the same as the second TTA. In some embodiments, the first TTA is selected from: EGFR, HER2, TROP2, CA9, FOLR1, and LyPD3 and the second TTA is selected from: EGFR, HER2, TROP2, CA9, FOLR1, and LyPD3, wherein the first TTA is the same as the second TTA.

In some embodiments, the first TTA is selected from: EGFR, HER2, TROP2, CA9, FOLR1, LyPD3, EpCAM, B7H3, CD19, CD20, CD22, CD38, BCMA, LRRC15 and FAP, and the second TTA is selected from: EGFR, HER2, TROP2, CA9, FOLR1, LyPD3, EpCAM, B7H3, CD19, CD20, CD22, CD38, BCMA, LRRC15 and FAP, and wherein the first TTA is different from the second TTA. In some embodiments, the first TTA is selected from: EGFR, HER2, TROP2, CA9, FOLR1, LyPD3, EpCAM, and B7H3 and the second TTA is selected from: EGFR, HER2, TROP2, CA9, FOLR1, LyPD3, EpCAM, and B7H3, wherein the first TTA is different from the second TTA. In some embodiments, the first TTA is selected from: EGFR, HER2, TROP2, CA9, FOLR1, and LyPD3 and the second TTA is selected from: EGFR, HER2, TROP2, CA9, FOLR1, and LyPD3, wherein the first TTA is different from the second TTA.

In some embodiments, the first TTA is EGFR and the second TTA is EGFR. In some embodiments, the first TTA is EGFR and the second TTA is EGFR, and the first sdABD binds to a different epitope of EGFR than the second sdABD. In some embodiments, the first TTA is EGFR and the second TTA is EGFR, and the first sdABD is the same as the second sdABD (e.g., having the same amino acid sequences).

In some embodiments, the first TTA is HER2 and the second TTA is HER2. In some embodiments, the first TTA is HER2 and the second TTA is HER2, and the first sdABD binds to a different epitope of HER2 than the second sdABD. In some embodiments, the first TTA is HER2 and the second TTA is HER2, and the first sdABD is the same as the second sdABD (e.g., having the same amino acid sequences).

In some embodiments, the first TTA is TROP2 and the second TTA is TROP2. In some embodiments, the first TTA is TROP2 and the second TTA is TROP2, and the first sdABD binds to a different epitope of TROP2 than the second sdABD. In some embodiments, the first TTA is TROP2 and the second TTA is TROP2, and the first sdABD is the same as the second sdABD (e.g., having the same amino acid sequences).

In some embodiments, the first TTA is FOLR1 and the second TTA is FOLR1. In some embodiments, the first TTA is FOLR1 and the second TTA is FOLR1, and the first sdABD binds to a different epitope of FOLR1 than the second sdABD. In some embodiments, the first TTA is FOLR1 and the second TTA is FOLR1, and the first sdABD is the same as the second sdABD (e.g., having the same amino acid sequences).

In some embodiments, the first TTA is CA9 and the second TTA is CA9. In some embodiments, the first TTA is CA9 and the second TTA is CA9, and the first sdABD binds to a different epitope of CA9 than the second sdABD. In some embodiments, the first TTA is CA9 and the second TTA is CA9 and the first sdABD is the same as the second sdABD (e.g., having the same amino acid sequences).

In some embodiments, the first TTA is LyPD3 and the second TTA is LyPD3. In some embodiments, the first TTA is LyPD3 and the second TTA is LyPD3, and the first sdABD binds to a different epitope of LyPD3 than the second sdABD. In some embodiments, the first TTA is LyPD3 and the second TTA is LyPD3 and the first sdABD is the same as the second sdABD (e.g., having the same amino acid sequences).

In some embodiments, the first TTA is EpCAM and the second TTA is EpCAM. In some embodiments, the first TTA is EpCAM and the second TTA is EpCAM, and the first sdABD binds to a different epitope of EpCAM than the second sdABD. In some embodiments, the first TTA is EpCAM and the second TTA is EpCAM, and the first sdABD is the same as the second sdABD (e.g., having the same amino acid sequences).

In some embodiments, the first TTA is B7H3 and the second TTA is B7H3. In some embodiments, the first TTA is B7H3 and the second TTA is B7H3, and the first sdABD binds to a different epitope of B7H3 than the second sdABD. In some embodiments, the first TTA is B7H3 and the second TTA is B7H3, and the first sdABD is the same as the second sdABD (e.g., having the same amino acid sequences).

In some embodiments, the first TTA is HER2 and the second TTA is EGFR. In some embodiments, the first TTA is EGFR and the second TTA is HER2. In some embodiments, the first TTA is EGFR and the second TTA is TROP2. In some embodiments, the first TTA is TROP2 and the second TTA is EGFR. In some embodiments, the first TTA is EGFR and the second TTA is FOLR1. In some embodiments, the first TTA is FOLR1 and the second TTA is EGFR. In some embodiments, the first TTA is EpCAM and the second TTA is EGFR. In some embodiments, the first TTA is EGFR and the second TTA is EpCAM.

Non-limiting examples of sdABDs that bind EGFR, and that may be used as the first sdABD of (i) and/or the second sdABD of (v) are provided in Table 3. In some embodiments, the first sdABD of (i) and/or the second sdABD of (v) comprises a CDR1, a CDR2, and a CDR3 of an anti-EGFR sdABD as set forth in any one of SEQ ID NOs: 4, 5, and 9-11. In some embodiments, the first sdABD of (i) and/or the second sdABD of (v) comprises an amino acid sequence that is at least 80% (e.g., at least 80%, at least 85%, at least 90%, at least 95%, or at least 99%) identical to the amino acid sequence of any one of SEQ ID NOs: 4, 5, and 9-11. In some embodiments, the first sdABD of (i) and/or the second sdABD of (v) comprises the amino acid sequence of any one of SEQ ID NOs: 4, 5, and 9-11. In some embodiments, the first sdABD of (i) and/or the second sdABD of (v) comprises the amino acid sequence of SEQ ID NO: 5. In some embodiments, the first sdABD of (i) and/or the second sdABD of (v) comprises the amino acid sequence of SEQ ID NO: 9.

Non-limiting examples of sdABDs that bind HER2, and that may be used as the first sdABD of (i) and/or the second sdABD of (v) are provided in Table 3. In some embodiments, the first sdABD of (i) and/or the second sdABD of (v) comprises a CDR1, a CDR2, and a CDR3 of an anti-HER2 sdABD as set forth in any one of SEQ ID NOs: 45, 48-52, 54, 58, 60, 63, 66, 68, 72, 75-78, 82, 85, 89, 95, 96, 99, 103, 104, 108, 112, 116, 117, 121, 299, and 300. In some embodiments, the first sdABD of (i) and/or the second sdABD of (v) comprises an amino acid sequence that is at least 80% (e.g., at least 80%, at least 85%, at least 90%, at least 95%, or at least 99%) identical to the amino acid sequence of any one of SEQ ID NOs: 45, 48-52, 54, 58, 60, 63, 66, 68, 72, 75-78, 82, 85, 89, 95, 96, 99, 103, 104, 108, 112, 116, 117, 121, 299, and 300. In some embodiments, the first sdABD of (i) and/or the second sdABD of (v) comprises the amino acid sequence of any one of SEQ ID NOs: 45, 48-52, 54, 58, 60, 63, 66, 68, 72, 75-78, 82, 85, 89, 95, 96, 99, 103, 104, 108, 112, 116, 117, 121, 299, and 300. In some embodiments, the first sdABD of (i) and/or the second sdABD of (v) comprises the amino acid sequence of SEQ ID NO: 96.

Non-limiting examples of sdABDs that bind TROP2, and that may be used as the first sdABD of (i) and/or the second sdABD of (v) are provided in Table 3. In some embodiments, the first sdABD of (i) and/or the second sdABD of (v) comprises a CDR1, a CDR2, and a CDR3 of an anti-TROP2 sdABD as set forth in any one of SEQ ID NOs: 145, 149, 153, 156, 160, and 164. In some embodiments, the first sdABD of (i) and/or the second sdABD of (v) comprises an amino acid sequence that is at least 80% (e.g., at least 80%, at least 85%, at least 90%, at least 95%, or at least 99%) identical to the amino acid sequence of any one of SEQ ID NOs: 145, 149, 153, 156, 160, and 164. In some embodiments, the first sdABD of (i) and/or the second sdABD of (v) comprises the amino acid sequence of any one of SEQ ID NOs: 145, 149, 153, 156, 160, and 164.

Non-limiting examples of sdABDs that bind CA9, and that may be used as the first sdABD of (i) and/or the second sdABD of (v) are provided in Table 3. In some embodiments, the first sdABD of (i) and/or the second sdABD of (v) comprises a CDR1, a CDR2, and a CDR3 of an anti-CA9 sdABD as set forth in any one of SEQ ID NOs: 186, 190, 194, and 198. In some embodiments, the first sdABD of (i) and/or the second sdABD of (v) comprises an amino acid sequence that is at least 80% (e.g., at least 80%, at least 85%, at least 90%, at least 95%, or at least 99%) identical to the amino acid sequence of any one of SEQ ID NOs: 186, 190, 194, and 198. In some embodiments, the first sdABD of (i) and/or the second sdABD of (v) comprises the amino acid sequence of any one of SEQ ID NOs: 186, 190, 194, and 198.

Non-limiting examples of sdABDs that bind FOLR1, and that may be used as the first sdABD of (i) and/or the second sdABD of (v) are provided in Table 3. In some embodiments, the first sdABD of (i) and/or the second sdABD of (v) comprises a CDR1, a CDR2, and a CDR3 of an anti-FOLR1 sdABD as set forth in any one of SEQ ID NOs: 33, 37, and 41. In some embodiments, the first sdABD of (i) and/or the second sdABD of (v) comprises an amino acid sequence that is at least 80% (e.g., at least 80%, at least 85%, at least 90%, at least 95%, or at least 99%) identical to the amino acid sequence of any one of SEQ ID NOs: 33, 37, and 41. In some embodiments, the first sdABD of (i) and/or the second sdABD of (v) comprises the amino acid sequence of any one of SEQ ID NOs: 33, 37, and 41.

Non-limiting examples of sdABDs that bind LyPD3, and that may be used as the first sdABD of (i) and/or the second sdABD of (v) are provided in Table 3. In some embodiments, the first sdABD of (i) and/or the second sdABD of (v) comprises a CDR1, a CDR2, and a CDR3 of an anti-LyPD3 sdABD as set forth in any one of SEQ ID NOs: 125, 128, 130, 134, 138, and 301. In some embodiments, the first sdABD of (i) and/or the second sdABD of (v) comprises an amino acid sequence that is at least 80% (e.g., at least 80%, at least 85%, at least 90%, at least 95%, or at least 99%) identical to the amino acid sequence of any one of SEQ ID NOs: 125, 128, 130, 134, 138, and 301. In some embodiments, the first sdABD of (i) and/or the second sdABD of (v) comprises the amino acid sequence of any one of SEQ ID NOs: 125, 128, 130, 134, 138, and 301.

Non-limiting examples of sdABDs that bind EpCAM, and that may be used as the first sdABD of (i) and/or the second sdABD of (v) are provided in Table 3. In some embodiments, the first sdABD of (i) and/or the second sdABD of (v) comprises a CDR1, a CDR2, and a CDR3 of an anti-EpCAM sdABD as set forth in any one of SEQ ID NOs: 15, 19, 23, 27, and 29. In some embodiments, the first sdABD of (i) and/or the second sdABD of (v) comprises an amino acid sequence that is at least 80% (e.g., at least 80%, at least 85%, at least 90%, at least 95%, or at least 99%) identical to the amino acid sequence of any one of SEQ ID NOS: 15, 19, 23, 27, and 29. In some embodiments, the first sdABD of (i) and/or the second sdABD of (v) comprises the amino acid sequence of any one of SEQ ID NOs: 15, 19, 23, 27, and 29.

Non-limiting examples of sdABDs that bind B7H3, and that may be used as the first sdABD of (i) and/or the second sdABD of (v) are provided in Table 3. In some embodiments, the first sdABD of (i) and/or the second sdABD of (v) comprises a CDR1, a CDR2, and a CDR3 of an anti-B7H3 sdABD as set forth in any one of SEQ ID NOs: 168, 172, 174, 176, 178, 180, and 182. In some embodiments, the first sdABD of (i) and/or the second sdABD of (v) comprises an amino acid sequence that is at least 80% (e.g., at least 80%, at least 85%, at least 90%, at least 95%, or at least 99%) identical to the amino acid sequence of any one of SEQ ID NOs: 168, 172, 174, 176, 178, 180, and 182. In some embodiments, the first sdABD of (i) and/or the second sdABD of (v) comprises the amino acid sequence of any one of SEQ ID NOs: 168, 172, 174, 176, 178, 180, and 182.

In some embodiments, the first human target tumor antigen is EGFR and the second human target tumor antigen is EGFR, wherein the first sdABD comprises the amino acid sequence of SEQ ID NO: 5 and the second sdABD comprises the amino acid sequence of SEQ ID NO: 5.

In some embodiments, the first human target tumor antigen is EGFR and the second human target tumor antigen is EGFR, wherein the first sdABD comprises the amino acid sequence of SEQ ID NO: 9 and the second sdABD comprises the amino acid sequence of SEQ ID NO: 9.

In some embodiments, the first human target tumor antigen is EGFR and the second human target tumor antigen is EGFR, wherein the first sdABD comprises the amino acid sequence of SEQ ID NO: 9 and the second sdABD comprises the amino acid sequence of SEQ ID NO: 5.

In some embodiments, the first human target tumor antigen is EGFR and the second human target tumor antigen is EGFR, wherein the first sdABD comprises the amino acid sequence of SEQ ID NO: 5 and the second sdABD comprises the amino acid sequence of SEQ ID NO: 9.

In some embodiments, the first human target tumor antigen is HER2 and the second human target tumor antigen is HER2, wherein the first sdABD comprises the amino acid sequence of SEQ ID NO: 96 and the second sdABD comprises the amino acid sequence of SEQ ID NO: 96.

In some embodiments, wherein the first human target tumor antigen is EGFR and the second human target tumor antigen is HER2, wherein the first sdABD comprises the amino acid sequence of SEQ ID NO: 5 or SEQ ID NO: 9 and the second sdABD comprises the amino acid sequence of SEQ ID NO: 96.

In some embodiments, the first human target tumor antigen is HER2 and the second human target tumor antigen is EGFR, wherein the first sdABD comprises the amino acid sequence of SEQ ID NO: 96 and the second sdABD comprises the amino acid sequence of SEQ ID NO: 5 or SEQ ID NO: 9.

In some embodiments, in a protein described herein, the first immune cell antigen is different from the second immune cell antigen. In some embodiments, the first immune cell antigen and the second immune cell antigen are expressed on the same immune cell, and the first immune cell antigen is different from the second immune cell antigen. In some embodiments, the first immune cell antigen and the second immune cell antigen are expressed on different immune cells, and the first immune cell antigen is different from the second immune cell antigen.

In some embodiments, the first immune cell antigen and/or the second immune cell antigen is an antigen expressed on an immune cell selected from a T-cell, a natural killer cell (NK cell), a macrophage, a B cell, a neutrophile, and a monocyte. In some embodiments, the first immune cell antigen and/or the second immune cell antigen is an antigen expressed on an immune cell selected from a T cell, a natural killer cell (NK cell), and a macrophage. In some embodiments, the first immune cell antigen is an antigen expressed on a T cell and the second immune cell antigen is a different antigen expressed on a T cell. In some embodiments, the first immune cell antigen is an antigen expressed on a T cell and the second immune cell antigen is a different antigen expressed on a NK cell. In some embodiments, the first immune cell antigen is an antigen expressed on a T cell and the second immune cell antigen is a different antigen expressed on a macrophage. In some embodiments, the first immune cell antigen is an antigen expressed on a NK cell and the second immune cell antigen is a different antigen expressed on a NK cell. In some embodiments, the first immune cell antigen is an antigen expressed on a NK cell and the second immune cell antigen is a different antigen expressed on a T-cell. In some embodiments, the first immune cell antigen is an antigen expressed on a NK cell and the second immune cell antigen is a different antigen expressed on a macrophage. In some embodiments, the first immune cell antigen is an antigen expressed on a macrophage and the second immune cell antigen is a different antigen expressed on a macrophage. In some embodiments, the first immune cell antigen is an antigen expressed on a macrophage and the second immune cell antigen is a different antigen expressed on a T-cell. In some embodiments, the first immune cell antigen is an antigen expressed on a macrophage and the second immune cell antigen is a different antigen expressed on a NK cell.

Non-limiting examples of antigens expressed on T cells that may be used as the first immune cell antigen and/or second immune cell antigen in a protein described herein include: CD3, CD28, T cell receptor, programmed cell death protein 1 (PD-1), cytotoxic T-lymphocyte-associated protein 4 (CTLA-4), T-cell immunoglobulin and mucin domain 3 (TIM-3), lymphocyte-activation gene 3 (LAG-3), killer-cell immunoglobulin-like receptor (KIR), CD137 (also known as 4-1BB), OX40, CD27, GITR (TNFRSF18), TIGIT, inducible T cell costimulatory (ICOS), NKG2D, CD226, CD96, and CD40L. Non-limiting examples of antigens expressed on NK cells that may be used as the first immune cell antigen and/or second immune cell antigen in a protein described herein include: CD16A, CD28, NKG2D, CD226, CRTAM, LFA-1, CD27, CD96, TIGIT, and KIR. Non-limiting examples of antigens expressed on macrophages that may be used as the first immune cell antigen and/or second immune cell antigen in a protein described herein include: CSF1R, CD40, MARCO, VSIG4, and CD163. In some embodiments, any one of the immune cell antigens provided herein is a human immune cell antigen.

In some embodiments, the first immune cell antigen is CD3 and the second immune cell antigen is CD28. In some embodiments, the first immune cell antigen is CD28 and the second immune cell antigen is CD3.

In some embodiments, in a protein described herein, the first constrained scFv domain of (iii) and/or the second constrained scFv domain of (vii) may comprise a heavy chain variable region (VH) and a light chain variable region (VL) of any known immune cell antigen antibodies (e.g., antibodies that bind to CD3 or CD28). As such, the first VH and first VL, if associated, are capable of binding the first immune cell antigen, and the second VH and second VL, if associated, are capable of binding the second immune cell antigen.

It is also understood that, in a protein described herein, preventing the first VH from being able to associate with the first VL within the first constrained scFv domain, e.g., by linking the first VH and the first VL with a first “constrained non-cleavable linker (CNCL)” results in a first constrained scFv domain that does not bind to the first immune cell antigen. Similarly, preventing the second VH from being able to associate with the second VL within the second constrained scFv domain, e.g., by linking the second VH and the second VL with a second “constrained non-cleavable linker” results in a second constrained scFv domain that does not bind to the second immune cell antigen. The constrained non-cleavable linkers are too short to allow the first VH and the first VL, or the second VH and the second VL to associate within the constrained scFv domains. In some embodiments, the first CNCL and/or the second CNCL is 6-10 amino acids long (e.g., 6, 7, 8, 9, or 10 amino acids long). Non-limiting examples of amino acid sequences of the first CNCL and/or second CNCL are provided in Table 3. In some embodiments the first CNCL and/or the second CNCL has a sequence of GGGSGGGS (SEQ ID NO: 302)

Non-limiting examples of antibodies that bind to CD3 from which the first VH and the first VL of the first constrained scFv domain of (iii) and/or the second VH and the second VL of the second constrained scFv domain of (vii) may be derived include: muromonab-CD3 (OKT3), otelixizumab (TRX4), teplizumab (MGA031), visilizumab (Nuvion), blinatumomab, foralumab, SP34 or I2C, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31. Non-limiting examples of VHs and VLs that, when associated, bind to CD3, and that may be used in the first constrained scFv domain of (iii) and/or the second constrained scFv domain of (vii) are provided in Table 3. In some embodiments, the first constrained scFv domain of (iii) and/or the second constrained scFv domain of (vii) comprises a VH comprising a vhCDR1 as set forth in SEQ ID NO: 199, a vhCDR2 as set forth in SEQ ID NO: 200, and a vhCDR3 as set forth in SEQ ID NO: 201, and a VL comprising vlCDR1 as set forth in SEQ ID NO: 202, a vlCDR2 as set forth in SEQ ID NO: 203, and a vlCDR3 as set forth in SEQ ID NO: 204. In some embodiments, the first constrained scFv domain of (iii) and/or the second constrained scFv domain of (vii) comprises a VH comprising an amino acid sequence that is at least 80% (e.g., at least 80%, at least 85%, at least 90%, at least 95%, or at least 99%) to the amino acid sequence of SEQ ID NO: 205 and a VL comprising an amino acid sequence that is at least 80% (e.g., at least 80%, at least 85%, at least 90%, at least 95%, or at least 99%) to the amino acid sequence of SEQ ID NO: 206. In some embodiments, the first constrained scFv domain of (iii) and/or the second constrained scFv domain of (vii) comprises a VH comprising the amino acid sequence of SEQ ID NO: 205 and a VL comprising the amino acid sequence of SEQ ID NO: 206.

Non-limiting examples of antibodies that bind to CD28 from which the first VH and the first VL of the first constrained scFv domain of (iii) and/or the second VH and the second VL of the second constrained scFv domain of (vii) may be derived include: theralizumab, utomilumab (PF-05082566), ES101 (a bispecific PD-L1×CD28 antibody), PRS-343 (a HER2×CD28 bispecific antibody) urelumab (BMS-663513), and TGN1412 and TGN1112 as described in U.S. Pat. No. 7,939,638, incorporated herein by reference. Non-limiting examples of VH and VLs that, when associated, bind to CD28, and that may be used in the first constrained scFv domain of (iii) and/or the second constrained scFv domain of (vii) are provided in Table 3. In some embodiments, the first constrained scFv domain of (iii) and/or the second constrained scFv domain of (vii) comprises a VH comprising a vhCDR1 as set forth in SEQ ID NO: 207, a vhCDR2 as set forth in SEQ ID NO: 208 or SEQ ID NO: 215, and a vhCDR3 as set forth in SEQ ID NO: 209, and a VL comprising vlCDR1 as set forth in SEQ ID NO: 210, a vlCDR2 as set forth in SEQ ID NO: 211, and a vlCDR3 as set forth in SEQ ID NO: 212. In some embodiments, the first constrained scFv domain of (iii) and/or the second constrained scFv domain of (vii) comprises a VH comprising an amino acid sequence that is at least 80% (e.g., at least 80%, at least 85%, at least 90%, at least 95%, or at least 99%) to the amino acid sequence of SEQ ID NO: 213 or SEQ ID NO: 216 and a VL comprising an amino acid sequence that is at least 80% (e.g., at least 80%, at least 85%, at least 90%, at least 95%, or at least 99%) to the amino acid sequence of SEQ ID NO: SEQ ID NO: 214. In some embodiments, the first constrained scFv domain of (iii) and/or the second constrained scFv domain of (vii) comprises a VH comprising the amino acid sequence of SEQ ID NO: 213 or SEQ ID NO: 216 and a VL comprising the amino acid sequence of SEQ ID NO: SEQ ID NO: 214.

Non-limiting examples of antibodies that bind to PD-1 from which the first VH and the first VL of the first constrained scFv domain of (iii) and/or the second VH and the second VL of the second constrained scFv domain of (vii) may be derived include: pembrolizumab, dostarlimab, and nivolumab.

Non-limiting examples of antibodies that bind to CTLA-4 from which the first VH and the first VL of the first constrained scFv domain of (iii) and/or the second VH and the second VL of the second constrained scFv domain of (vii) may be derived include: ipilimumab.

Non-limiting examples of antibodies that bind to TIM-3 from which the first VH and the first VL of the first constrained scFv domain of (iii) and/or the second VH and the second VL of the second constrained scFv domain of (vii) may be derived include: TSR-022 and Sym023.

Non-limiting examples of antibodies that bind to LAG-3 from which the first VH and the first VL of the first constrained scFv domain of (iii) and/or the second VH and the second VL of the second constrained scFv domain of (vii) may be derived include: BMS-986016.

Non-limiting examples of antibodies that bind to KIR from which the first VH and the first VL of the first constrained scFv domain of (iii) and/or the second VH and the second VL of the second constrained scFv domain of (vii) may be derived include: lirilumab.

Non-limiting examples of antibodies that bind to CD137 from which the first VH and the first VL of the first constrained scFv domain of (iii) and/or the second VH and the second VL of the second constrained scFv domain of (vii) may be derived include: utomilumab and urelumab.

Non-limiting examples of antibodies that bind to OX40 from which the first VH and the first VL of the first constrained scFv domain of (iii) and/or the second VH and the second VL of the second constrained scFv domain of (vii) may be derived include: PF-045-18600 and BMS-986178.

Non-limiting examples of antibodies that bind to CD27 from which the first VH and the first VL of the first constrained scFv domain of (iii) and/or the second VH and the second VL of the second constrained scFv domain of (vii) may be derived include: varlilumab.

Non-limiting examples of antibodies that bind to GITR from which the first VH and the first VL of the first constrained scFv domain of (iii) and/or the second VH and the second VL of the second constrained scFv domain of (vii) may be derived include: GWN323 or BMS-986156.

Non-limiting examples of antibodies that bind to TIGIT from which the first VH and the first VL of the first constrained scFv domain of (iii) and/or the second VH and the second VL of the second constrained scFv domain of (vii) may be derived include: OMP-313M32, MTIG7192A, BMS-986207, and MK-7684.

Non-limiting examples of antibodies that bind to ICOS from which the first VH and the first VL of the first constrained scFv domain of (iii) and/or the second VH and the second VL of the second constrained scFv domain of (vii) may be derived include: JTX-2011.

Non-limiting examples of antibodies that bind to CSF1R from which the first VH and the first VL of the first constrained scFv domain of (iii) and/or the second VH and the second VL of the second constrained scFv domain of (vii) may be derived include: mactuzumab/RG7155 and IMC-CS4.

Non-limiting examples of antibodies that bind to CD40 from which the first VH and the first VL of the first constrained scFv domain of (iii) and/or the second VH and the second VL of the second constrained scFv domain of (vii) may be derived include: CP-870,893.

Non-limiting examples of antibodies that bind to CD16A from which the first VH and the first VL of the first constrained scFv domain of (iii) and/or the second VH and the second VL of the second constrained scFv domain of (vii) may be derived include: NTM-1633 and AFM13.

Non-limiting examples of antibodies that bind to CD96 from which the first VH and the first VL of the first constrained scFv domain of (iii) and/or the second VH and the second VL of the second constrained scFv domain of (vii) may be derived include: GSK6097608.

Non-limiting examples of antibodies that bind to CD40L from which the first VH and the first VL of the first constrained scFv domain of (iii) and/or the second VH and the second VL of the second constrained scFv domain of (vii) may be derived include: BG9588.

Non-limiting examples of antibodies that bind to LFA-1 from which the first VH and the first VL of the first constrained scFv domain of (iii) and/or the second VH and the second VL of the second constrained scFv domain of (vii) may be derived include: efalizumab.

In some embodiments, in a protein described herein, the first constrained scFv domain of (iii) comprises the first VH linked to the N-terminus of the first VL. In some embodiments, in a protein described herein, the first constrained scFv domain of (iii) comprises the first VH linked to the C-terminus of the first VL.

In some embodiments, in a protein described herein, the second constrained scFv domain of (vii) comprises the second VH linked to the N-terminus of the second VL. In some embodiments, in a protein described herein, the second constrained scFv domain of (vii) comprises the second VH linked to the C-terminus of the second VL.

In some embodiments, in a protein described herein, the first VH and the first VL of the first constrained scFv domain of (iii) is linked via a first CNCL. In some embodiments, in a protein described herein, the first constrained scFv domain of (iii) comprises, from N- to C-terminus: first VH-first CNCL-first VL. In some embodiments, in a protein described herein, the first constrained scFv domain of (iii) comprises, from N- to C-terminus: first VL-first CNCL-first VH.

In some embodiments, in a protein described herein, the second VH and the second VL of the second constrained scFv domain of (vii) is linked via a second CNCL. In some embodiments, in a protein described herein, the second constrained scFv domain of (vii) comprises, from N- to C-terminus: second VH-second CNCL-second VL. In some embodiments, in a protein described herein, the second constrained scFv domain of (vii) comprises, from N- to C-terminus: second VL-second CNCL-second VH.

In some embodiments, in a protein described herein, the first constrained scFv domain of (iii) comprises, from N- to C-terminus: first VH-first CNCL-first VL, and the second constrained scFv domain of (vii) comprises, from N- to C-terminus: second VH-second CNCL-second VL.

In some embodiments, in a protein described herein, the first constrained scFv domain of (iii) comprises, from N- to C-terminus: first VH-first CNCL-first VL, and the second constrained scFv domain of (vii) comprises, from N- to C-terminus: second VL-second CNCL-second VH.

In some embodiments, in a protein described herein, the first constrained scFv domain of (iii) comprises, from N- to C-terminus: first VL-first CNCL-first VH, and the second constrained scFv domain of (vii) comprises, from N- to C-terminus: second VH-second CNCL-second VL.

In some embodiments, in a protein described herein, the first constrained scFv domain of (iii) comprises, from N- to C-terminus: first VL-first CNCL-first VH, and the second constrained scFv domain of (vii) comprises, from N- to C-terminus: second VL-second CNCL-second VH.

In some embodiments, in a protein described herein, the first constrained scFv domain of (iii) comprises, from N- to C-terminus: first VH-first CNCL-first VL, and the second constrained scFv domain of (vii) comprises, from N- to C-terminus: second VH-second CNCL-second VL, and wherein the first immune cell antigen is CD3 and the second immune cell antigen is CD28.

In some embodiments, in a protein described herein, the first constrained scFv domain of (iii) comprises, from N- to C-terminus: first VH-first CNCL-first VL, and the second constrained scFv domain of (vii) comprises, from N- to C-terminus: second VH-second CNCL-second VL, and wherein the first immune cell antigen is CD28 and the second immune cell antigen is CD3.

In some embodiments, in a protein described herein, the first constrained scFv domain of (iii) comprises, from N- to C-terminus: first VH-first CNCL-first VL, and the second constrained scFv domain of (vii) comprises, from N- to C-terminus: second VL-second CNCL-second VH, and wherein the first immune cell antigen is CD3 and the second immune cell antigen is CD28.

In some embodiments, in a protein described herein, the first constrained scFv domain of (iii) comprises, from N- to C-terminus: first VH-first CNCL-first VL, and the second constrained scFv domain of (vii) comprises, from N- to C-terminus: second VL-second CNCL-second VH, and wherein the first immune cell antigen is CD28 and the second immune cell antigen is CD3.

In some embodiments, in a protein described herein, the first constrained scFv domain of (iii) comprises, from N- to C-terminus: first VL-first CNCL-first VH, and the second constrained scFv domain of (vii) comprises, from N- to C-terminus: second VH-second CNCL-second VL, and wherein the first immune cell antigen is CD3 and the second immune cell antigen is CD28.

In some embodiments, in a protein described herein, the first constrained scFv domain of (iii) comprises, from N- to C-terminus: first VL-first CNCL-first VH, and the second constrained scFv domain of (vii) comprises, from N- to C-terminus: second VH-second CNCL-second VL, and wherein the first immune cell antigen is CD28 and the second immune cell antigen is CD3.

In some embodiments, in a protein described herein, the first constrained scFv domain of (iii) comprises, from N- to C-terminus: first VL-first CNCL-first VH, and the second constrained scFv domain of (vii) comprises, from N- to C-terminus: second VL-second CNCL-second VH, and wherein the first immune cell antigen is CD3 and the second immune cell antigen is CD28.

In some embodiments, in a protein described herein, the first constrained scFv domain of (iii) comprises, from N- to C-terminus: first VL-first CNCL-first VH, and the second constrained scFv domain of (vii) comprises, from N- to C-terminus: second VL-second CNCL-second VH, and wherein the first immune cell antigen is CD28 and the second immune cell antigen is CD3.

In some embodiments, a protein described herein comprises at least one protease cleavage site comprising an amino acid sequence that is cleaved by at least one protease. In some cases, a protein described herein comprises 1, 2, 3 or 4 or more protease cleavage sites that are cleaved by at least one protease. In some embodiments, the proteins described herein comprise 2 protease cleavage sites that are cleaved by at least one protease. In some embodiments, in a protein described herein the first cleavable linker of (iv) and the second cleavable linker of (viii) are different (e.g., cleavable by different proteases or cleavable by a single protease but having different amino acid sequences). In some embodiments, in a protein described herein, the first cleavable linker of (iv) and the second cleavable linker of (viii) are the same (i.e., cleavable by a single protease).

Proteases are known to be secreted by some diseased cells and tissues, for example tumor or cancer cells, creating a microenvironment that is rich in proteases or a protease-rich microenvironment. In some embodiments, in a protein described herein, the first cleavable linker of (iv) and/or the second cleavable linker of (viii) is cleavable by a protease in the blood of a subject. In some embodiments, in a protein described herein, the first cleavable linker of (iv) and/or the second cleavable linker of (viii) is cleavable by a protease secreted by a tumor secrete into the tumor microenvironment. Proteases include but are not limited to serine proteases, cysteine proteases, aspartate proteases, threonine proteases, glutamic acid proteases, metalloproteases, asparagine peptide lyases, serum proteases, cathepsins (e.g., Cathepsin B, Cathepsin C, Cathepsin D, Cathepsin E, Cathepsin K, Cathepsin L, CathepsinS), kallikreins, hK1, hK10, hK15, KLK7, GranzymeB, plasmin, collagenase, Type IV collagenase, stromelysin, factor XA, chymotrypsin-like protease, trypsin-like protease, elastase-like protease, subtilisin-like protease, actinidain, bromelain, calpain, caspases (e.g. Caspase-3), Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, meprin, urokinase plasminogen activator (uPA), enterokinase, prostate-specific antigen (PSA, hK3), interleukin-1β converting enzyme, thrombin, FAP (FAP-α), dipeptidyl peptidase, and dipeptidyl peptidase IV (DPPIV/CD26). In some embodiments, in a protein described herein, the first cleavable linker of (iv) and/or the second cleavable linker of (viii) is cleavable by MMP9. Non-limiting examples of cleavable linkers that may be used in a protein described herein as the first cleavable linker of (iv) and/or the second cleavable linker of (viii) are provided in Table 3.

In some embodiments, in a protein described herein, the first domain linker of (ii) and/or the second domain linker of (vi) is a non-cleavable linker. In some embodiments, the first domain linker of (ii) is the same as the second domain linker of (vi). In some embodiments, the first domain linker of (ii) is different from the second domain linker of (vi). In some embodiments, domain linkers used to join domains to preserve the functionality of the domains, are generally longer, flexible linkers that are not cleaved (e.g., by proteases in a subject). Examples of linkers suitable for use as domain linkers of the protein described herein include but are not limited to (GS)n (SEQ ID NO: 303), (GGS)n (SEQ ID NO: 304), (GGGS)n (SEQ ID NO:305), (GGSG)n (SEQ ID NO:306), (GGSGG)n (SEQ ID NO:307), or (GGGGS)n (SEQ ID NO:308), wherein n is 1, 2, 3, 4, 5, 6, 7, 8, 9, or 10. In some embodiments, the length of the first domain linker of (ii) and/or the second domain linker of (vi) is about 15 amino acids.

In some embodiments, in a protein disclosed herein, a half-life extension domain of (ix) can be any known half-life extension domains known in the art including, without limitation, HSA binding domains, Fc domains, and small molecules.

Human serum albumin (HSA) (molecular mass ˜67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma. Noncovalent association with albumin extends the elimination half-time of short lived proteins. For example, a recombinant fusion of an albumin binding domain to a Fab fragment resulted in a reduced in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In some embodiments, in a protein described herein, a half-life extension domain of (xi) comprises a domain which specifically binds to HSA. In some embodiments, the HSA binding domain is a peptide. In further embodiments, the HSA binding domain is a small molecule. It is contemplated that the HSA binding domain of an antigen binding protein is fairly small and no more than 25 kD, no more than 20 kD, no more than 15 kD, or no more than 10 kD in some embodiments. In certain instances, the HSA binding domain is 5 kD or less if it is a peptide or small molecule.

In some embodiments, a half-life extension domain is a single domain antigen binding domain from a sdABD that binds to HSA. Non-limiting examples of sdABDs that bind to HSA are provided in Table 3. In some embodiments, in a protein described herein, the third sdABD of (ix) that binds to HSA comprises a CDR1 as set forth in SEQ ID NO: 217, a CDR2 as set forth in SEQ ID NO: 218, and a CDR3 as set forth in SEQ ID NO: 219. In some embodiments, the third sdABD of (ix) comprises an amino acid sequence that is at least 80% (e.g., at least 80%, at least 85%, at least 90%, at least 95%, or at least 99%) identical to the amino acid sequence of SEQ ID NO: 220. In some embodiments, the third sdABD of (ix) comprises the amino acid sequence of SEQ ID NO: 220.

The half-life extension domain of the protein provides for altered pharmacodynamics and pharmacokinetics of the antigen binding protein itself. As above, the half-life extension domain extends the elimination half-time. The half-life extension domain also alters pharmacodynamic properties including alteration of tissue distribution, penetration, and diffusion of the antigen-binding protein. In some embodiments, the half-life extension domain provides for improved tissue (including tumor) targeting, tissue penetration, tissue distribution, diffusion within the tissue, and enhanced efficacy as compared with a protein without a half-life extension binding domain. In one embodiment, therapeutic methods effectively and efficiently utilize a reduced amount of the antigen-binding protein, resulting in reduced side effects, such as reduced chance of cytokine release syndrome or cytokine storm.

Further, characteristics of the half-life extension domain, for example a HSA binding domain, include the binding affinity of the HSA binding domain for HSA. Affinity of said HSA binding domain can be selected so as to target a specific elimination half-time in a particular polypeptide construct. Thus, in some embodiments, the HSA binding domain has a high binding affinity. In other embodiments, the HSA binding domain has a medium binding affinity. In yet other embodiments, the HSA binding domain has a low or marginal binding affinity. Exemplary binding affinities include KD concentrations at 10 nM or less (high), between 10 nM and 100 nM (medium), and greater than 100 nM (low). As above, binding affinities to HSA are determined by known methods such as Surface Plasmon Resonance (SPR).

Additionally, it is known in the art that there can be immunogenicity in humans originating from the C-terminal sequences of certain ABDs. Accordingly, in general, particularly when the C-terminus of the protein terminates in an sdABD (for example, the sdABD-HSA domains of many of the constructs, a C-terminal capping sequence is added to reduce the likelihood of clearance of the proteins by the innate immune system of the patient. After cleavage the residual linker amino acids act as blocking peptides against human serum antibodies. Non-limiting examples of C-terminal capping sequences are provided in U.S. Pat. No. 10,858,418, incorporated herein by reference.

In some embodiments, a histidine tag (either His6 or His10) can be used. Any one of the proteins described herein (e.g., a protein comprising the amino acid sequence of any one of SEQ ID NOs: 234-249 listed in Table 3) may further comprise a His6 C-terminal tags (e.g., at the C-terminus) for purification reason, but these sequences can also be used to reduce immunogenicity in humans, as is shown by Holland et al., DOI 10.1007/s10875-013-9915-0 and U.S. Pat. No. 10,808,040, incorporated herein by reference.

Non-limiting examples of proteins in prodrug form described herein are provided below.

### A. Configuration 1 (e.g., Pro1136, Pro1184, Pro 1265, Pro 1267, and Pro1269 in Table 3)

In some embodiments, a protein described herein comprises, from N- to C-terminus:
(i) a first single domain antigen binding domain (sdABD) that binds to a first human target tumor antigen (TTA), wherein the first human TTA is selected from EGFR, HER2, Trop2, CA9, LyPD3, FOLR1, EpCAM, and B7H3;
(ii) a first domain linker (e.g., a non-cleavable linker);
(iii) a first constrained scFv domain comprising a first VH linked to the N-terminus of a first VL via a first CNCL (e.g., a CNCL that is 8 amino acids in length), wherein the first VH and first VL, if associated, are capable of binding a first human immune cell antigen (e.g., CD3), and wherein the first heavy chain variable region and the first light chain variable region are not associated in the first constrained scFv domain and the first constrained scFv domain does not bind to the first human immune cell antigen (e.g., CD3);
(iv) a first cleavable linker (e.g., a cleavable linker comprising a protease cleavage site provided in Table 3, such as a MMP9 cleavage site or variants thereof);
(v) a second single domain antigen binding domain (sdABD) that binds to a second human TTA, wherein the second human TTA is selected from the group consisting of EGFR, HER2, Trop2, CA9, LyPD3, FOLR1, EpCAM, and B7H3;
(vi) a second domain linker (e.g., a non-cleavable linker);
(vii) a second constrained scFv domain comprising a second VH linked to the N-terminus of a second VL via a second CNCL (e.g., a CNCL that is 8 amino acids in length), wherein the second VH and the second VL, if associated, are capable of binding a second human immune cell antigen (e.g., CD28), and wherein the first heavy chain variable region and the first light chain variable region are not associated in the second constrained scFv domain, and the second constrained scFv domain does not bind to the second human immune cell antigen (e.g., CD28);
(viii) a second cleavable linker (e.g., a cleavable linker comprising a protease cleavage site provided in Table 3, such as a MMP9 cleavage site or variants thereof); and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first TTA is the same as the second TTA. In some embodiments, the first TTA is the same as the second TTA, and the first sdABD binds a different epitope in the TTA than the second sdABD. In some embodiments, the first TTA is different from the second TTA. In some embodiments, the first TTA is EGFR and the second TTA is EGFR. In some embodiments, the first TTA is EGFR and the second TTA is HER2. In some embodiments, the first TTA is HER2 and the second TTA is EGFR. In some embodiments, the first TTA is HER2 and the second TTA is HER2.

In some embodiments, the first sdABD of (i) comprises the amino acid sequence of SEQ ID NO: 5, SEQ ID NO: 9, or SEQ ID NO: 96. In some embodiments, the second sdABD of (v) comprises the amino acid sequence of SEQ ID NO: 5, SEQ ID NO: 9, or SEQ ID NO: 96. In some embodiments, the first sdABD of (i) comprises the amino acid sequence of SEQ ID NO: 5 and the second sdABD of (v) comprises the amino acid sequence of SEQ ID NO: 5. In some embodiments, the first sdABD of (i) comprises the amino acid sequence of SEQ ID NO: 9 and the second sdABD of (v) comprises the amino acid sequence of SEQ ID NO: 9. In some embodiments, the first sdABD of (i) comprises the amino acid sequence of SEQ ID NO: 5 or SEQ ID NO 9 and the second sdABD of (v) comprises the amino acid sequence of SEQ ID NO: 96. In some embodiments, the first sdABD of (i) comprises the amino acid sequence of SEQ ID NO: 96 and the second sdABD of (v) comprises the amino acid sequence of SEQ ID NO: 5 or SEQ ID NO: 9.

In some embodiments, the first immune cell antigen is CD3 and the first constrained scFv domain of (iii) comprises a first VH comprising the amino acid sequence of SEQ ID NO: 205 fused to the N-terminus of a first VL comprising the amino acid sequence of SEQ ID NO: 206.

In some embodiments, the second immune cell antigen is CD28 and the second constrained scFv domain of (vii) comprises a second VH comprising the amino acid sequence of SEQ ID NO: 213 or SEQ ID NO: 216 fused to the N-terminus of a second VL comprising the amino acid sequence of SEQ ID NO: 214.

In some embodiments, the third sdABD of (ix) comprises the amino acid sequence of SEQ ID NO: 220.

In some embodiments, the protein comprises an amino acid sequence that is at least 85% (e.g., at least 85%, at least 90%, at least 95%, at least 98%, or at least 99% identical to any one of SEQ ID NOs: 234, 242, 244, 246, and 248. In some embodiments, the protein comprises an amino acid sequence of any one of SEQ ID NOs: 234, 242, 244, 246, and 248. In some embodiments, the protein consists of an amino acid sequence of any one of SEQ ID NOs: 234, 242, 244, 246, and 248.

### B. Configuration 2 (e.g., Pro1137 in Table 3)

In some embodiments, a protein described herein comprises, from N- to C-terminus:
(i) a first single domain antigen binding domain (sdABD) that binds to a first human target tumor antigen (TTA), wherein the first human TTA is selected from EGFR, HER2, Trop2, CA9, LyPD3, FOLR1, EpCAM, and B7H3;
(ii) a first domain linker (e.g., a non-cleavable linker);
(iii) a first constrained scFv domain comprising a first VH linked to the N-terminus of a first VL via a first CNCL (e.g., a CNCL that is 8 amino acids in length), wherein the first VH and first VL, if associated, are capable of binding a first human immune cell antigen (e.g., CD3), and wherein the first heavy chain variable region and the first light chain variable region are not associated in the first constrained scFv domain and the first constrained scFv domain does not bind to the first human immune cell antigen (e.g., CD3);
(iv) a first cleavable linker (e.g., a cleavable linker comprising a protease cleavage site provided in Table 3, such as a MMP9 cleavage site or variants thereof);
(v) a second single domain antigen binding domain (sdABD) that binds to a second human TTA, wherein the second human TTA is selected from EGFR, HER2, Trop2, CA9, LyPD3, FOLR1, EpCAM, and B7H3;
(vi) a second domain linker (e.g., a non-cleavable linker);
(vii) a second constrained scFv domain comprising a second VH linked to the C-terminus of a second VL via a second CNCL (e.g., a CNCL that is 8 amino acids in length), wherein the second VH and the second VL, if associated, are capable of binding a second human immune cell antigen (e.g., CD28), and wherein the first heavy chain variable region and the first light chain variable region are not associated in the second constrained scFv domain, and the second constrained scFv domain does not bind to the second human immune cell antigen (e.g., CD28);
(viii) a second cleavable linker (e.g., a cleavable linker comprising a protease cleavage site provided in Table 3, such as a MMP9 cleavage site or variants thereof); and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first TTA is the same as the second TTA. In some embodiments, the first TTA is the same as the second TTA, and the first sdABD binds a different epitope in the TTA than the second sdABD. In some embodiments, the first TTA is different from the second TTA. In some embodiments, the first TTA is EGFR and the second TTA is EGFR. In some embodiments, the first TTA is EGFR and the second TTA is HER2. In some embodiments, the first TTA is HER2 and the second TTA is EGFR. In some embodiments, the first TTA is HER2 and the second TTA is HER2.

In some embodiments, the first sdABD of (i) comprises the amino acid sequence of SEQ ID NO: 5, SEQ ID NO: 9, or SEQ ID NO: 96. In some embodiments, the second sdABD of (v) comprises the amino acid sequence of SEQ ID NO: 5, SEQ ID NO: 9, or SEQ ID NO: 96. In some embodiments, the first sdABD of (i) comprises the amino acid sequence of SEQ ID NO: 5 and the second sdABD of (v) comprises the amino acid sequence of SEQ ID NO: 5. In some embodiments, the first sdABD of (i) comprises the amino acid sequence of SEQ ID NO: 9 and the second sdABD of (v) comprises the amino acid sequence of SEQ ID NO: 9. In some embodiments, the first sdABD of (i) comprises the amino acid sequence of SEQ ID NO: 5 or SEQ ID NO 9 and the second sdABD of (v) comprises the amino acid sequence of SEQ ID NO: 96. In some embodiments, the first sdABD of (i) comprises the amino acid sequence of SEQ ID NO: 96 and the second sdABD of (v) comprises the amino acid sequence of SEQ ID NO: 5 or SEQ ID NO: 9.

In some embodiments, the first immune cell antigen is CD3 and the first constrained scFv domain of (iii) comprises a first VH comprising the amino acid sequence of SEQ ID NO: 205 fused to the N-terminus of a first VL comprising the amino acid sequence of SEQ ID NO: 206.

In some embodiments, the second immune cell antigen is CD28 and the second constrained scFv domain of (vii) comprises a second VH comprising the amino acid sequence of SEQ ID NO: 213 or SEQ ID NO: 216 fused to the C-terminus of a second VL comprising the amino acid sequence of SEQ ID NO: 214.

In some embodiments, the third sdABD of (ix) comprises the amino acid sequence of SEQ ID NO: 220.

In some embodiments, the protein comprises an amino acid sequence that is at least 85% (e.g., at least 85%, at least 90%, at least 95%, at least 98%, or at least 99%) identical to SEQ ID NO: 235. In some embodiments, the protein comprises an amino acid sequence of SEQ ID NO: 235. In some embodiments, the protein consists of an amino acid sequence of SEQ ID NO: 235.

### C. Configuration 3 (e.g., Pro1138 in Table 3)

In some embodiments, a protein described herein comprises, from N- to C-terminus:
(i) a first single domain antigen binding domain (sdABD) that binds to a first human target tumor antigen (TTA), wherein the first human TTA is selected from EGFR, HER2, Trop2, CA9, LyPD3, FOLR1, EpCAM, and B7H3;
(ii) a first domain linker (e.g., a non-cleavable linker);
(iii) a first constrained scFv domain comprising a first VH linked to the C-terminus of a first VL via a first CNCL (e.g., a CNCL that is 8 amino acids in length), wherein the first VH and first VL, if associated, are capable of binding a first human immune cell antigen (e.g., CD3), and wherein the first heavy chain variable region and the first light chain variable region are not associated in the first constrained scFv domain and the first constrained scFv domain does not bind to the first human immune cell antigen (e.g., CD3);
(iv) a first cleavable linker (e.g., a cleavable linker comprising a protease cleavage site provided in Table 3, such as a MMP9 cleavage site or variants thereof);
(v) a second single domain antigen binding domain (sdABD) that binds to a second human TTA, wherein the second human TTA is selected from EGFR, HER2, Trop2, CA9, LyPD3, FOLR1, EpCAM, and B7H3;
(vi) a second domain linker (e.g., a non-cleavable linker);
(vii) a second constrained scFv domain comprising a second VH linked to the N-terminus of a second VL via a second CNCL (e.g., a CNCL that is 8 amino acids in length), wherein the second VH and the second VL, if associated, are capable of binding a second human immune cell antigen (e.g., CD28), and wherein the first heavy chain variable region and the first light chain variable region are not associated in the second constrained scFv domain, and the second constrained scFv domain does not bind to the second human immune cell antigen (e.g., CD28);
(viii) a second cleavable linker (e.g., a cleavable linker comprising a protease cleavage site provided in Table 3, such as a MMP9 cleavage site or variants thereof); and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first TTA is the same as the second TTA. In some embodiments, the first TTA is the same as the second TTA, and the first sdABD binds a different epitope in the TTA than the second sdABD. In some embodiments, the first TTA is different from the second TTA. In some embodiments, the first TTA is EGFR and the second TTA is EGFR. In some embodiments, the first TTA is EGFR and the second TTA is HER2. In some embodiments, the first TTA is HER2 and the second TTA is EGFR. In some embodiments, the first TTA is HER2 and the second TTA is HER2.

In some embodiments, the first sdABD of (i) comprises the amino acid sequence of SEQ ID NO: 5, SEQ ID NO: 9, or SEQ ID NO: 96. In some embodiments, the second sdABD of (v) comprises the amino acid sequence of SEQ ID NO: 5, SEQ ID NO: 9, or SEQ ID NO: 96. In some embodiments, the first sdABD of (i) comprises the amino acid sequence of SEQ ID NO: 5 and the second sdABD of (v) comprises the amino acid sequence of SEQ ID NO: 5. In some embodiments, the first sdABD of (i) comprises the amino acid sequence of SEQ ID NO: 9 and the second sdABD of (v) comprises the amino acid sequence of SEQ ID NO: 9. In some embodiments, the first sdABD of (i) comprises the amino acid sequence of SEQ ID NO: 5 or SEQ ID NO 9 and the second sdABD of (v) comprises the amino acid sequence of SEQ ID NO: 96. In some embodiments, the first sdABD of (i) comprises the amino acid sequence of SEQ ID NO: 96 and the second sdABD of (v) comprises the amino acid sequence of SEQ ID NO: 5 or SEQ ID NO: 9.

In some embodiments, the first immune cell antigen is CD3 and the first constrained scFv domain of (iii) comprises a first VH comprising the amino acid sequence of SEQ ID NO: 205 fused to the C-terminus of a first VL comprising the amino acid sequence of SEQ ID NO: 206.

In some embodiments, the second immune cell antigen is CD28 and the second constrained scFv domain of (vii) comprises a second VH comprising the amino acid sequence of SEQ ID NO: 213 or SEQ ID NO: 216 fused to the N-terminus of a second VL comprising the amino acid sequence of SEQ ID NO: 214.

In some embodiments, the third sdABD of (ix) comprises the amino acid sequence of SEQ ID NO: 220.

In some embodiments, the protein comprises an amino acid sequence that is at least 85% (e.g., at least 85%, at least 90%, at least 95%, at least 98%, or at least 99%) identical to SEQ ID NO: 236. In some embodiments, the protein comprises an amino acid sequence of SEQ ID NO: 236. In some embodiments, the protein consists of an amino acid sequence of SEQ ID NO: 236.

### D. Configuration 4 (e.g., Pro1139 in Table 3)

In some embodiments, a protein described herein comprises, from N- to C-terminus:
(i) a first single domain antigen binding domain (sdABD) that binds to a first human target tumor antigen (TTA), wherein the first human TTA is selected from EGFR, HER2, Trop2, CA9, LyPD3, FOLR1, EpCAM, and B7H3;
(ii) a first domain linker (e.g., a non-cleavable linker);
(iii) a first constrained scFv domain comprising a first VH linked to the C-terminus of a first VL via a first CNCL (e.g., a CNCL that is 8 amino acids in length), wherein the first VH and first VL, if associated, are capable of binding a first human immune cell antigen (e.g., CD3), and wherein the first heavy chain variable region and the first light chain variable region are not associated in the first constrained scFv domain and the first constrained scFv domain does not bind to the first human immune cell antigen (e.g., CD3);
(iv) a first cleavable linker (e.g., a cleavable linker comprising a protease cleavage site provided in Table 3, such as a MMP9 cleavage site or variants thereof);
(v) a second single domain antigen binding domain (sdABD) that binds to a second human TTA, wherein the second human TTA is selected from EGFR, HER2, Trop2, CA9, LyPD3, FOLR1, EpCAM, and B7H3;
(vi) a second domain linker (e.g., a non-cleavable linker);
(vii) a second constrained scFv domain comprising a second VH linked to the C-terminus of a second VL via a second CNCL (e.g., a CNCL that is 8 amino acids in length), wherein the second VH and the second VL, if associated, are capable of binding a second human immune cell antigen (e.g., CD28), and wherein the first heavy chain variable region and the first light chain variable region are not associated in the second constrained scFv domain, and the second constrained scFv domain does not bind to the second human immune cell antigen (e.g., CD28);
(viii) a second cleavable linker (e.g., a cleavable linker comprising a protease cleavage site provided in Table 3, such as a MMP9 cleavage site or variants thereof); and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first TTA is the same as the second TTA. In some embodiments, the first TTA is the same as the second TTA, and the first sdABD binds a different epitope in the TTA than the second sdABD. In some embodiments, the first TTA is different from the second TTA. In some embodiments, the first TTA is EGFR and the second TTA is EGFR. In some embodiments, the first TTA is EGFR and the second TTA is HER2. In some embodiments, the first TTA is HER2 and the second TTA is EGFR. In some embodiments, the first TTA is HER2 and the second TTA is HER2.

In some embodiments, the first sdABD of (i) comprises the amino acid sequence of SEQ ID NO: 5, SEQ ID NO: 9, or SEQ ID NO: 96. In some embodiments, the second sdABD of (v) comprises the amino acid sequence of SEQ ID NO: 5, SEQ ID NO: 9, or SEQ ID NO: 96. In some embodiments, the first sdABD of (i) comprises the amino acid sequence of SEQ ID NO: 5 and the second sdABD of (v) comprises the amino acid sequence of SEQ ID NO: 5. In some embodiments, the first sdABD of (i) comprises the amino acid sequence of SEQ ID NO: 9 and the second sdABD of (v) comprises the amino acid sequence of SEQ ID NO: 9. In some embodiments, the first sdABD of (i) comprises the amino acid sequence of SEQ ID NO: 5 or SEQ ID NO 9 and the second sdABD of (v) comprises the amino acid sequence of SEQ ID NO: 96. In some embodiments, the first sdABD of (i) comprises the amino acid sequence of SEQ ID NO: 96 and the second sdABD of (v) comprises the amino acid sequence of SEQ ID NO: 5 or SEQ ID NO: 9.

In some embodiments, the first immune cell antigen is CD3 and the first constrained scFv domain of (iii) comprises a first VH comprising the amino acid sequence of SEQ ID NO: 205 fused to the C-terminus of a first VL comprising the amino acid sequence of SEQ ID NO: 206.

In some embodiments, the second immune cell antigen is CD28 and the second constrained scFv domain of (vii) comprises a second VH comprising the amino acid sequence of SEQ ID NO: 213 or SEQ ID NO: 216 fused to the C-terminus of a second VL comprising the amino acid sequence of SEQ ID NO: 214.

In some embodiments, the third sdABD of (ix) comprises the amino acid sequence of SEQ ID NO: 220.

In some embodiments, the protein comprises an amino acid sequence that is at least 85% (e.g., at least 85%, at least 90%, at least 95%, at least 98%, at least 99% identical to SEQ ID NO: 239. In some embodiments, the protein comprises an amino acid sequence of SEQ ID NO: 239. In some embodiments, the protein consists of an amino acid sequence of SEQ ID NO: 239.

### E. Configuration 5 (e.g., Pro1140, Pro1192, Pro1266, Pro1268, and Pro1270 in Table 3)

In some embodiments, a protein described herein comprises, from N- to C-terminus:
(i) a first single domain antigen binding domain (sdABD) that binds to a first human target tumor antigen (TTA), wherein the first human TTA is selected from EGFR, HER2, Trop2, CA9, LyPD3, FOLR1, EpCAM, and B7H3;
(ii) a first domain linker (e.g., a non-cleavable linker);
(iii) a first constrained scFv domain comprising a first VH linked to the N-terminus of a first VL via a first CNCL (e.g., a CNCL that is 8 amino acids in length), wherein the first VH and first VL, if associated, are capable of binding a first human immune cell antigen (e.g., CD28), and wherein the first heavy chain variable region and the first light chain variable region are not associated in the first constrained scFv domain and the first constrained scFv domain does not bind to the first human immune cell antigen (e.g., CD28);
(iv) a first cleavable linker (e.g., a cleavable linker comprising a protease cleavage site provided in Table 3, such as a MMP9 cleavage site or variants thereof);
(v) a second single domain antigen binding domain (sdABD) that binds to a second human TTA, wherein the second human TTA is selected from EGFR, HER2, Trop2, CA9, LyPD3, FOLR1, EpCAM, and B7H3;
(vi) a second domain linker (e.g., a non-cleavable linker);
(vii) a second constrained scFv domain comprising a second VH linked to the N-terminus of a second VL via a second CNCL (e.g., a CNCL that is 8 amino acids in length), wherein the second VH and the second VL, if associated, are capable of binding a second human immune cell antigen (e.g., CD3), and wherein the first heavy chain variable region and the first light chain variable region are not associated in the second constrained scFv domain, and the second constrained scFv domain does not bind to the second human immune cell antigen (e.g., CD3);
(viii) a second cleavable linker (e.g., a cleavable linker comprising a protease cleavage site provided in Table 3, such as a MMP9 cleavage site or variants thereof); and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first TTA is the same as the second TTA. In some embodiments, the first TTA is the same as the second TTA, and the first sdABD binds a different epitope in the TTA than the second sdABD. In some embodiments, the first TTA is different from the second TTA. In some embodiments, the first TTA is EGFR and the second TTA is EGFR. In some embodiments, the first TTA is EGFR and the second TTA is HER2. In some embodiments, the first TTA is HER2 and the second TTA is EGFR. In some embodiments, the first TTA is HER2 and the second TTA is HER2.

In some embodiments, the first sdABD of (i) comprises the amino acid sequence of SEQ ID NO: 5, SEQ ID NO: 9, or SEQ ID NO: 96. In some embodiments, the second sdABD of (v) comprises the amino acid sequence of SEQ ID NO: 5, SEQ ID NO: 9, or SEQ ID NO: 96. In some embodiments, the first sdABD of (i) comprises the amino acid sequence of SEQ ID NO: 5 and the second sdABD of (v) comprises the amino acid sequence of SEQ ID NO: 5. In some embodiments, the first sdABD of (i) comprises the amino acid sequence of SEQ ID NO: 9 and the second sdABD of (v) comprises the amino acid sequence of SEQ ID NO: 9. In some embodiments, the first sdABD of (i) comprises the amino acid sequence of SEQ ID NO: 5 or SEQ ID NO 9 and the second sdABD of (v) comprises the amino acid sequence of SEQ ID NO: 96. In some embodiments, the first sdABD of (i) comprises the amino acid sequence of SEQ ID NO: 96 and the second sdABD of (v) comprises the amino acid sequence of SEQ ID NO: 5 or SEQ ID NO: 9.

In some embodiments, the first immune cell antigen is CD28 and the first constrained scFv domain of (iii) comprises a first VH comprising the amino acid sequence of SEQ ID NO: 213 or SEQ ID NO: 216 fused to the N-terminus of a first VL comprising the amino acid sequence of SEQ ID NO: 214.

In some embodiments, the second immune cell antigen is CD3 and the second constrained scFv domain of (vii) comprises a second VH comprising the amino acid sequence of SEQ ID NO: 205 fused to the N-terminus of a second VL comprising the amino acid sequence of SEQ ID NO: 206.

In some embodiments, the third sdABD of (ix) comprises the amino acid sequence of SEQ ID NO: 220.

In some embodiments, the protein comprises an amino acid sequence that is at least 85% (e.g., at least 85%, at least 90%, at least 95%, at least 98%, or at least 99%) identical to any one of SEQ ID NOs: 238, 243, 245, 247, and 249. In some embodiments, the protein comprises an amino acid sequence of any one of SEQ ID NOs: Pro1140, Pro1192, Pro1266, Pro1268, and Pro1270. In some embodiments, the protein consists of an amino acid sequence of any one of SEQ ID NOs: X.

### F. Configuration 6 (e.g., Pro1141 in Table 3)

In some embodiments, a protein described herein comprises, from N- to C-terminus:
(i) a first single domain antigen binding domain (sdABD) that binds to a first human target tumor antigen (TTA), wherein the first human TTA is selected from EGFR, HER2, Trop2, CA9, LyPD3, FOLR1, EpCAM, and B7H3;
(ii) a first domain linker (e.g., a non-cleavable linker);
(iii) a first constrained scFv domain comprising a first VH linked to the C-terminus of a first VL via a first CNCL (e.g., a CNCL that is 8 amino acids in length), wherein the first VH and first VL, if associated, are capable of binding a first human immune cell antigen (e.g., CD28), and wherein the first heavy chain variable region and the first light chain variable region are not associated in the first constrained scFv domain and the first constrained scFv domain does not bind to the first human immune cell antigen (e.g., CD28);
(iv) a first cleavable linker (e.g., a cleavable linker comprising a protease cleavage site provided in Table 3, such as a MMP9 cleavage site or variants thereof);
(v) a second single domain antigen binding domain (sdABD) that binds to a second human TTA, wherein the second human TTA is selected from the group consisting of EGFR, HER2, Trop2, CA9, LyPD3, FOLR1, EpCAM, and B7H3;
(vi) a second domain linker (e.g., a non-cleavable linker);
(vii) a second constrained scFv domain comprising a second VH linked to the N-terminus of a second VL via a second CNCL (e.g., a CNCL that is 8 amino acids in length), wherein the second VH and the second VL, if associated, are capable of binding a second human immune cell antigen (e.g., CD3), and wherein the first heavy chain variable region and the first light chain variable region are not associated in the second constrained scFv domain, and the second constrained scFv domain does not bind to the second human immune cell antigen (e.g., CD3);
(viii) a second cleavable linker (e.g., a cleavable linker comprising a protease cleavage site provided in Table 3, such as a MMP9 cleavage site or variants thereof); and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first TTA is the same as the second TTA. In some embodiments, the first TTA is the same as the second TTA, and the first sdABD binds a different epitope in the TTA than the second sdABD. In some embodiments, the first TTA is different from the second TTA. In some embodiments, the first TTA is EGFR and the second TTA is EGFR. In some embodiments, the first TTA is EGFR and the second TTA is HER2. In some embodiments, the first TTA is HER2 and the second TTA is EGFR. In some embodiments, the first TTA is HER2 and the second TTA is HER2.

In some embodiments, the first sdABD of (i) comprises the amino acid sequence of SEQ ID NO: 5, SEQ ID NO: 9, or SEQ ID NO: 96. In some embodiments, the second sdABD of (v) comprises the amino acid sequence of SEQ ID NO: 5, SEQ ID NO: 9, or SEQ ID NO: 96. In some embodiments, the first sdABD of (i) comprises the amino acid sequence of SEQ ID NO: 5 and the second sdABD of (v) comprises the amino acid sequence of SEQ ID NO: 5. In some embodiments, the first sdABD of (i) comprises the amino acid sequence of SEQ ID NO: 9 and the second sdABD of (v) comprises the amino acid sequence of SEQ ID NO: 9. In some embodiments, the first sdABD of (i) comprises the amino acid sequence of SEQ ID NO: 5 or SEQ ID NO 9 and the second sdABD of (v) comprises the amino acid sequence of SEQ ID NO: 96. In some embodiments, the first sdABD of (i) comprises the amino acid sequence of SEQ ID NO: 96 and the second sdABD of (v) comprises the amino acid sequence of SEQ ID NO: 5 or SEQ ID NO: 9.

In some embodiments, the first immune cell antigen is CD28 and the first constrained scFv domain of (iii) comprises a first VH comprising the amino acid sequence of SEQ ID NO: 213 or SEQ ID NO: 216 fused to the C-terminus of a first VL comprising the amino acid sequence of SEQ ID NO: 214.

In some embodiments, the second immune cell antigen is CD3 and the second constrained scFv domain of (vii) comprises a second VH comprising the amino acid sequence of SEQ ID NO: 205 fused to the N-terminus of a second VL comprising the amino acid sequence of SEQ ID NO: 206.

In some embodiments, the third sdABD of (ix) comprises the amino acid sequence of SEQ ID NO: 220.

In some embodiments, the protein comprises an amino acid sequence that is at least 85% (e.g., at least 85%, at least 90%, at least 95%, at least 98%, or at least 99%) identical to SEQ ID NO: 239. In some embodiments, the protein comprises an amino acid sequence of SEQ ID NO: 239. In some embodiments, the protein consists of an amino acid sequence of SEQ ID NO: 239.

### G. Configuration (e.g., Pro1142 in Table 3)

In some embodiments, a protein described herein comprises, from N- to C-terminus:
(i) a first single domain antigen binding domain (sdABD) that binds to a first human target tumor antigen (TTA), wherein the first human TTA is selected from EGFR, HER2, Trop2, CA9, LyPD3, FOLR1, EpCAM, and B7H3;
(ii) a first domain linker (e.g., a non-cleavable linker);
(iii) a first constrained scFv domain comprising a first VH linked to the N-terminus of a first VL via a first CNCL (e.g., a CNCL that is 8 amino acids in length), wherein the first VH and first VL, if associated, are capable of binding a first human immune cell antigen (e.g., CD28), and wherein the first heavy chain variable region and the first light chain variable region are not associated in the first constrained scFv domain and the first constrained scFv domain does not bind to the first human immune cell antigen (e.g., CD28);
(iv) a first cleavable linker (e.g., a cleavable linker comprising a protease cleavage site provided in Table 3, such as a MMP9 cleavage site or variants thereof);
(v) a second single domain antigen binding domain (sdABD) that binds to a second human TTA, wherein the second human TTA is selected from EGFR, HER2, Trop2, CA9, LyPD3, FOLR1, EpCAM, and B7H3;
(vi) a second domain linker (e.g., a non-cleavable linker);
(vii) a second constrained scFv domain comprising a second VH linked to the C-terminus of a second VL via a second CNCL (e.g., a CNCL that is 8 amino acids in length), wherein the second VH and the second VL, if associated, are capable of binding a second human immune cell antigen (e.g., CD3), and wherein the first heavy chain variable region and the first light chain variable region are not associated in the second constrained scFv domain, and the second constrained scFv domain does not bind to the second human immune cell antigen (e.g., CD3);
(viii) a second cleavable linker (e.g., a cleavable linker comprising a protease cleavage site provided in Table 3, such as a MMP9 cleavage site or variants thereof); and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first TTA is the same as the second TTA. In some embodiments, the first TTA is the same as the second TTA, and the first sdABD binds a different epitope in the TTA than the second sdABD. In some embodiments, the first TTA is different from the second TTA. In some embodiments, the first TTA is EGFR and the second TTA is EGFR. In some embodiments, the first TTA is EGFR and the second TTA is HER2. In some embodiments, the first TTA is HER2 and the second TTA is EGFR. In some embodiments, the first TTA is HER2 and the second TTA is HER2.

In some embodiments, the first sdABD of (i) comprises the amino acid sequence of SEQ ID NO: 5, SEQ ID NO: 9, or SEQ ID NO: 96. In some embodiments, the second sdABD of (v) comprises the amino acid sequence of SEQ ID NO: 5, SEQ ID NO: 9, or SEQ ID NO: 96. In some embodiments, the first sdABD of (i) comprises the amino acid sequence of SEQ ID NO: 5 and the second sdABD of (v) comprises the amino acid sequence of SEQ ID NO: 5. In some embodiments, the first sdABD of (i) comprises the amino acid sequence of SEQ ID NO: 9 and the second sdABD of (v) comprises the amino acid sequence of SEQ ID NO: 9. In some embodiments, the first sdABD of (i) comprises the amino acid sequence of SEQ ID NO: 5 or SEQ ID NO 9 and the second sdABD of (v) comprises the amino acid sequence of SEQ ID NO: 96. In some embodiments, the first sdABD of (i) comprises the amino acid sequence of SEQ ID NO: 96 and the second sdABD of (v) comprises the amino acid sequence of SEQ ID NO: 5 or SEQ ID NO: 9.

In some embodiments, the first immune cell antigen is CD28 and the first constrained scFv domain of (iii) comprises a first VH comprising the amino acid sequence of SEQ ID NO: 213 or SEQ ID NO: 216 fused to the N-terminus of a first VL comprising the amino acid sequence of SEQ ID NO: 214.

In some embodiments, the second immune cell antigen is CD3 and the second constrained scFv domain of (vii) comprises a second VH comprising the amino acid sequence of SEQ ID NO: 205 fused to the C-terminus of a second VL comprising the amino acid sequence of SEQ ID NO: 206.

In some embodiments, the third sdABD of (ix) comprises the amino acid sequence of SEQ ID NO: 220.

In some embodiments, the protein comprises an amino acid sequence that is at least 85% (e.g., at least 85%, at least 90%, at least 95%, at least 98%, or at least 99%) identical to SEQ ID NO: 240. In some embodiments, the protein comprises an amino acid sequence of SEQ ID NO: 240. In some embodiments, the protein consists of an amino acid sequence of SEQ ID NO: 240.

### H. Configuration (e.g., Pro1143 in Table 3)

In some embodiments, a protein described herein comprises, from N- to C-terminus:
(i) a first single domain antigen binding domain (sdABD) that binds to a first human target tumor antigen (TTA), wherein the first human TTA is selected from EGFR, HER2, Trop2, CA9, LyPD3, FOLR1, EpCAM, and B7H3;
(ii) a first domain linker (e.g., a non-cleavable linker);
(iii) a first constrained scFv domain comprising a first VH linked to the C-terminus of a first VL via a first CNCL (e.g., a CNCL that is 8 amino acids in length), wherein the first VH and first VL, if associated, are capable of binding a first human immune cell antigen (e.g., CD28), and wherein the first heavy chain variable region and the first light chain variable region are not associated in the first constrained scFv domain and the first constrained scFv domain does not bind to the first human immune cell antigen (e.g., CD28);
(iv) a first cleavable linker (e.g., a cleavable linker comprising a protease cleavage site provided in Table 3, such as a MMP9 cleavage site or variants thereof);
(v) a second single domain antigen binding domain (sdABD) that binds to a second human TTA, wherein the second human TTA is selected from the group consisting of EGFR, HER2, Trop2, CA9, LyPD3, FOLR1, EpCAM, and B7H3;
(vi) a second domain linker (e.g., a non-cleavable linker);
(vii) a second constrained scFv domain comprising a second VH linked to the C-terminus of a second VL via a second CNCL (e.g., a CNCL that is 8 amino acids in length), wherein the second VH and the second VL, if associated, are capable of binding a second human immune cell antigen (e.g., CD3), and wherein the first heavy chain variable region and the first light chain variable region are not associated in the second constrained scFv domain, and the second constrained scFv domain does not bind to the second human immune cell antigen (e.g., CD3);
(viii) a second cleavable linker (e.g., a cleavable linker comprising a protease cleavage site provided in Table 3, such as a MMP9 cleavage site or variants thereof); and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first TTA is the same as the second TTA. In some embodiments, the first TTA is the same as the second TTA, and the first sdABD binds a different epitope in the TTA than the second sdABD. In some embodiments, the first TTA is different from the second TTA. In some embodiments, the first TTA is EGFR and the second TTA is EGFR. In some embodiments, the first TTA is EGFR and the second TTA is HER2. In some embodiments, the first TTA is HER2 and the second TTA is EGFR. In some embodiments, the first TTA is HER2 and the second TTA is HER2.

In some embodiments, the first sdABD of (i) comprises the amino acid sequence of SEQ ID NO: 5, SEQ ID NO: 9, or SEQ ID NO: 96. In some embodiments, the second sdABD of (v) comprises the amino acid sequence of SEQ ID NO: 5, SEQ ID NO: 9, or SEQ ID NO: 96. In some embodiments, the first sdABD of (i) comprises the amino acid sequence of SEQ ID NO: 5 and the second sdABD of (v) comprises the amino acid sequence of SEQ ID NO: 5. In some embodiments, the first sdABD of (i) comprises the amino acid sequence of SEQ ID NO: 9 and the second sdABD of (v) comprises the amino acid sequence of SEQ ID NO: 9. In some embodiments, the first sdABD of (i) comprises the amino acid sequence of SEQ ID NO: 5 or SEQ ID NO 9 and the second sdABD of (v) comprises the amino acid sequence of SEQ ID NO: 96. In some embodiments, the first sdABD of (i) comprises the amino acid sequence of SEQ ID NO: 96 and the second sdABD of (v) comprises the amino acid sequence of SEQ ID NO: 5 or SEQ ID NO: 9.

In some embodiments, the first immune cell antigen is CD28 and the first constrained scFv domain of (iii) comprises a first VH comprising the amino acid sequence of SEQ ID NO: 213 or SEQ ID NO: 216 fused to the C-terminus of a first VL comprising the amino acid sequence of SEQ ID NO: 214.

In some embodiments, the second immune cell antigen is CD3 and the second constrained scFv domain of (vii) comprises a second VH comprising the amino acid sequence of SEQ ID NO: 205 fused to the C-terminus of a second VL comprising the amino acid sequence of SEQ ID NO: 206.

In some embodiments, the third sdABD of (ix) comprises the amino acid sequence of SEQ ID NO: 220.

In some embodiments, the protein comprises an amino acid sequence that is at least 85% (e.g., at least 85%, at least 90%, at least 95%, at least 98%, or at least 99%) identical to SEQ ID NO: 241. In some embodiments, the protein comprises an amino acid sequence of SEQ ID NO: 241. In some embodiments, the protein consists of an amino acid sequence of SEQ ID NO: 241.

### III. Production Methods

In some aspects, the present disclosure provides nucleic acid molecules comprising a nucleotide sequence encoding any one of the proteins described herein. In some embodiments, the nucleic acid molecule is a vector. In some embodiments, the nucleic acid molecule is an expression vector (e.g., an expression vector suitable for expression of the protein in mammalian cells such as human cells).

As will be appreciated by those in the art, the nucleic acid compositions will depend on the format of the proteins. In general, a protein described herein is encoded by a single nucleic acid molecule in a single expression vector for production.

As is known in the art, the nucleic acids encoding the components of the protein can be incorporated into expression vectors, and depending on the host cells used to produce the prodrug compositions disclosed herein. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors encoding the prodrugs disclosed herein are then transformed into any number of different types of host cells known in the art, including mammalian, bacterial, yeast, insect and/or fungal cells, with mammalian cells (e.g., CHO cells, 293 cells), finding use in many embodiments.

The prodrug compositions described herein are made by culturing host cells comprising the expression vector(s). Once produced, traditional antibody purification steps are done, including a Protein A affinity chromatography step and/or an ion exchange chromatography step.

In some embodiments, activities of proteins described herein may be determined via Co-Stimulation Assays. For example, human T-cells isolated from healthy donors may be pre-labeled with Cell Trace Violet then incubated with antibodies or proteins at varying concentrations along with target bearing beads or on plates. Proliferation can be measured by proportional loss of staining by FACS.

In some embodiments, activities of proteins described herein may be determined via T-cell dependent cellular cytotoxicity Assay. For example, human T-cells isolated from healthy donors can be incubated with proteins at varying concentrations along with target bearing tumor cell lines that have been pre-labelled with firefly luciferase. Cytotoxicity can be measured by observing changes in luciferase levels using a luminometer.

### IV. Compositions and Methods of Use

The present disclosure, in some aspects, provide compositions comprising any one or more of the proteins described herein. In some embodiments, compositions comprising the proteins described herein are prepared for storage by mixing the proteins having the desired degree of purity with optional pharmaceutically acceptable carriers, excipients or stabilizers (as generally outlined in Remington's Pharmaceutical Sciences 16th edition, Osol, A. Ed. [1980]), in the form of lyophilized formulations or aqueous solutions. In some embodiments, the composition comprising the proteins described herein are prepared for administration, e.g., to a subject for treating a disease (e.g., cancer).

Other aspects of the present disclosure provide methods of treating cancer by administering to a subject in need thereof a composition comprising proteins described herein. In some embodiments, a composition for administration to a subject described herein comprises a first protein and a second protein, wherein the first protein and the second protein are the same (identical construct), wherein each of the first protein and the second protein comprises, from N- to C-terminus:
(i) a first single domain antigen binding domain (sdABD) that binds to a first human target tumor antigen (TTA);
(ii) a first domain linker;
(iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first human immune cell antigen, and wherein the first heavy chain variable region and the first light chain variable region are not associated in the first constrained scFv domain and the first constrained scFv domain does not bind to the first human immune cell antigen;
(iv) a first cleavable linker;
(v) a second single domain antigen binding domain (sdABD) that binds to a second human target tumor antigen (TTA);
(vi) a second domain linker;
(vii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second human immune cell antigen, and wherein the first heavy chain variable region and the first light chain variable region are not associated in the second constrained scFv domain, and the second constrained scFv domain does not bind to the second human immune cell antigen;
(viii) a second cleavable linker; and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen.

In some embodiments, the first immune cell antigen is CD3 or CD28. In some embodiments, the second immune cell antigen is CD3 or CD28. In some embodiments, the first immune cell antigen is CD3 and the second immune cell antigen is CD28. In some embodiments, the first immune cell antigen is CD28 and the second immune cell antigen is CD3.

In some embodiments, the first and the second cleavable linker each comprises a cleavage site of a protease that is in a tumor microenvironment of the subject (e.g., a MMP9 cleavage site).

In some embodiments, upon cleavage of the first cleavable linker of (iv) and the second cleavable linker of (viii) in the first protein and the second protein: the first heavy chain variable region of the first protein associates with the first light chain variable region of the second protein, forming an active binding site that binds to the first human immune cell antigen; the first light chain variable region of the first protein associates with the first heavy chain variable region of the second protein, forming an active binding site that binds that binds to the first human immune cell antigen; the second heavy chain variable region of the first protein associates with the second light chain variable region of the second protein, forming an active binding site that binds to the second human immune cell antigen; and the second light chain variable region of the first protein associates with the second heavy chain variable region of the second protein, forming an active binding site that binds that binds to the second human immune cell antigen. As such, the cleavage fragments of the prodrug (inactive) proteins assemble into two homodimers (a first homodimer and a second homodimer), each homodimer being a bi-specific molecule capable of binding a TTA and an immune cell antigen.

In some embodiments, a first homodimer forms via the homodimerization of a first polypeptide (cleavage product of the prodrug protein) comprising:
(i) a first single domain antigen binding domain (sdABD) that binds to a first human target tumor antigen (TTA);
(ii) a first domain linker;
(iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first human immune cell antigen, and wherein the first heavy chain variable region and the first light chain variable region are not associated in the first constrained scFv domain and the first constrained scFv domain does not bind to the first human immune cell antigen;
wherein in the first homodimer, the first VH of one polypeptide associates with the first VL of the other polypeptide, and the first VL of one polypeptide associates with the first VH of the other polypeptide, forming two active variable fragments (Fvs) each capable of binding to the first immune antigen.

In some embodiments, a second homodimer forms via the homodimerization of a second polypeptide (cleavage product of the prodrug protein) comprising:
(i) a second single domain antigen binding domain (sdABD) that binds to a second human target tumor antigen (TTA);
(ii) a second domain linker;
(iii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second human immune cell antigen, and wherein the first heavy chain variable region and the first light chain variable region are not associated in the second constrained scFv domain, and the second constrained scFv domain does not bind to the second human immune cell antigen;
wherein in the second homodimer, the second VH of one polypeptide associates with the second VL of the other polypeptide, and the second VL of one polypeptide associates with the second VH of the other polypeptide, forming two active variable fragments (Fvs) each capable of binding to the second immune antigen.

In some embodiments, a composition described herein is administered to a subject in need thereof via one or more suitable routes of administration, using one or more of a variety of methods known in the art. The route and/or mode of administration will vary depending upon the desired results. An acceptable route of administration may refer to any administration pathway known in the art which may be taken into consideration by a clinician in conjunction with the intended therapeutic use, such as by parenteral administration which is typically associated with injection at or in communication with the intended site of action (e.g., intravenous administration).

In some embodiments, a composition is administered to the same subject once or on multiple occasions.

As used herein, the terms “treat,” “treating,” or “treatment”, and grammatical variants thereof, have the same meaning as commonly understood by those of ordinary skill in the art. In some embodiments, these terms refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. For the purposes of this disclosure, beneficial or desired clinical results include, but are not limited to, reduction or alleviation of symptoms, diminishment of extent of disease, stabilization (e.g., not worsening) of state of disease, delay or slowing of disease progression, amelioration or palliation of the disease state, and remission (whether partial or total), whether detectable or undetectable. “Treat,” “treating,” or “treatment” can also include prolonging survival relative to expected survival time if not receiving treatment. A subject (e.g., a human) in need of treatment may thus be a subject already afflicted with the disease or disorder in question. The terms “treat,” “treating,” or “treatment” include inhibition or reduction of an increase in severity of a pathological state or symptoms relative to the absence of treatment, and is not necessarily meant to imply complete cessation of the relevant disease or condition.

In some embodiments, a composition is administered to a subject in an effective amount. An “effective amount” is an amount effective for treating and/or preventing a disease, disorder, or condition as disclosed herein. In some embodiments, an effective amount is an amount or dose of a composition (e.g., a therapeutic composition, compound, or agent) that produces at least one desired therapeutic effect in a subject, such as preventing or treating a target condition or beneficially alleviating a symptom associated with the condition. This amount will vary depending upon a variety of factors, including but not limited to the characteristics of the therapeutic composition (including activity, pharmacokinetics, pharmacodynamics, and bioavailability), the physiological condition of the subject (including age, sex, disease type, disease stage, general physical condition, responsiveness to a given dosage, and type of medication), the nature of the pharmaceutically acceptable carrier or carriers in the formulation, and the route of administration. One skilled in the clinical and pharmacological arts can assess an effective amount, for example, by monitoring a subject's response to administration of a composition and adjusting the dosage accordingly (see e.g., Remington: The Science and Practice of Pharmacy (Gennaro A, ed., Mack Publishing Co., Easton, PA, U.S., 19th ed., 1995)).

As used herein, the term “subject” refers to any organism, commonly mammalian subjects, such as humans and animals. The terms “subject” and “patient” are used interchangeably. In some embodiments, the subject is a mammal, such as a primate (e.g., a human or non-human primate), or a livestock animal (e.g., cow, horse, pig, sheep, goat, etc.).

### EXAMPLES

### Example 1: Conditionally Active Multi-Specific Immune Cell Engagers Comprising CD3 and CD28 Immune Cell Binding Domains

This example demonstrates that active homodimers produced by cleavage of the conditionally active multi-specific proteins described herein are capable of inducing T cell proliferation and killing cancer cells.

Recent studies have shown that T cells can be recruited to tumors using bispecific molecules comprising a tumor targeted antigen binding domain and an immune cell binding domain. These bispecific molecules have at least two challenges in their implementation in the clinic. First, many bispecific molecules are active outside of the tumor microenvironment resulting in negative side effects. Second, recruited T cells can become exhausted, which lessons the T-cell ability to kill cancer cells.

Here, conditionally active multi-specific proteins have been designed and tested to overcome these challenges. These multi-specific proteins are prodrugs that become activated in the tumor microenvironment when cleaved by a tumor associated protease. These proteins comprise a targeted tumor antigen antibody binding domain (TTA-ABD) and conditionally active αCD3 and αCD28 immune cell binding domains. As shown below, binding of CD3 and CD28 results increased T cell proliferation and cancer killing potency.

In general, the CD3 binding domain (“Fv”) is in a constrained format, wherein a linker between the CD3 variable heavy domain and the CD3 variable light domain that traditionally form an Fv is too short to allow the two active variable domains to bind each other intramolecularly; this is referred to as “constrained non-cleavable linker” (CNCL). This is referred to as a “anti-CD3 constrained Fv”. Similarly, in the prodrug (e.g., uncleaved) format, the protein also comprises an anti-CD28 Fv domain which is also in a constrained format, with a CNCL linker between the anti-CD28 variable heavy domain and the anti-CD28 variable light domain that is too short to allow the two active variable domains to bind each other intramolecularly. This is referred to as a “anti-CD28 constrained Fv”.

Variable heavy (VH) and variable light (VL) domains prefer to be paired. Without being bound by theory, it is believed herein, as supported by the in vitro and in vivo data presented and described below, that the protein of FIG. 1A intramolecularly assembles such that the anti-CD3 constrained Fv VH interacts with the anti-CD28 constrained Fv VL to form a first “non-productive Fv” or “inert Fv”, and the anti-CD28 constrained Fv VH interacts with the anti-CD3 constrained Fv VL to form a second “non-productive Fv” or “inert Fv”, which, while stable, do not bind to either antigen (FIG. 1D). That is, while these inert Fvs contain both a VH and VL, they are incapable of binding an antigen because the VH has CDRs with specificity to one antigen (e.g., CD3) and the VL has CDRs with specificity to a different antigen (e.g., CD28).

In the presence of tumor proteases, the cleavage site(s) are cleaved, allowing intermolecular homodimerization of identical constrained Fvs to form an active anti-CD3 binding Fv and an active anti-CD28 Fv, respectively (FIGS. 1B-1C). That is, the inert Fvs come apart and then form Fvs that can bind antigens and are thus antigen binding domains, e.g., either to CD3 or to CD28. Cleavage may occur before or after the protein is bound to a target cell through the TTA-ABD.

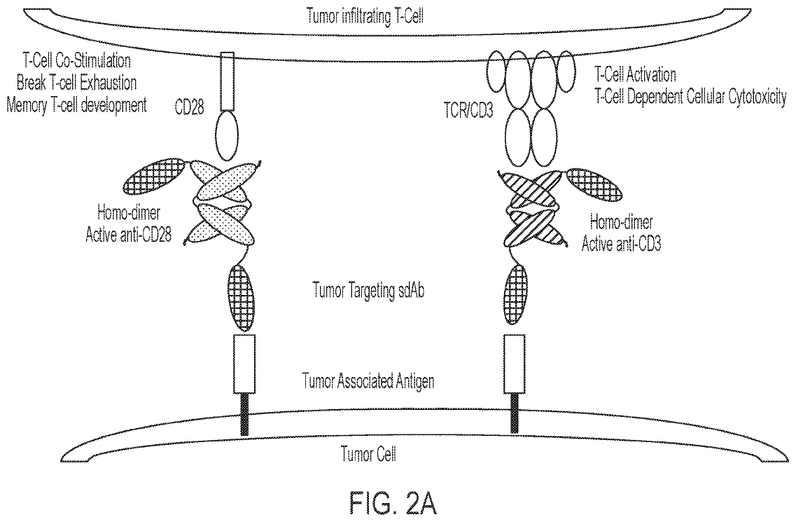

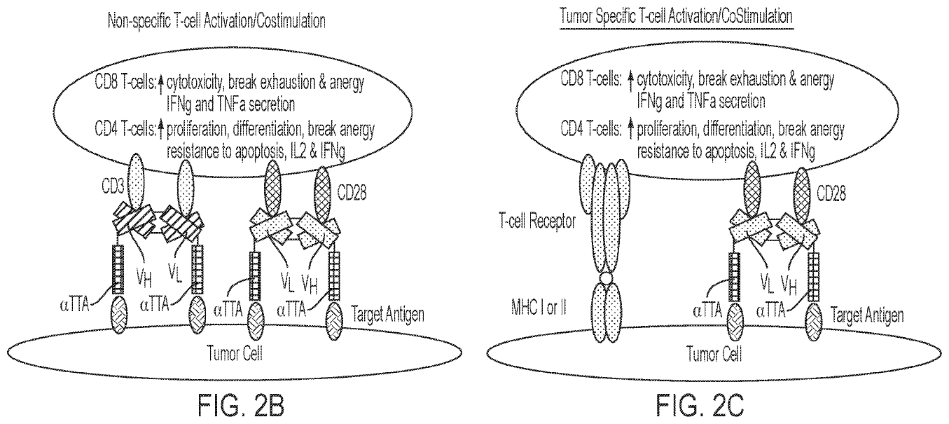

FIGS. 2A and 2B show the mode of action of the cleaved (activated) proteins. In FIG. 2A, the two new constructs, e.g., the homodimeric anti-CD3 and the homodimeric anti-CD28, can bind to a target T cell. In this case, T cells that are CD8+ can be activated for increased toxicity, to break exhaustion, and increase either or both of IFNγ and/or TNFα secretion. Similarly, for T-cells that are CD4+, these cells can be activated to increase proliferation, increase differentiation, increase resistance to apoptosis, and increase either or both of IFNγ and/or IL-2 secretion. Suitable assays for determining the activity of the T cell engagers outlined herein include TDCC assays and Jurkat NFAT Luc assays.

In FIG. 2B, the anti-CD28 homodimers can bind to a different target cell. In some cases, these can be NK cells, to enhance cytotoxicity and IFNγ secretion. In some cases, these can be dendritic cells or macrophages, to stimulate the cells and/or increase maturation. In some cases, these can be tumor endothelial cells to enhance T-cell recruitment. Suitable assays for assessing CD28 activity include HEK CD28 assays, NFAT Luc assays, NK cell assays for IL2 and IFNγ secretion.

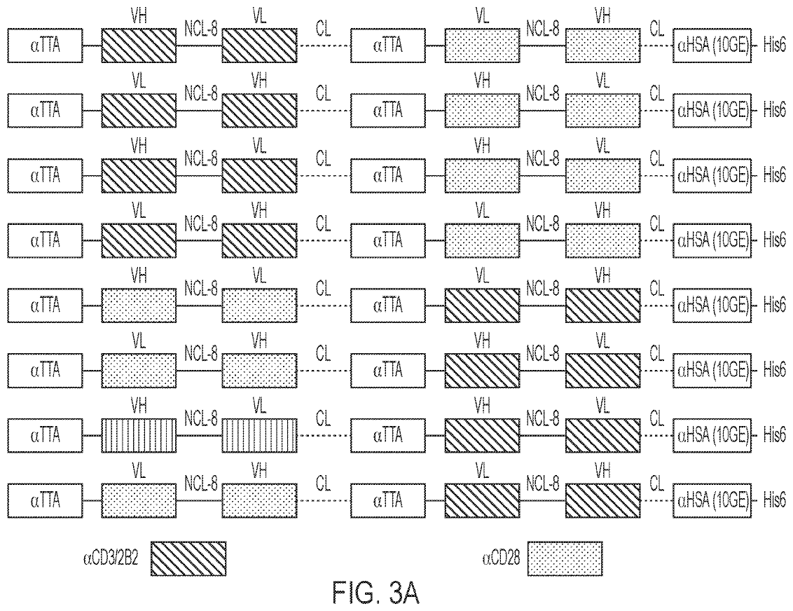

Eight different protein variants were considered, each comprising an anti-CD3 VH and VL in a constrained format and an anti-CD28 VH and VL in a constrained format (FIG. 3A). The protein variants reconfigure domains of the protein from N-terminal to C-terminal by switching the N-terminal to C-terminal orientation of the anti-CD28 VH and VL, switching the N-terminal to C-terminal orientation of the anti-CD3 VH and VL, switching the N-terminal to C-terminal orientations of the anti-CD3 constrained Fv and the anti-CD28 constrained Fv, and combinations thereof. The proteins in FIG. 3A are envisioned to comprise any TTA-ABD described herein. FIG. 3B comprises similar reconfigured domains as FIG. 3A, but each comprise a TTA single domain antibody binding domain (TTA-sdABD) that is EGFR binding.

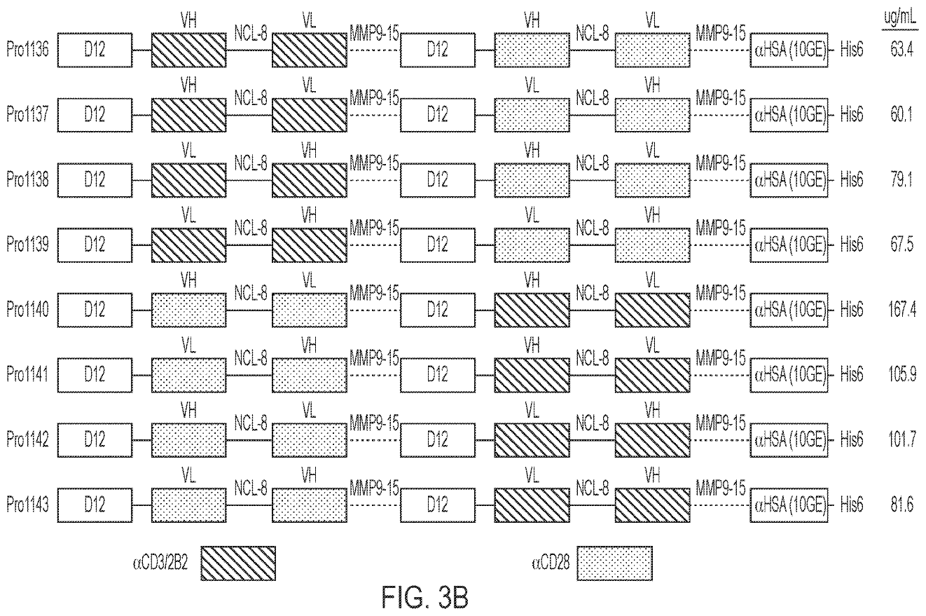

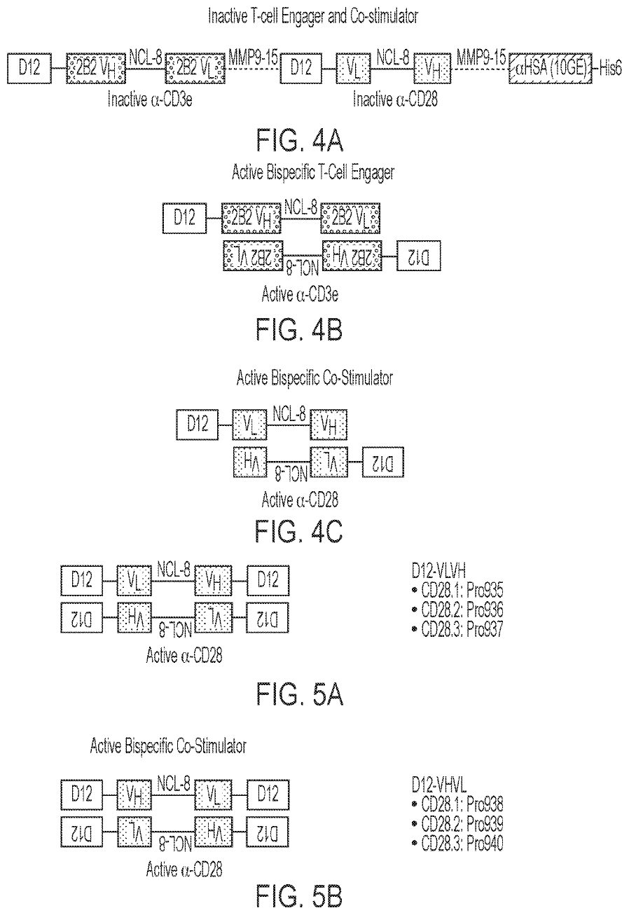

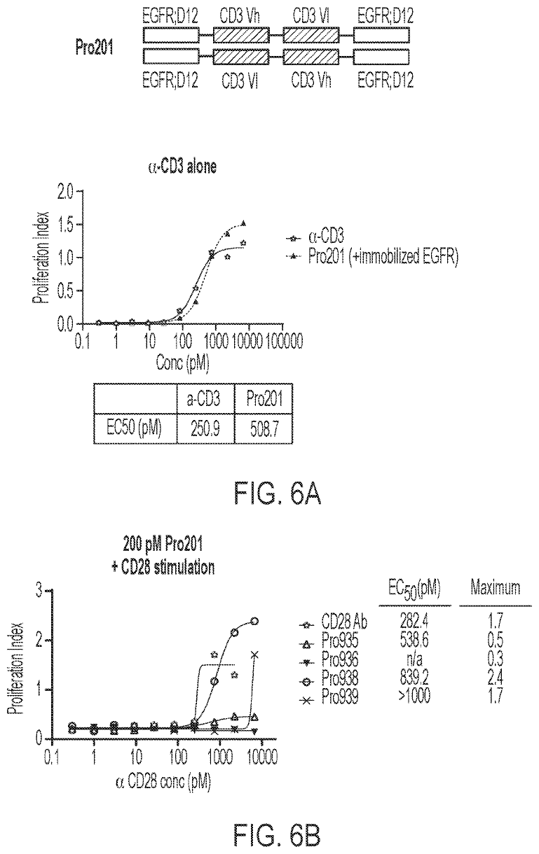

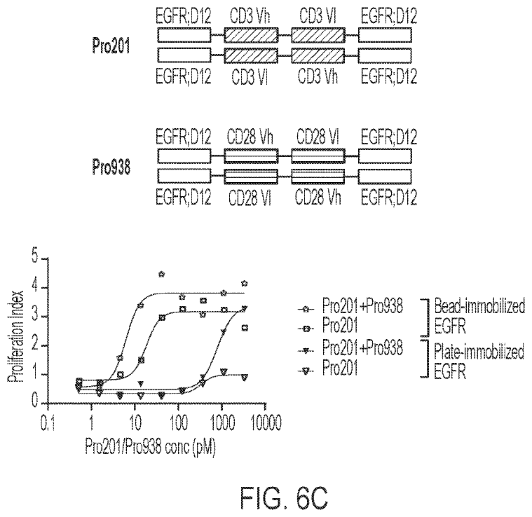

Upon protease cleavage, the proteins described in FIGS. 3A and 3B form two active homodimers each comprising TTA-ABD (or TTA-sdABD) and an immune cell binding domain. For example, FIGS. 4A-4C show a protein before cleavage (FIG. 4A) and the two homodimers produced after cleavage (FIG. 4B-4C). One homodimer has the anti-CD28 constrained Fv dimerized and two EGFR TTA-sdABDs, one on each half of the dimer. Another homodimer has the anti-CD3 constrained Fv dimerized and two EGFR TTA-sdABDs, one on each half of the dimer. In another example, a cleaved protein results in two homodimers similar to the homodimers in FIGS. 4B and 4C except each half of the homodimer comprises two EGFR TTA-sdABDs, one on the N-terminal of the VL and one on the C-terminal of the VH (FIGS. 5A-5B). The efficacy of each homodimer was then tested. Efficacy of the αCD3 homodimer and αCD28 homodimer tested using a T cell proliferation assay. The Pro201 protein comprises a constrained αCD3 Fv that forms an αCD3 homodimer comprising EGFR TTA-sdABDs. The Pro201 homodimer was first compared to an anti-CD3 antibody. Results shows that Pro201 induced T cell proliferation within about 2-fold of the EC50 of the anti-CD3 antibody (FIG. 6A). Multiple constrained αCD28 Fvs (Pro935, Pro936, Pro937, Pro938 and Pro939) that form homodimers were also tested for an ability to induce T cell proliferation in the presence of suboptimal amounts of Pro201 (200 pM Pro201). Results showed that Pro938 induced the greatest levels of T cell proliferation with an EC50 of about 839.2 pM (FIG. 6B). The anti-CD28 antibody did not induce as much T cell proliferation as Pro938, but the anti-CD28 antibody did have an EC50 of 282.4 pM. FIG. 6C shows that the activity of Pro201 and Pro201+Pro938 in in vitro T cell proliferation assays is dependent on how the EGFR is immobilized. Bead immobilized EGFR had significantly lower EC50s (about 2 orders of magnitude) than plate-immobilized EGFR.

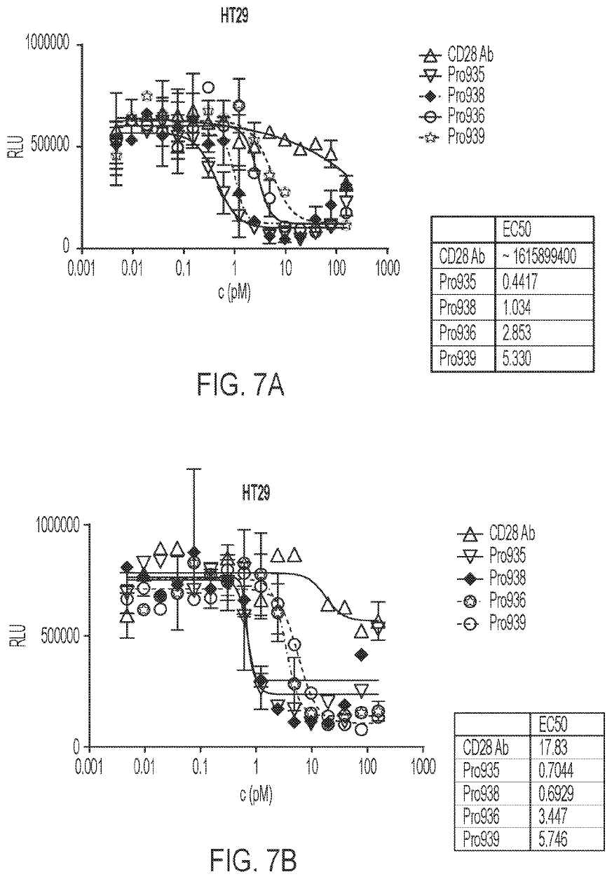

Efficacy of constrained αCD28 Fvs (Pro935, Pro936, Pro938 and Pro939) in the presence of suboptimal concentrations of Pro201 was further determined using a cytotoxicity assay with HT29 cells (human colorectal adenocarcinoma) (FIG. 7A). In FIG. 7A, HT29 cells were pretreated with Pro201 prior to treatment with anti-CD28 homodimers. Results showed that all constrained αCD28 Fvs tested had higher efficacy than an anti-CD28 binding antibody alone. Pro935 and Pro938 had the highest cytotoxicity with EC50's from about 0.4 to 1.0 pM. In FIG. 7B, HT29 cells were treated with αCD28 homodimers prior to treatment with Pro201. Results were similar to those shown in FIG. 7A suggesting that the order in which αCD28 and αCD3 homodimers bind to the cancer may not be important to cancer cell killing.

Overall, these results demonstrate that the active homodimers produced by cleavage of the conditionally active multi-specific proteins described herein, are capable of inducing T-cell proliferation and killing cancer cells.

### Example 2: Continued Development of Conditionally Active Multi-Specific Immune Cell Engagers Comprising CD3 and CD28 Immune Cell Binding Domains

This example demonstrates that immune cell engagers comprising CD3 and CD28 immune cell binding domains enhance T-cell proliferation and viability, and exhibit higher potency in cancer killing.

A substantial challenge when treating solid cancer is slowing or stopping T cells from exhibiting an exhausted phenotype, whereby they seem unable to develop any anti-cancer responses and present T cell-surface exhaustion markers, such as PD1, LAG3, and TIM3. This is likely due to extensive TCR/CD3 stimulation without additional co-stimulatory signals, which are down-modulated by many tumors to evade the host's immune system. It was hypothesized that stimulating exhausted T cells with conditional active molecule comprising an anti-CD3 constrained Fv, an anti-CD28 constrained Fv, and a TTA-sdABD may be able to reverse the exhaustion phenotype and target T-cells to a tumor.

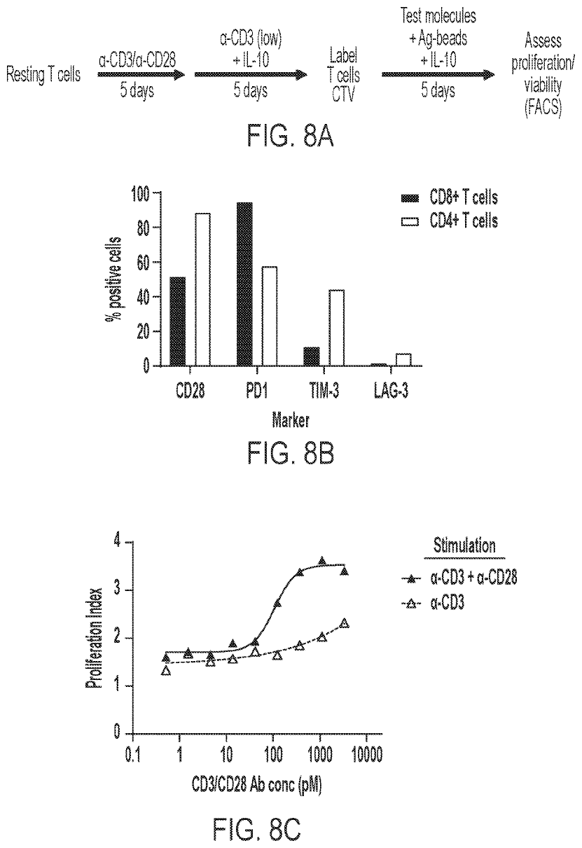

Exhausted T cell-types can be generated in vitro by isolating human primary resting T cells and incubating them with anti-CD3 and IL-10 for long periods of time (FIG. 8A). Before testing, in vitro prepared exhausted T cells are pre-labeled with Cell-Trace Violet in order to track both viability and proliferation via FACS analysis. These cells are then incubated with the described molecules at various concentrations in the presence of EGFR-conjugated beads. Exhausted T cells do not respond unless in the presence of test molecules and the EGFR (the sdABD target). Subsequently, not only do these T cells express the expected cell-surface markers (FIG. 8B), but they also fail to respond to anti-CD3 activation alone. However, these exhausted T cells do respond to a combination of anti-CD3 and anti-CD28 stimulation (FIG. 8C).

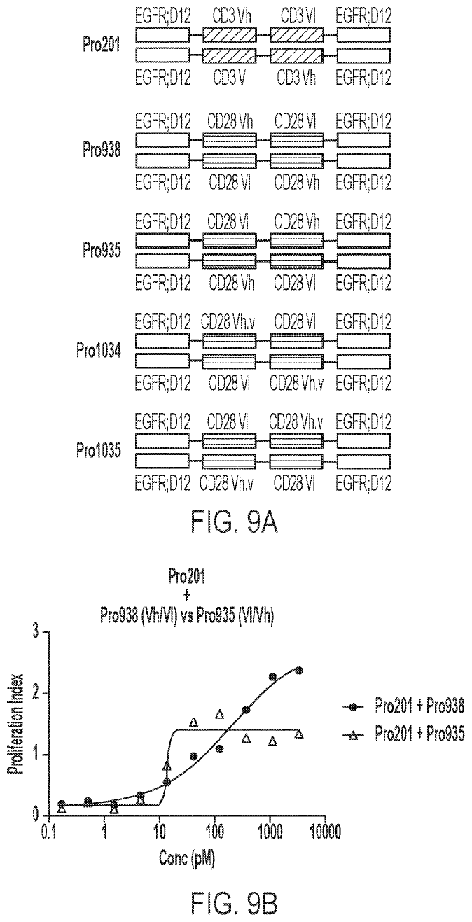

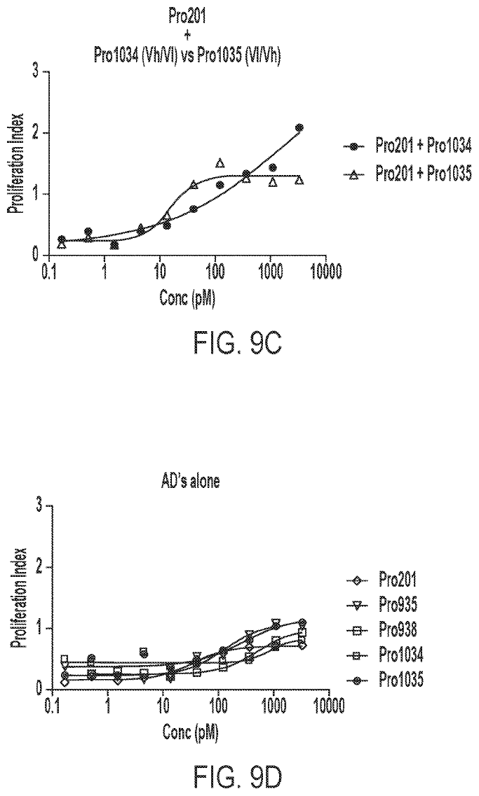

The efficacy of anti-CD3 and anti-CD28 construct on rescuing T cell exhaustion was studied in vitro using the protein constructs described in FIG. 6 and Table 1 and Table 2. In vitro prepared human exhausted T cells were stimulated in the presence of EGFR-coated beads and with anti-CD3-EGFR (Pro201) and/or anti-CD28-EGFR active dimer molecules containing 2 EGFR sdABDs Pro938 (VH/VL), Pro935 (VL/VH), Pro1034 (VH/VL), and Pro1035 (VL/VH) at equal and increasing concentrations (FIGS. 9A-9C). Results show that Pro938+Pro201 induces greater total T cell proliferation than Pro935+Pro201, but Pro935+Pro201 has a lower EC50 (FIG. 9B). Results also show that that Pro1034+Pro201 induced greater total T cell proliferation than Pro1035+Pro201, but Pro1035+Pro201 has a lower EC50 (FIG. 9C). Administration of any of these constrained Fvs alone induced T cell proliferation to a much lesser extent and with less potency compared to administration of αCD28 constrained Fv with an αCD3 constrained Fv (FIG. 9D).

TABLE 1
anti-CD3/2B2 active Dimers

| | Architecture | mg/L | Tm | TDCC |
|---|---|---|---|---|
| Pro201 | D12-CD3/VHVL-D12 | 19.5 | 53.4 | 0.1568 |
| Pro579 | D12-CD3/VHVL-HEL | 24.9 | 54.2 | 0.5515 |
| Pro861 | D12-CD3/VHVL | 2.5 | 53 | 0.7114 |
| Pro863 | D12-CD3/VLVH | 2.4 | 52.7 | 6.912 |

TABLE 2
anti-CD28 active Dimers

| | Architecture | mg/L | Tm | Co-Stim “Rank” |
|---|---|---|---|---|
| Pro1034 | D12-CD28/VHVL-D12 | 256.8 | 57.5 | 3 |
| Pro1035 | D12-CD28/VLVH-D12 | 239.4 | 57.9 | 4 |
| Pro1134 | D12-CD28/VHVL | 222.7 | 62.6 | 1 |
| Pro1135 | D12-CD28/VLVH | 217 | 62.5 | 2 |

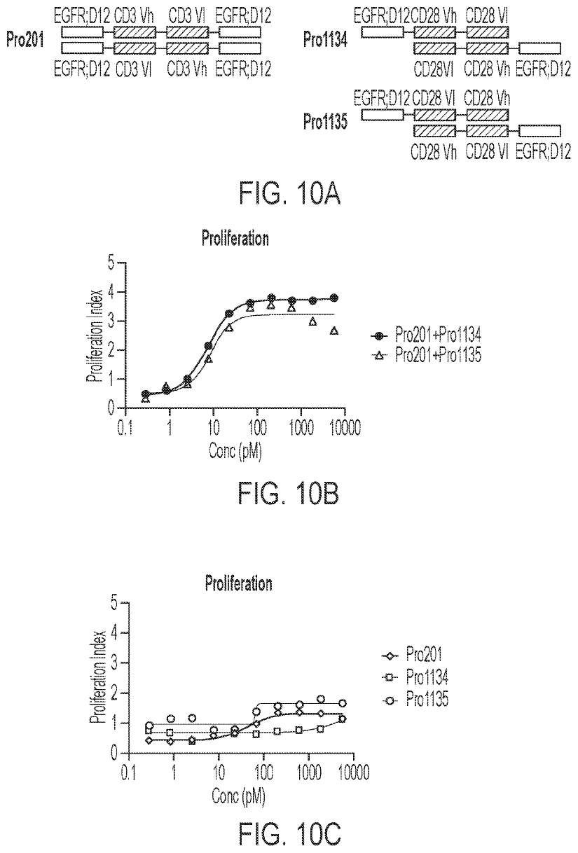

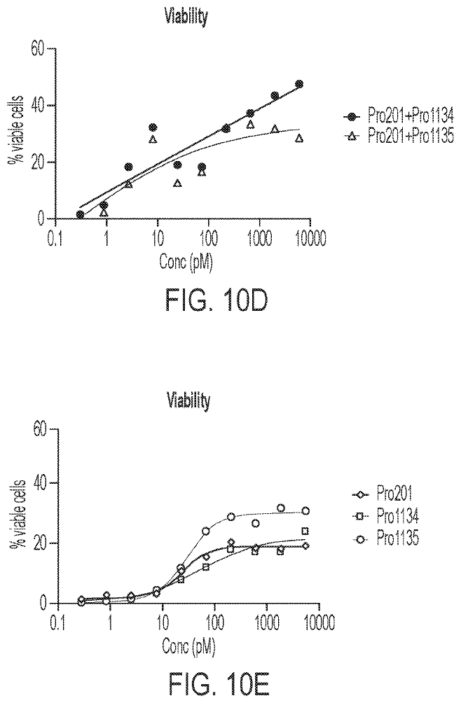

T cell proliferation and activation were further studied using Pro201 combined with anti-CD28-EGFR active dimer molecules containing 1 EGFR sdABD on each monomer, Pro1134 (VH/VL) or Pro1135 (VL/VH) (FIG. 10A, Table 2). Results show that Pro1134+Pro201 resulted in increased T cell proliferation (FIG. 10B) and T cell activation (FIG. 10D) compared to Pro1135+Pro201. As seen previously, administration of Pro201, Pro1134 or Pro1135 alone induced significantly less, if any, T cell proliferation (FIG. 10C). However, administration of Pro201, Pro1134 or Pro1135 alone was sufficient to increase T cell viability (FIG. 10E). Overall these results suggest that Pro1134 (VH/VL) has higher activity than Pro1135 (VL/VH).

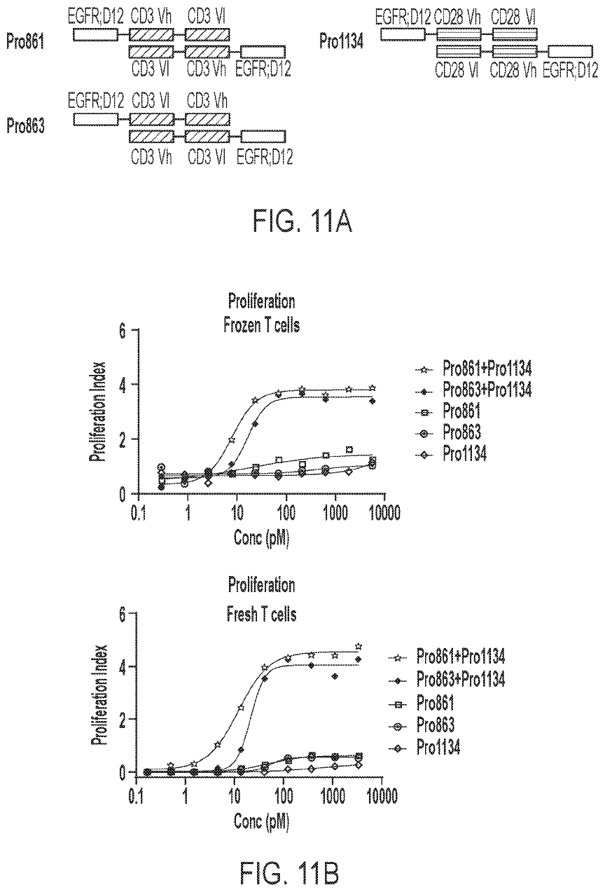

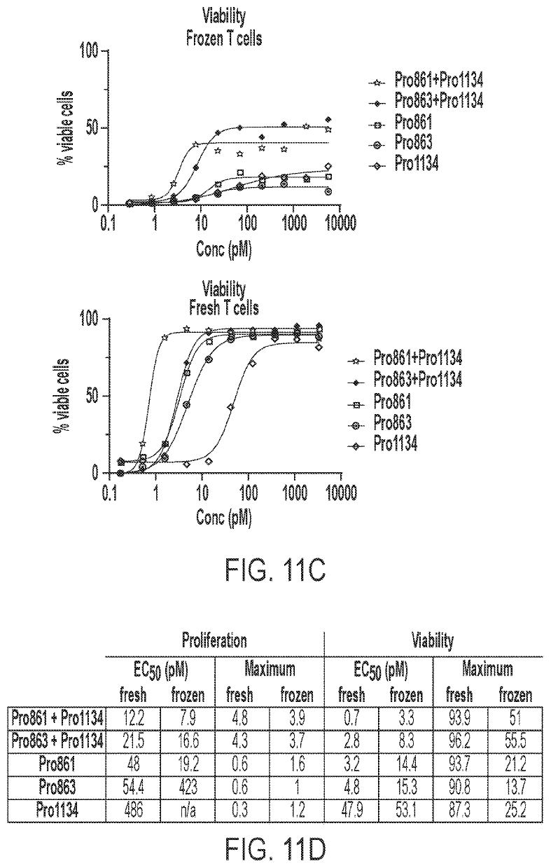

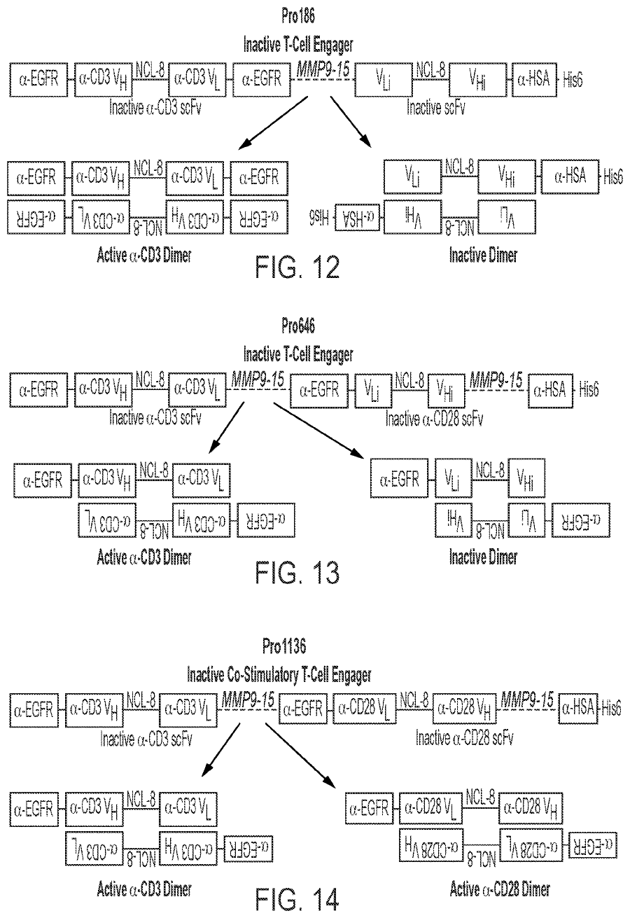

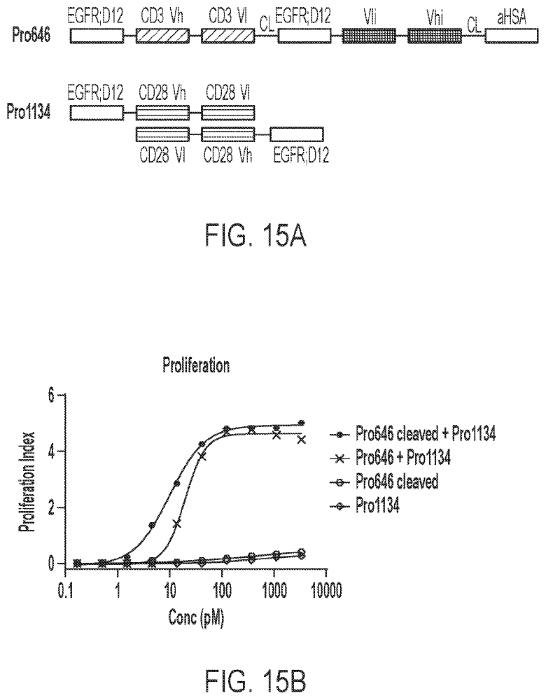

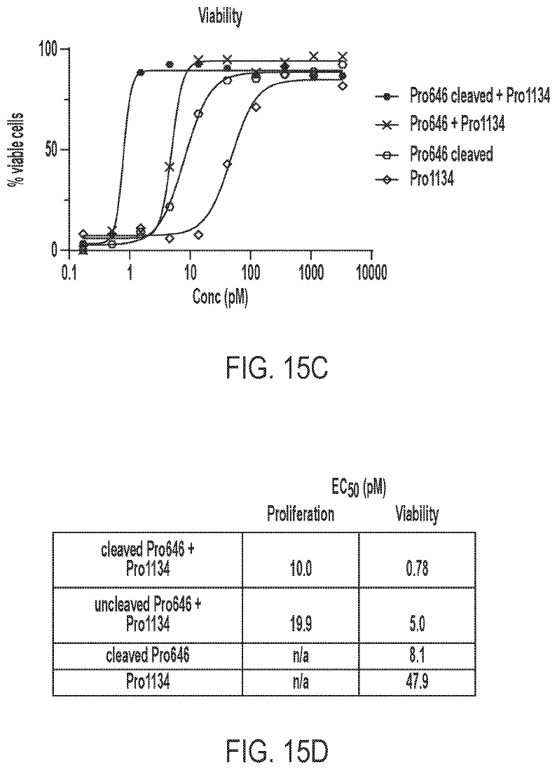

To further evaluate the combination of CD3 and CD28 signaling on the proliferation and viability of exhausted T cells, two additional constructs were designed that comprised a single anti-EGFR sdABD and anti-CD3 (VH/VL), Pro861, or anti-CD3 (VL/VH), Pro863 (FIG. 11A and Table 1). When these anti-CD3 molecules are tested in combination with Pro1134, which comprises a single anti-EGFR sdAb and anti-CD28 (VH/VL), the pairs of molecules would mimic the two active dimers formed upon proteolytic cleavage of a subset of the multi-specific molecules described in FIG. 3A and FIG. 3B. Results show that Pro861, Pro863, or Pro1134 alone was incapable of inducing substantial T cell proliferation, but Pro861+Pro1134 or Pro863+Pro1134 induced marked increases in proliferation in T cells that were either fresh or had been frozen prior to induction of exhaustion (FIG. 11B). Proliferation of exhausted T cells only occurred when both active dimers were provided. Although maximal viability was induced on fresh T cells with either Pro861, Pro863, or Pro1134 alone, the highest potency was achieved when Pro861+Pro1134 or Pro863+Pro1134 were added (FIGS. 11C and 11D). Like Pro186, Pro646 comprises two anti-EGFR domains flanking anti-CD3 VH and VL domains fused to an inactive VHi and VLi domain and a Human Serum Albumin (HSA) sdABD that increases half-life (FIGS. 12 and 13). However, Pro646 is a Co-Stimulatory COBRA intermediate molecule where the cleavage site is in front of the internal anti-EGFR sdABD (FIG. 13). Pro646 is protease conditionally activated and can form the anti-CD3-EGFR active homodimer after cleavage (FIG. 13). The cleaved version of this molecule alone was not able to induce proliferation in exhausted T cells (FIG. 15B). Only when added equally with the Pro1134, the anti-CD28-EGFR active dimer (FIG. 15A), was Pro646 able to stimulate maximal proliferation. Either Pro646 or Pro1134 alone was able to increase viability, but both molecules together had highest potency (FIGS. 15C-15D).

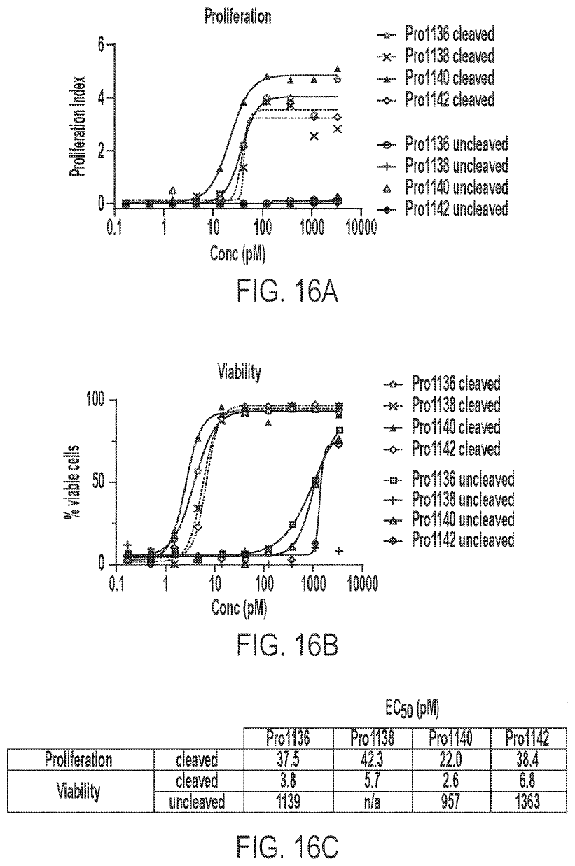

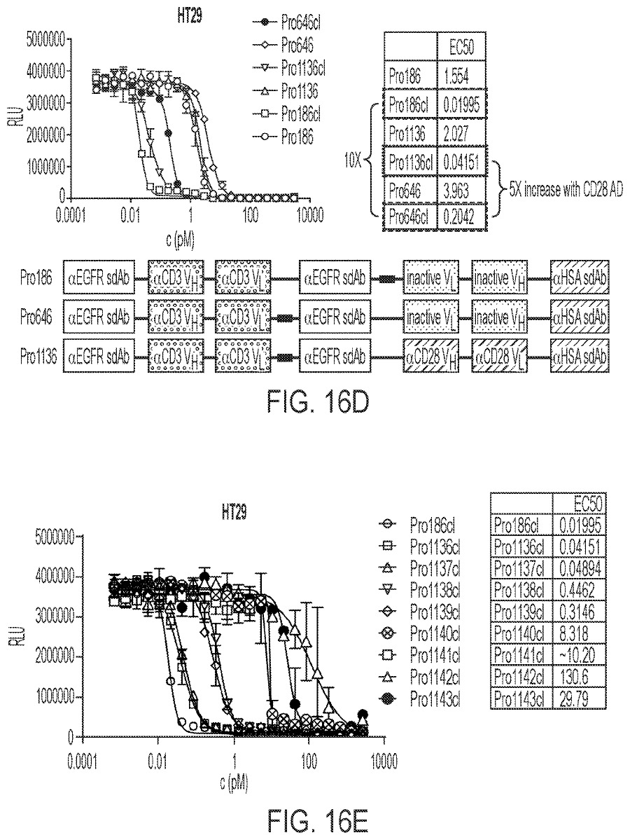

Next, it was determined how the relative positions and orientations of the anti-CD3 scFv and anti-CD28 scFv within the co-stimulatory COBRA molecules outlined in FIG. 3B affected protein production, conditional activation, T cell proliferation, T cell survival, and T cell-dependent cytotoxicity. Parameters varied were the N-terminal to C-terminal order of the anti-CD3 VH and VL, the anti-CD28 VH and VL, and order of anti-CD28 scFv and anti-CD3 scFv (FIG. 3B). Results showed that each construct tested can be obtained in sufficient quantities for practical research use (FIG. 3B). Pro1136, Pro1138, Pro1140 and Pro1142 were then tested for conditional activation and induction of T cell proliferation and survival of exhausted T cells (FIGS. 16A-16B). Results showed all proteins tested were highly conditional at stimulating proliferation, as uncleaved molecules had no activity in this assay. In contrast, the pre-cleaved proteins exhibited potencies within 2-fold of each other, although the maximum proliferation observed differed somewhat between the molecules. The four molecules were also highly conditional in stimulating T cell survival, as the potencies of the cleaved proteins were more than 200-fold higher than those of their cognate uncleaved molecules. Results from these experiments show that Pro1136 and Pro 1140 have the greatest combined effect on both T cell proliferation and T-cell viability (FIG. 16C).

Next, the tumor killing efficiency of Pro186, a T cell engager wherein the MMP9 cleavage site is located between the second EGFR binding domain and the inactive VLi domain and which uses inactive VLi-VHi to block formation of active anti-CD3 dimers, (FIG. 13) was compared to the Prol 136 protein described herein that comprise both αCD3 constrained Fvs and αCD28 constrained Fvs (FIG. 15). Pro1136 kills HT29 cells (colorectal adenocarcinoma) with similar potency as Pro186 (FIG. 16D). Native and pre-cleaved proteins were tested at various concentrations in a standard TDCC assay at a 10:1 (Human T cells:HT29 tumor cell line) ratio. All the MMP9 cleaved molecules are at least 20-fold more active than their native, non-cleaved proteins. In this series of molecules tested, pre-cleaved Pro186 was the most potent (FIG. 16D). Pre-cleaved Pro646 was at least 10-fold less active, probably because its CD3 active dimer only has two anti-EGFR sdAbs, versus the four that Pro186 can form in the active dimer. On the other hand, even though Pro1136 also creates the same αCD3 active homodimer as Pro646, it is five-fold more potent than Pro646 and just two-fold less potent than Pro186. This is most likely because Pro1136 can also form the anti-EGFR CD28 active homodimer, which can further co-stimulate T cells through the CD28 receptor and enhance their cytotoxic activity.

The series of 8 co-stimulatory COBRA molecules outlined in FIG. 3B were also tested for their activity in a T cell-dependent cytotoxicity assay on HT29 target cells. Results show that all 8 molecules, with different anti-CD3 VL and VH and anti-CD28 VL and VH orientations, as well as the relative positions of the anti-CD3 Fv and anti-CD28 Fv in the molecule, were active in the assay (FIG. 16E). Pro1136 and Pro1137, with anti-CD3 (VH/VL) Fv in the first position and anti-CD28 (VH/VL or VL/VH) Fv in the 2^{nd} position, were the most potent, and Pro1138 and Pro1139, with anti-CD3 (VL/VH) Fv in the first position and anti-CD28 (VH/VL or VL/VH) Fv in the 2^{nd} position, showed approximately 10-fold lower potency (FIG. 16E).

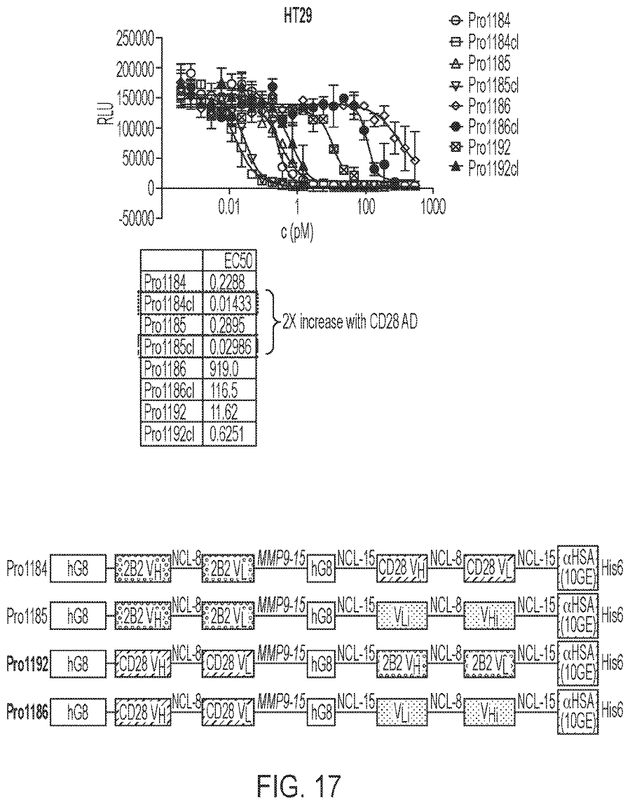

Molecules similar to Pro1136 and Pro646, but comprising a different anti-EGFR sdABD (hG8) were designed and tested in T cell-dependent cytotoxicity assay on HT29 target cells. The anti-EGFR domains of Pro646 and Pro1136 were replaced with hG8 anti-EGFR domains to create Pro1185 and Pro1184, respectively (FIG. 17). Similar to Pro646 and Pro1136, cleavage of Pro1185 and Pro1184 increased cancer cell killing by about 10-fold. Pro1184 has about a 2-fold increase in killing capacity than Pro1185. Pro1186 comprising only an anti-CD28 VH-VL, showed minimal TDCC activity, thousands of fold less than Pro1184 and Pro1185. Overall, these results demonstrate the utility of immune cell engagers comprising CD3 and CD28 immune cell binding domains (e.g., Pro1136) to activate immune cells and kill cancer.

### Methods

### The Exhausted T Cell Assay:

Fresh T cells isolated from human donors via negative selection using StemCell Technology Kit were first incubated with anti-CD3 (SP34) and anti-CD28 (in-house produced TGN1412) antibodies at 50 ng/mL each for 5 days. After washing the stimulated T cells, they were then induced to exhaustion by treating with anti-CD3 at 50 ng/ml and IL-10 (R&D Systems) at 10 ng/ml for 5 days, replenishing the latter after two days. After 5 days, the now exhausted T cells were washed gently then stained with Cell Trace Violet (Life Technologies) for 10 minutes at 37 C. Exhausted and stained T cells were washed then resuspended to 5×10⁵-1×10⁶ cells/mL. Cells were incubated with test molecules and antigen coated beads, in the constant presence of IL-10 at 10 ng/mL for 5 days, replenishing fresh IL-10 after two days. Cells were washed and resuspend in FACS buffer with 0.5 ug/mL propidium iodide for flow cytometry. Percent viability and proliferation are analyzed.

### Example 3: Conditionally Active Proteins Comprising Anti-HER2 sdABDs

The conditionally active multi-specific proteins described herein have potential to target numerous different cancer types by exchanging the sdABDs to bind to different tumor antigens. To demonstrate this, the proteins were modified to comprise anti-HER2 sdABDs, or anti-HER2 sdABDs and anti-EGFR sdABDs and then tested for potency in killing HER2 expressing cancer cells. Results demonstrate that conditionally active multi-specific proteins can be altered to target and kill multiple different cancer cell types.

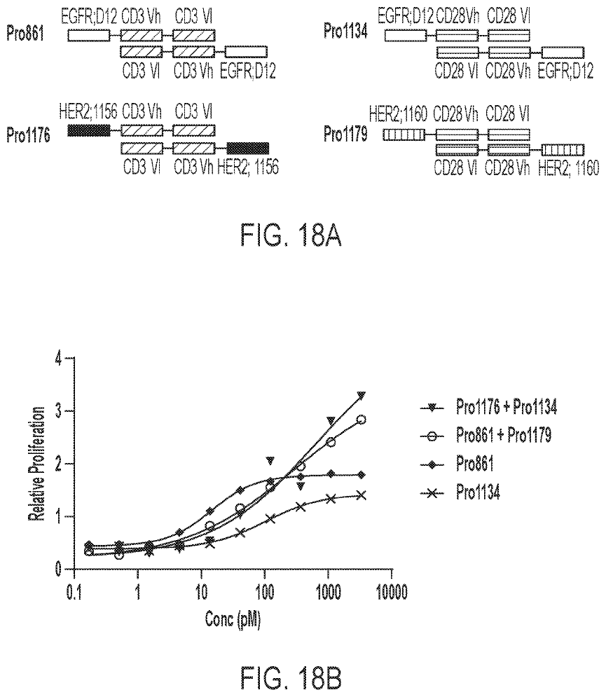

In an initial assay, two active dimer molecules comprising HER2 sdABDs were generated: Pro1176, which contains a HER2 sdABD and anti-CD3 (VH/VL), and Pro1179, which contains a HER2 sdABD and anti-CD28 (VH/VL) (FIG. 18A). Pro1176 was tested in combination with Pro1134, containing anti-EGFR sdABD and anti-CD28 (VH/VL), and Pro1179 was tested in combination with Pro861, containing anti-EGFR sdABD and CD3 (VH/VL) for their activity in the T cell proliferation assay. Treatment with the combination of Pro861+Pro1179 showed higher proliferation than with Pro861 alone, and treatment with the combination of Pro1176+Pro1134 showed higher proliferation than with Pro1134 alone (FIG. 18B). These results indicate that active dimer molecules can stimulate T cell proliferation by engaging EGFR or HER2.

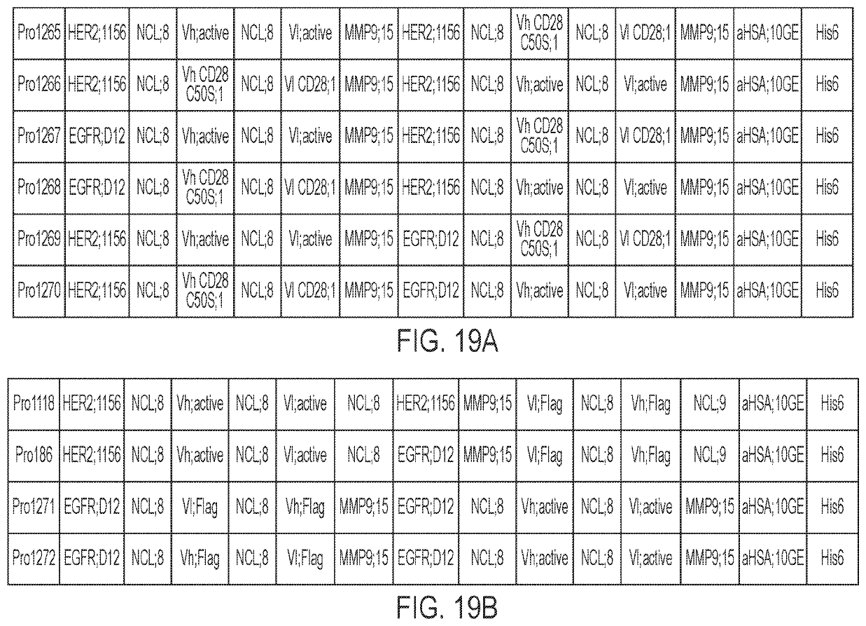

A panel of conditionally active multi-specific proteins constructs comprising anti-HER2 sdABDs was designed (FIG. 19A). These proteins comprise an anti-CD3 VH-VL, an CD28 VH-VL, an anti-HER2 sdABD, and in some cases, an anti-EGFR sdABD. Pro1265 and Pro1266 both comprise an anti-CD3 VH-VL, an CD28 VH-VL, and anti-HER2 sdABDs, but the N-terminal to C-terminal positions of the anti-CD3 VH-VL and the CD28 VH-VL are switched between the proteins. Pro1267 and Pro1268 both comprise an anti-CD3 VH-VL, an CD28 VH-VL, an anti-HER2 sdABD and an anti-EGFR sdABD, but the N-terminal to C-terminal positions of the anti-CD3 VH-VL and the CD28 VH-VL are switched between the proteins. Additionally, on Pro1267 and Pro1268, the anti-EGFR sdABD is on the N-terminus of each protein and the anti-HER2 sdABD is located C-terminal to the MMP9-15 cleavage site and between the MMP9-15 cleavage site and a NCL. Pro1269 and Pro1270 are similar to Pro1267 and Pro1268 except the anti-HER2 sdABD is on the N-terminus of each protein and the anti-EGFR sdABD is located C-terminal to the MMP9-15 cleavage site and between the MMP9-15 cleavage site and a NCL. FIG. 19B shows control proteins used in assays where the anti-CD28 VH-VL are replaced by FLAG-inactivated VH-VL.

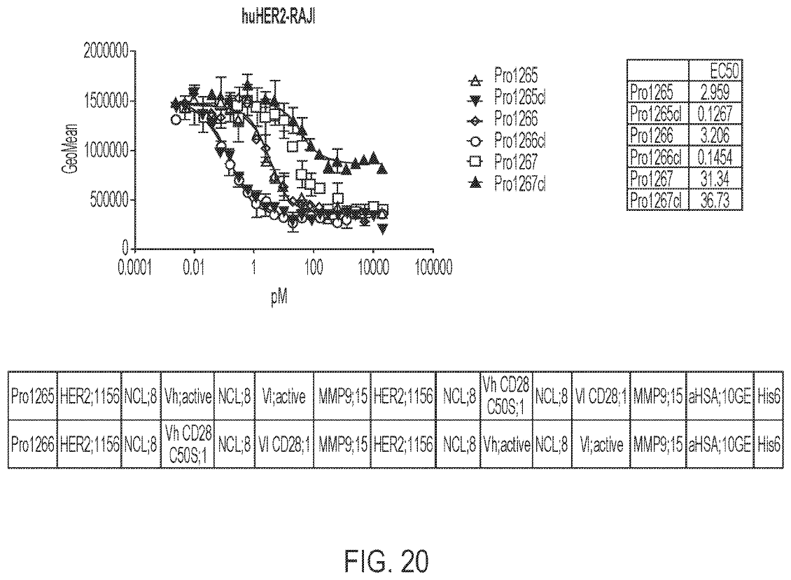

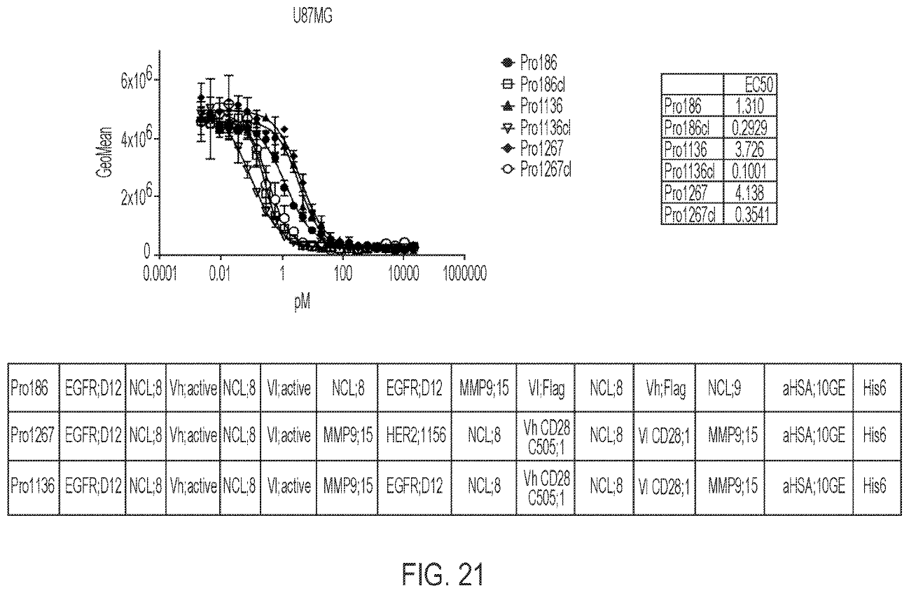

The potency of the anti-HER2 conditionally active multi-specific proteins were quantified using T cell dependent cellular cytotoxicity (TDCC) assays on human HER2-RAJI cells, which overexpress HER2, and do not express EGFR. The potency of Pro1265 and Pro1266 were compared to determine how the N-terminal to C-terminal position of the anti-CD28 VH-VL and anti-CD3 VH-VL impacted potency (FIG. 20). Results show that Pro1265 and Pro1266 kill cancer cells with an EC50 of 0.1267 and 0.1454, respectively. Additionally, the N-terminal to C-terminal position of the anti-CD28 VH-VL and anti-CD3 VH-VL had little to no impact on potency. The Pro1267 construct, which comprises an anti-HER2 sdABD that is linked to anti-CD28 Fv after cleavage and an anti-EGFR sdABD that is linked to anti-CD3 Fv after cleavage, was about 100-fold less potent than Pro1265 and Pro1266.

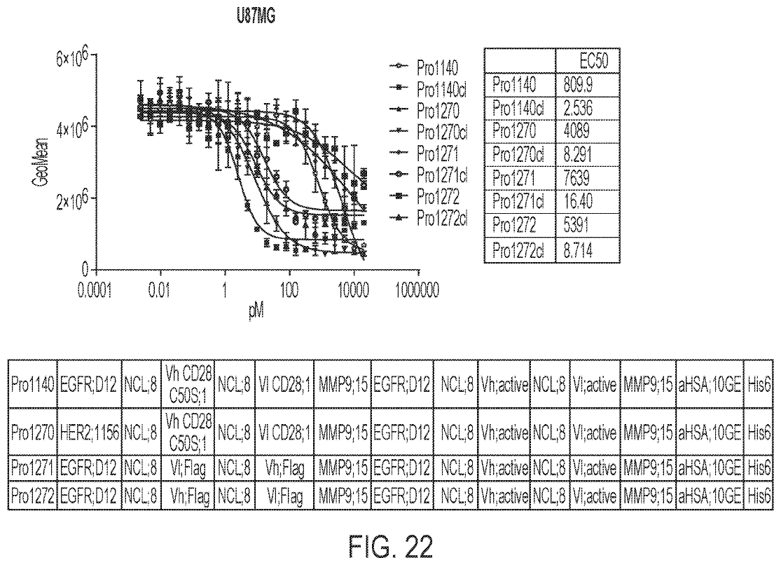

Next, the potency of proteins comprising an anti-HER2 sdABD and an anti-EGFR sdABD was further studied using cancer cells that express EGFR and not HER2. Potency was determined with TDCC assays using EGFR expressing (U87MG) cells (FIG. 21). Results showed Pro1136 (see FIG. 3B, comprising an anti-EGFR sdABD that is linked to anti-CD3 Fv after cleavage and an anti-EGFR sdABD that is linked to anti-CD28 Fv after cleavage) was more potent than Pro1267 (comprising an anti-HER2 sdABD that is linked to anti-CD28 after cleavage and an anti-EGFR sdABD that is linked to anti-CD3 Fv after cleavage). However, Pro1267 was similar in potency to Pro186, which comprises two anti-EGFR sdABDs linked to anti-CD3 after cleavage, but lacks an anti-CD28 VH-VL. Similar results were observed comparing Pro1140 to Pro1270. Pro1140, which has EGFR sdABDs linked to both anti-CD3 Fv and anti-CD28 Fv after cleavage, was at least 3-fold more potent than Pro1270, which contains an EGFR sdABD that is linked to anti-CD3 Fv after cleavage and a HER2 sdABD that is linked to anti-CD28 Fv after cleavage (FIG. 22). Pro1140 was also at least 3-fold more potent than Pro1271 or Pro1272, both of which contain one EGFR sdABD linked to anti-CD3 Fv and a second EGFR sdABD linked to inactive VL/VH or VH/VL, respectively (FIG. 22). These results suggest that molecules comprising anti-CD3 and anti-CD28 are more active than molecules containing only anti-CD3. Additionally, molecules containing both anti-CD3 and anti-CD28 are more potent only if the dimers formed after cleavage are able to bind target cells.

In conclusion, anti-HER2 conditionally active multi-specific proteins appear to have slightly higher potency when the anti-HER2 sdABD is located on the N-terminus of the protein as compared to between the MMP9 cleavage site and the NCL. Anti-EGFR conditionally active multi-specific proteins containing anti-CD3 VH-VL and anti-CD28 VH-VL have at least 3-7 fold higher potency than ones without an anti-CD28 VH-VL on U87MG cells (EGFR only).

TABLE 3
Amino Acid Sequences
TTA binding sdAbs

| | | |
|---|---|---|
| αEGFR1 | CDR1: | VH: |
| | GRTFSSYAMG | EVQLVESGGGLVQAGGSLRLSCAASGRTFSSYAMG |
| | (SEQ ID NO: 1) | WFRQAPGKEREFVVAINWSSGSTYYADSVKGRFTI |
| | CDR2: | SRDNAKNTMYLQMNSLKPEDTAVYYCAAGYQINSG |
| | INWSSGSTYYADSVK | NYNFKDYEYDYWGQGTQVTVSS |
| | G | (SEQ ID NO: 4) |
| | (SEQ ID NO: 2) | |
| | CDR3: | |
| | GYQINSGNYNFKDYE | |
| | YDY | |
| | (SEQ ID NO: 3) | |
| hαEGFR1 - hg8 | CDR1: | VH: |
| | GRTFSSYAMG | EVQLVESGGGLVQPGGSLRLSCAASGRTFSSYAMG |
| | (SEQ ID NO: 1) | WFRQAPGKEREFVVAINWSSGSTYYADSVKGRFTI |
| | CDR2: | SRDNSKNTLYLQMNSLRAEDTAVYYCAAGYQINSG |
| | INWSSGSTYYADSVK | NYNFKDYEYDYWGQGTLVTVSS |
| | G | (SEQ ID NO: 5) |
| | (SEQ ID NO: 2) | |
| | CDR3: | |
| | GYQINSGNYNFKDYE | |
| | YDY | |
| | (SEQ ID NO: 3) | |
| αEGFR2 - D12 | CDR1: | VH: |
| | GRTSRSYGMG | QVKLEESGGGSVQTGGSLRLTCAASGRTSRSYGMG |
| | (SEQ ID NO: 6) | WFRQAPGKEREFVSGISWRGDSTGYADSVKGRFTI |
| | CDR2: | SRDNAKNTVDLQMNSLKPEDTAIYYCAAAAGSAWY |
| | GISWRGDSTGYADSV | GTLYEYDYWGQGTQVTVSS |
| | KG | (SEQ ID NO: 9) |
| | (SEQ ID NO: 7) | |
| | CDR3: | |
| | AAGSAWYGTLYEYDY | |
| | (SEQ ID NO: 8) | |
| hαEGFR2 | CDR1: | VH: |
| | GRTSRSYGMG | QVKLVESGGGVVRPGGSLTLSCAASGRTSRSYGMG |
| | (SEQ ID NO: 6) | WFRQAPGKEREFVSGISWRGDSTGYADSVKGRFTI |
| | CDR2: | SRDNAKNSLYLQMNSLRAEDTALYYCAAAAGSAWY |
| | GISWRGDSTGYADSV | GTLYEYDYWGQGTLVTVSS |
| | KG | (SEQ ID NO: 10) |
| | (SEQ ID NO: 7) | |
| | CDR3: | |
| | AAGSAWYGTLYEYDY | |
| | (SEQ ID NO: 8) | |
| aEGFR2a | CDR1: | VH: |
| | GRTSRSYGMG | EVQLVESGGGVVRPGGSLRLSCAASGRTSRSYGMG |
| | (SEQ ID NO: 6) | WFRQAPGKEREFVSGISWRGDSTGYADSVKGRFTI |
| | CDR2: | SRDNAKNSLYLQMNSLRAEDTALYYCAAAAGSAWY |
| | GISWRGDSTGYADSV | GTLYEYDYWGQGTLVTVSS |
| | KG | (SEQ ID NO: 11) |
| | (SEQ ID NO: 7) | |
| | CDR3: | |
| | AAGSAWYGTLYEYDY | |
| | (SEQ ID NO: 8) | |
| αEpCAM h13 | CDR1: | VH: |
| | GTGSIFSINLMG | QVQLVESGGGLVQPGGSLTLSCAASGTGSIFSINL |
| | (SEQ ID NO: 12) | MGWYRQAPGKQRELVARITSGDSTVYADSVKGRFT |
| | CDR2: | ISRDNSKNTLYLQMNSLRPEDTAVYYCNLLLRSSP |
| | RITSGDSTVYADSVK | GATTPYWGQGTLVTVSS |
| | G | (SEQ ID NO: 15) |
| | (SEQ ID NO: 13) | |
| | CDR3: | |
| | LLRSSPGATTPY | |
| | (SEQ ID NO: 14) | |
| EpCAM h23 | CDR1: | VH: |
| | GSFSALWAMR | QVQLVESGGGLVQPGGSLTLSCVISGSFSALWAMR |
| | (SEQ ID NO: 16) | WYRQAPGQQRELVASSRGGTTSYADSVKGRFTISR |
| | CDR2: | DNSKNTLYLQMNSLRPEDTAVYYCNAIDGHLAYWG |
| | SSRGGTTSYADSVKG | QGTLVTVSS |
| | (SEQ ID NO: 17) | (SEQ ID NO: 19) |
| | CDR3: | |
| | IDGHLAY | |
| | (SEQ ID NO: 18) | |
| EpCAM hVIB665 | CDR1: | VH: |
| | GRTFSDYDMG | QVQLLESGGGLVQPGGSLRLSCAASGRTFSDYDMG |
| | (SEQ ID NO: 20) | WFRQGPGKEREFVAAISWSGGHTNYADSVKGRFTI |
| | CDR2: | SRDNSKNTLYLQMNSLRAEDTAVYYCAADLRFTGG |
| | AISWSGGHTNYADSV | DTTTPETYDYWGQGTLVTVSS |
| | KG | (SEQ ID NO: 23) |
| | (SEQ ID NO: 21) | |
| | CDR3: | |
| | DLRFTGGDTTTPETY | |
| | DY | |
| | (SEQ ID NO: 22) | |
| acEpCAM | CDR1: | VH: |
| hVIB666 | GRTLDNYDMG | QVQLVESGGGLVQPGRSLRLSCAASGRTLDNYDMG |
| | (SEQ ID NO: 24) | WFRQGPGKEREFVAAISWSGGSTDYAYSVTGRFTI |
| | CDR2: | SRDNAKNSLYLQMNSLRAEDTALYYCAADLRFTGG |
| | AISWSGGSTDYAYSV | DTMTPETYDYWGQGTLVTVSS |
| | TG | (SEQ ID NO: 27) |
| | (SEQ ID NO: 25) | |
| | CDR3: | |
| | DLRFTGGDTMTPETY | |
| | DY | |
| | (SEQ ID NO: 26) | |
| humanized | CDR1: | VH: |
| EpCAM hVIB664 | GRTLDNYDMG | QVQLLESGGGLVQPGGSLRLSCAASGRTLDNYDMG |
| | (SEQ ID NO: 24) | WFRQGPGKEREFVAAISWSGGSTDYAYSVKGRFTI |
| | CDR2: | SRDNSKNTLYLQMNSLRAEDTAVYYCAADLRFTGG |
| | AISWSGGSTDYAYSV | DTMTPETYDYWGQGTLVTVSS |
| | KG | (SEQ ID NO: 29) |
| | (SEQ ID NO: 28) | |
| | CDR3: | |
| | DLRFTGGDTMTPETY | |
| | DY | |
| | (SEQ ID NO: 26) | |
| αFOLR1 h77-2 | CDR1: | VH: |
| | GFTVSNSVMA | QVQLVESGGGLVQPGGSLRLSCAASGFTVSNSVMA |
| | (SEQ ID NO: 30) | WYRQTPGNEREFVAIINSIGITNYADSVKGRFTIS |
| | CDR2: | RDNSKNTLYLQMNSLRAEDTAVYVCNRNFDRIYWG |
| | IINSIGITNYADSVK | QGTLVTVSS |
| | G | (SEQ ID NO: 33) |
| | (SEQ ID NO: 31) | |
| | CDR3: | |
| | NEDRIY | |
| | (SEQ ID NO: 32) | |
| αFOLR1 h59.3 | CDR1: | VH: |
| | GNTFSISAMG | QVQLVESGGGLVQPGGSLRLSCAAPGNTFSISAMG |
| | (SEQ ID NO: 34) | WYRQAPGKQREWVAVTHSDYSTNYADSVKGRFTIS |
| | CDR2: | RDNSKNTLYLQMNSLRAEDTAVYYCKHYGIDYWGQ |
| | VTHSDYSTNYADSVK | GTLVTVSS |
| | G | (SEQ ID NO: 37) |
| | (SEQ ID NO: 35) | |
| | CDR3: | |
| | YGIDY | |
| | (SEQ ID NO: 36) | |
| αFOLR1 h22-4 | CDR1: | VH: |
| | GTTFSRDVMG | QVQLVESGGGLVQPGGSLRLSCEASGTTFSRDVMG |
| | (SEQ ID NO: 38) | WYRQAPGKQRELVAIISRGGSTNYADSVKGRFTIS |
| | CDR2: | RDNSKNTLYLQMNSLRAEDTAVYYCNANTATWGRV |
| | IISRGGSTNYADSVK | FWGQGTLVTVSS |
| | G | (SEQ ID NO: 41) |
| | (SEQ ID NO: 39) | |
| | CDR3: | |
| | NTATWGRVF | |
| | (SEQ ID NO: 40) | |
| αHER2 1054 | CDR1: | VH: |
| | GRTFSNYAMG | QVQLQESGGGLVQAGGSLILSCAASGRTFSNYAMG |
| | (SEQ ID NO: 42) | WFRQAPGKEREFVAAINWSGTHTDYADSVKGRFTI |
| | CDR2: | SRDNVKRTVYLQMNSLKPEDTATYFCSRGWSVFDP |
| | AINWSGTHTDYADSV | DYRGQGTQVTVSS |
| | KG | (SEQ ID NO: 45) |
| | (SEQ ID NO: 43) | |
| | CDR3: | |
| | GWSVFDPDY | |
| | (SEQ ID NO: 44) | |
| αHER2 1055 | CDR1: | VH: |
| | GRAISSYAIS | QVQLQESGGGLVQPGGSLRLTCEVSGRAISSYAIS |
| | (SEQ ID NO: 46) | WFRQAPGEEREFVAAINWSGTHTNYADSVKGRFTI |
| | CDR2: | SRDNVKNTVYLQMNSLKPEDTATYFCSRGWSVFDP |
| | AINWSGTHTNYADSV | DYRGQGTQVTVSS |
| | KG | (SEQ ID NO: 48) |
| | (SEQ ID NO: 47) | |
| | CDR3: | |
| | GWSVFDPDY | |
| | (SEQ ID NO: 44) | |
| αHER2 1058 | CDR1: | VH |
| | GRAISSYAIS | QVQLQESGGGLVQAGGSLRLTCAASGRAISSYAIS |
| | (SEQ ID NO: 46) | WFRQAPGEEREFVAAINWSGTHTDYADSVKGRFTI |
| | CDR2: | SRDNVKRTVYLQMNSLKPEDTATYFCSRGWSVFDP |
| | AINWSGTHTDYADSV | DYRGQGTQVTVSS |
| | KG | (SEQ ID NO: 49) |
| | (SEQ ID NO: 43) | |
| | CDR3: | |
| | GWSVFDPDY | |
| | (SEQ ID NO: 44) | |
| αHER2 | CDR1: | VH: |
| humanized 1058 | GRAISSYAIS | QVQLLESGGGLVQAGGSLRLSCAASGRAISSYAIS |
| | (SEQ ID NO: 46) | WFRQAPGEEREFVAAINWSGTHTDYADSVKGRFTI |
| | CDR2: | SRDNSKRTVYLQMNSLRAEDTAVYFCSRGWSVFDP |
| | AINWSGTHTDYADSV | DYRGQGTLVTVSS |
| | KG | (SEQ ID NO: 50) |
| | (SEQ ID NO: 43) | |
| | CDR3: | |
| | GWSVFDPDY | |
| | (SEQ ID NO: 44) | |
| αHER2 1059 | CDR1: | VH: |
| | GRAISSYAIS | QVQLQESGGGLVQPGGSLRLTCAASGRAISSYAIS |
| | (SEQ ID NO: 46) | WFRQAPGEEREFVAAINWSGTHTDYADSVKGRFTI |
| | CDR2: | SRDNAKNTVYLQMNSLKPEDTAVYFCSRGWSVFDP |
| | AINWSGTHTDYADSV | DYRGQGTQVTVSS |
| | KG | (SEQ ID NO: 51) |
| | (SEQ ID NO: 43) | |
| | CDR3: | |
| | GWSVFDPDY | |
| | (SEQ ID NO: 44) | |
| αHER2 1065 | CDR1: | VH: |
| | GRAISSYAIS | QVQLQESGGGLVQPGGSLRLTCEVSGRAISSYAIS |
| | (SEQ ID NO: 46) | WFRQAPGEEREFVAAINWSGTHTNYADSVKGRFTI |
| | CDR2: | SRDNVKRTVYLQMNSLKPEDTATYFCSRGWSVFDP |
| | AINWSGTHTNYADSV | DYRGQGTQVTVSS |
| | KG | (SEQ ID NO: 52) |
| | (SEQ ID NO: 47) | |
| | CDR3: | |
| | GWSVFDPDY | |
| | (SEQ ID NO: 44) | |
| αHER2 1074 | CDR1: | VH: |
| | GRAISSYAIS | QVQLQESGGGLVQPGGSLRLTCAASGRAISSYAIS |
| | (SEQ ID NO: 46) | WFRQAPGEEREFVAAINWSGTHTNYADSVKGRFTI |
| | CDR2: | SRDNVKNTVYLQMNSLKPEDTATYFCSRGWSVFDP |
| | AINWSGTHTNYADSV | DYRGQGTQVTVSS |
| | KG | (SEQ ID NO: 299) |
| | (SEQ ID NO: 47) | |
| | CDR3: | |
| | GWSVFDPDY | |
| | (SEQ ID NO: 44) | |
| αHER2 1090 | CDR1: | VH: |
| | GRAISSYAIS | QVQLQESGGGLVQTGGSLRLTCEVSGRAISSYAIS |
| | (SEQ ID NO: 46) | WFRQAPGEEREFVAAINWSGTHTNYADSVKGRFTI |
| | CDR2: | SRDNVKNTVYLQMNSLKPEDTATYFCSRGWSLFDP |
| | AINWSGTHTNYADSV | DYRGQGTQVTVSS (SEQ ID NO: 54) |
| | KG | |
| | (SEQ ID NO: 47) | |
| | CDR3: | |
| | GWSLFDPDY | |
| | (SEQ ID NO: 53) | |
| αHER2 1091 | CDR1: | VH: |
| | GFTFDDYAMS | QVQLQESGGGLVQPGGSLRLSCAASGFTFDDYAMS |
| | (SEQ ID NO: 55) | WVRQAPGKGLEWVSSINWSGTHTDYADSVKGRFTI |
| | CDR2: | SRDNAKNTLYLQMNSLKSEDSAVYYCVKGWRLTGG |
| | SINWSGTHTDYADSV | YYDSVQGQGTQVTVSS |
| | KG | (SEQ ID NO: 58) |
| | (SEQ ID NO: 56) | |
| | CDR3: | |
| | GWRLTGGYYDSV | |
| | (SEQ ID NO: 57) | |
| αHER2 1092 | CDR1: | VH: |
| | GFTEDDYALS | QVQLQESGGGLVQAGASLRLSCAASGFTEDDYALS |
| | (SEQ ID NO: 59) | WVRQAPGKGLEWVSSINWSGTHTDYADSVKGRFTI |
| | CDR2: | SRDNAKNTLYLQMNSLKSEDSAVYYCVKGWRLTGG |
| | SINWSGTHTDYADSV | YYDSVQGQGTQVTVSS |
| | KG | (SEQ ID NO: 60) |
| | (SEQ ID NO: 56) | |
| | CDR3: | |
| | GWRLTGGYYDSV | |
| | (SEQ ID NO: 57) | |
| αHER2 1097 | CDR1: | VH: |
| | GFIFDDYAMT | QVQLQESGGGLVQPGGSLRLSCAASGFIFDDYAMT |
| | (SEQ ID NO: 61) | WVRQAPGKGLEWVSSINWSGTHTDYADSVKGRFTI |
| | CDR2: | SRDNAKNTLYLQMNSLKSEDSAVYYCVKGWRHTDN |
| | SINWSGTHTDYADSV | YYAGVQGQGTQVTVSS |
| | KG | (SEQ ID NO: 63) |
| | (SEQ ID NO: 56) | |
| | CDR3: | |
| | GWRHTDNYYAGV | |
| | (SEQ ID NO: 62) | |
| αHER2 1118 | CDR1: | VH: |
| | GFTFDDYAMS | QVQLQESGGGLVQAGGSLRLSCAASGFTEDDYAMS |
| | (SEQ ID NO: 55) | WVRQTPGKGLEWVSSINWSGTHTNYADSVKGRFTI |
| | CDR2: | SRDNAKKTLYLQMNSLKSEDTAVYYCARGWMVVPD |
| | SINWSGTHTNYADSV | STRGQGTQVTVSS |
| | KG | (SEQ ID NO: 66) |
| | (SEQ ID NO: 64) | |
| | CDR3: | |
| | GWMVVPDST | |
| | (SEQ ID NO: 65) | |
| αHER2 1121 | CDR1: | VH: |
| | GFIFSDYAMT | QVQLQESGGGLVQAGGSLRLSCAASGFIFSDYAMT |
| | (SEQ ID NO: 67) | WVRQAPGKGLEWVSSINWSGTHTNYADSVKGRFTI |
| | CDR2: | SRDNAKKTLYLQMNSLKSEDTAVYYCARGWMVVPD |
| | SINWSGTHTNYADSV | STRGQGTQVTVSS |
| | KG | (SEQ ID NO: 68) |
| | (SEQ ID NO: 64) | |
| | CDR3: | |
| | GWMVVPDST | |
| | (SEQ ID NO: 65) | |
| αHER2 1134 | CDR1: | VH: |
| | GRIFSAAVMA | QVQLQESGGGLVQAGGSLRLSCAASGRIFSAAVMA |
| | (SEQ ID NO: 69) | WYRQPPGEQRELVASITHRGFTRYADSVKDRFTIS |
| | SITHRGFTRYADSVK | RDNVNNTMYLQMNRLSPEDTAIYYCRLLNGDYWGQ |
| | D | GTQVTVSS |
| | CDR2: | (SEQ ID NO: 72) |
| | (SEQ ID NO: 70) | |
| | CDR3: | |
| | LNGDY | |
| | (SEQ ID NO: 71) | |
| αHER2 1138 | CDR1: | VH: |
| | GFTFDDYAMS | QVQLQESGGGLVQPGGSLRLSCAASGFTFDDYAMS |
| | (SEQ ID NO: 55) | WVRQAPGKEMEWVSGINWSGTHTRYADSVKGRFTI |
| | CDR2: | SRDNAKNTLDLQMNSLISEDTAVYYCARGFAADSR |
| | GINWSGTHTRYADSV | STRGQGTQVTVSS |
| | KG | (SEQ ID NO: 75) |
| | (SEQ ID NO: 73) | |
| | CDR3: | |
| | GFAADSRST | |
| | (SEQ ID NO: 74) | |
| αHER2 1139 | CDR1: | VH: |
| | GFTFDDYAMS | QVQLQESGGGLVQPGGSLRLSCAASGFTFDDYAMS |
| | (SEQ ID NO: 55) | WIRQAPGKEMEWVSGINWSGTHTRYADSVKGRFTI |
| | CDR2: | SRDNAKNTLDLQMNSLISEDTAVYYCARGFAADSR |
| | GINWSGTHTRYADSV | STRGQGTQVTVSS |
| | KG | (SEQ ID NO: 76) |
| | (SEQ ID NO: 73) | |
| | CDR3: | |
| | GFAADSRST | |
| | (SEQ ID NO: 74) | |
| humanized aHER2 | CDR1: | VH: |
| sdAb h1139 | GFTFDDYAMS | QVQLVESGGGVVRPGGSLRLSCAASGFTFDDYAMS |
| | (SEQ ID NO: 55) | WIRQAPGKEMEWVSGINWSGTHTRYADSVKGRFTI |
| | CDR2: | SRDNAKNSLYLQMNSLRAEDTALYYCARGFAADSR |
| | GINWSGTHTRYADSV | STRGQGTLVTVSS |
| | KG | (SEQ ID NO: 77) |
| | (SEQ ID NO: 73) | |
| | CDR3: | |
| | GFAADSRST | |
| | (SEQ ID NO: 74) | |
| αHER2 1140 | CDR1: | VH: |
| | GFTFDDYAMS | QVQLQESGGGLVQPGGSLRLSCVASGFTFDDYAMS |
| | (SEQ ID NO: 55) | WVRQAPGKEMEWVSGINWSGTHTRYADSVKGRFTI |
| | CDR2: | SRDNAKNTLDLQMNSLISEDTAVYYCARGFAADSR |
| | GINWSGTHTRYADSV | STRGQGTQVTVSS |
| | KG | (SEQ ID NO: 78) |
| | (SEQ ID NO: 73) | |
| | CDR3: | |
| | GFAADSRST | |
| | (SEQ ID NO: 74) | |
| αHER2 1145 | CDR1: | VH: |
| | GRIFKNYPMG | QVQLQESGGGLVQAGGSLRLSCAASGRIFKNYPMG |
| | (SEQ ID NO: 79) | WFRQPPGKDREFVAAITSSGGTTHYRDSVKGRFTI |
| | CDR2: | SRDNAKNTVYLQMNSLKPDDTAVYYCAGSSTPWDI |
| | AITSSGGTTHYRDSV | PLGPNEVGYWGQGTQVTVSS |
| | KG | (SEQ ID NO: 82) |
| | (SEQ ID NO: 80) | |
| | CDR3: | |
| | SSTPWDIPLGPNEVG | |
| | Y | |
| | (SEQ ID NO: 81) | |
| αHER2 1146 | CDR1: | VH: |
| | GRIFKNYPMG | QVQLQESGGGLVQAGGSLRLSCAASGRIFKNYPMG |
| | (SEQ ID NO: 79) | WFRQPPGKDREFVAAITSSGGTTPYRDSVKGRFTI |
| | CDR2: | SRDNAKNTVYLQMNSLKPEDTAVYYCAGSSTPWDI |
| | AITSSGGTTPYRDSV | PLGPNEVGFWGQGTQVTVSS |
| | KG | (SEQ ID NO: 85) |
| | (SEQ ID NO: 83) | |
| | CDR3: | |
| | SSTPWDIPLGPNEVG | |
| | F | |
| | (SEQ ID NO: 84) | |
| αHER2 1149 | CDR1: | VH: |
| | GNIFGFNVMG | QVQLQESGGGLVQAGGSLRLSCVASGNIFGFNVMG |
| | (SEQ ID NO: 86) | WYRQVPGKQRELVAISTGGRSTDYADSAKGRFTIS |
| | CDR2: | RNNAKDTVYLQMNSLKPEDTAVYYCKMTMASSDYW |
| | ISTGGRSTDYADSAK | GQGTQVTVSS (SEQ ID NO: 89) |
| | G | |
| | (SEQ ID NO: 87) | |
| | CDR3: | |
| | TMASSDY | |
| | (SEQ ID NO: 88) | |
| αHER2 1150 | CDR1: | VH: |
| | GNIFGFNVMG | QVQLQESGGGLVQAGGSLRLSCVASGNIFGFNVMG |
| | (SEQ ID NO: 86) | WYRQVPGKQRELVAISTGGGSTNYADSAKGRFTVS |
| | CDR2: | RNNAKDTVYLQMNSLKPEDTAVYYCKMTMASVDYW |
| | ISTGGGSTNYADSAK | GQGTQVTVSS |
| | G | (SEQ ID NO: 300) |
| | (SEQ ID NO: 90) | |
| | CDR3: | |
| | TMASVDY | |
| | (SEQ ID NO: 91) | |
| αHER2 1156 | CDR1: | VH: |
| | RNIGSNYAVG | QVQLQESGGGLVQAGGSLKVSCAASRNIGSNYAVG |
| | (SEQ ID NO: 92) | WFRQAPGKEREPVAALRWTGSIIGYDDSLRGRFTI |
| | CDR2: | SKDDAKNTVYLQMNSLKPEDTGVYYCAARILDRSS |
| | ALRWTGSIIGYDDSL | YDYWGQGTQVTVSS |
| | RG | (SEQ ID NO: 95) |
| | (SEQ ID NO: 93) | |
| | CDR3: | |
| | RILDRSSYDY | |
| | (SEQ ID NO: 94) | |
| humanized aHER2 | CDR1: | VH: |
| sdAb h1156 | RNIGSNYAVG | QVQLLESGGGLVQPGGSLRLSCAASRNIGSNYAVG |
| | (SEQ ID NO: 92) | WFRQAPGKEREPVAALRWTGSIIGYDDSLRGRFTI |
| | CDR2: | SKDNSKNTLYLQMNSLKPEDTAVYYCAARILDRSS |
| | ALRWTGSIIGYDDSL | YDYWGQGTLVTVSS |
| | RG | (SEQ ID NO: 96) |
| | (SEQ ID NO: 93) | |
| | CDR3: | |
| | RILDRSSYDY | |
| | (SEQ ID NO: 94) | |
| αHER2 1158 | CDR1: | VH: |
| | GRTFSSYAMG | QVQLQESGGGLVQAGGSLRLSCTASGRTFSSYAMG |
| | (SEQ ID NO: 1) | WFRQAPEKEREFVAAITWSGGTTHYAESVKGRFTI |
| | CDR2: | SRDNAKNTVYLQMNTLEPEDTAVYYCAARINYSTI |
| | AITWSGGTTHYAESV | SSNEKMYHYWGQGTQVTVSS |
| | KG | (SEQ ID NO: 99) |
| | (SEQ ID NO: 97) | |
| | CDR3: | |
| | RINYSTISSNEKMYH | |
| | Y | |
| | (SEQ ID NO: 98) | |
| αHER2 1159 | CDR1: | VH: |
| | GRTFSSLAMG | QVQLQESGGGLVQAGGSLRLSCATSGRTFSSLAMG |
| | (SEQ ID NO: 100) | WFRQAPGKERELVAGISWSGSTTYYADSVKGRFAI |
| | CDR2: | SRDNAKNTVYLQMNSLKPEDTAVYYCAARTSSIAT |
| | GISWSGSTTYYADSV | IGREYDYWGQGTQVTVSS |
| | KG | (SEQ ID NO: 103) |
| | (SEQ ID NO: 101) | |
| | CDR3: | |
| | RTSSIATIGREYDY | |
| | (SEQ ID NO: 102) | |
| humanized aHER2 | CDR1: | QVQLLESGGGLVQPGGSLRLSCATSGRTFSSLAMG |
| sdAb h1159 | GRTFSSLAMG | WFRQAPGKERELVAGISWSGSTTYYADSVKGRFAI |
| | (SEQ ID NO: 100) | SRDNSKNTLYLQMNSLRAEDTAVYYCAARTSSIAT |
| | CDR2: | IGREYDYWGQGTLVTVSS |
| | GISWSGSTTYYADSV | (SEQ ID NO: 104) |
| | KG | |
| | (SEQ ID NO: 101) | |
| | CDR3: | |
| | RTSSIATIGREYDY | |
| | (SEQ ID NO: 102) | |
| αHER2 1160 | CDR1: | VH: |
| | GRTFSNERLG | QVQLQESGGGLVQAGGSLRLSCAASGRTFSNERLG |
| | (SEQ ID NO: 105) | WFRQAPGKERELVAAIRWSGVIIGYADSVRGRFTI |
| | CDR2: | SRDNAKSTVYLQMNSLRPEDTAVYFCAADRGVYGT |
| | AIRWSGVIIGYADSV | WDYWGQGTQVTVSS |
| | RG | (SEQ ID NO: 108) |
| | (SEQ ID NO: 106) | |
| | CDR3: | |
| | DRGVYGTWDY | |
| | (SEQ ID NO: 107) | |
| αHER2 1161 | CDR1: | VH: |
| | GDIFGTYGMA | QVQLQESGGGLVQPGKSLRLSCAVSGDIFGTYGMA |
| | (SEQ ID NO: 109) | WYRQAPGKQREPVASISIGGIINYADSVKGRFTIS |
| | CDR2: | RDNVKNTVYLQMNSLKPEDTAVYYCKLLDWNYWGR |
| | SISIGGIINYADSVK | GTQVTVSS |
| | G | (SEQ ID NO: 112) |
| | (SEQ ID NO: 110) | |
| | CDR3: | |
| | LDWNY | |
| | (SEQ ID NO: 111) | |
| αHER2 1162 | CDR1: | VH: |
| | GRPFSGNAMG | QVQLQESGGGLVQAGGSLRLSCAASGRPFSGNAMG |
| | (SEQ ID NO: 113) | WFRQAPGKEREFVAAISWSGDSTYYADSVKGRFTI |
| | CDR2: | SRDNDENTAYLQMNSLKPEDTAVYYCAATRSFYSR |
| | AISWSGDSTYYADSV | TYYTRPSDYNYWGPGTQVTVSS |
| | KG | (SEQ ID NO: 116) |
| | (SEQ ID NO: 114) | |
| | CDR3: | |
| | TRSFYSRTYYTRPSD | |
| | YNY | |
| | (SEQ ID NO: 115) | |
| humanized aHER2 | CDR1: | VH: |
| sdAb h1162 | GRPFSGNAMG | QVQLLESGGGLVQPGGSLRLSCAASGRPFSGNAMG |
| | (SEQ ID NO: 113) | WFRQAPGKEREFVAAISWSGDSTYYADSVKGRFTI |
| | CDR2: | SRDNSKNTLYLQMNSLRAEDTAVYYCAATRSFYSR |
| | AISWSGDSTYYADSV | TYYTRPSDYNYWGPGTLVTVSS |
| | KG | (SEQ ID NO: 117) |
| | (SEQ ID NO: 114) | |
| | CDR3: | |
| | TRSFYSRTYYTRPSD | |
| | YNY | |
| | (SEQ ID NO: 115) | |
| αHER2 1163 | CDR1: | VH: |
| | GRRFNNYFMG | QVQLQESGGGLVQAGGSLRLSCAASGRRFNNYFMG |
| | (SEQ ID NO: 118) | WFRQAPGKEREFVALLRESGDNTYYTNSVKGRFTI |
| | CDR2: | SRDNARNTVYLQMNSLKPEDTAVYYCAARLDTGFV |
| | LLRESGDNTYYTNSV | LYSANSYAYWGQGTQVTVSS |
| | KG | (SEQ ID NO: 121) |
| | SEQ ID NO: 119) | |
| | CDR3: | |
| | RLDTGFVLYSANSYA | |
| | Y | |
| | (SEQ ID NO: 120) | |
| αLyPD3 h787 | CDR1: | VH: |
| | GFTLDYYAIG | QVQLLESGGGLVQPGGSLRLSCAASGFTLDYYAIG |
| | (SEQ ID NO: 122) | WFRQAPGKEHEAVSCISSSDGSTYYADSVKGRFTI |
| | CDR2: | SRDNSKNTLYLQMNSLRAEDTAVYYCATVKSCYSD |
| | CISSSDGSTYYADSV | GYALWGSWGQGTLVTVSS |
| | KG | (SEQ ID NO: 125) |
| | (SEQ ID NO: 123) | |
| | CDR3: | |
| | VKSCYSDGYALWGS | |
| | (SEQ ID NO: 124) | |
| αLyPD3 h790 | CDR1: | VH: |
| | GFTLDYYSIG | QVQLLESGGGLVQPGGSLRLSCAASGFTLDYYSIG |
| | (SEQ ID NO: 126) | WFRQAPGKEREGVSCISSSDGSTYYADSVKGRFTI |
| | CDR2: | SRDNSKNTLYLQMNSLRAEDTAVYYCATVKSCYVD |
| | CISSSDGSTYYADSV | GYILWGSWGQGTLVTVSS |
| | KG | (SEQ ID NO: 128) |
| | (SEQ ID NO: 123) | |
| | CDR3: | |
| | VKSCYVDGYILWGS | |
| | (SEQ ID NO: 127) | |
| αLyPD3 H804 | CDR1: | VH: |
| | GFTLDYYAIG | QVQLLESGGGLVQPGGSLRLSCAASGFTLDYYAIG |
| | (SEQ ID NO: 122) | WFRQAPGKEHEGVSCISSSDGSTYYADSVKGRFTI |
| | CDR2: | SRDNSKNTLYLQMNSLRAEDTAVYYCATVRSCASP |
| | CISSSDGSTYYADSV | YELGSWGQGTLVTVSS |
| | KG | (SEQ ID NO: 130) |
| | (SEQ ID NO: 123) | |
| | CDR3: | |
| | VRSCASPYELGS | |
| | (SEQ ID NO: 129) | |
| αLyPD3 h773 | CDR1: | VH: |
| | GRSFSSYGMG | QVQLVESGGGLVQPGGSLRLSCAASGRSFSSYGMG |
| | (SEQ ID NO: 131) | WFRQAPGKEREIVAAISWGGSAVYYANSVKGRFTI |
| | CDR2: | SRDNSKNTLYLQMNSLRAEDTAVYYCAANRQGRAL |
| | AISWGGSAVYYANSV | VRKEFYDYWGQGTLVTVSS |
| | KG | (SEQ ID NO: 134) |
| | (SEQ ID NO: 132) | |
| | CDR3: | |
| | NRQGRALVRKEFYDY | |
| | (SEQ ID NO: 133) | |
| αLyPD3 h840 | CDR1: | VH: |
| | GDTSSIYLMG | QVQLLESGGGKVQPGGSLRLSCVVSGDTSSIYLMG |
| | (SEQ ID NO: 135) | WYRQTPGKQRELVAHIRGNGRTNYADSVKGRFTIS |
| | CDR2: | RDNSKNTLYLQMNSLRAEDTAVYYCNVRNFWGSAE |
| | HIRGNGRTNYADSVK | YWGQGTLVTVSS |
| | G | (SEQ ID NO: 138) |
| | (SEQ ID NO: 136) | |
| | CDR3: | |
| | RNFWGSAEY | |
| | (SEQ ID NO: 137) | |
| αLyPD3 h885 | CDR1: | VH: |
| | RGTFSRYSMG | QVQLLESGGGLVQPGGSLRLSCVASRGTFSRYSMG |
| | (SEQ ID NO: 139) | WFRQAPGKEREFVGRISWSGDMTYYADSVKGRFTI |
| | CDR2: | SRDNSKNTLYLQMNSLRAEDTAVYYCAASTLGYVW |
| | RISWSGDMTYYADSV | NHPNMYGYWGHGTLVTVSS |
| | KG | (SEQ ID NO: 301) |
| | (SEQ ID NO: 140) | |
| | CDR3: | |
| | STLGYVWNHPNMYGY | |
| | (SEQ ID NO: 141) | |
| αTrop2 hVIB557 | CDR1: | VH: |
| | GRTFSSQSMG | QVQLLESGGGLVQPGGSLRLSCAASGRTFSSQSMG |
| | (SEQ ID NO: 142) | WFRQAPGKEREFVSAISWTGANPTYADSVKGRFTI |
| | CDR2: | SRDNSKNTLYLQMNSLRAEDTAVYYCAADTSGGSY |
| | AISWTGANPTYADSV | YYERATAETSYDYWGQGTLVTVSS |
| | KG | (SEQ ID NO: 145) |
| | (SEQ ID NO: 143) | |
| | CDR3: | |
| | DTSGGSYYYERATAE | |
| | TSYDY | |
| | (SEQ ID NO: 144) | |
| αTrop2 hVIB565 | CDR1: | VH: |
| | GFTFDYYAIG | QVQLLESGGGLVQPGGSLRLSCAASGFTEDYYAIG |
| | (SEQ ID NO: 146) | WFRQAPGKEREGVSCISSSHGSTYYADSVKGRFTI |
| | CDR2: | SRDNSKNTVYLQMNSLRAEDTAVYYCATAGDGGDY |
| | CISSSHGSTYYADSV | HCSGLVDYGMDYWGKGTLVTVSS |
| | KG | (SEQ ID NO: 149) |
| | (SEQ ID NO: 147) | |
| | CDR3: | |
| | AGDGGDYHCSGLVDY | |
| | GMDY | |
| | (SEQ ID NO: 148) | |
| αTrop2 hVIB575 | CDR1: | VH: |
| | GRTVGRTAMG | QVQLLESGGGLVQPGGSLRLSCLASGRTVGRTAMG |
| | (SEQ ID NO: 150) | WFRQPPGKEREFVATISWAGGTTYYADFVKGRFTI |
| | CDR2: | SRDNSKNTLYLQMNSLRAEDTAVYYCAASEPYSDY |
| | TISWAGGTTYYADFV | DPSGMVYWGKGTLVTVSS |
| | KG | (SEQ ID NO: 153) |
| | (SEQ ID NO: 151) | |
| | CDR3: | |
| | SEPYSDYDPSGMVY | |
| | (SEQ ID NO: 152) | |
| αTrop2 hVIB578 | CDR1: | VH: |
| | GRTFGRAAMG | QVQLLESGGGLVQPGGSLRLSCAASGRTFGRAAMG |
| | (SEQ ID NO: 154) | WFRQPPGKEREFAATISWSGSNTYYADFVKGRFTI |
| | CDR2: | SRDNSKNTVYLQMNSLRAEDTAVYYCAASEPYSDY |
| | TISWSGSNTYYADFV | DPSGMVYWGKGTLVTVSS |
| | KG | (SEQ ID NO: 156) |
| | (SEQ ID NO: 155) | |
| | CDR3: | |
| | SEPYSDYDPSGMVY | |
| | (SEQ ID NO: 152) | |
| αTrop2 hVIB609 | CDR1: | VH: |
| | GLTFNTYPMA | QVQLLESGGGLVQPGGSLRLSCALSGLTFNTYPMA |
| | (SEQ ID NO: 157) | WFRQPPGQEREFVADMSWSGTNTYYADSVKGRFTI |
| | CDR2: | SRDNSKNTLYLQMNSLRAEDTAVYYCAAGWPYSGT |
| | DMSWSGTNTYYADSV | GRSTTDYTYWGQGTLVTVSS |
| | KG | (SEQ ID NO: 160) |
| | (SEQ ID NO: 158) | |
| | CDR3: | |
| | GWPYSGTGRSTTDYT | |
| | Y | |
| | (SEQ ID NO: 159) | |
| αTrop2 hVIB619 | CDR1: | VH: |
| | GRSFSRYGMG | QVQLLESGGGLVQPGGSLRLSCAASGRSFSRYGMG |
| | (SEQ ID NO: 161) | WLRQAPGKERELVASISWSGHSTYYADSVKGRFTI |
| | CDR2: | SRDNSKNTLYLQMNSLRAEDTAVYYCAAESLPYES |
| | SISWSGHSTYYADSV | GSPRLTDFASWGQGTLVTVSS |
| | KG | (SEQ ID NO: 164) |
| | (SEQ ID NO: 162) | |
| | CDR3: | |
| | ESLPYESGSPRLTDF | |
| | AS | |
| | (SEQ ID NO: 163) | |
| αB7H3 hF7 | CDR1: | VH: |
| | RRTFHTYHMG | QVQLQESGGGLVQPGGSLRLSCAPSRRTFHTYHMG |
| | (SEQ ID NO: 165) | WFRQAPGKEREFVAVINWSGGSTVYADSVKGRFTI |
| | CDR2: | SRDNSKNTLYLQMNSLRAEDTAVYYCAAGGATTQR |
| | VINWSGGSTVYADSV | ATEASYDYWGQGTLVTVSS |
| | KG | (SEQ ID NO: 168) |
| | (SEQ ID NO: 166) | |
| | CDR3: | |
| | GGATTQRATEASYDY | |
| | (SEQ ID NO: 167) | |
| αB7H3 hF12 | CDR1: | VH: |
| | PRTFSTYSMA | QVQLQESGGGLVQPGGSLRLSCEASPRTESTYSMA |
| | (SEQ ID NO: 169) | WFRQAPGKERSFVAAINWSGGNTSYADSVKGRFTI |
| | CDR2: | SRDNSKNTLYLQMNSLRAEDTAVYYCAAGGVLAHH |
| | AINWSGGNTSYADSV | NYEYDYWGQGTLVTVSS |
| | KG | (SEQ ID NO: 172) |
| | (SEQ ID NO: 170) | |
| | CDR3: | |
| | GGVLAHHNYEYDY | |
| | (SEQ ID NO: 171) | |
| αB7H3 hF12 | CDR1: | VH: |
| (N57Q) | PRTESTYSMA | QVQLQESGGGLVQPGGSLRLSCEASPRTESTYSMA |
| | (SEQ ID NO: 169) | WFRQAPGKERSFVAAINWSGGQTSYADSVKGRFTI |
| | CDR2: | SRDNSKNTLYLQMNSLRAEDTAVYYCAAGGVLAHH |
| | AINWSGGQTSYADSV | NYEYDYWGQGTLVTVSS |
| | KG | (SEQ ID NO: 174) |
| | (SEQ ID NO: 173) | |
| | CDR3: | |
| | GGVLAHHNYEYDY | |
| | (SEQ ID NO: 171) | |
| αB7H3 HF12 | CDR1: | VH: |
| (N57E) | PRTFSTYSMA | QVQLQESGGGLVQPGGSLRLSCEASPRTESTYSMA |
| | (SEQ ID NO: 169) | WFRQAPGKERSFVAAINWSGGETSYADSVKGRFTI |
| | CDR2: | SRDNSKNTLYLQMNSLRAEDTAVYYCAAGGVLAHH |
| | AINWSGGETSYADSV | NYEYDYWGQGTLVTVSS |
| | KG | (SEQ ID NO: 176) |
| | (SEQ ID NO: 175) | |
| | CDR3: | |
| | GGVLAHHNYEYDY | |
| | (SEQ ID NO: 171) | |
| αB7H3 hF12 | CDR1: | VH: |
| (N57D) | PRTFSTYSMA | QVQLQESGGGLVQPGGSLRLSCEASPRTESTYSMA |
| | (SEQ ID NO: 169) | WFRQAPGKERSFVAAINWSGGDTSYADSVKGRFTI |
| | CDR2: | SRDNSKNTLYLQMNSLRAEDTAVYYCAAGGVLAHH |
| | AINWSGGDTSYADSV | NYEYDYWGQGTLVTVSS |
| | KG | (SEQ ID NO: 178) |
| | (SEQ ID NO: 177) | |
| | CDR3: | |
| | GGVLAHHNYEYDY | |
| | (SEQ ID NO: 171) | |
| αB7H3 | CDR1: | VH: |
| hF12(S59A) | PRTFSTYSMA | QVQLQESGGGLVQPGGSLRLSCEASPRTESTYSMA |
| | (SEQ ID NO: 169) | WFRQAPGKERSFVAAINWSGGNTAYADSVKGRFTI |
| | CDR2: | SRDNSKNTLYLQMNSLRAEDTAVYYCAAGGVLAHH |
| | AINWSGGNTAYADSV | NYEYDYWGQGTLVTVSS |
| | KG | (SEQ ID NO: 180) |
| | (SEQ ID NO: 179) | |
| | CDR3: | |
| | GGVLAHHNYEYDY | |
| | (SEQ ID NO: 171) | |
| αB7H3 hF12 | CDR1: | VH: |
| (S59Y) | PRTFSTYSMA | QVQLQESGGGLVQPGGSLRLSCEASPRTESTYSMA |
| | (SEQ ID NO: 169) | WFRQAPGKERSFVAAINWSGGNTYYADSVKGRFTI |
| | CDR2: | SRDNSKNTLYLQMNSLRAEDTAVYYCAAGGVLAHH |
| | AINWSGGNTYYADSV | NYEYDYWGQGTLVTVSS |
| | KG | (SEQ ID NO: 182) |
| | (SEQ ID NO: 181) | |
| | CDR3: | |
| | GGVLAHHNYEYDY | |
| | (SEQ ID NO: 171) | |
| αCA9 hVIB456 | CDR1: | VH: |
| | GSALIINAMG | QVQLVESGGGLVQPGGSLRLSCAASGSALIINAMG |
| | (SEQ ID NO: 183) | WYRQAPGKQRELVATVTRSGRTNYADSVKGRFTIS |
| | CDR2: | RDNSKNTLYLQMNSLRAEDTAVYYCNVALWIADGE |
| | TVTRSGRTNYADSVK | YDYWGQGTLVTVSS |
| | G | (SEQ ID NO: 186) |
| | (SEQ ID NO: 184) | |
| | CDR3: | |
| | ALWIADGEYDY | |
| | (SEQ ID NO: 185) | |
| αCA9 hVIB476 | CDR1: | VH: |
| | GNIFIINVMG | QVQLVESGGGLVQPGGSLRLSCAASGNIFIINVMG |
| | (SEQ ID NO: 187) | WYRQAPGKQRELVATITNGGRTHYADSVKGRFTIS |
| | CDR2: | RDNSKNTLYLQMNSLRAEDTAVYYCNANHIELGDY |
| | TITNGGRTHYADSVK | WGQGTLVTVSS |
| | G | (SEQ ID NO: 190) |
| | (SEQ ID NO: 188) | |
| | CDR3: | |
| | NHIELGDY | |
| | (SEQ ID NO: 189) | |
| αCA9 hVIB407 | CDR1: | VH: |
| | GIIFSVYDMG | QVQLVESGGGLVQPGGSLRLSCTASGIIFSVYDMG |
| | (SEQ ID NO: 191) | WYRQTPGKQREFVARITAGGGTYLTDSVKGRFTIS |
| | CDR2: | RDNSKNTLYLQMNSLRAEDTGVYYCNAAWIGDDYW |
| | RITAGGGTYLTDSVK | GQGTLVTVSS |
| | G | (SEQ ID NO: 194) |
| | (SEQ ID NO: 192) | |
| | CDR3: | |
| | AWIGDDY | |
| | (SEQ ID NO: 193) | |
| αCA9 hVIB445 | CDR1: | VH: |
| | GITFNLHAMR | QVQLVESGGGLVKPGGSLRLSCAASGITFNLHAMR |
| | (SEQ ID NO: 195) | WYRRAPGKQRELVAYISARDWTNYADSVKGRFTIS |
| | CDR2: | RDNAKNSLYLQMNSLRAEDTAVYYCNTDLVGEDYW |
| | YISARDWTNYADSVK | GRGTLVTVSS |
| | G | (SEQ ID NO: 198) |
| | (SEQ ID NO: 196) | |
| | CDR3: | |
| | DLVGEDY | |
| | (SEQ ID NO: 197) | |

Immune cell antigen directed scFvs

| | | |
|---|---|---|
| αCD3 2B2 | vhCDR1: | VH: |
| | GFTFNKYAIN | EVQLVESGGGLVQPGGSLKLSCAASGFTENKYAIN |
| | (SEQ ID NO: 199) | WVRQAPGKGLEWVARIRSKYNNYATYYADQVKDRF |
| | vhCDR2: | TISRDDSKNTAYLQMNNLKTEDTAVYYCVRHANFG |
| | RIRSKYNNYATYYAD | NSYISYWAYWGQGTLVTVSS |
| | QVKD | (SEQ ID NO: 205) |
| | (SEQ ID NO: 200) | |
| | vhCDR3: | |
| | HANFGNSYISYWAY | |
| | (SEQ ID NO: 201) | |
| | vlCDR1: | VL: |
| | ASSTGAVTSGNYPN | QTVVTQEPSLTVSPGGTVTLTCASSTGAVTSGNYP |
| | (SEQ ID NO: 202) | NWVQQKPGQAPRGLIGGTKFLVPGTPARFSGSLLG |
| | vlCDR2: | GKAALTLSGVQPEDEAEYYCTLWYSNRWVFGGGTK |
| | GTKFLVP | LTVL |
| | (SEQ ID NO: 203) | (SEQ ID NO: 206) |
| | vlCDR3: | |
| | TLWYSNRWV | |
| | (SEQ ID NO: 204) | |
| αCD28 (TGN | vhCDR1: | VH: |
| 1412) | GYTFTSYYIH | QVQLVQSGAEVKKPGASVKVSCKASGYTFTSYYIH |
| | (SEQ ID NO: 207) | WVRQAPGQGLEWIGCIYPGNVNTNYNEKFKDRATL |
| | vhCDR2: | TVDTSISTAYMELSRLRSDDTAVYFCTRSHYGLDW |
| | CIYPGNVNTNYNEKF | NFDVWGQGTTVTVSS |
| | KD | (SEQ ID NO: 213) |
| | (SEQ ID NO: 208) | |
| | vhCDR3: | |
| | SHYGLDWNFDV | |
| | (SEQ ID NO: 209) | |
| | vlCDR1: | VL: |
| | HASQNIYVWLN | DIQMTQSPSSLSASVGDRVTITCHASQNIYVWLNW |
| | (SEQ ID NO: 210) | YQQKPGKAPKLLIYKASNLHTGVPSRFSGSGSGTD |
| | vlCDR2: | FTLTISSLQPEDFATYYCQQGQTYPYTFGGGTKVE |
| | KASNLHT | IKR |
| | (SEQ ID NO: 211) | (SEQ ID NO: 214) |
| | vlCDR3: | |
| | QQGQTYPYT | |
| | (SEQ ID NO: 212) | |
| αCD28 (TGN | vhCDR1: | VH: |
| 1412 mutant | GYTFTSYYIH | QVQLVQSGAEVKKPGASVKVSCKASGYTFTSYYIH |
| with C50S in | (SEQ ID NO: 207) | WVRQAPGQGLEWIGSIYPGNVNTNYNEKFKDRATL |
| the VH; | vhCDR2: | TVDTSISTAYMELSRLRSDDTAVYFCTRSHYGLDW |
| indicated as | SIYPGNVNTNYNEKF | NFDVWGQGTTVTVSS |
| “C-S” in | KD | (SEQ ID NO: 216) |
| Table 3) | (SEQ ID NO: 215) | |
| | vhCDR3: | |
| | SHYGLDWNFDV | |
| | (SEQ ID NO: 209) | |
| | vlCDR1: | VL: |
| | HASQNIYVWLN | DIQMTQSPSSLSASVGDRVTITCHASQNIYVWLNW |
| | (SEQ ID NO: 210) | YQQKPGKAPKLLIYKASNLHTGVPSRFSGSGSGTD |
| | vlCDR2: | FTLTISSLQPEDFATYYCQQGQTYPYTFGGGTKVE |
| | KASNLHT | IKR |
| | (SEQ ID NO: 211) | (SEQ ID NO: 214) |
| | vlCDR3: | |
| | QQGQTYPYT | |
| | (SEQ ID NO: 212) | |

Half-life extenders

| | | |
|---|---|---|
| aHSA 10GE | CDR1: | sdAb: |
| | GFTFSKFGMS | EVQLVESGGGLVQPGNSLRLSCAASGFTFSKFGMS |
| | (SEQ ID NO: 217) | WVRQAPGKGLEWVSSISGSGRDTLYAESVKGRFTI |
| | CDR2: | SRDNAKTTLYLQMNSLRPEDTAVYYCTIGGSLSVS |
| | SISGSGRDTLYAESV | SQGTLVTVSS |
| | KG | (SEQ ID NO: 220) |
| | (SEQ ID NO: 218) | |
| | CDR3: | |
| | GGSLSV | |
| | (SEQ ID NO: 219) | |

Examples of proteins
*A C-terminal affinity tag (e.g., 6XHis tag or TwinStrep tag), if
present in any one of the examples of proteins provided herein, is
optionally and can be replaced by other appropriate affinity tag(s).

| | |
|---|---|
| Pro201 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD3e | AAGSAWYGTLYEYDYWGQGTQVTVSS/GGGSGGGS/EVQLVESGGGLVQ |
| VH/NCL/aCD3 | PGGSLKLSCAASGFTFNKYAINWVRQAPGKGLEWVARIRSKYNNYATYY |
| VL/aEGFR D12/ | ADQVKDRFTISRDDSKNTAYLQMNNLKTEDTAVYYCVRHANFGNSYISY |
| | WAYWGQGTLVTVSS/GGGSGGGS/QTVVTQEPSLTVSPGGTVTLTCASS |
| | TGAVTSGNYPNWVQQKPGQAPRGLIGGTKFLVPGTPARFSGSLLGGKAA |
| | LTLSGVQPEDEAEYYCTLWYSNRWVFGGGTKLTVL/GGGSGGGS/QVKL |
| | EESGGGSVQTGGSLRLTCAASGRTSRSYGMGWFRQAPGKEREFVSGISW |
| | RGDSTGYADSVKGRFTISRDNAKNTVDLQMNSLKPEDTAIYYCAAAAGS |
| | AWYGTLYEYDYWGQGTQVTVSS/SGGPGPAG |
| | (SEQ ID NO: 221) |
| Pro579 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD3e | AAGSAWYGTLYEYDYWGQGTQVTVSS/GGGSGGGS/EVQLVESGGGLVQ |
| VH/NCL/aCD3 | PGGSLKLSCAASGFTFNKYAINWVRQAPGKGLEWVARIRSKYNNYATYY |
| VL/aHEL/6XHis | ADQVKDRFTISRDDSKNTAYLQMNNLKTEDTAVYYCVRHANFGNSYISY |
| | WAYWGQGTLVTVSS/GGGSGGGS/QTVVTQEPSLTVSPGGTVTLTCASS |
| | TGAVTSGNYPNWVQQKPGQAPRGLIGGTKFLVPGTPARFSGSLLGGKAA |
| | LTLSGVQPEDEAEYYCTLWYSNRWVFGGGTKLTVL/GGGSGGGS/DVQL |
| | VESGGGSVQAGGSLRLSCAASGSTDSIEYMTWFRQAPGKAREGVAALYT |
| | HTGNTYYTDSVKGRFTISQDKAKNMAYLRMDSVKSEDTAIYTCGATRKY |
| | VPVRFALDQSSYDYWGQGTQVTVSS/HHHHHH |
| | (SEQ ID NO: 222) |
| Pro646 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD3 | AAGSAWYGTLYEYDYWGQGTQVTVSS/GGGSGGGS/EVQLVESGGGLVQ |
| VH/NCL/aCD3 | PGGSLKLSCAASGFTFNKYAINWVRQAPGKGLEWVARIRSKYNNYATYY |
| VL/MMP9-15/ | ADQVKDRFTISRDDSKNTAYLQMNNLKTEDTAVYYCVRHANFGNSYISY |
| aEGFR D12/ | WAYWGQGTLVTVSS/GGGSGGGS/QTVVTQEPSLTVSPGGTVTLTCASS |
| NCL-15/VLi | TGAVTSGNYPNWVQQKPGQAPRGLIGGTKFLVPGTPARFSGSLLGGKAA |
| Flag/NCL/VHi | LTLSGVQPEDEAEYYCTLWYSNRWVFGGGTKLTVL/SGGPGPAGMKGLP |
| Flag/MMP9- | GS/QVKLEESGGGSVQTGGSLRLTCAASGRTSRSYGMGWFRQAPGKERE |
| 15/aHSA/6XHis | FVSGISWRGDSTGYADSVKGRFTISRDNAKNTVDLQMNSLKPEDTAIYY |
| | CAAAAGSAWYGTLYEYDYWGQGTQVTVSS/GGGSGGGS/QTVVTQEPSL |
| | TVSPGGTVTLTCGSSTGAVTSGNYPNWVQQKPGQAPRGLIGDYKDDDDK |
| | GTPARFSGSLLGGKAALTLSGVQPEDEAEYYCVLWYSNRWVFGGGTKLT |
| | VL/GGGSGGGS/EVQLVESGGGLVQPGGSLKLSCAASGFTFNKYAMNWV |
| | RQAPGKGLEWVARIRSKYDYKDDDDKADSVKDRFTISRDDSKNTAYLQM |
| | NNLKTEDTAVYYCVRHGNFGNSYISYWAYWGQGTLVTVSS/SGGPGPAG |
| | MKGLPGS/EVQLVESGGGLVQPGNSLRLSCAASGFTFSKFGMSWVRQAP |
| | GKGLEWVSSISGSGRDTLYAESVKGRFTISRDNAKTTLYLQMNSLRPED |
| | TAVYYCTIGGSLSVSSQGTLVTVSS/HHHHHH |
| | (SEQ ID NO: 223) |
| Pro861 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD3e | AAGSAWYGTLYEYDYWGQGTQVTVSS/GGGSGGGS/EVQLVESGGGLVQ |
| VH/NCL/aCD3 | PGGSLKLSCAASGFTFNKYAINWVRQAPGKGLEWVARIRSKYNNYATYY |
| VL/TwinStrep | ADQVKDRFTISRDDSKNTAYLQMNNLKTEDTAVYYCVRHANFGNSYISY |
| | WAYWGQGTLVTVSS/GGGSGGGS/QTVVTQEPSLTVSPGGTVTLTCASS |
| | TGAVTSGNYPNWVQQKPGQAPRGLIGGTKFLVPGTPARFSGSLLGGKAA |
| | LTLSGVQPEDEAEYYCTLWYSNRWVFGGGTKLTVL/SGSAWSHPQFEKG |
| | GGSGGGSGGSSAWSHPQFEK |
| | (SEQ ID NO: 224) |
| Pro863 | QVKLEESGGGSVQTGGSLRLTCAASGRTSRSYGMGWFRQAPGKEREFVS |
| aEGFR D12/NCL/ | GISWRGDSTGYADSVKGRFTISRDNAKNTVDLQMNSLKPEDTAIYYCAA |
| aCD3 VL/NCL/ | AAGSAWYGTLYEYDYWGQGTQVTVSS/GGGSGGGS/QTVVTQEPSLTVS |
| aCD3e VH/ | PGGTVTLTCASSTGAVTSGNYPNWVQQKPGQAPRGLIGGTKFLVPGTPA |
| TwinStrep | RFSGSLLGGKAALTLSGVQPEDEAEYYCTLWYSNRWVFGGGTKLTVL/G |
| | GGSGGGS/EVQLVESGGGLVQPGGSLKLSCAASGFTFNKYAINWVRQAP |
| | GKGLEWVARIRSKYNNYATYYADQVKDRFTISRDDSKNTAYLQMNNLKT |
| | EDTAVYYCVRHANFGNSYISYWAYWGQGTLVTVSS/SGSAWSHPQFEKG |
| | GGSGGGSGGSSAWSHPQFEK |
| | (SEQ ID NO: 225) |
| Pro935 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD28.1 | AAGSAWYGTLYEYDYWGQGTQVTVSS/GGGSGGGS/DIQMTQSPSSLSA |
| VL/NCL/aCD28.1 | SVGDRVTITCHASQNIYVWLNWYQQKPGKAPKLLIYKASNLHTGVPSRF |
| VH/NCL/aEGFR | SGSGSGTDFTLTISSLQPEDFATYYCQQGQTYPYTFGGGTKVEIKR/GG |
| D12/6XHis | GSGGGS/QVQLVQSGAEVKKPGASVKVSCKASGYTFTSYYIHWVRQAPG |
| | QGLEWIGCIYPGNVNTNYNEKFKDRATLTVDTSISTAYMELSRLRSDDT |
| | AVYFCTRSHYGLDWNFDVWGQGTTVTVSSGGGSGGGSQVKLEESGGGSV |
| | QTGGSLRLTCAASGRTSRSYGMGWFRQAPGKEREFVSGISWRGDSTGYA |
| | DSVKGRFTISRDNAKNTVDLQMNSLKPEDTAIYYCAAAAGSAWYGTLYE |
| | YDYWGQGTQVTVSS/HHHHHH |
| | (SEQ ID NO: 226) |
| Pro936 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD28.2 | AAGSAWYGTLYEYDYWGQGTQVTVSS/GGGSGGGS/DIQMTQSPSSLSA |
| VL/NCL/aCD28.2 | SVGDRVTITCQASQNIYVWLNWYQQKPGKAPKLLIYKASNLHTGVPSRF |
| VH/NCL/aEGFR | SGSGSGTDFTLTISSLQPEDIATYYCQQGQTYPYTFGQGTKLEIKR/GG |
| D12/6XHis | GSGGGS/QVQLVQSGAEVVKPGASVKVSCKASGYTFTSYYIHWVRQAPG |
| | QGLEWIGSIYPGNVNTNYAQKFQGRATLTVDTSISTAYMELSRLRSDDT |
| | AVYYCTRSHYGLDWNFDVWGKGTTVTVSS/GGGSGGGS/QVKLEESGGG |
| | SVQTGGSLRLTCAASGRTSRSYGMGWFRQAPGKEREFVSGISWRGDSTG |
| | YADSVKGRFTISRDNAKNTVDLQMNSLKPEDTAIYYCAAAAGSAWYGTL |
| | YEYDYWGQGTQVTVSS/HHHHHH |
| | (SEQ ID NO: 227) |
| Pro938 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD28.1 | AAGSAWYGTLYEYDYWGQGTQVTVSS/GGGSGGGS/QVQLVQSGAEVKK |
| VH/NCL/aCD28.1 | PGASVKVSCKASGYTFTSYYIHWVRQAPGQGLEWIGCIYPGNVNTNYNE |
| VL/NCL/aEGFR | KFKDRATLTVDTSISTAYMELSRLRSDDTAVYFCTRSHYGLDWNFDVWG |
| D12/6XHis | QGTTVTVSS/GGGSGGGS/DIQMTQSPSSLSASVGDRVTITCHASQNIY |
| | VWLNWYQQKPGKAPKLLIYKASNLHTGVPSRFSGSGSGTDFTLTISSLQ |
| | PEDFATYYCQQGQTYPYTFGGGTKVEIKR/GGGSGGGS/QVKLEESGGG |
| | SVQTGGSLRLTCAASGRTSRSYGMGWFRQAPGKEREFVSGISWRGDSTG |
| | YADSVKGRFTISRDNAKNTVDLQMNSLKPEDTAIYYCAAAAGSAWYGTL |
| | YEYDYWGQGTQVTVSS/HHHHHH |
| | (SEQ ID NO: 228) |
| Pro939 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD28.2 | AAGSAWYGTLYEYDYWGQGTQVTVSS/GGGSGGGS/QVQLVQSGAEVVK |
| VH/NCL/aCD28.2 | PGASVKVSCKASGYTFTSYYIHWVRQAPGQGLEWIGSIYPGNVNTNYAQ |
| VL/NCL/aEGFR | KFQGRATLTVDTSISTAYMELSRLRSDDTAVYYCTRSHYGLDWNFDVWG |
| D12/6XHis | KGTTVTVSS/GGGSGGGS/DIQMTQSPSSLSASVGDRVTITCQASQNIY |
| | VWLNWYQQKPGKAPKLLIYKASNLHTGVPSRFSGSGSGTDFTLTISSLQ |
| | PEDIATYYCQQGQTYPYTFGQGTKLEIKR/GGGSGGGS/QVKLEESGGG |
| | SVQTGGSLRLTCAASGRTSRSYGMGWFRQAPGKEREFVSGISWRGDSTG |
| | YADSVKGRFTISRDNAKNTVDLQMNSLKPEDTAIYYCAAAAGSAWYGTL |
| | YEYDYWGQGTQVTVSS/HHHHHH |
| | (SEQ ID NO: 229) |
| Pro1034 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD28.1 | AAGSAWYGTLYEYDYWGQGTQVTVSS/GGGSGGGS/QVQLVQSGAEVKK |
| VH C-S/NCL/ | PGASVKVSCKASGYTFTSYYIHWVRQAPGQGLEWIGSIYPGNVNTNYNE |
| aCD28.1 VL/ | KFKDRATLTVDTSISTAYMELSRLRSDDTAVYFCTRSHYGLDWNFDVWG |
| NCL/aEGFR | QGTTVTVSS/GGGSGGGS/DIQMTQSPSSLSASVGDRVTITCHASQNIY |
| D12/6XHis | VWLNWYQQKPGKAPKLLIYKASNLHTGVPSRFSGSGSGTDFTLTISSLQ |
| | PEDFATYYCQQGQTYPYTFGGGTKVEIKR/GGGSGGGS/QVKLEESGGG |
| | SVQTGGSLRLTCAASGRTSRSYGMGWFRQAPGKEREFVSGISWRGDSTG |
| | YADSVKGRFTISRDNAKNTVDLQMNSLKPEDTAIYYCAAAAGSAWYGTL |
| | YEYDYWGQGTQVTVSS/HHHHHH |
| | (SEQ ID NO: 230) |
| Pro1035 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD28.1 | AAGSAWYGTLYEYDYWGQGTQVTVSS/GGGSGGGS/DIQMTQSPSSLSA |
| VL/NCL/aCD28.1 | SVGDRVTITCHASQNIYVWLNWYQQKPGKAPKLLIYKASNLHTGVPSRF |
| VH C-S/NCL/ | SGSGSGTDFTLTISSLQPEDFATYYCQQGQTYPYTFGGGTKVEIKR/GG |
| aEGFR D12/ | GSGGGS/QVQLVQSGAEVKKPGASVKVSCKASGYTFTSYYIHWVRQAPG |
| 6XHis | QGLEWIGSIYPGNVNTNYNEKFKDRATLTVDTSISTAYMELSRLRSDDT |
| | AVYFCTRSHYGLDWNFDVWGQGTTVTVSS/GGGSGGGS/QVKLEESGGG |
| | SVQTGGSLRLTCAASGRTSRSYGMGWFRQAPGKEREFVSGISWRGDSTG |
| | YADSVKGRFTISRDNAKNTVDLQMNSLKPEDTAIYYCAAAAGSAWYGTL |
| | YEYDYWGQGTQVTVSS/HHHHHH |
| | (SEQ ID NO: 231) |
| Pro1134 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD28.1 | AAGSAWYGTLYEYDYWGQGTQVTVSS/GGGSGGGS/QVQLVQSGAEVKK |
| VH C-S/NCL/ | PGASVKVSCKASGYTFTSYYIHWVRQAPGQGLEWIGSIYPGNVNTNYNE |
| aCD28.1 VL/ | KFKDRATLTVDTSISTAYMELSRLRSDDTAVYFCTRSHYGLDWNFDVWG |
| 6XHis | QGTTVTVSS/GGGSGGGS/DIQMTQSPSSLSASVGDRVTITCHASQNIY |
| | VWLNWYQQKPGKAPKLLIYKASNLHTGVPSRFSGSGSGTDFTLTISSLQ |
| | PEDFATYYCQQGQTYPYTFGGGTKVEIKR/HHHHHH |
| | (SEQ ID NO: 232) |
| Pro1135 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD28.1 | AAGSAWYGTLYEYDYWGQGTQVTVSS/GGGSGGGS/DIQMTQSPSSLSA |
| VL/NCL/aCD28.1 | SVGDRVTITCHASQNIYVWLNWYQQKPGKAPKLLIYKASNLHTGVPSRF |
| VH C-S/6XHis | SGSGSGTDFTLTISSLQPEDFATYYCQQGQTYPYTFGGGTKVEIKR/GG |
| | GSGGGS/QVQLVQSGAEVKKPGASVKVSCKASGYTFTSYYIHWVRQAPG |
| | QGLEWIGSIYPGNVNTNYNEKFKDRATLTVDTSISTAYMELSRLRSDDT |
| | AVYFCTRSHYGLDWNFDVWGQGTTVTVSS/HHHHHH |
| | (SEQ ID NO: 233) |
| Pro1136 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD3 | AAGSAWYGTLYEYDYWGQGTQVTVSS/GGGSGGGS/EVQLVESGGGLVQ |
| VH/NCL/aCD3 | PGGSLKLSCAASGFTFNKYAINWVRQAPGKGLEWVARIRSKYNNYATYY |
| VL/ [symbol] [symbol] | ADQVKDRFTISRDDSKNTAYLQMNNLKTEDTAVYYCVRHANFGNSYISY |
| aEGFR D12/ | WAYWGQGTLVTVSS/GGGSGGGS/QTVVTQEPSLTVSPGGTVTLTCASS |
| NCL/aCD28.1 | TGAVTSGNYPNWVQQKPGQAPRGLIGGTKFLVPGTPARFSGSLLGGKAA |
| VH C-S/NCL/ | LTLSGVQPEDEAEYYCTLWYSNRWVFGGGTKLTVL/ [symbol] |
| aCD28.1 | [symbol] QVKLEESGGGSVQTGGSLRLTCAASGRTSRSYGMGWFRQAPGKERE |
| VL/ [symbol] / | FVSGISWRGDSTGYADSVKGRFTISRDNAKNTVDLQMNSLKPEDTAIYY |
| aHSA/6XHis | CAAAAGSAWYGTLYEYDYWGQGTQVTVSS/GGGSGGGS/QVQLVQSGAE |
| | VKKPGASVKVSCKASGYTFTSYYIHWVRQAPGQGLEWIGSIYPGNVNTN |
| | YNEKFKDRATLTVDTSISTAYMELSRLRSDDTAVYFCTRSHYGLDWNFD |
| | VWGQGTTVTVSS/GGGSGGGS/DIQMTQSPSSLSASVGDRVTITCHASQ |
| | NIYVWLNWYQQKPGKAPKLLIYKASNLHTGVPSRFSGSGSGTDFTLTIS |
| | SLQPEDFATYYCQQGQTYPYTFGGGTKVEIKR/ [symbol] |
| | EVQLVESGGGLVQPGNSLRLSCAASGFTFSKFGMSWVRQAPGKGLEWVS |
| | SISGSGRDTLYAESVKGRFTISRDNAKTTLYLQMNSLRPEDTAVYYCTI |
| | GGSLSVSSQGTLVTVSS/HHHHHH |
| | (SEQ ID NO: 234) |
| Pro1137 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD3 | AAGSAWYGTLYEYDYWGQGTQVTVSS/GGGSGGGS/EVQLVESGGGLVQ |
| VH/NCL/aCD3 | PGGSLKLSCAASGFTFNKYAINWVRQAPGKGLEWVARIRSKYNNYATYY |
| VL/ [symbol] / | ADQVKDRFTISRDDSKNTAYLQMNNLKTEDTAVYYCVRHANFGNSYISY |
| aEGFR D12/ | WAYWGQGTLVTVSS/GGGSGGGS/QTVVTQEPSLTVSPGGTVTLTCASS |
| NCL/aCD28.1 | TGAVTSGNYPNWVQQKPGQAPRGLIGGTKFLVPGTPARFSGSLLGGKAA |
| VL/NCL/aCD28.1 | LTLSGVQPEDEAEYYCTLWYSNRWVFGGGTKLTVL/ [symbol] |
| VH C-S/ [symbol] | [symbol] QVKLEESGGGSVQTGGSLRLTCAASGRTSRSYGMGWFRQAPGKERE |
| [symbol] aHSA/6XHis | FVSGISWRGDSTGYADSVKGRFTISRDNAKNTVDLQMNSLKPEDTAIYY |
| | CAAAAGSAWYGTLYEYDYWGQGTQVTVSS/GGGSGGGS/DIQMTQSPSS |
| | LSASVGDRVTITCHASQNIYVWLNWYQQKPGKAPKLLIYKASNLHTGVP |
| | SRFSGSGSGTDFTLTISSLQPEDFATYYCQQGQTYPYTFGGGTKVEIKR |
| | /GGGSGGGS/QVQLVQSGAEVKKPGASVKVSCKASGYTFTSYYIHWVRQ |
| | APGQGLEWIGSIYPGNVNTNYNEKFKDRATLTVDTSISTAYMELSRLRS |
| | DDTAVYFCTRSHYGLDWNFDVWGQGTTVTVSS/ [symbol] |
| | EVQLVESGGGLVQPGNSLRLSCAASGFTFSKFGMSWVRQAPGKGLEWVS |
| | SISGSGRDTLYAESVKGRFTISRDNAKTTLYLQMNSLRPEDTAVYYCTI |
| | GGSLSVSSQGTLVTVSS/HHHHHH |
| | (SEQ ID NO: 235) |
| Pro1138 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD3 | AAGSAWYGTLYEYDYWGQGTQVTVSS/GGGSGGGS/QTVVTQEPSLTVS |
| VL/NCL/aCD3 | PGGTVTLTCASSTGAVTSGNYPNWVQQKPGQAPRGLIGGTKFLVPGTPA |
| VH/ [symbol] | RFSGSLLGGKAALTLSGVQPEDEAEYYCTLWYSNRWVFGGGTKLTVL/G |
| aEGFR D12/ | GGSGGGS/EVQLVESGGGLVQPGGSLKLSCAASGFTFNKYAINWVRQAP |
| NCL/aCD28.1 | GKGLEWVARIRSKYNNYATYYADQVKDRFTISRDDSKNTAYLQMNNLKT |
| VH C-S/NCL/ | EDTAVYYCVRHANFGNSYISYWAYWGQGTLVTVSS/ [symbol] |
| aCD28.1 VL/ | [symbol] QVKLEESGGGSVQTGGSLRLTCAASGRTSRSYGMGWFRQAPGKER |
| [symbol] [symbol] | EFVSGISWRGDSTGYADSVKGRFTISRDNAKNTVDLQMNSLKPEDTAIY |
| aHSA/6XHis | YCAAAAGSAWYGTLYEYDYWGQGTQVTVSS/GGGSGGGS/QVQLVQSGA |
| | EVKKPGASVKVSCKASGYTFTSYYIHWVRQAPGQGLEWIGSIYPGNVNT |
| | NYNEKFKDRATLTVDTSISTAYMELSRLRSDDTAVYFCTRSHYGLDWNF |
| | DVWGQGTTVTVSS/GGGSGGGS/DIQMTQSPSSLSASVGDRVTITCHAS |
| | QNIYVWLNWYQQKPGKAPKLLIYKASNLHTGVPSRFSGSGSGTDFTLTI |
| | SSLQPEDFATYYCQQGQTYPYTFGGGTKVEIKR/ |
| | [symbol] EVQLVESGGGLVQPGNSLRLSCAASGFTFSKFG |
| | MSWVRQAPGKGLEWVSSISGSGRDTLYAESVKGRFTISRDNAKTTLYLQ |
| | MNSLRPEDTAVYYCTIGGSLSVSSQGTLVTVSS/HHHHHH |
| | (SEQ ID NO: 236) |
| Pro1139 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD3 | AAGSAWYGTLYEYDYWGQGTQVTVSS/GGGSGGGS/QTVVTQEPSLTVS |
| VL/NCL/aCD3 | PGGTVTLTCASSTGAVTSGNYPNWVQQKPGQAPRGLIGGTKFLVPGTPA |
| VH/ [symbol] | RFSGSLLGGKAALTLSGVQPEDEAEYYCTLWYSNRWVFGGGTKLTVL/G |
| aEGFR D12/ | GGSGGGS/EVQLVESGGGLVQPGGSLKLSCAASGFTFNKYAINWVRQAP |
| NCL/ aCD28.1 | GKGLEWVARIRSKYNNYATYYADQVKDRFTISRDDSKNTAYLQMNNLKT |
| VL/NCL/ | EDTAVYYCVRHANFGNSYISYWAYWGQGTLVTVSS/ [symbol] |
| aCD28.1 VH | [symbol] QVKLEESGGGSVQTGGSLRLTCAASGRTSRSYGMGWFRQAPGKER |
| C-S/ [symbol] [symbol] | EFVSGISWRGDSTGYADSVKGRFTISRDNAKNTVDLQMNSLKPEDTAIY |
| aHSA/6XHis | YCAAAAGSAWYGTLYEYDYWGQGTQVTVSS/GGGSGGGS/DIQMTQSPS |
| | SLSASVGDRVTITCHASQNIYVWLNWYQQKPGKAPKLLIYKASNLHTGV |
| | PSRFSGSGSGTDFTLTISSLQPEDFATYYCQQGQTYPYTFGGGTKVEIK |
| | R/GGGSGGGS/QVQLVQSGAEVKKPGASVKVSCKASGYTFTSYYIHWVR |
| | QAPGQGLEWIGSIYPGNVNTNYNEKFKDRATLTVDTSISTAYMELSRLR |
| | SDDTAVYFCTRSHYGLDWNFDVWGQGTTVTVSS/ |
| | [symbol] EVQLVESGGGLVQPGNSLRLSCAASGFTFSKFG |
| | MSWVRQAPGKGLEWVSSISGSGRDTLYAESVKGRFTISRDNAKTTLYLQ |
| | MNSLRPEDTAVYYCTIGGSLSVSSQGTLVTVSS/HHHHHH |
| | (SEQ ID NO: 237) |
| Pro1140 | QVKLEESGGGSVQTGGSLRLTCAASGRTSRSYGMGWFRQAPGKEREFVS |
| aEGFR D12/NCL/ | GISWRGDSTGYADSVKGRFTISRDNAKNTVDLQMNSLKPEDTAIYYCAA |
| aCD28.1 VH | AAGSAWYGTLYEYDYWGQGTQVTVSS/GGGSGGGS/QVQLVQSGAEVKK |
| C-S/NCL/aCD28.1 | PGASVKVSCKASGYTFTSYYIHWVRQAPGQGLEWIGSIYPGNVNTNYNE |
| VL/ [symbol] [symbol] aEGFR | KFKDRATLTVDTSISTAYMELSRLRSDDTAVYFCTRSHYGLDWNFDVWG |
| D12/NCL/aCD3 | QGTTVTVSS/GGGSGGGS/DIQMTQSPSSLSASVGDRVTITCHASQNIY |
| VH/NCL/aCD3 | VWLNWYQQKPGKAPKLLIYKASNLHTGVPSRFSGSGSGTDFTLTISSLQ |
| VL/ [symbol] [symbol] aHSA/ | PEDFATYYCQQGQTYPYTFGGGTKVEIKR/ [symbol] /QVK |
| 6XHis | LEESGGGSVQTGGSLRLTCAASGRTSRSYGMGWFRQAPGKEREFVSGIS |
| | WRGDSTGYADSVKGRFTISRDNAKNTVDLQMNSLKPEDTAIYYCAAAAG |
| | SAWYGTLYEYDYWGQGTQVTVSS/GGGSGGGS/EVQLVESGGGLVQPGG |
| | SLKLSCAASGFTFNKYAINWVRQAPGKGLEWVARIRSKYNNYATYYADQ |
| | VKDRFTISRDDSKNTAYLQMNNLKTEDTAVYYCVRHANFGNSYISYWAY |
| | WGQGTLVTVSS/GGGSGGGS/QTVVTQEPSLTVSPGGTVTLTCASSTGA |
| | VTSGNYPNWVQQKPGQAPRGLIGGTKFLVPGTPARFSGSLLGGKAALTL |
| | SGVQPEDEAEYYCTLWYSNRWVFGGGTKLTVL/ [symbol] / |
| | EVQLVESGGGLVQPGNSLRLSCAASGFTFSKFGMSWVRQAPGKGLEWVS |
| | SISGSGRDTLYAESVKGRFTISRDNAKTTLYLQMNSLRPEDTAVYYCTI |
| | GGSLSVSSQGTLVTVSS/HHHHHH |
| | (SEQ ID NO: 238) |
| Pro1141 | QVKLEESGGGSVQTGGSLRLTCAASGRTSRSYGMGWFRQAPGKEREFVS |
| aEGFR D12/NCL/ | GISWRGDSTGYADSVKGRFTISRDNAKNTVDLQMNSLKPEDTAIYYCAA |
| aCD28.1 Vl/ | AAGSAWYGTLYEYDYWGQGTQVTVSS/GGGSGGSS/DIQMTQSPSSLSA |
| NCL/aCD28.1 | SVGDRVTITCHASQNIYVWLNWYQQKPGKAPKLLIYKASNLHTGVPSRF |
| Vh C-S/ [symbol] [symbol] | SGSGSGTDFTLTISSLQPEDFATYYCQQGQTYPYTFGGGTKVEIKR/GG |
| aEGFR D12/aCD3 | GSGGGS/QVQLVQSGAEVKKPGASVKVSCKASGYTFTSYYIHWVRQAPG |
| VH/NCL/aCD3 VL/ | QGLEWIGSIYPGNVNTNYNEKFKDRATLTVDTSISTAYMELSRLRSDDT |
| NCL/ [symbol] [symbol] | AVYFCTRSHYGLDWNFDVWGQGTTVTVSS/ [symbol] /QVK |
| aHSA/6XHis | LEESGGGSVQTGGSLRLTCAASGRTSRSYGMGWFRQAPGKEREFVSGIS |
| | WRGDSTGYADSVKGRFTISRDNAKNTVDLQMNSLKPEDTAIYYCAAAAG |
| | SAWYGTLYEYDYWGQGTQVTVSS/GGGSGGGS/EVQLVESGGGLVQPGG |
| | SLKLSCAASGFTFNKYAINWVRQAPGKGLEWVARIRSKYNNYATYYADQ |
| | VKDRFTISRDDSKNTAYLQMNNLKTEDTAVYYCVRHANFGNSYISYWAY |
| | WGQGTLVTVSS/GGGSGGGS/QTVVTQEPSLTVSPGGTVTLTCASSTGA |
| | VTSGNYPNWVQQKPGQAPRGLIGGTKFLVPGTPARFSGSLLGGKAALTL |
| | SGVQPEDEAEYYCTLWYSNRWVFGGGTKLTVL/ [symbol] |
| | EVQLVESGGGLVQPGNSLRLSCAASGFTFSKFGMSWVRQAPGKGLEWVS |
| | SISGSGRDTLYAESVKGRFTISRDNAKTTLYLQMNSLRPEDTAVYYCTI |
| | GGSLSVSSQGTLVTVSS/HHHHHH |
| | (SEQ ID NO: 239) |
| Pro1142 | QVKLEESGGGSVQTGGSLRLTCAASGRTSRSYGMGWFRQAPGKEREFVS |
| aEGFR D12/NCL/ | GISWRGDSTGYADSVKGRFTISRDNAKNTVDLQMNSLKPEDTAIYYCAA |
| aCD28.1 VH C-S/ | AAGSAWYGTLYEYDYWGQGTQVTVSS/GGGSGGGS/QVQLVQSGAEVKK |
| NCL/aCD28.1 VL/ | PGASVKVSCKASGYTFTSYYIHWVRQAPGQGLEWIGSIYPGNVNTNYNE |
| [symbol] [symbol] aEGFR | KFKDRATLTVDTSISTAYMELSRLRSDDTAVYFCTRSHYGLDWNFDVWG |
| D12/NCL/aCD3 | QGTTVTVSS/GGGSGGGS/DIQMTQSPSSLSASVGDRVTITCHASQNIY |
| VL/NCL/aCD3 | VWLNWYQQKPGKAPKLLIYKASNLHTGVPSRFSGSGSGTDFTLTISSLQ |
| VH/ [symbol] [symbol] / | PEDFATYYCQQGQTYPYTFGGGTKVEIKR/ [symbol] /QVK |
| aHSA/6XHis | LEESGGGSVQTGGSLRLTCAASGRTSRSYGMGWFRQAPGKEREFVSGIS |
| | WRGDSTGYADSVKGRFTISRDNAKNTVDLQMNSLKPEDTAIYYCAAAAG |
| | SAWYGTLYEYDYWGQGTQVTVSS/GGGSGGGS/QTVVTQEPSLTVSPGG |
| | TVTLTCASSTGAVTSGNYPNWVQQKPGQAPRGLIGGTKFLVPGTPARFS |
| | GSLLGGKAALTLSGVQPEDEAEYYCTLWYSNRWVFGGGTKLTVL/GGGS |
| | GGGS/EVQLVESGGGLVQPGGSLKLSCAASGFTFNKYAINWVRQAPGKG |
| | LEWVARIRSKYNNYATYYADQVKDRFTISRDDSKNTAYLQMNNLKTEDT |
| | AVYYCVRHANFGNSYISYWAYWGQGTLVTVSS/ [symbol] / |
| | EVQLVESGGGLVQPGNSLRLSCAASGFTFSKFGMSWVRQAPGKGLEWVS |
| | SISGSGRDTLYAESVKGRFTISRDNAKTTLYLQMNSLRPEDTAVYYCTI |
| | GGSLSVSSQGTLVTVSS/HHHHHH |
| | (SEQ ID NO: 240) |
| Pro1143 | QVKLEESGGGSVQTGGSLRLTCAASGRTSRSYGMGWFRQAPGKEREFVS |
| aEGFR D12/NCL/ | GISWRGDSTGYADSVKGRFTISRDNAKNTVDLQMNSLKPEDTAIYYCAA |
| aCD28.1 VL/ | AAGSAWYGTLYEYDYWGQGTQVTVSS/GGGSGGGS/DIQMTQSPSSLSA |
| NCL/aCD28.1 | SVGDRVTITCHASQNIYVWLNWYQQKPGKAPKLLIYKASNLHTGVPSRF |
| VH C-S / [symbol] | SGSGSGTDFTLTISSLQPEDFATYYCQQGQTYPYTFGGGTKVEIKR/GG |
| [symbol] aEGFR D12/ | GSGGGS/QVQLVQSGAEVKKPGASVKVSCKASGYTFTSYYIHWVRQAPG |
| aCD3 VL/ | QGLEWIGSIYPGNVNTNYNEKFKDRATLTVDTSISTAYMELSRLRSDDT |
| NCL/aCD3 VH/ | AVYFCTRSHYGLDWNFDVWGQGTTVTVSS/ [symbol] /QVK |
| NCL/ [symbol] [symbol] | LEESGGGSVQTGGSLRLTCAASGRTSRSYGMGWFRQAPGKEREFVSGIS |
| aHSA/6XHis | WRGDSTGYADSVKGRFTISRDNAKNTVDLQMNSLKPEDTAIYYCAAAAG |
| | SAWYGTLYEYDYWGQGTQVTVSS/GGGSGGGS/QTVVTQEPSLTVSPGG |
| | TVTLTCASSTGAVTSGNYPNWVQQKPGQAPRGLIGGTKFLVPGTPARFS |
| | GSLLGGKAALTLSGVQPEDEAEYYCTLWYSNRWVFGGGTKLTVL/GGGS |
| | GGGS/EVQLVESGGGLVQPGGSLKLSCAASGFTFNKYAINWVRQAPGKG |
| | LEWVARIRSKYNNYATYYADQVKDRFTISRDDSKNTAYLQMNNLKTEDT |
| | AVYYCVRHANFGNSYISYWAYWGQGTLVTVSS/ [symbol] / |
| | EVQLVESGGGLVQPGNSLRLSCAASGFTFSKFGMSWVRQAPGKGLEWVS |
| | SISGSGRDTLYAESVKGRFTISRDNAKTTLYLQMNSLRPEDTAVYYCTI |
| | GGSLSVSSQGTLVTVSS/HHHHHH |
| | (SEQ ID NO: 241) |
| Pro1184 | EVQLVESGGGLVQPGGSLRLSCAASGRTFSSYAMGWFRQAPGKEREFVV |
| aEGFR hG8/NCL/ | AINWSSGSTYYADSVKGRFTISRDNSKNTLYLQMNSLRAEDTAVYYCAA |
| aCD3 VH/NCL/ | GYQINSGNYNFKDYEYDYWGQGTLVTVSS/GGGSGGGS/EVQLVESGGG |
| aCD3 VL/ [symbol] | LVQPGGSLKLSCAASGFTFNKYAINWVRQAPGKGLEWVARIRSKYNNYA |
| [symbol] aEGFR hG8 | TYYADQVKDRFTISRDDSKNTAYLQMNNLKTEDTAVYYCVRHANFGNSY |
| /NCL-15/aCD28.1 | ISYWAYWGQGTLVTVSS/GGGSGGGS/QTVVTQEPSLTVSPGGTVTLTC |
| VH C-S/NCL/ | ASSTGAVTSGNYPNWVQQKPGQAPRGLIGGTKFLVPGTPARFSGSLLGG |
| aCD28.1 VL/NCL- | KAALTLSGVQPEDEAEYYCTLWYSNRWVFGGGTKLTVL/ [symbol] |
| 15/aHSA/6XHis | [symbol] EVQLVESGGGLVQPGGSLRLSCAASGRTFSSYAMGWFRQAPGK |
| | EREFVVAINWSSGSTYYADSVKGRFTISRDNSKNTLYLQMNSLRAEDTA |
| | VYYCAAGYQINSGNYNFKDYEYDYWGQGTLVTVSS/GGGGSGGGGSGGG |
| | GS/QVQLVQSGAEVKKPGASVKVSCKASGYTFTSYYIHWVRQAPGQGLE |
| | WIGSIYPGNVNTNYNEKFKDRATLTVDTSISTAYMELSRLRSDDTAVYF |
| | CTRSHYGLDWNFDVWGQGTTVTVSS/GGGSGGGS/DIQMTQSPSSLSAS |
| | VGDRVTITCHASQNIYVWLNWYQQKPGKAPKLLIYKASNLHTGVPSRFS |
| | GSGSGTDFTLTISSLQPEDFATYYCQQGQTYPYTFGGGTKVEIKR/GGG |
| | GSGGGGSGGGGS/EVQLVESGGGLVQPGNSLRLSCAASGFTFSKFGMSW |
| | VRQAPGKGLEWVSSISGSGRDTLYAESVKGRFTISRDNAKTTLYLQMNS |
| | LRPEDTAVYYCTIGGSLSVSSQGTLVTVSS/HHHHHH |
| | (SEQ ID NO: 242) |
| Pro1192 | EVQLVESGGGLVQPGGSLRLSCAASGRTFSSYAMGWFRQAPGKEREFVV |
| aEGFR hG8/NCL/ | AINWSSGSTYYADSVKGRFTISRDNSKNTLYLQMNSLRAEDTAVYYCAA |
| aCD28.1 VH | GYQINSGNYNFKDYEYDYWGQGTLVTVSS/GGGSGGGS/QVQLVQSGAE |
| C-S/NCL/ | VKKPGASVKVSCKASGYTFTSYYIHWVRQAPGQGLEWIGSIYPGNVNTN |
| aCD28.1 VL/ | YNEKFKDRATLTVDTSISTAYMELSRLRSDDTAVYFCTRSHYGLDWNFD |
| [symbol] [symbol] aEGFR | VWGQGTTVTVSS/GGGSGGGS/DIQMTQSPSSLSASVGDRVTITCHASQ |
| hG8/NCL-15/aCD3 | NIYVWLNWYQQKPGKAPKLLIYKASNLHTGVPSRFSGSGSGTDFTLTIS |
| VH/NCL/aCD3 | SLQPEDFATYYCQQGQTYPYTFGGGTKVEIKR/ [symbol] |
| VL/NCL-15/ | EVQLVESGGGLVQPGGSLRLSCAASGRTFSSYAMGWFRQAPGKEREFVV |
| aHSA/6XHis | AINWSSGSTYYADSVKGRFTISRDNSKNTLYLQMNSLRAEDTAVYYCAA |
| | GYQINSGNYNFKDYEYDYWGQGTLVTVSS/GGGGSGGGGSGGGGS/EVQ |
| | LVESGGGLVQPGGSLKLSCAASGFTFNKYAINWVRQAPGKGLEWVARIR |
| | SKYNNYATYYADQVKDRFTISRDDSKNTAYLQMNNLKTEDTAVYYCVRH |
| | ANFGNSYISYWAYWGQGTLVTVSS/GGGSGGGS/QTVVTQEPSLTVSPG |
| | GTVTLTCASSTGAVTSGNYPNWVQQKPGQAPRGLIGGTKFLVPGTPARF |
| | SGSLLGGKAALTLSGVQPEDEAEYYCTLWYSNRWVFGGGTKLTVL/GGG |
| | GSGGGGSGGGGS/EVQLVESGGGLVQPGNSLRLSCAASGFTFSKFGMSW |
| | VRQAPGKGLEWVSSISGSGRDTLYAESVKGRFTISRDNAKTTLYLQMNS |
| | LRPEDTAVYYCTIGGSLSVSSQGTLVTVSS/HHHHHH |
| | (SEQ ID NO: 243) |
| Pro1265 | QVQLLESGGGLVQPGGSLRLSCAASRNIGSNYAVGWFRQAPGKEREPVA |
| aHER2 hu1156/ | ALRWTGSIIGYDDSLRGRFTISKDNSKNTLYLQMNSLKPEDTAVYYCAA |
| NCL/aCD3 VH/ | RILDRSSYDYWGQGTLVTVSS/GGGSGGGS/EVQLVESGGGLVQPGGSL |
| NCL/aCD3 VL/ | KLSCAASGFTFNKYAINWVRQAPGKGLEWVARIRSKYNNYATYYADQVK |
| [symbol] [symbol] aHER2 | DRFTISRDDSKNTAYLQMNNLKTEDTAVYYCVRHANFGNSYISYWAYWG |
| hu1156/NCL/aCD | QGTLVTVSS/GGGSGGGS/QTVVTQEPSLTVSPGGTVTLTCASSTGAVT |
| 28.1 VH C-S | SGNYPNWVQQKPGQAPRGLIGGTKFLVPGTPARFSGSLLGGKAALTLSG |
| /NCL/aCD28.1 | VQPEDEAEYYCTLWYSNRWVFGGGTKLTVL [symbol] QV |
| VL/ [symbol] [symbol] | QLLESGGGLVQPGGSLRLSCAASRNIGSNYAVGWFRQAPGKEREPVAAL |
| aHSA/6XHis | RWTGSIIGYDDSLRGRFTISKDNSKNTLYLQMNSLKPEDTAVYYCAARI |
| | LDRSSYDYWGQGTLVTVSS/GGGSGGGS/QVQLVQSGAEVKKPGASVKV |
| | SCKASGYTFTSYYIHWVRQAPGQGLEWIGSIYPGNVNTNYNEKFKDRAT |
| | LTVDTSISTAYMELSRLRSDDTAVYFCTRSHYGLDWNFDVWGQGTTVTV |
| | SS/GGGSGGGS/DIQMTQSPSSLSASVGDRVTITCHASQNIYVWLNWYQ |
| | QKPGKAPKLLIYKASNLHTGVPSRFSGSGSGTDFTLTISSLQPEDFATY |
| | YCQQGQTYPYTFGGGTKVEIKR/ [symbol] EVQLVESGGG |
| | LVQPGNSLRLSCAASGFTFSKFGMSWVRQAPGKGLEWVSSISGSGRDTL |
| | YAESVKGRFTISRDNAKTTLYLQMNSLRPEDTAVYYCTIGGSLSVSSQG |
| | TLVTVSS/HHHHHH |
| | (SEQ ID NO: 244) |
| Pro1266 | QVQLLESGGGLVQPGGSLRLSCAASRNIGSNYAVGWFRQAPGKEREPVA |
| aHER2 hu1156 | ALRWTGSIIGYDDSLRGRFTISKDNSKNTLYLQMNSLKPEDTAVYYCAA |
| /NCL/aCD28.1 | RILDRSSYDYWGQGTLVTVSS/GGGSGGGS/QVQLVQSGAEVKKPGASV |
| VH C-S/NCL/ | KVSCKASGYTFTSYYIHWVRQAPGQGLEWIGSIYPGNVNTNYNEKFKDR |
| aCD28.1 VL/ | ATLTVDTSISTAYMELSRLRSDDTAVYFCTRSHYGLDWNFDVWGQGTTV |
| [symbol] | TVSS/GGGSGGGS/DIQMTQSPSSLSASVGDRVTITCHASQNIYVWLNW |
| aHER2 hu1156/ | YQQKPGKAPKLLIYKASNLHTGVPSRFSGSGSGTDFTLTISSLQPEDFA |
| NCL/aCD3 VH/ | TYYCQQGQTYPYTFGGGTKVEIKR/ [symbol] QVQLLESG |
| NCL/aCD3 VL/ | GGLVQPGGSLRLSCAASRNIGSNYAVGWFRQAPGKEREPVAALRWTGSI |
| [symbol] [symbol] aHSA/ | IGYDDSLRGRFTISKDNSKNTLYLQMNSLKPEDTAVYYCAARILDRSSY |
| 6XHis | DYWGQGTLVTVSS/GGGSGGGS/EVQLVESGGGLVQPGGSLKLSCAASG |
| | FTFNKYAINWVRQAPGKGLEWVARIRSKYNNYATYYADQVKDRFTISRD |
| | DSKNTAYLQMNNLKTEDTAVYYCVRHANFGNSYISYWAYWGQGTLVTVS |
| | S/GGGSGGGS/QTVVTQEPSLTVSPGGTVTLTCASSTGAVTSGNYPNWV |
| | QQKPGQAPRGLIGGTKFLVPGTPARFSGSLLGGKAALTLSGVQPEDEAE |
| | YYCTLWYSNRWVFGGGTKLTVL/ [symbol] EVQLVESGGG |
| | LVQPGNSLRLSCAASGFTFSKFGMSWVRQAPGKGLEWVSSISGSGRDTL |
| | YAESVKGRFTISRDNAKTTLYLQMNSLRPEDTAVYYCTIGGSLSVSSQG |
| | TLVTVSS/HHHHHH |
| | (SEQ ID NO: 245) |
| Pro1267 | QVKLEESGGGSVQTGGSLRLTCAASGRTSRSYGMGWFRQAPGKEREFVS |
| aEGFR D12/ | GISWRGDSTGYADSVKGRFTISRDNAKNTVDLQMNSLKPEDTAIYYCAA |
| NCL/aCD3 | AAGSAWYGTLYEYDYWGQGTQVTVSS/GGGSGGGS/EVQLVESGGGLVQ |
| VH/NCL/aCD3 | PGGSLKLSCAASGFTFNKYAINWVRQAPGKGLEWVARIRSKYNNYATYY |
| VL/ [symbol] [symbol] | ADQVKDRFTISRDDSKNTAYLQMNNLKTEDTAVYYCVRHANFGNSYISY |
| aHER2 hu1156/ | WAYWGQGTLVTVSS/GGGSGGGS/QTVVTQEPSLTVSPGGTVTLTCASS |
| NCL/aCD28.1 | TGAVTSGNYPNWVQQKPGQAPRGLIGGTKFLVPGTPARFSGSLLGGKAA |
| VH C-S/NCL/ | LTLSGVQPEDEAEYYCTLWYSNRWVFGGGTKLTVL/ [symbol] |
| aCD28.1 VL/ | [symbol] QVQLLESGGGLVQPGGSLRLSCAASRNIGSNYAVGWFRQAPGKERE |
| [symbol] aHSA/ | PVAALRWTGSIIGYDDSLRGRFTISKDNSKNTLYLQMNSLKPEDTAVYY |
| 6XHis | CAARILDRSSYDYWGQGTLVTVSS/GGGSGGGS/QVQLVQSGAEVKKPG |
| | ASVKVSCKASGYTFTSYYIHWVRQAPGQGLEWIGSIYPGNVNTNYNEKF |
| | KDRATLTVDTSISTAYMELSRLRSDDTAVYFCTRSHYGLDWNFDVWGQG |
| | TTVTVSS/GGGSGGGS/DIQMTQSPSSLSASVGDRVTITCHASQNIYVW |
| | LNWYQQKPGKAPKLLIYKASNLHTGVPSRFSGSGSGTDFTLTISSLQPE |
| | DFATYYCQQGQTYPYTFGGGTKVEIKR/ [symbol] EVQLV |
| | ESGGGLVQPGNSLRLSCAASGFTFSKFGMSWVRQAPGKGLEWVSSISGS |
| | GRDTLYAESVKGRFTISRDNAKTTLYLQMNSLRPEDTAVYYCTIGGSLS |
| | VSSQGTLVTVSS/HHHHHH |
| | (SEQ ID NO: 246) |
| Pro1268 | QVKLEESGGGSVQTGGSLRLTCAASGRTSRSYGMGWFRQAPGKEREFVS |
| aEGFR D12/NCL/ | GISWRGDSTGYADSVKGRFTISRDNAKNTVDLQMNSLKPEDTAIYYCAA |
| aCD28.1 VH | AAGSAWYGTLYEYDYWGQGTQVTVSS/GGGSGGGS/QVQLVQSGAEVKK |
| C-S/NCL/aCD28.1 | PGASVKVSCKASGYTFTSYYIHWVRQAPGQGLEWIGSIYPGNVNTNYNE |
| VL/ [symbol] | KFKDRATLTVDTSISTAYMELSRLRSDDTAVYFCTRSHYGLDWNFDVWG |
| aHER2 hu1156/ | QGTTVTVSS/GGGSGGGS/DIQMTQSPSSLSASVGDRVTITCHASQNIY |
| NCL/aCD3 VH/ | VWLNWYQQKPGKAPKLLIYKASNLHTGVPSRFSGSGSGTDFTLTISSLQ |
| NCL/aCD3 VL/ | PEDFATYYCQQGQTYPYTFGGGTKVEIKR/ [symbol] /QVQ |
| [symbol] [symbol] aHSA/ | LLESGGGLVQPGGSLRLSCAASRNIGSNYAVGWFRQAPGKEREPVAALR |
| 6XHis | WTGSIIGYDDSLRGRFTISKDNSKNTLYLQMNSLKPEDTAVYYCAARIL |
| | DRSSYDYWGQGTLVTVSS/GGGSGGGS/EVQLVESGGGLVQPGGSLKLS |
| | CAASGFTFNKYAINWVRQAPGKGLEWVARIRSKYNNYATYYADQVKDRF |
| | TISRDDSKNTAYLQMNNLKTEDTAVYYCVRHANFGNSYISYWAYWGQGT |
| | LVTVSS/GGGSGGGS/QTVVTQEPSLTVSPGGTVTLTCASSTGAVTSGN |
| | YPNWVQQKPGQAPRGLIGGTKFLVPGTPARFSGSLLGGKAALTLSGVQP |
| | EDEAEYYCTLWYSNRWVFGGGTKLTVL/ [symbol] EVQLV |
| | ESGGGLVQPGNSLRLSCAASGFTFSKFGMSWVRQAPGKGLEWVSSISGS |
| | GRDTLYAESVKGRFTISRDNAKTTLYLQMNSLRPEDTAVYYCTIGGSLS |
| | VSSQGTLVTVSS/HHHHHH |
| | (SEQ ID NO: 247) |
| Pro1269 | QVQLLESGGGLVQPGGSLRLSCAASRNIGSNYAVGWFRQAPGKEREPVA |
| aHER2 hu1156 | ALRWTGSIIGYDDSLRGRFTISKDNSKNTLYLQMNSLKPEDTAVYYCAA |
| /NCL/aCD3 | RILDRSSYDYWGQGTLVTVSS/GGGSGGGS/EVQLVESGGGLVQPGGSL |
| VH/NCL/aCD3 | KLSCAASGFTFNKYAINWVRQAPGKGLEWVARIRSKYNNYATYYADQVK |
| VL/ [symbol] [symbol] | DRFTISRDDSKNTAYLQMNNLKTEDTAVYYCVRHANFGNSYISYWAYWG |
| aEGFRD12/NCL/ | QGTLVTVSS/GGGSGGGS/QTVVTQEPSLTVSPGGTVTLTCASSTGAVT |
| aCD28.1 VH | SGNYPNWVQQKPGQAPRGLIGGTKFLVPGTPARFSGSLLGGKAALTLSG |
| C-S/NCL/ | VQPEDEAEYYCTLWYSNRWVFGGGTKLTVL/ [symbol] QV |
| aCD28.1 VL/ | KLEESGGGSVQTGGSLRLTCAASGRTSRSYGMGWFRQAPGKEREFVSGI |
| [symbol] [symbol] aHSA/ | SWRGDSTGYADSVKGRFTISRDNAKNTVDLQMNSLKPEDTAIYYCAAAA |
| 6XHis | GSAWYGTLYEYDYWGQGTQVTVSS/GGGSGGGS/QVQLVQSGAEVKKPG |
| | ASVKVSCKASGYTFTSYYIHWVRQAPGQGLEWIGSIYPGNVNTNYNEKF |
| | KDRATLTVDTSISTAYMELSRLRSDDTAVYFCTRSHYGLDWNFDVWGQG |
| | TTVTVSS/GGGSGGGS/DIQMTQSPSSLSASVGDRVTITCHASQNIYVW |
| | LNWYQQKPGKAPKLLIYKASNLHTGVPSRFSGSGSGTDFTLTISSLQPE |
| | DFATYYCQQGQTYPYTFGGGTKVEIKR/ [symbol] EVQLV |
| | ESGGGLVQPGNSLRLSCAASGFTFSKFGMSWVRQAPGKGLEWVSSISGS |
| | GRDTLYAESVKGRFTISRDNAKTTLYLQMNSLRPEDTAVYYCTIGGSLS |
| | VSSQGTLVTVSS/HHHHHH |
| | (SEQ ID NO: 248) |
| Pro1270 | QVQLLESGGGLVQPGGSLRLSCAASRNIGSNYAVGWFRQAPGKEREPVA |
| aHER2 hu1156 | ALRWTGSIIGYDDSLRGRFTISKDNSKNTLYLQMNSLKPEDTAVYYCAA |
| /NCL/ aCD28.1 | RILDRSSYDYWGQGTLVTVSS/GGGSGGGS/QVQLVQSGAEVKKPGASV |
| VH C-S/NCL/ | KVSCKASGYTFTSYYIHWVRQAPGQGLEWIGSIYPGNVNTNYNEKFKDR |
| aCD28.1 VL/ | ATLTVDTSISTAYMELSRLRSDDTAVYFCTRSHYGLDWNFDVWGQGTTV |
| [symbol] /aEGFR | TVSS/GGGSGGGS/DIQMTQSPSSLSASVGDRVTITCHASQNIYVWLNW |
| D12/NCL/aCD3 | YQQKPGKAPKLLIYKASNLHTGVPSRFSGSGSGTDFTLTISSLQPEDFA |
| VH/NCL/aCD3 | TYYCQQGQTYPYTFGGGTKVEIKR/ [symbol] QVKLEESG |
| VL/ [symbol] [symbol] | GGSVQTGGSLRLTCAASGRTSRSYGMGWFRQAPGKEREFVSGISWRGDS |
| aHSA/6XHis | TGYADSVKGRFTISRDNAKNTVDLQMNSLKPEDTAIYYCAAAAGSAWYG |
| | TLYEYDYWGQGTQVTVSS/GGGSGGGS/EVQLVESGGGLVQPGGSLKLS |
| | CAASGFTFNKYAINWVRQAPGKGLEWVARIRSKYNNYATYYADQVKDRF |
| | TISRDDSKNTAYLQMNNLKTEDTAVYYCVRHANFGNSYISYWAYWGQGT |
| | LVTVSS/GGGSGGGS/QTVVTQEPSLTVSPGGTVTLTCASSTGAVTSGN |
| | YPNWVQQKPGQAPRGLIGGTKFLVPGTPARFSGSLLGGKAALTLSGVQP |
| | EDEAEYYCTLWYSNRWVFGGGTKLTVL/ [symbol] EVQLV |
| | ESGGGLVQPGNSLRLSCAASGFTFSKFGMSWVRQAPGKGLEWVSSISGS |
| | GRDTLYAESVKGRFTISRDNAKTTLYLQMNSLRPEDTAVYYCTIGGSLS |
| | VSSQGTLVTVSS/HHHHHH |
| | (SEQ ID NO: 249) |
| Pro1185 | EVQLVESGGGLVQPGGSLRLSCAASGRTFSSYAMGWFRQAPGKEREFVV |
| aEGFR hG8/NCL/ | AINWSSGSTYYADSVKGRFTISRDNSKNTLYLQMNSLRAEDTAVYYCAA |
| aCD3 VH/NCL/ | GYQINSGNYNFKDYEYDYWGQGTLVTVSS/GGGSGGGS/EVQLVESGGG |
| aCD3 VL/MMP9- | LVQPGGSLKLSCAASGFTFNKYAINWVRQAPGKGLEWVARIRSKYNNYA |
| 15/aEGFR hG8/ | TYYADQVKDRFTISRDDSKNTAYLQMNNLKTEDTAVYYCVRHANFGNSY |
| NCL-15/VLi | ISYWAYWGQGTLVTVSS/GGGSGGGS/QTVVTQEPSLTVSPGGTVTLTC |
| Flag/NCL/VHi | ASSTGAVTSGNYPNWVQQKPGQAPRGLIGGTKFLVPGTPARFSGSLLGG |
| Flag/NCL-15/ | KAALTLSGVQPEDEAEYYCTLWYSNRWVFGGGTKLTVL/SGGPGPAGMK |
| aHSA/6XHis | GLPGS/EVQLVESGGGLVQPGGSLRLSCAASGRTFSSYAMGWFRQAPGK |
| | EREFVVAINWSSGSTYYADSVKGRFTISRDNSKNTLYLQMNSLRAEDTA |
| | VYYCAAGYQINSGNYNFKDYEYDYWGQGTLVTVSS/GGGGSGGGGSGGG |
| | GS/QTVVTQEPSLTVSPGGTVTLTCGSSTGAVTSGNYPNWVQQKPGQAP |
| | RGLIGDYKDDDDKGTPARFSGSLLGGKAALTLSGVQPEDEAEYYCVLWY |
| | SNRWVFGGGTKLTVL/GGGSGGGS/EVQLVESGGGLVQPGGSLKLSCAA |
| | SGFTFNKYAMNWVRQAPGKGLEWVARIRSKYDYKDDDDKADSVKDRFTI |
| | SRDDSKNTAYLQMNNLKTEDTAVYYCVRHGNFGNSYISYWAYWGQGTLV |
| | TVSS/GGGGSGGGGSGGGGS/EVQLVESGGGLVQPGNSLRLSCAASGFT |
| | FSKFGMSWVRQAPGKGLEWVSSISGSGRDTLYAESVKGRFTISRDNAKT |
| | TLYLQMNSLRPEDTAVYYCTIGGSLSVSSQGTLVTVSS/HHHHHH |
| | (SEQ ID NO: 250) |
| Pro1186 | EVQLVESGGGLVQPGGSLRLSCAASGRTFSSYAMGWFRQAPGKEREFVV |
| aEGFR hG8/NCL/ | AINWSSGSTYYADSVKGRFTISRDNSKNTLYLQMNSLRAEDTAVYYCAA |
| aCD28.1 VH C-S/ | GYQINSGNYNFKDYEYDYWGQGTLVTVSS/GGGSGGGS/QVQLVQSGAE |
| NCL/aCD28.1 | VKKPGASVKVSCKASGYTFTSYYIHWVRQAPGQGLEWIGSIYPGNVNTN |
| VL/MMP9-15/aEGFR | YNEKFKDRATLTVDTSISTAYMELSRLRSDDTAVYFCTRSHYGLDWNFD |
| hG8/NCL-15/VLi | VWGQGTTVTVSS/GGGSGGGS/DIQMTQSPSSLSASVGDRVTITCHASQ |
| Flag /NCL/VHi | NIYVWLNWYQQKPGKAPKLLIYKASNLHTGVPSRFSGSGSGTDFTLTIS |
| Flag/NCL-15/ | SLQPEDFATYYCQQGQTYPYTFGGGTKVEIKR/SGGPGPAGMKGLPGS/ |
| aHSA/6XHis | EVQLVESGGGLVQPGGSLRLSCAASGRTFSSYAMGWFRQAPGKEREFVV |
| | AINWSSGSTYYADSVKGRFTISRDNSKNTLYLQMNSLRAEDTAVYYCAA |
| | GYQINSGNYNFKDYEYDYWGQGTLVTVSS/GGGGSGGGGSGGGGS/QTV |
| | VTQEPSLTVSPGGTVTLTCGSSTGAVTSGNYPNWVQQKPGQAPRGLIGD |
| | YKDDDDKGTPARFSGSLLGGKAALTLSGVQPEDEAEYYCVLWYSNRWVF |
| | GGGTKLTVL/GGGSGGGS/EVQLVESGGGLVQPGGSLKLSCAASGFTFN |
| | KYAMNWVRQAPGKGLEWVARIRSKYDYKDDDDKADSVKDRFTISRDDSK |
| | NTAYLQMNNLKTEDTAVYYCVRHGNFGNSYISYWAYWGQGTLVTVSS/G |
| | GGGSGGGGSGGGGS/EVQLVESGGGLVQPGNSLRLSCAASGFTFSKFGM |
| | SWVRQAPGKGLEWVSSISGSGRDTLYAESVKGRFTISRDNAKTTLYLQM |
| | NSLRPEDTAVYYCTIGGSLSVSSQGTLVTVSS/HHHHHH |
| | (SEQ ID NO: 251) |
| Pro1271 | QVKLEESGGGSVQTGGSLRLTCAASGRTSRSYGMGWFRQAPGKEREFVS |
| aEGFR D12/NCL/ | GISWRGDSTGYADSVKGRFTISRDNAKNTVDLQMNSLKPEDTAIYYCAA |
| VLi Flag/NCL/ | AAGSAWYGTLYEYDYWGQGTQVTVSS/GGGSGGGS/QTVVTQEPSLTVS |
| VHi Flag/ [symbol] | PGGTVTLTCGSSTGAVTSGNYPNWVQQKPGQAPRGLIGDYKDDDDKGTP |
| [symbol] aEGFR D12/ | ARFSGSLLGGKAALTLSGVQPEDEAEYYCVLWYSNRWVFGGGTKLTVL/ |
| NCL/aCD3 VH/ | GGGSGGGS/EVQLVESGGGLVQPGGSLKLSCAASGFTFNKYAMNWVRQA |
| NCL/aCD3VL/ [symbol] | PGKGLEWVARIRSKYDYKDDDDKADSVKDRFTISRDDSKNTAYLQMNNL |
| [symbol] aHSA/6XHis | KTEDTAVYYCVRHGNFGNSYISYWAYWGQGTLVTVSS/ [symbol] |
| | [symbol] QVKLEESGGGSVQTGGSLRLTCAASGRTSRSYGMGWFRQAPG |
| | KEREFVSGISWRGDSTGYADSVKGRFTISRDNAKNTVDLQMNSLKPEDT |
| | AIYYCAAAAGSAWYGTLYEYDYWGQGTQVTVSS/GGGSGGGS/EVQLVE |
| | SGGGLVQPGGSLKLSCAASGFTFNKYAINWVRQAPGKGLEWVARIRSKY |
| | NNYATYYADQVKDRFTISRDDSKNTAYLQMNNLKTEDTAVYYCVRHANF |
| | GNSYISYWAYWGQGTLVTVSS/GGGSGGGS/QTVVTQEPSLTVSPGGTV |
| | TLTCASSTGAVTSGNYPNWVQQKPGQAPRGLIGGTKFLVPGTPARFSGS |
| | LLGGKAALTLSGVQPEDEAEYYCTLWYSNRWVFGGGTKLTVL/ |
| | [symbol] [symbol] EVQLVESGGGLVQPGNSLRLSCAASGFTFSKFG |
| | MSWVRQAPGKGLEWVSSISGSGRDTLYAESVKGRFTISRDNAKTTLYLQ |
| | MNSLRPEDTAVYYCTIGGSLSVSSQGTLVTVSS/HHHHHH |
| | (SEQ ID NO: 252) |
| Pro1272 | QVKLEESGGGSVQTGGSLRLTCAASGRTSRSYGMGWFRQAPGKEREFVS |
| aEGFR D12/NCL/ | GISWRGDSTGYADSVKGRFTISRDNAKNTVDLQMNSLKPEDTAIYYCAA |
| VHi Flag/NCL/ | AAGSAWYGTLYEYDYWGQGTQVTVSS/GGGSGGGS/EVQLVESGGGLVQ |
| VLi Flag/ [symbol] | PGGSLKLSCAASGFTFNKYAMNWVRQAPGKGLEWVARIRSKYDYKDDDD |
| [symbol] aEGFR D12/ | KADSVKDRFTISRDDSKNTAYLQMNNLKTEDTAVYYCVRHGNFGNSYIS |
| NCL/aCD3 VH/ | YWAYWGQGTLVTVSS/GGGSGGGS/QTVVTQEPSLTVSPGGTVTLTCGS |
| NCL/aCD3 VL/ | STGAVTSGNYPNWVQQKPGQAPRGLIGDYKDDDDKGTPARFSGSLLGGK |
| [symbol] [symbol] aHSA/ | AALTLSGVQPEDEAEYYCVLWYSNRWVFGGGTKLTVL/ [symbol] |
| 6XHis | [symbol] QVKLEESGGGSVQTGGSLRLTCAASGRTSRSYGMGWFRQAP |
| | GKEREFVSGISWRGDSTGYADSVKGRFTISRDNAKNTVDLQMNSLKPED |
| | TAIYYCAAAAGSAWYGTLYEYDYWGQGTQVTVSS/GGGSGGGS/EVQLV |
| | ESGGGLVQPGGSLKLSCAASGFTFNKYAINWVRQAPGKGLEWVARIRSK |
| | YNNYATYYADQVKDRFTISRDDSKNTAYLQMNNLKTEDTAVYYCVRHAN |
| | FGNSYISYWAYWGQGTLVTVSS/GGGSGGGS/QTVVTQEPSLTVSPGGT |
| | VTLTCASSTGAVTSGNYPNWVQQKPGQAPRGLIGGTKFLVPGTPARFSG |
| | SLLGGKAALTLSGVQPEDEAEYYCTLWYSNRWVFGGGTKLTVL/ |
| | [symbol] [symbol] EVQLVESGGGLVQPGNSLRLSCAASGFTFSKFG |
| | MSWVRQAPGKGLEWVSSISGSGRDTLYAESVKGRFTISRDNAKTTLYLQ |
| | MNSLRPEDTAVYYCTIGGSLSVSSQGTLVTVSS/HHHHHH |
| | (SEQ ID NO: 253) |
| Pro1176 | MKGLPGS/QVQLLESGGGLVQPGGSLRLSCAASRNIGSNYAVGWFRQAP |
| aHER2 hu1156/ | GKEREPVAALRWTGSIIGYDDSLRGRFTISKDNSKNTLYLQMNSLKPED |
| NCL/aCD3 VH/ | TAVYYCAARILDRSSYDYWGQGTLVTVSS/GGGGSGGGGSGGGGS/EVQ |
| NCL/aCD3 VL/ | LVESGGGLVQPGGSLKLSCAASGFTFNKYAINWVRQAPGKGLEWVARIR |
| NCL/aHSA/ | SKYNNYATYYADQVKDRFTISRDDSKNTAYLQMNNLKTEDTAVYYCVRH |
| 6XHis | ANFGNSYISYWAYWGQGTLVTVSS/GGGSGGGS/QTVVTQEPSLTVSPG |
| | GTVTLTCASSTGAVTSGNYPNWVQQKPGQAPRGLIGGTKFLVPGTPARF |
| | SGSLLGGKAALTLSGVQPEDEAEYYCTLWYSNRWVFGGGTKLTVL/GGG |
| | GSGGGGSGGGGS/EVQLVESGGGLVQPGNSLRLSCAASGFTFSKFGMSW |
| | VRQAPGKGLEWVSSISGSGRDTLYAESVKGRFTISRDNAKTTLYLQMNS |
| | LRPEDTAVYYCTIGGSLSVSSQGTLVTVSS/HHHHHH |
| | (SEQ ID NO: 297) |
| Pro1179 | MKGLPGS/QVQLVESGGGLVQPGRSLRLSCAASGRTFSNERLGWFRQAP |
| aHER2 hu1160/ | GKERELVAAIRWSGVIIGYADSVRGRFTISRDNAKNTLYLQMNSLRAED |
| NCL/aCD28.1 | TAVYFCAADRGVYGTWDYWGQGTLVTVSS/GGGGSGGGGSGGGGS/QVQ |
| VH C-S/NCL/ | LVQSGAEVKKPGASVKVSCKASGYTFTSYYIHWVRQAPGQGLEWIGSIY |
| aCD28.1 VL/ | PGNVNTNYNEKFKDRATLTVDTSISTAYMELSRLRSDDTAVYFCTRSHY |
| NCL/aHSA/ | GLDWNFDVWGQGTTVTVSS/GGGSGGGS/DIQMTQSPSSLSASVGDRVT |
| 6XHis | ITCHASQNIYVWLNWYQQKPGKAPKLLIYKASNLHTGVPSRFSGSGSGT |
| | DFTLTISSLQPEDFATYYCQQGQTYPYTFGGGTKVEIKR/GGGGSGGGG |
| | SGGGGS/EVQLVESGGGLVQPGNSLRLSCAASGFTFSKFGMSWVRQAPG |
| | KGLEWVSSISGSGRDTLYAESVKGRFTISRDNAKTTLYLQMNSLRPEDT |
| | AVYYCTIGGSLSVSSQGTLVTVSS/HHHHHH |
| | (SEQ ID No: 298) |

Cleavable Linkers (cleavage sites bolded)

| | |
|---|---|
| MMP 2/9 | GPAG/MKGL (SEQ ID NO: 254) |
| | SGGPGPAG/MKGLPGS (SEQ ID NO: 255) |
| | SGGGPGPAG/MKGLPGGS (SEQ ID NO: 256) |
| Meprin A/8 | GGGGKKLA/DEPEGGGS (SEQ ID NO: 257) |
| | KKLA/DEPE (SEQ ID NO: 258) |
| Meprin A/8 | GGGKFLA/DEPEGG (SEQ ID NO: 259) |
| (Variant, high | |
| efficiency) | |
| Meprin/ | SGGGGVYADSLEDGGGGS (SEQ ID NO: 260) |
| Granzyme B | GVYADSLEDG (SEQ ID NO: 261) |
| Matriptase/uPA | GGGSLSGR/SDNHGGGS (SEQ ID NO: 262) |
| (MS) | GLSGR/SDNHG (SEQ ID NO: 263) |
| Matriptase (MV) | SGGGSFTR/QARVVGGGS (SEQ ID NO: 264) |
| | SFTR/QARVV (SEQ ID NO: 265) |
| CathepsinS/MMP9/ | ARLQ/SAAPAG/LKGA (SEQ ID NO: 266) |
| Meprin A | |
| MMP9 (Variant, | GGPGPAG/MHGLPG (SEQ ID NO: 267) |
| high efficiency) | |
| MMP9 (Variant, | GGPGPAG/MEGLPG (SEQ ID NO: 268) |
| low efficiency) | |
| Thrombin 1 | GGGGLVPR/GSLGGGGS (SEQ ID NO: 269) |
| Thrombin 2 | SSGGGMPR/SFRGGGS (SEQ ID NO: 270) |
| Enterokinase/Flag | GGGGDYKDDDDK/GGGS (SEQ ID NO: 271) |
| KLK7-6 | SGGGQNPY/SAGRGGGS (SEQ ID NO: 272) |
| KLK7-13 | SGGGQNPY/SAGGGSGG (SEQ ID NO: 273) |
| KLK7-11 | SGGGRNVY/SAGGGSGG (SEQ ID NO: 274) |
| KLK7-10 | SGGGQNTW/SAGKGGGS (SEQ ID NO: 275) |
| uPA | GGGSHTGR/SAYFGGGS (SEQ ID NO: 276) |
| MMP9-2 | SGGPGPAG/LKGAPGS (SEQ ID NO: 277) |
| Cathepsin S, K, L | SGGGARLQ/SAAPGGGS (SEQ ID NO: 278) |
| | ARLQ/SAAP (SEQ ID NO: 279) |

Antigen sequences

| | |
|---|---|
| HUMAN EGFR | MRPSGTAGAALLALLAALCPASRALEEKKVCQGTSNKLTQLGTFEDHFL |
| | SLQRMFNNCEVVLGNLEITYVQRNYDLSFLKTIQEVAGYVLIALNTVER |
| | IPLENLQIIRGNMYYENSYALAVLSNYDANKTGLKELPMRNLQEILHGA |
| | VRFSNNPALCNVESIQWRDIVSSDFLSNMSMDFQNHLGSCQKCDPSCPN |
| | GSCWGAGEENCQKLTKIICAQQCSGRCRGKSPSDCCHNQCAAGCTGPRE |
| | SDCLVCRKFRDEATCKDTCPPLMLYNPTTYQMDVNPEGKYSFGATCVKK |
| | CPRNYVVTDHGSCVRACGADSYEMEEDGVRKCKKCEGPCRKVCNGIGIG |
| | EFKDSLSINATNIKHFKNCTSISGDLHILPVAFRGDSFTHTPPLDPQEL |
| | DILKTVKEITGFLLIQAWPENRTDLHAFENLEIIRGRTKQHGQFSLAVV |
| | SLNITSLGLRSLKEISDGDVIISGNKNLCYANTINWKKLFGTSGQKTKI |
| | ISNRGENSCKATGQVCHALCSPEGCWGPEPRDCVSCRNVSRGRECVDKC |
| | KLLEGEPREFVENSECIQCHPECLPQAMNITCTGRGPDNCIQCAHYIDG |
| | PHCVKTCPAGVMGENNTLVWKYADAGHVCHLCHPNCTYGCTGPGLEGCP |
| | TNGPKIPSIATGMVGALLLLLVVALGIGLFMRRRHIVRKRTLRRLLQER |
| | ELVEPLTPSGEAPNQALLRILKETEFKKIKVLGSGAFGTVYKGLWIPEG |
| | EKVKIPVAIKELREATSPKANKEILDEAYVMASVDNPHVCRLLGICLTS |
| | TVQLITQLMPFGCLLDYVREHKDNIGSQYLLNWCVQIAKGMNYLEDRRL |
| | VHRDLAARNVLVKTPQHVKITDFGLAKLLGAEEKEYHAEGGKVPIKWMA |
| | LESILHRIYTHQSDVWSYGVTVWELMTFGSKPYDGIPASEISSILEKGE |
| | RLPQPPICTIDVYMIMVKCWMIDADSRPKFRELIIEFSKMARDPQRYLV |
| | IQGDERMHLPSPTDSNFYRALMDEEDMDDVVDADEYLIPQQGFFSSPST |
| | SRTPLLSSLSATSNNSTVACIDRNGLQSCPIKEDSFLQRYSSDPTGALT |
| | EDSIDDTFLPVPEYINQSVPKRPAGSVQNPVYHNQPLNPAPSRDPHYQD |
| | PHSTAVGNPEYLNTVQPTCVNSTFDSPAHWAQKGSHQISLDNPDYQQDF |
| | FPKEAKPNGIFKGSTAENAEYLRVAPQSSEFIGA |
| | (SEQ ID NO: 280) |
| HUMAN | MQSGTHWRVLGLCLLSVGVWGQDGNEEMGGITQTPYKVSISGTTVILTC |
| CD3EPSILON | PQYPGSEILWOHNDKNIGGDEDDKNIGSDEDHLSLKEFSELEQSGYYVC |
| | YPRGSKPEDANFYLYLRARVCENCMEMDVMSVATIVIVDICITGGLLLL |
| | VYYWSKNRKAKAKPVTRGAGAGGRQRGQNKERPPPVPNPDYEPIRKGQR |
| | DLYSGLNQRRI |
| | (SEQ ID NO: 281) |
| HUMAN 4-1 BB | MGNSCYNIVATLLLVLNFERTRSLQDPCSNCPAGTFCDNNRNQICSPCP |
| | PNSFSSAGGQRTCDICRQCKGVFRTRKECSSTSNAECDCTPGFHCLGAG |
| | CSMCEQDCKQGQELTKKGCKDCCFGTFNDQKRGICRPWTNCSLDGKSVL |
| | VNGTKERDVVCGPSPADLSPGASSVTPPAPAREPGHSPQIISFFLALTS |
| | TALLFLLFFLTLRFSVVKRGRKKLLYIFKQPFMRPVQTTQEEDGCSCRF |
| | PEEEEGGCEL |
| | (SEQ ID NO: 282) |
| HUMAN B7-H32IG | MLRRRGSPGMGVHVGAALGALWFCLTGALEVQVPEDPVVALVGTDATLC |
| | CSFSPEPGFSLAQLNLIWQLTDTKQLVHSFAEGQDQGSAYANRTALFPD |
| | LLAQGNASLRLQRVRVADEGSFTCFVSIRDFGSAAVSLQVAAPYSKPSM |
| | TLEPNKDLRPGDTVTITCSSYRGYPEAEVFWQDGQGVPLTGNVTTSQMA |
| | NEQGLFDVHSVLRVVLGANGTYSCLVRNPVLQQDAHGSVTITGQPMTFP |
| | PEALWVTVGLSVCLIALLVALAFVCWRKIKQSCEEENAGAEDQDGEGEG |
| | SKTALQPLKHSDSKEDDGQEIA |
| | (SEQ ID NO: 283) |
| HUMAN B7-H341G | MLRHRGSPGMGVHVGAALGALWFCLTGALEVQVPEDPVVALVGTDATLR |
| | CSFSPEPGFSLAQLNLIWQLTDTKQLVHSFTEGRDQGSAYANRTALFLD |
| | LLAQGNASLRLQRVRVADEGSFTCFVSIRDFGSAAVSLQVAAPYSKPSM |
| | TLEPNKDLRPGDTVTITCSSYRGYPEAEVFWQDGQGAPLTGNVTTSQMA |
| | NEQGLFDVHSVLRVVLGANGTYSCLVRNPVLQQDAHGSITITPQRSPTG |
| | AVEVQVPEDPVVALVGTDATLRCSFSPEPGFSLAQLNLIWQLTDTKQLV |
| | HSFTEGRDQGSAYANRTALFLDLLAQGNASLRLQRVRVADEGSFTCFVS |
| | IRDFGSAAVSLQVAAPYSKPSMTLEPNKDLRPGDTVTITCSSYRGYPEA |
| | EVFWQDGQGAPLTGNVTTSQMANEQGLFDVHSVLRVVLGANGTYSCLVR |
| | NPVLQQDAHGSVTITGQPMTFPPEALWVTVGLSVCLIALLVALAFVCWR |
| | KIKQSCEEENAGAEDQDGEGEGSKTALQPLKHSDSKEDDGQEIA |
| | (SEQ ID NO: 284) |
| HUMAN EPCAM | MAPPQVLAFGLLLAAATATFAAAQEECVCENYKLAVNCFVNNNRQCQCT |
| | SVGAQNTVICSKLAAKCLVMKAEMNGSKLGRRAKPEGALQNNDGLYDPD |
| | CDESGLFKAKQCNGTSTCWCVNTAGVRRTDKDTEITCSERVRTYWIIIE |
| | LKHKAREKPYDSKSLRTALQKEITTRYQLDPKFITSILYENNVITIDLV |
| | QNSSQKTQNDVDIADVAYYFEKDVKGESLFHSKKMDLTVNGEQLDLDPG |
| | QTLIYYVDEKAPEFSMQGLKAGVIAVIVVVVIAVVAGIVVLVISRKKRM |
| | AKYEKAEIKEMGEMHRELNA |
| | (SEQ ID NO: 285) |
| HUMAN TROP2 | MARGPGLAPPPLRLPLLLLVLAAVTGHTAAQDNCTCPTNKMTVCSPDGP |
| | GGRCQCRALGSGMAVDCSTLTSKCLLLKARMSAPKNARTLVRPSEHALV |
| | DNDGLYDPDCDPEGRFKARQCNQTSVCWCVNSVGVRRTDKGDLSLRCDE |
| | LVRTHHILIDLRHRPTAGAFNHSDLDAELRRLFRERYRLHPKFVAAVHY |
| | EQPTIQIELRQNTSQKAAGDVDIGDAAYYFERDIKGESLFQGRGGLDLR |
| | VRGEPLQVERTLIYYLDEIPPKFSMKRLTAGLIAVIVVVVVALVAGMAV |
| | LVITNRRKSGKYKKVEIKELGELRKEPSL |
| | (SEQ ID NO: 286) |
| HUMAN FOLR1 | MAQRMTTQLLLLLVWVAVVGEAQTRIAWARTELLNVCMNAKHHKEKPGP |
| | EDKLHEQCRPWRKNACCSTNTSQEAHKDVSYLYRFNWNHCGEMAPACKR |
| | HFIQDTCLYECSPNLGPWIQQVDQSWRKERVLNVPLCKEDCEQWWEDCR |
| | TSYTCKSNWHKGWNWTSGFNKCAVGAACQPFHFYFPTPTVLCNEIWTHS |
| | YKVSNYSRGSGRCIQMWFDPAQGNPNEEVARFYAAAMSGAGPWAAWPFL |
| | LSLALMLLWLLS |
| | (SEQ ID NO: 287) |
| HUMAN LYPD3 | MDPARKAGAQAMIWTAGWLLLLLLRGGAQALECYSCVQKADDGCSPNKM |
| | KTVKCAPGVDVCTEAVGAVETIHGQFSLAVRGCGSGLPGKNDRGLDLHG |
| | LLAFIQLQQCAQDRCNAKLNLTSRALDPAGNESAYPPNGVECYSCVGLS |
| | REACQGTSPPVVSCYNASDHVYKGCFDGNVTLTAANVTVSLPVRGCVQD |
| | EFCTRDGVTGPGFTLSGSCCQGSRCNSDLRNKTYFSPRIPPLVRLPPPE |
| | PTTVASTTSVTTSTSAPVRPTSTTKPMPAPTSQTPRQGVEHEASRDEEP |
| | RLTGGAAGHQDRSNSGQYPAKGGPQQPHNKGCVAPTAGLAALLLAVAAG |
| | VLL |
| | (SEQ ID NO: 288) |
| HUMAN SERUM | MKWVTFISLLFLFSSAYSRGVFRRDAHKSEVAHRFKDLGEENFKALVLI |
| ALBUMIN | AFAQYLQQCPFEDHVKLVNEVTEFAKTCVADESAENCDKSLHTLFGDKL |
| | CTVATLRETYGEMADCCAKQEPERNECFLQHKDDNPNLPRLVRPEVDVM |
| | CTAFHDNEETFLKKYLYEIARRHPYFYAPELLFFAKRYKAAFTECCQAA |
| | DKAACLLPKLDELRDEGKASSAKQRLKCASLQKFGERAFKAWAVARLSQ |
| | RFPKAEFAEVSKLVTDLTKVHTECCHGDLLECADDRADLAKYICENQDS |
| | ISSKLKECCEKPLLEKSHCIAEVENDEMPADLPSLAADFVESKDVCKNY |
| | AEAKDVFLGMFLYEYARRHPDYSVVLLLRLAKTYETTLEKCCAAADPHE |
| | CYAKVFDEFKPLVEEPQNLIKQNCELFEQLGEYKFQNALLVRYTKKVPQ |
| | VSTPTLVEVSRNLGKVGSKCCKHPEAKRMPCAEDYLSVVLNQLCVLHEK |
| | TPVSDRVTKCCTESLVNRRPCFSALEVDETYVPKEFNAETFTFHADICT |
| | LSEKERQIKKQTALVELVKHKPKATKEQLKAVMDDFAAFVEKCCKADDK |
| | ETCFAEEGKKLVAASQAALGL |
| | (SEQ ID NO: 289) |
| HUMAN CD19 | MPPPRLLFFLLFLTPMEVRPEEPLVVKVEEGDNAVLQCLKGTSDGPTQQ |
| | LTWSRESPLKPFLKLSLGLPGLGIHMRPLAIWLFIFNVSQQMGGFYLCQ |
| | PGPPSEKAWQPGWTVNVEGSGELFRWNVSDLGGLGCGLKNRSSEGPSSP |
| | SGKLMSPKLYVWAKDRPEIWEGEPPCLPPRDSLNQSLSQDLTMAPGSTL |
| | WLSCGVPPDSVSRGPLSWTHVHPKGPKSLLSLELKDDRPARDMWVMETG |
| | LLLPRATAQDAGKYYCHRGNLTMSFHLEITARPVLWHWLLRTGGWKVSA |
| | VTLAYLIFCLCSLVGILHLQRALVLRRKRKRMTDPTRRFFKVTPPPGSG |
| | PQNQYGNVLSLPTPTSGLGRAQRWAAGLGGTAPSYGNPSSDVQADGALG |
| | SRSPPGVGPEEEEGEGYEEPDSEEDSEFYENDSNLGQDQLSQDGSGYEN |
| | PEDEPLGPEDEDSFSNAESYENEDEELTQPVARTMDFLSPHGSAWDPSR |
| | EATSLAGSQSYEDMRGILYAAPQLRSIRGQPGPNHEEDADSYENMDNPD |
| | GPDPAWGGGGRMGTWSTR |
| | (SEQ ID NO: 290) |
| HUMAN CD22 | MHLLGPWLLLLVLEYLAFSDSSKWVFEHPETLYAWEGACVWIPCTYRAL |
| | DGDLESFILFHNPEYNKNTSKFDGTRLYESTKDGKVPSEQKRVQFLGDK |
| | NKNCTLSIHPVHLNDSGQLGLRMESKTEKWMERIHLNVSERPFPPHIQL |
| | PPEIQESQEVTLTCLLNFSCYGYPIQLQWLLEGVPMRQAAVTSTSLTIK |
| | SVFTRSELKFSPQWSHHGKIVTCQLQDADGKFLSNDTVQLNVKHTPKLE |
| | IKVTPSDAIVREGDSVTMTCEVSSSNPEYTTVSWLKDGTSLKKQNTFTL |
| | NLREVTKDQSGKYCCQVSNDVGPGRSEEVFLQVQYAPEPSTVQILHSPA |
| | VEGSQVEFLCMSLANPLPTNYTWYHNGKEMQGRTEEKVHIPKILPWHAG |
| | TYSCVAENILGTGQRGPGAELDVQYPPKKVTTVIQNPMPIREGDTVTLS |
| | CNYNSSNPSVTRYEWKPHGAWEEPSLGVLKIQNVGWDNTTIACAACNSW |
| | CSWASPVALNVQYAPRDVRVRKIKPLSEIHSGNSVSLQCDFSSSHPKEV |
| | QFFWEKNGRLLGKESQLNFDSISPEDAGSYSCWVNNSIGQTASKAWTLE |
| | VLYAPRRLRVSMSPGDQVMEGKSATLTCESDANPPVSHYTWFDWNNQSL |
| | PYHSQKLRLEPVKVQHSGAYWCQGTNSVGKGRSPLSTLTVYYSPETIGR |
| | RVAVGLGSCLAILILAICGLKLQRRWKRTQSQQGLQENSSGQSFFVRNK |
| | KVRRAPLSEGPHSLGCYNPMMEDGISYTTLRFPEMNIPRTGDAESSEMQ |
| | RPPPDCDDTVTYSALHKRQVGDYENVIPDFPEDEGIHYSELIQFGVGER |
| | PQAQENVDYVILKH |
| | (SEQ ID NO: 291) |
| HUMAN BCMA | MLQMAGQCSQNEYFDSLLHACIPCQLRCSSNTPPLTCQRYCNASVTNSV |
| | KGTNAILWTCLGLSLIISLAVFVLMFLLRKINSEPLKDEFKNTGSGLLG |
| | MANIDLEKSRTGDEIILPRGLEYTVEECTCEDCIKSKPKVDSDHCFPLP |
| | AMEEGATILVTTKTNDYCKSLPAALSATEIEKSISAR |
| | (SEQ ID NO: 292) |
| HUMAN | MPLKHYLLLLVGCQAWGAGLAYHGCPSECTCSRASQVECTGARIVAVPT |
| LRRC15 | PLPWNAMSLQILNTHITELNESPFLNISALIALRIEKNELSRITPGAFR |
| | NLGSLRYLSLANNKLQVLPIGLFQGLDSLESLLLSSNQLLQIQPAHFSQ |
| | CSNLKELQLHGNHLEYIPDGAFDHLVGLTKLNLGKNSLTHISPRVFQHL |
| | GNLQVLRLYENRLTDIPMGTFDGLVNLQELALQQNQIGLLSPGLFHNNH |
| | NLQRLYLSNNHISQLPPSVFMQLPQLNRLTLFGNSLKELSPGIFGPMPN |
| | LRELWLYDNHISSLPDNVFSNLRQLQVLILSRNQISFISPGAFNGLTEL |
| | RELSLHTNALQDLDGNVFRMLANLQNISLQNNRLRQLPGNIFANVNGLM |
| | AIQLQNNQLENLPLGIFDHLGKLCELRLYDNPWRCDSDILPLRNWLLLN |
| | QPRLGTDTVPVCFSPANVRGQSLIIINVNVAVPSVHVPEVPSYPETPWY |
| | PDTPSYPDTTSVSSTTELTSPVEDYTDLTTIQVTDDRSVWGMTQAQSGL |
| | AIAAIVIGIVALACSLAACVGCCCCKKRSQAVLMQMKAPNEC |
| | (SEQ ID NO: 293) |
| HUMAN CD38 | MANCEFSPVSGDKPCCRLSRRAQLCLGVSILVLILVVVLAVVVPRWRQQ |
| | WSGPGTTKRFPETVLARCVKYTEIHPEMRHVDCQSVWDAFKGAFISKHP |
| | CNITEEDYQPLMKLGTQTVPCNKILLWSRIKDLAHQFTQVQRDMFTLED |
| | TLLGYLADDLTWCGEFNTSKINYQSCPDWRKDCSNNPVSVFWKTVSRRF |
| | AEAACDVVHVMLNGSRSKIFDKNSTFGSVEVHNLQPEKVQTLEAWVIHG |
| | GREDSRDLCQDPTIKELESIISKRNIQFSCKNIYRPDKFLQCVKNPEDS |
| | SCTSEI |
| | (SEQ ID NO: 294) |
| HUMAN FAP | MKTWVKIVFGVATSAVLALLVMCIVLRPSRVHNSEENTMRALTLKDILN |
| | GTFSYKTFFPNWISGQEYLHQSADNNIVLYNIETGQSYTILSNRTMKSV |
| | NASNYGLSPDRQFVYLESDYSKLWRYSYTATYYIYDLSNGEFVRGNELP |
| | RPIQYLCWSPVGSKLAYVYQNNIYLKQRPGDPPFQITFNGRENKIFNGI |
| | PDWVYEEEMLATKYALWWSPNGKFLAYAEFNDTDIPVIAYSYYGDEQYP |
| | RTINIPYPKAGAKNPVVRIFIIDTTYPAYVGPQEVPVPAMIASSDYYFS |
| | WLTWVTDERVCLQWLKRVQNVSVLSICDFREDWQTWDCPKTQEHIEESR |
| | TGWAGGFFVSTPVFSYDAISYYKIFSDKDGYKHIHYIKDTVENAIQITS |
| | GKWEAINIFRVTQDSLFYSSNEFEEYPGRRNIYRISIGSYPPSKKCVTC |
| | HLRKERCQYYTASFSDYAKYYALVCYGPGIPISTLHDGRTDQEIKILEE |
| | NKELENALKNIQLPKEEIKKLEVDEITLWYKMILPPQFDRSKKYPLLIQ |
| | VYGGPCSQSVRSVFAVNWISYLASKEGMVIALVDGRGTAFQGDKLLYAV |
| | YRKLGVYEVEDQITAVRKFIEMGFIDEKRIAIWGWSYGGYVSSLALASG |
| | TGLFKCGIAVAPVSSWEYYASVYTERFMGLPTKDDNLEHYKNSTVMARA |
| | EYFRNVDYLLIHGTADDNVHFQNSAQIAKALVNAQVDFQAMWYSDQNHG |
| | LSGLSTNHLYTHMTHFLKQCFSLSD |
| | (SEQ ID NO: 295) |
| Human CD28 | MLRLLLALNLFPSIQVTGNKILVKQSPMLVAYDNAVNLSCKYSYNLFSR |
| | EFRASLHKGLDSAVEVCVVYGNYSQQLQVYSKTGFNCDGKLGNESVTFY |
| | LQNLYVNQTDIYFCKIEVMYPPPYLDNEKSNGTIIHVKGKHLCPSPLFP |
| | GPSKPFWVLVVVGGVLACYSLLVTVAFIIFWVRSKRSRLLHSDYMNMTP |
| | RRPGPTRKHYQPYAPPRDFAAYRS |
| | (SEQ ID NO: 296) |

1. A protein comprising: from N- to C-terminus:
(i) a first single domain antigen binding domain (sdABD) that binds to a first human target tumor antigen (TTA);
(ii) a first domain linker;
(iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first human immune cell antigen, and wherein the first heavy chain variable region and the first light chain variable region are not associated in the first constrained scFv domain and the first constrained scFv domain does not bind to the first human immune cell antigen;
(iv) a first cleavable linker;
(v) a second single domain antigen binding domain (sdABD) that binds to a second human target tumor antigen (TTA);
(vi) a second domain linker;
(vii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second human immune cell antigen, and wherein the second heavy chain variable region and the second light chain variable region are not associated in the second constrained scFv domain, and the second constrained scFv domain does not bind to the second human immune cell antigen;
(viii) a second cleavable linker; and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen;
wherein the first human immune cell antigen and the second human immune cell antigen are different.

2. The protein of claim 1, wherein the first immune cell is a T cell, a natural killer (NK) cell, a neutrophil, or a macrophage and/or the second immune cell is a T cell, a natural killer (NK) cell, or a macrophage.

3. (canceled)

4. The protein of claim 1, wherein the first immune antigen is selected from: CD3, CD28, T cell receptor, programmed cell death protein 1 (PD-1), cytotoxic T-lymphocyte-associated protein 4 (CTLA-4), T-cell immunoglobulin and mucin domain 3 (TIM-3), lymphocyte-activation gene 3 (LAG-3), killer-cell immunoglobulin-like receptor (KIR), CD137, OX40, CD27, GITR (TNFRSF18), TIGIT, inducible T cell costimulatory (ICOS), CD16A, CD226, CD96, CD40L, CD226, CRTAM, LFA-1, CD27, CD96, TIGIT, KIR, NKG2D, CSF1R, CD40, MARCO, VSIG4, and CD163; and/or
wherein the second immune antigen is selected from the group consisting of: CD3, CD28, T cell receptor, programmed cell death protein 1 (PD-1), cytotoxic T-lymphocyte-associated protein 4 (CTLA-4), T cell immunoglobulin and mucin domain 3 (TIM-3), lymphocyte-activation gene 3 (LAG-3), killer-cell immunoglobulin-like receptor (KIR), CD137, OX40, CD27, GITR (TNFRSF18), TIGIT, inducible T cell costimulatory (ICOS), CD16A, CD226, CD96, CD40L, CD226, CRTAM, LFA-1, CD27, CD96, TIGIT, KIR, NKG2D, CSF1R, CD40, MARCO, VSIG4 and CD163.

5. (canceled)

6. The protein of claim 1, wherein the first human immune cell antigen is CD3 and the second immune cell antigen is CD28, or
the first human immune cell antigen is CD28 and the second immune cell antigen is CD3.

7. (canceled)

8. The protein of any one of claim 1, wherein the first heavy chain variable region is linked to the N-terminus of the first light chain variable region in the first constrained scFv domain of (iii); or
the first heavy chain variable region is linked to the C-terminus of the first light chain variable region in the first constrained scFv domain of (iii); and/or
the second heavy chain variable region is linked to the N-terminus of the second light chain variable region in the second constrained scFv domain of (vii); or
the second heavy chain variable region is linked to the C-terminus of the second light chain variable region in the second constrained scFv domain of (vii).

9.-13. (canceled)

14. The protein of claim 1, wherein the first human target tumor antigen is selected from EGFR, HER2, Trop2, CA9, LyPD3, FOLR1, EpCAM, and B7H3 and/or the second human target tumor antigen is selected from EGFR, HER2, Trop2, CA9, LyPD3, FOLR1, EpCAM, and B7H3.

15.-17. (canceled)

18. The protein of claim 1, wherein the first cleavable linker comprises a cleavage site for a protease that is present in a tumor microenvironment and/or the second cleavable linker comprises a cleavage site for a protease that is present in a tumor microenvironment.

19. (canceled)

20. The protein of claim 18, wherein the protease is selected from: MMP2, MMP9, Meprin, Cathepsin, granzyme, Matriplase, thrombin, enterokinase, KLK7-6, KLK7-13, KLK7-11, KLK7-10, and uPA.

21.-22. (canceled)

23. The protein of claim 1, wherein the first human target tumor antigen is EGFR and the first sdABD comprises the amino acid sequence of any one of SEQ ID NOs: 4, 5, and 9-11.

24. The protein of claim 23, wherein the second human target tumor antigen is EGFR and the second sdABD comprises the amino acid sequence of any one of SEQ ID NOs: 4, 5, and 9-11.

25. The protein of claim 23, wherein the second human target tumor antigen is HER2 and the second sdABD comprises the amino acid sequence of any one of SEQ ID NOs: 45, 48-52, 54, 58, 60, 63, 66, 68, 72, 75-78, 82, 85, 89, 95, 96, 99, 103, 104, 108, 112, 116, 117, 121, 299, and 300.

26. The protein of claim 1, wherein the first human target tumor antigen is HER2 and the first sdABD comprises the amino acid sequence of any one of SEQ ID NOs: 45, 48-52, 54, 58, 60, 63, 66, 68, 72, 75-78, 82, 85, 89, 95, 96, 99, 103, 104, 108, 112, 116, 117, 121, 299, and 300.

27. The protein of claim 26, wherein the second human target tumor antigen is EGFR and the second sdABD comprises the amino acid sequence of any one of SEQ ID NOs: 4, 5, and 9-11.

28. The protein of claim 26, wherein the second human target tumor antigen is HER2 and the second sdABD comprises the amino acid sequence of any one of SEQ ID NOs: 45, 48-52, 54, 58, 60, 63, 66, 68, 72, 75-78, 82, 85, 89, 95, 96, 99, 103, 104, 108, 112, 116, 117, 121, 299, and 300.

29.-33. (canceled)

34. The protein of claim 1, wherein the first human immune cell antigen is CD3, the first heavy chain variable region comprises the amino acid sequence of SEQ ID NO: 205, and the first light chain variable region comprises the amino acid sequence of SEQ ID NO: 206 and/or the second human immune cell antigen is CD28, the second heavy chain variable region comprises the amino acid sequence of SEQ ID NO: 213 or SEQ ID NO: 216, and the second light chain variable region comprises the amino acid sequence of SEQ ID NO: 214.

35. (canceled)

36. The protein of claim 1, wherein the first human immune cell antigen is CD28, the first heavy chain variable region comprises the amino acid sequence of SEQ ID NO: 213 or SEQ ID NO: 216, and the first light chain variable region comprises the amino acid sequence of SEQ ID NO: 214 and/or second human immune cell antigen is CD3, the second heavy chain variable region comprises the amino acid sequence of SEQ ID NO: 205, and the second light chain variable region comprises the amino acid sequence of SEQ ID NO: 206.

37. (canceled)

38. The protein of claim 1, wherein the third sdABD comprises the amino acid sequence of SEQ ID NO: 220.

39. The protein of claim 1, comprising the amino acid sequence of any one of SEQ ID NOs: 234-249.

40.-47. (canceled)

48. A composition comprising:
a first protein and a second protein, each of which comprising, from N- to C-terminus:
(i) a first single domain antigen binding domain (sdABD) that binds to a first human target tumor antigen (TTA);
(ii) a first domain linker;
(iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first human immune cell antigen, and wherein the first heavy chain variable region and the first light chain variable region are not associated in the first constrained scFv domain and the first constrained scFv domain does not bind to the first human immune cell antigen;
(iv) a first cleavable linker;
(v) a second single domain antigen binding domain (sdABD) that binds to a second human target tumor antigen (TTA);
(vi) a second domain linker;
(vii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second human immune cell antigen, and wherein the second heavy chain variable region and the second light chain variable region are not associated in the second constrained scFv domain, and the second constrained scFv domain does not bind to the second human immune cell antigen;
(viii) a second cleavable linker; and
(ix) a third sdABD that binds to human serum albumin (HSA);
wherein the first heavy chain variable region of (iii) associates with the second light chain variable region of (vii) intramolecularly, forming a variable fragment (Fv) that does not bind the first immune cell antigen or the second immune cell antigen; and
wherein the second heavy chain variable region of (vii) associates with the first light chain variable region of (iii) intramolecularly, forming a variable fragment (Fv) that does not bind the first immune cell antigen or the second immune cell antigen:
wherein the first human immune cell antigen and the second human immune cell antigen are different.

49.-51. (canceled)

52. A composition comprising:
(a) a first homodimer of a first polypeptide, wherein the first polypeptide comprises:
(i) a first single domain antigen binding domain (sdABD) that binds to a first human target tumor antigen (TTA);
(ii) a first domain linker;
(iii) a first constrained single chain variable fragment (scFv) domain comprising a first heavy chain variable region linked to a first light chain variable region via a first constrained non-cleavable linker (CNCL), wherein the first heavy chain variable region and the first light chain variable region, if associated, are capable of binding a first human immune cell antigen, and wherein the first heavy chain variable region and the first light chain variable region are not associated in the first constrained scFv domain and the first constrained scFv domain does not bind to the first human immune cell antigen;
wherein in the first homodimer, the first VH of one polypeptide associates with the first VL of the other polypeptide, and the first VL of one polypeptide associates with the first VH of the other polypeptide, forming two active variable fragments (Fvs) each capable of binding to the first immune antigen;
(b) a second homodimer of a second polypeptide, wherein the second polypeptide comprises:
(i) a second single domain antigen binding domain (sdABD) that binds to a second human target tumor antigen (TTA);
(ii) a second domain linker;
(iii) a second constrained single chain variable fragment (scFv) domain comprising a second heavy chain variable region linked to a second light chain variable region via a second constrained non-cleavable linker (CNCL), wherein the second heavy chain variable region and the second light chain variable region, if associated, are capable of binding a second human immune cell antigen, and wherein the second heavy chain variable region and the second light chain variable region are not associated in the second constrained scFv domain, and the second constrained scFv domain does not bind to the second human immune cell antigen;
wherein in the second homodimer, the second VH of one polypeptide associates with the second VL of the other polypeptide, and the second VL of one polypeptide associates with the second VH of the other polypeptide, forming two active variable fragments (Fvs) each capable of binding to the second immune antigen;
wherein the first human immune cell antigen and the second human immune cell antigen are different.

53.-57. (canceled)


SEQUENCE LISTING (308 sequences)

SEQ ID NO: 1 - protein, 10 aa, Artificial Sequence
  Synthetic
GRTFSSYAMG

SEQ ID NO: 2 - protein, 16 aa, Artificial Sequence
  Synthetic
INWSSGSTYYADSVKG

SEQ ID NO: 3 - protein, 18 aa, Artificial Sequence
  Synthetic
GYQINSGNYNFKDYEYDY

SEQ ID NO: 4 - protein, 127 aa, Artificial Sequence
  Synthetic
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 5 - protein, 127 aa, Artificial Sequence
  Synthetic
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSS

SEQ ID NO: 6 - protein, 10 aa, Artificial Sequence
  Synthetic
GRTSRSYGMG

SEQ ID NO: 7 - protein, 17 aa, Artificial Sequence
  Synthetic
GISWRGDSTGYADSVKG

SEQ ID NO: 8 - protein, 15 aa, Artificial Sequence
  Synthetic
AAGSAWYGTLYEYDY

SEQ ID NO: 9 - protein, 124 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 10 - protein, 124 aa, Artificial Sequence
  Synthetic
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSS

SEQ ID NO: 11 - protein, 124 aa, Artificial Sequence
  Synthetic
EVQLVESGGGVVRPGGSLRLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSS

SEQ ID NO: 12 - protein, 12 aa, Artificial Sequence
  Synthetic
GTGSIFSINLMG

SEQ ID NO: 13 - protein, 16 aa, Artificial Sequence
  Synthetic
RITSGDSTVYADSVKG

SEQ ID NO: 14 - protein, 12 aa, Artificial Sequence
  Synthetic
LLRSSPGATTPY

SEQ ID NO: 15 - protein, 122 aa, Artificial Sequence
  Synthetic
QVQLVESGGGLVQPGGSLTLSCAASGTGSIFSINLMGWYRQAPGKQRELVARITSGDSTVYADSVKGRFTISRDNSKNTLYLQMNSLRPEDTAVYYCNLLLRSSPGATTPYWGQGTLVTVSS

SEQ ID NO: 16 - protein, 10 aa, Artificial Sequence
  Synthetic
GSFSALWAMR

SEQ ID NO: 17 - protein, 15 aa, Artificial Sequence
  Synthetic
SSRGGTTSYADSVKG

SEQ ID NO: 18 - protein, 7 aa, Artificial Sequence
  Synthetic
IDGHLAY

SEQ ID NO: 19 - protein, 114 aa, Artificial Sequence
  Synthetic
QVQLVESGGGLVQPGGSLTLSCVISGSFSALWAMRWYRQAPGQQRELVASSRGGTTSYADSVKGRFTISRDNSKNTLYLQMNSLRPEDTAVYYCNAIDGHLAYWGQGTLVTVSS

SEQ ID NO: 20 - protein, 10 aa, Artificial Sequence
  Synthetic
GRTFSDYDMG

SEQ ID NO: 21 - protein, 17 aa, Artificial Sequence
  Synthetic
AISWSGGHTNYADSVKG

SEQ ID NO: 22 - protein, 17 aa, Artificial Sequence
  Synthetic
DLRFTGGDTTTPETYDY

SEQ ID NO: 23 - protein, 126 aa, Artificial Sequence
  Synthetic
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSS

SEQ ID NO: 24 - protein, 10 aa, Artificial Sequence
  Synthetic
GRTLDNYDMG

SEQ ID NO: 25 - protein, 17 aa, Artificial Sequence
  Synthetic
AISWSGGSTDYAYSVTG

SEQ ID NO: 26 - protein, 17 aa, Artificial Sequence
  Synthetic
DLRFTGGDTMTPETYDY

SEQ ID NO: 27 - protein, 126 aa, Artificial Sequence
  Synthetic
QVQLVESGGGLVQPGRSLRLSCAASGRTLDNYDMGWFRQGPGKEREFVAAISWSGGSTDYAYSVTGRFTISRDNAKNSLYLQMNSLRAEDTALYYCAADLRFTGGDTMTPETYDYWGQGTLVTVSS

SEQ ID NO: 28 - protein, 17 aa, Artificial Sequence
  Synthetic
AISWSGGSTDYAYSVKG

SEQ ID NO: 29 - protein, 126 aa, Artificial Sequence
  Synthetic
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSS

SEQ ID NO: 30 - protein, 10 aa, Artificial Sequence
  Synthetic
GFTVSNSVMA

SEQ ID NO: 31 - protein, 16 aa, Artificial Sequence
  Synthetic
IINSIGITNYADSVKG

SEQ ID NO: 32 - protein, 6 aa, Artificial Sequence
  Synthetic
NFDRIY

SEQ ID NO: 33 - protein, 114 aa, Artificial Sequence
  Synthetic
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSS

SEQ ID NO: 34 - protein, 10 aa, Artificial Sequence
  Synthetic
GNTFSISAMG

SEQ ID NO: 35 - protein, 16 aa, Artificial Sequence
  Synthetic
VTHSDYSTNYADSVKG

SEQ ID NO: 36 - protein, 5 aa, Artificial Sequence
  Synthetic
YGIDY

SEQ ID NO: 37 - protein, 113 aa, Artificial Sequence
  Synthetic
QVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSS

SEQ ID NO: 38 - protein, 10 aa, Artificial Sequence
  Synthetic
GTTFSRDVMG

SEQ ID NO: 39 - protein, 16 aa, Artificial Sequence
  Synthetic
IISRGGSTNYADSVKG

SEQ ID NO: 40 - protein, 9 aa, Artificial Sequence
  Synthetic
NTATWGRVF

SEQ ID NO: 41 - protein, 117 aa, Artificial Sequence
  Synthetic
QVQLVESGGGLVQPGGSLRLSCEASGTTFSRDVMGWYRQAPGKQRELVAIISRGGSTNYADSVKGRFTISRDNSKNTLYLQMNSLRAEDTAVYYCNANTATWGRVFWGQGTLVTVSS

SEQ ID NO: 42 - protein, 10 aa, Artificial Sequence
  Synthetic
GRTFSNYAMG

SEQ ID NO: 43 - protein, 17 aa, Artificial Sequence
  Synthetic
AINWSGTHTDYADSVKG

SEQ ID NO: 44 - protein, 9 aa, Artificial Sequence
  Synthetic
GWSVFDPDY

SEQ ID NO: 45 - protein, 118 aa, Artificial Sequence
  Synthetic
QVQLQESGGGLVQAGGSLILSCAASGRTFSNYAMGWFRQAPGKEREFVAAINWSGTHTDYADSVKGRFTISRDNVKRTVYLQMNSLKPEDTATYFCSRGWSVFDPDYRGQGTQVTVSS

SEQ ID NO: 46 - protein, 10 aa, Artificial Sequence
  Synthetic
GRAISSYAIS

SEQ ID NO: 47 - protein, 17 aa, Artificial Sequence
  Synthetic
AINWSGTHTNYADSVKG

SEQ ID NO: 48 - protein, 118 aa, Artificial Sequence
  Synthetic
QVQLQESGGGLVQPGGSLRLTCEVSGRAISSYAISWFRQAPGEEREFVAAINWSGTHTNYADSVKGRFTISRDNVKNTVYLQMNSLKPEDTATYFCSRGWSVFDPDYRGQGTQVTVSS

SEQ ID NO: 49 - protein, 118 aa, Artificial Sequence
  Synthetic
QVQLQESGGGLVQAGGSLRLTCAASGRAISSYAISWFRQAPGEEREFVAAINWSGTHTDYADSVKGRFTISRDNVKRTVYLQMNSLKPEDTATYFCSRGWSVFDPDYRGQGTQVTVSS

SEQ ID NO: 50 - protein, 118 aa, Artificial Sequence
  Synthetic
QVQLLESGGGLVQAGGSLRLSCAASGRAISSYAISWFRQAPGEEREFVAAINWSGTHTDYADSVKGRFTISRDNSKRTVYLQMNSLRAEDTAVYFCSRGWSVFDPDYRGQGTLVTVSS

SEQ ID NO: 51 - protein, 118 aa, Artificial Sequence
  Synthetic
QVQLQESGGGLVQPGGSLRLTCAASGRAISSYAISWFRQAPGEEREFVAAINWSGTHTDYADSVKGRFTISRDNAKNTVYLQMNSLKPEDTAVYFCSRGWSVFDPDYRGQGTQVTVSS

SEQ ID NO: 52 - protein, 118 aa, Artificial Sequence
  Synthetic
QVQLQESGGGLVQPGGSLRLTCEVSGRAISSYAISWFRQAPGEEREFVAAINWSGTHTNYADSVKGRFTISRDNVKRTVYLQMNSLKPEDTATYFCSRGWSVFDPDYRGQGTQVTVSS

SEQ ID NO: 53 - protein, 9 aa, Artificial Sequence
  Synthetic
GWSLFDPDY

SEQ ID NO: 54 - protein, 118 aa, Artificial Sequence
  Synthetic
QVQLQESGGGLVQTGGSLRLTCEVSGRAISSYAISWFRQAPGEEREFVAAINWSGTHTNYADSVKGRFTISRDNVKNTVYLQMNSLKPEDTATYFCSRGWSLFDPDYRGQGTQVTVSS

SEQ ID NO: 55 - protein, 10 aa, Artificial Sequence
  Synthetic
GFTFDDYAMS

SEQ ID NO: 56 - protein, 17 aa, Artificial Sequence
  Synthetic
SINWSGTHTDYADSVKG

SEQ ID NO: 57 - protein, 12 aa, Artificial Sequence
  Synthetic
GWRLTGGYYDSV

SEQ ID NO: 58 - protein, 121 aa, Artificial Sequence
  Synthetic
QVQLQESGGGLVQPGGSLRLSCAASGFTFDDYAMSWVRQAPGKGLEWVSSINWSGTHTDYADSVKGRFTISRDNAKNTLYLQMNSLKSEDSAVYYCVKGWRLTGGYYDSVQGQGTQVTVSS

SEQ ID NO: 59 - protein, 10 aa, Artificial Sequence
  Synthetic
GFTFDDYALS

SEQ ID NO: 60 - protein, 121 aa, Artificial Sequence
  Synthetic
QVQLQESGGGLVQAGASLRLSCAASGFTFDDYALSWVRQAPGKGLEWVSSINWSGTHTDYADSVKGRFTISRDNAKNTLYLQMNSLKSEDSAVYYCVKGWRLTGGYYDSVQGQGTQVTVSS

SEQ ID NO: 61 - protein, 10 aa, Artificial Sequence
  Synthetic
GFIFDDYAMT

SEQ ID NO: 62 - protein, 12 aa, Artificial Sequence
  Synthetic
GWRHTDNYYAGV

SEQ ID NO: 63 - protein, 121 aa, Artificial Sequence
  Synthetic
QVQLQESGGGLVQPGGSLRLSCAASGFIFDDYAMTWVRQAPGKGLEWVSSINWSGTHTDYADSVKGRFTISRDNAKNTLYLQMNSLKSEDSAVYYCVKGWRHTDNYYAGVQGQGTQVTVSS

SEQ ID NO: 64 - protein, 17 aa, Artificial Sequence
  Synthetic
SINWSGTHTNYADSVKG

SEQ ID NO: 65 - protein, 9 aa, Artificial Sequence
  Synthetic
GWMVVPDST

SEQ ID NO: 66 - protein, 118 aa, Artificial Sequence
  Synthetic
QVQLQESGGGLVQAGGSLRLSCAASGFTFDDYAMSWVRQTPGKGLEWVSSINWSGTHTNYADSVKGRFTISRDNAKKTLYLQMNSLKSEDTAVYYCARGWMVVPDSTRGQGTQVTVSS

SEQ ID NO: 67 - protein, 10 aa, Artificial Sequence
  Synthetic
GFIFSDYAMT

SEQ ID NO: 68 - protein, 118 aa, Artificial Sequence
  Synthetic
QVQLQESGGGLVQAGGSLRLSCAASGFIFSDYAMTWVRQAPGKGLEWVSSINWSGTHTNYADSVKGRFTISRDNAKKTLYLQMNSLKSEDTAVYYCARGWMVVPDSTRGQGTQVTVSS

SEQ ID NO: 69 - protein, 10 aa, Artificial Sequence
  Synthetic
GRIFSAAVMA

SEQ ID NO: 70 - protein, 16 aa, Artificial Sequence
  Synthetic
SITHRGFTRYADSVKD

SEQ ID NO: 71 - protein, 5 aa, Artificial Sequence
  Synthetic
LNGDY

SEQ ID NO: 72 - protein, 113 aa, Artificial Sequence
  Synthetic
QVQLQESGGGLVQAGGSLRLSCAASGRIFSAAVMAWYRQPPGEQRELVASITHRGFTRYADSVKDRFTISRDNVNNTMYLQMNRLSPEDTAIYYCRLLNGDYWGQGTQVTVSS

SEQ ID NO: 73 - protein, 17 aa, Artificial Sequence
  Synthetic
GINWSGTHTRYADSVKG

SEQ ID NO: 74 - protein, 9 aa, Artificial Sequence
  Synthetic
GFAADSRST

SEQ ID NO: 75 - protein, 118 aa, Artificial Sequence
  Synthetic
QVQLQESGGGLVQPGGSLRLSCAASGFTFDDYAMSWVRQAPGKEMEWVSGINWSGTHTRYADSVKGRFTISRDNAKNTLDLQMNSLISEDTAVYYCARGFAADSRSTRGQGTQVTVSS

SEQ ID NO: 76 - protein, 118 aa, Artificial Sequence
  Synthetic
QVQLQESGGGLVQPGGSLRLSCAASGFTFDDYAMSWIRQAPGKEMEWVSGINWSGTHTRYADSVKGRFTISRDNAKNTLDLQMNSLISEDTAVYYCARGFAADSRSTRGQGTQVTVSS

SEQ ID NO: 77 - protein, 118 aa, Artificial Sequence
  Synthetic
QVQLVESGGGVVRPGGSLRLSCAASGFTFDDYAMSWIRQAPGKEMEWVSGINWSGTHTRYADSVKGRFTISRDNAKNSLYLQMNSLRAEDTALYYCARGFAADSRSTRGQGTLVTVSS

SEQ ID NO: 78 - protein, 118 aa, Artificial Sequence
  Synthetic
QVQLQESGGGLVQPGGSLRLSCVASGFTFDDYAMSWVRQAPGKEMEWVSGINWSGTHTRYADSVKGRFTISRDNAKNTLDLQMNSLISEDTAVYYCARGFAADSRSTRGQGTQVTVSS

SEQ ID NO: 79 - protein, 10 aa, Artificial Sequence
  Synthetic
GRIFKNYPMG

SEQ ID NO: 80 - protein, 17 aa, Artificial Sequence
  Synthetic
AITSSGGTTHYRDSVKG

SEQ ID NO: 81 - protein, 16 aa, Artificial Sequence
  Synthetic
SSTPWDIPLGPNEVGY

SEQ ID NO: 82 - protein, 125 aa, Artificial Sequence
  Synthetic
QVQLQESGGGLVQAGGSLRLSCAASGRIFKNYPMGWFRQPPGKDREFVAAITSSGGTTHYRDSVKGRFTISRDNAKNTVYLQMNSLKPDDTAVYYCAGSSTPWDIPLGPNEVGYWGQGTQVTVSS

SEQ ID NO: 83 - protein, 17 aa, Artificial Sequence
  Synthetic
AITSSGGTTPYRDSVKG

SEQ ID NO: 84 - protein, 16 aa, Artificial Sequence
  Synthetic
SSTPWDIPLGPNEVGF

SEQ ID NO: 85 - protein, 125 aa, Artificial Sequence
  Synthetic
QVQLQESGGGLVQAGGSLRLSCAASGRIFKNYPMGWFRQPPGKDREFVAAITSSGGTTPYRDSVKGRFTISRDNAKNTVYLQMNSLKPEDTAVYYCAGSSTPWDIPLGPNEVGFWGQGTQVTVSS

SEQ ID NO: 86 - protein, 10 aa, Artificial Sequence
  Synthetic
GNIFGFNVMG

SEQ ID NO: 87 - protein, 16 aa, Artificial Sequence
  Synthetic
ISTGGRSTDYADSAKG

SEQ ID NO: 88 - protein, 7 aa, Artificial Sequence
  Synthetic
TMASSDY

SEQ ID NO: 89 - protein, 115 aa, Artificial Sequence
  Synthetic
QVQLQESGGGLVQAGGSLRLSCVASGNIFGFNVMGWYRQVPGKQRELVAISTGGRSTDYADSAKGRFTISRNNAKDTVYLQMNSLKPEDTAVYYCKMTMASSDYWGQGTQVTVSS

SEQ ID NO: 90 - protein, 16 aa, Artificial Sequence
  Synthetic
ISTGGGSTNYADSAKG

SEQ ID NO: 91 - protein, 7 aa, Artificial Sequence
  Synthetic
TMASVDY

SEQ ID NO: 92 - protein, 10 aa, Artificial Sequence
  Synthetic
RNIGSNYAVG

SEQ ID NO: 93 - protein, 17 aa, Artificial Sequence
  Synthetic
ALRWTGSIIGYDDSLRG

SEQ ID NO: 94 - protein, 10 aa, Artificial Sequence
  Synthetic
RILDRSSYDY

SEQ ID NO: 95 - protein, 119 aa, Artificial Sequence
  Synthetic
QVQLQESGGGLVQAGGSLKVSCAASRNIGSNYAVGWFRQAPGKEREPVAALRWTGSIIGYDDSLRGRFTISKDDAKNTVYLQMNSLKPEDTGVYYCAARILDRSSYDYWGQGTQVTVSS

SEQ ID NO: 96 - protein, 119 aa, Artificial Sequence
  Synthetic
QVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSS

SEQ ID NO: 97 - protein, 17 aa, Artificial Sequence
  Synthetic
AITWSGGTTHYAESVKG

SEQ ID NO: 98 - protein, 16 aa, Artificial Sequence
  Synthetic
RINYSTISSNEKMYHY

SEQ ID NO: 99 - protein, 125 aa, Artificial Sequence
  Synthetic
QVQLQESGGGLVQAGGSLRLSCTASGRTFSSYAMGWFRQAPEKEREFVAAITWSGGTTHYAESVKGRFTISRDNAKNTVYLQMNTLEPEDTAVYYCAARINYSTISSNEKMYHYWGQGTQVTVSS

SEQ ID NO: 100 - protein, 10 aa, Artificial Sequence
  Synthetic
GRTFSSLAMG

SEQ ID NO: 101 - protein, 17 aa, Artificial Sequence
  Synthetic
GISWSGSTTYYADSVKG

SEQ ID NO: 102 - protein, 14 aa, Artificial Sequence
  Synthetic
RTSSIATIGREYDY

SEQ ID NO: 103 - protein, 123 aa, Artificial Sequence
  Synthetic
QVQLQESGGGLVQAGGSLRLSCATSGRTFSSLAMGWFRQAPGKERELVAGISWSGSTTYYADSVKGRFAISRDNAKNTVYLQMNSLKPEDTAVYYCAARTSSIATIGREYDYWGQGTQVTVSS

SEQ ID NO: 104 - protein, 123 aa, Artificial Sequence
  Synthetic
QVQLLESGGGLVQPGGSLRLSCATSGRTFSSLAMGWFRQAPGKERELVAGISWSGSTTYYADSVKGRFAISRDNSKNTLYLQMNSLRAEDTAVYYCAARTSSIATIGREYDYWGQGTLVTVSS

SEQ ID NO: 105 - protein, 10 aa, Artificial Sequence
  Synthetic
GRTFSNERLG

SEQ ID NO: 106 - protein, 17 aa, Artificial Sequence
  Synthetic
AIRWSGVIIGYADSVRG

SEQ ID NO: 107 - protein, 10 aa, Artificial Sequence
  Synthetic
DRGVYGTWDY

SEQ ID NO: 108 - protein, 119 aa, Artificial Sequence
  Synthetic
QVQLQESGGGLVQAGGSLRLSCAASGRTFSNERLGWFRQAPGKERELVAAIRWSGVIIGYADSVRGRFTISRDNAKSTVYLQMNSLRPEDTAVYFCAADRGVYGTWDYWGQGTQVTVSS

SEQ ID NO: 109 - protein, 10 aa, Artificial Sequence
  Synthetic
GDIFGTYGMA

SEQ ID NO: 110 - protein, 16 aa, Artificial Sequence
  Synthetic
SISIGGIINYADSVKG

SEQ ID NO: 111 - protein, 5 aa, Artificial Sequence
  Synthetic
LDWNY

SEQ ID NO: 112 - protein, 113 aa, Artificial Sequence
  Synthetic
QVQLQESGGGLVQPGKSLRLSCAVSGDIFGTYGMAWYRQAPGKQREPVASISIGGIINYADSVKGRFTISRDNVKNTVYLQMNSLKPEDTAVYYCKLLDWNYWGRGTQVTVSS

SEQ ID NO: 113 - protein, 10 aa, Artificial Sequence
  Synthetic
GRPFSGNAMG

SEQ ID NO: 114 - protein, 17 aa, Artificial Sequence
  Synthetic
AISWSGDSTYYADSVKG

SEQ ID NO: 115 - protein, 18 aa, Artificial Sequence
  Synthetic
TRSFYSRTYYTRPSDYNY

SEQ ID NO: 116 - protein, 127 aa, Artificial Sequence
  Synthetic
QVQLQESGGGLVQAGGSLRLSCAASGRPFSGNAMGWFRQAPGKEREFVAAISWSGDSTYYADSVKGRFTISRDNDENTAYLQMNSLKPEDTAVYYCAATRSFYSRTYYTRPSDYNYWGPGTQVTVSS

SEQ ID NO: 117 - protein, 127 aa, Artificial Sequence
  Synthetic
QVQLLESGGGLVQPGGSLRLSCAASGRPFSGNAMGWFRQAPGKEREFVAAISWSGDSTYYADSVKGRFTISRDNSKNTLYLQMNSLRAEDTAVYYCAATRSFYSRTYYTRPSDYNYWGPGTLVTVSS

SEQ ID NO: 118 - protein, 10 aa, Artificial Sequence
  Synthetic
GRRFNNYFMG

SEQ ID NO: 119 - protein, 17 aa, Artificial Sequence
  Synthetic
LLRESGDNTYYTNSVKG

SEQ ID NO: 120 - protein, 16 aa, Artificial Sequence
  Synthetic
RLDTGFVLYSANSYAY

SEQ ID NO: 121 - protein, 125 aa, Artificial Sequence
  Synthetic
QVQLQESGGGLVQAGGSLRLSCAASGRRFNNYFMGWFRQAPGKEREFVALLRESGDNTYYTNSVKGRFTISRDNARNTVYLQMNSLKPEDTAVYYCAARLDTGFVLYSANSYAYWGQGTQVTVSS

SEQ ID NO: 122 - protein, 10 aa, Artificial Sequence
  Synthetic
GFTLDYYAIG

SEQ ID NO: 123 - protein, 17 aa, Artificial Sequence
  Synthetic
CISSSDGSTYYADSVKG

SEQ ID NO: 124 - protein, 14 aa, Artificial Sequence
  Synthetic
VKSCYSDGYALWGS

SEQ ID NO: 125 - protein, 123 aa, Artificial Sequence
  Synthetic
QVQLLESGGGLVQPGGSLRLSCAASGFTLDYYAIGWFRQAPGKEHEAVSCISSSDGSTYYADSVKGRFTISRDNSKNTLYLQMNSLRAEDTAVYYCATVKSCYSDGYALWGSWGQGTLVTVSS

SEQ ID NO: 126 - protein, 10 aa, Artificial Sequence
  Synthetic
GFTLDYYSIG

SEQ ID NO: 127 - protein, 14 aa, Artificial Sequence
  Synthetic
VKSCYVDGYILWGS

SEQ ID NO: 128 - protein, 123 aa, Artificial Sequence
  Synthetic
QVQLLESGGGLVQPGGSLRLSCAASGFTLDYYSIGWFRQAPGKEREGVSCISSSDGSTYYADSVKGRFTISRDNSKNTLYLQMNSLRAEDTAVYYCATVKSCYVDGYILWGSWGQGTLVTVSS

SEQ ID NO: 129 - protein, 12 aa, Artificial Sequence
  Synthetic
VRSCASPYELGS

SEQ ID NO: 130 - protein, 121 aa, Artificial Sequence
  Synthetic
QVQLLESGGGLVQPGGSLRLSCAASGFTLDYYAIGWFRQAPGKEHEGVSCISSSDGSTYYADSVKGRFTISRDNSKNTLYLQMNSLRAEDTAVYYCATVRSCASPYELGSWGQGTLVTVSS

SEQ ID NO: 131 - protein, 10 aa, Artificial Sequence
  Synthetic
GRSFSSYGMG

SEQ ID NO: 132 - protein, 17 aa, Artificial Sequence
  Synthetic
AISWGGSAVYYANSVKG

SEQ ID NO: 133 - protein, 15 aa, Artificial Sequence
  Synthetic
NRQGRALVRKEFYDY

SEQ ID NO: 134 - protein, 124 aa, Artificial Sequence
  Synthetic
QVQLVESGGGLVQPGGSLRLSCAASGRSFSSYGMGWFRQAPGKEREIVAAISWGGSAVYYANSVKGRFTISRDNSKNTLYLQMNSLRAEDTAVYYCAANRQGRALVRKEFYDYWGQGTLVTVSS

SEQ ID NO: 135 - protein, 10 aa, Artificial Sequence
  Synthetic
GDTSSIYLMG

SEQ ID NO: 136 - protein, 16 aa, Artificial Sequence
  Synthetic
HIRGNGRTNYADSVKG

SEQ ID NO: 137 - protein, 9 aa, Artificial Sequence
  Synthetic
RNFWGSAEY

SEQ ID NO: 138 - protein, 117 aa, Artificial Sequence
  Synthetic
QVQLLESGGGKVQPGGSLRLSCVVSGDTSSIYLMGWYRQTPGKQRELVAHIRGNGRTNYADSVKGRFTISRDNSKNTLYLQMNSLRAEDTAVYYCNVRNFWGSAEYWGQGTLVTVSS

SEQ ID NO: 139 - protein, 10 aa, Artificial Sequence
  Synthetic
RGTFSRYSMG

SEQ ID NO: 140 - protein, 17 aa, Artificial Sequence
  Synthetic
RISWSGDMTYYADSVKG

SEQ ID NO: 141 - protein, 15 aa, Artificial Sequence
  Synthetic
STLGYVWNHPNMYGY

SEQ ID NO: 142 - protein, 10 aa, Artificial Sequence
  Synthetic
GRTFSSQSMG

SEQ ID NO: 143 - protein, 17 aa, Artificial Sequence
  Synthetic
AISWTGANPTYADSVKG

SEQ ID NO: 144 - protein, 20 aa, Artificial Sequence
  Synthetic
DTSGGSYYYERATAETSYDY

SEQ ID NO: 145 - protein, 129 aa, Artificial Sequence
  Synthetic
QVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSS

SEQ ID NO: 146 - protein, 10 aa, Artificial Sequence
  Synthetic
GFTFDYYAIG

SEQ ID NO: 147 - protein, 17 aa, Artificial Sequence
  Synthetic
CISSSHGSTYYADSVKG

SEQ ID NO: 148 - protein, 19 aa, Artificial Sequence
  Synthetic
AGDGGDYHCSGLVDYGMDY

SEQ ID NO: 149 - protein, 128 aa, Artificial Sequence
  Synthetic
QVQLLESGGGLVQPGGSLRLSCAASGFTFDYYAIGWFRQAPGKEREGVSCISSSHGSTYYADSVKGRFTISRDNSKNTVYLQMNSLRAEDTAVYYCATAGDGGDYHCSGLVDYGMDYWGKGTLVTVSS

SEQ ID NO: 150 - protein, 10 aa, Artificial Sequence
  Synthetic
GRTVGRTAMG

SEQ ID NO: 151 - protein, 17 aa, Artificial Sequence
  Synthetic
TISWAGGTTYYADFVKG

SEQ ID NO: 152 - protein, 14 aa, Artificial Sequence
  Synthetic
SEPYSDYDPSGMVY

SEQ ID NO: 153 - protein, 123 aa, Artificial Sequence
  Synthetic
QVQLLESGGGLVQPGGSLRLSCLASGRTVGRTAMGWFRQPPGKEREFVATISWAGGTTYYADFVKGRFTISRDNSKNTLYLQMNSLRAEDTAVYYCAASEPYSDYDPSGMVYWGKGTLVTVSS

SEQ ID NO: 154 - protein, 10 aa, Artificial Sequence
  Synthetic
GRTFGRAAMG

SEQ ID NO: 155 - protein, 17 aa, Artificial Sequence
  Synthetic
TISWSGSNTYYADFVKG

SEQ ID NO: 156 - protein, 123 aa, Artificial Sequence
  Synthetic
QVQLLESGGGLVQPGGSLRLSCAASGRTFGRAAMGWFRQPPGKEREFAATISWSGSNTYYADFVKGRFTISRDNSKNTVYLQMNSLRAEDTAVYYCAASEPYSDYDPSGMVYWGKGTLVTVSS

SEQ ID NO: 157 - protein, 10 aa, Artificial Sequence
  Synthetic
GLTFNTYPMA

SEQ ID NO: 158 - protein, 17 aa, Artificial Sequence
  Synthetic
DMSWSGTNTYYADSVKG

SEQ ID NO: 159 - protein, 16 aa, Artificial Sequence
  Synthetic
GWPYSGTGRSTTDYTY

SEQ ID NO: 160 - protein, 125 aa, Artificial Sequence
  Synthetic
QVQLLESGGGLVQPGGSLRLSCALSGLTFNTYPMAWFRQPPGQEREFVADMSWSGTNTYYADSVKGRFTISRDNSKNTLYLQMNSLRAEDTAVYYCAAGWPYSGTGRSTTDYTYWGQGTLVTVSS

SEQ ID NO: 161 - protein, 10 aa, Artificial Sequence
  Synthetic
GRSFSRYGMG

SEQ ID NO: 162 - protein, 17 aa, Artificial Sequence
  Synthetic
SISWSGHSTYYADSVKG

SEQ ID NO: 163 - protein, 17 aa, Artificial Sequence
  Synthetic
ESLPYESGSPRLTDFAS

SEQ ID NO: 164 - protein, 126 aa, Artificial Sequence
  Synthetic
QVQLLESGGGLVQPGGSLRLSCAASGRSFSRYGMGWLRQAPGKERELVASISWSGHSTYYADSVKGRFTISRDNSKNTLYLQMNSLRAEDTAVYYCAAESLPYESGSPRLTDFASWGQGTLVTVSS

SEQ ID NO: 165 - protein, 10 aa, Artificial Sequence
  Synthetic
RRTFHTYHMG

SEQ ID NO: 166 - protein, 17 aa, Artificial Sequence
  Synthetic
VINWSGGSTVYADSVKG

SEQ ID NO: 167 - protein, 15 aa, Artificial Sequence
  Synthetic
GGATTQRATEASYDY

SEQ ID NO: 168 - protein, 124 aa, Artificial Sequence
  Synthetic
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSS

SEQ ID NO: 169 - protein, 10 aa, Artificial Sequence
  Synthetic
PRTFSTYSMA

SEQ ID NO: 170 - protein, 17 aa, Artificial Sequence
  Synthetic
AINWSGGNTSYADSVKG

SEQ ID NO: 171 - protein, 13 aa, Artificial Sequence
  Synthetic
GGVLAHHNYEYDY

SEQ ID NO: 172 - protein, 122 aa, Artificial Sequence
  Synthetic
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSS

SEQ ID NO: 173 - protein, 17 aa, Artificial Sequence
  Synthetic
AINWSGGQTSYADSVKG

SEQ ID NO: 174 - protein, 122 aa, Artificial Sequence
  Synthetic
QVQLQESGGGLVQPGGSLRLSCEASPRTFSTYSMAWFRQAPGKERSFVAAINWSGGQTSYADSVKGRFTISRDNSKNTLYLQMNSLRAEDTAVYYCAAGGVLAHHNYEYDYWGQGTLVTVSS

SEQ ID NO: 175 - protein, 17 aa, Artificial Sequence
  Synthetic
AINWSGGETSYADSVKG

SEQ ID NO: 176 - protein, 122 aa, Artificial Sequence
  Synthetic
QVQLQESGGGLVQPGGSLRLSCEASPRTFSTYSMAWFRQAPGKERSFVAAINWSGGETSYADSVKGRFTISRDNSKNTLYLQMNSLRAEDTAVYYCAAGGVLAHHNYEYDYWGQGTLVTVSS

SEQ ID NO: 177 - protein, 17 aa, Artificial Sequence
  Synthetic
AINWSGGDTSYADSVKG

SEQ ID NO: 178 - protein, 122 aa, Artificial Sequence
  Synthetic
QVQLQESGGGLVQPGGSLRLSCEASPRTFSTYSMAWFRQAPGKERSFVAAINWSGGDTSYADSVKGRFTISRDNSKNTLYLQMNSLRAEDTAVYYCAAGGVLAHHNYEYDYWGQGTLVTVSS

SEQ ID NO: 179 - protein, 17 aa, Artificial Sequence
  Synthetic
AINWSGGNTAYADSVKG

SEQ ID NO: 180 - protein, 122 aa, Artificial Sequence
  Synthetic
QVQLQESGGGLVQPGGSLRLSCEASPRTFSTYSMAWFRQAPGKERSFVAAINWSGGNTAYADSVKGRFTISRDNSKNTLYLQMNSLRAEDTAVYYCAAGGVLAHHNYEYDYWGQGTLVTVSS

SEQ ID NO: 181 - protein, 17 aa, Artificial Sequence
  Synthetic
AINWSGGNTYYADSVKG

SEQ ID NO: 182 - protein, 122 aa, Artificial Sequence
  Synthetic
QVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSS

SEQ ID NO: 183 - protein, 10 aa, Artificial Sequence
  Synthetic
GSALIINAMG

SEQ ID NO: 184 - protein, 16 aa, Artificial Sequence
  Synthetic
TVTRSGRTNYADSVKG

SEQ ID NO: 185 - protein, 11 aa, Artificial Sequence
  Synthetic
ALWIADGEYDY

SEQ ID NO: 186 - protein, 119 aa, Artificial Sequence
  Synthetic
QVQLVESGGGLVQPGGSLRLSCAASGSALIINAMGWYRQAPGKQRELVATVTRSGRTNYADSVKGRFTISRDNSKNTLYLQMNSLRAEDTAVYYCNVALWIADGEYDYWGQGTLVTVSS

SEQ ID NO: 187 - protein, 10 aa, Artificial Sequence
  Synthetic
GNIFIINVMG

SEQ ID NO: 188 - protein, 16 aa, Artificial Sequence
  Synthetic
TITNGGRTHYADSVKG

SEQ ID NO: 189 - protein, 8 aa, Artificial Sequence
  Synthetic
NHIELGDY

SEQ ID NO: 190 - protein, 116 aa, Artificial Sequence
  Synthetic
QVQLVESGGGLVQPGGSLRLSCAASGNIFIINVMGWYRQAPGKQRELVATITNGGRTHYADSVKGRFTISRDNSKNTLYLQMNSLRAEDTAVYYCNANHIELGDYWGQGTLVTVSS

SEQ ID NO: 191 - protein, 10 aa, Artificial Sequence
  Synthetic
GIIFSVYDMG

SEQ ID NO: 192 - protein, 16 aa, Artificial Sequence
  Synthetic
RITAGGGTYLTDSVKG

SEQ ID NO: 193 - protein, 7 aa, Artificial Sequence
  Synthetic
AWIGDDY

SEQ ID NO: 194 - protein, 115 aa, Artificial Sequence
  Synthetic
QVQLVESGGGLVQPGGSLRLSCTASGIIFSVYDMGWYRQTPGKQREFVARITAGGGTYLTDSVKGRFTISRDNSKNTLYLQMNSLRAEDTGVYYCNAAWIGDDYWGQGTLVTVSS

SEQ ID NO: 195 - protein, 10 aa, Artificial Sequence
  Synthetic
GITFNLHAMR

SEQ ID NO: 196 - protein, 16 aa, Artificial Sequence
  Synthetic
YISARDWTNYADSVKG

SEQ ID NO: 197 - protein, 7 aa, Artificial Sequence
  Synthetic
DLVGEDY

SEQ ID NO: 198 - protein, 115 aa, Artificial Sequence
  Synthetic
QVQLVESGGGLVKPGGSLRLSCAASGITFNLHAMRWYRRAPGKQRELVAYISARDWTNYADSVKGRFTISRDNAKNSLYLQMNSLRAEDTAVYYCNTDLVGEDYWGRGTLVTVSS

SEQ ID NO: 199 - protein, 10 aa, Artificial Sequence
  Synthetic
GFTFNKYAIN

SEQ ID NO: 200 - protein, 19 aa, Artificial Sequence
  Synthetic
RIRSKYNNYATYYADQVKD

SEQ ID NO: 201 - protein, 14 aa, Artificial Sequence
  Synthetic
HANFGNSYISYWAY

SEQ ID NO: 202 - protein, 14 aa, Artificial Sequence
  Synthetic
ASSTGAVTSGNYPN

SEQ ID NO: 203 - protein, 7 aa, Artificial Sequence
  Synthetic
GTKFLVP

SEQ ID NO: 204 - protein, 9 aa, Artificial Sequence
  Synthetic
TLWYSNRWV

SEQ ID NO: 205 - protein, 125 aa, Artificial Sequence
  Synthetic
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 206 - protein, 109 aa, Artificial Sequence
  Synthetic
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 207 - protein, 10 aa, Artificial Sequence
  Synthetic
GYTFTSYYIH

SEQ ID NO: 208 - protein, 17 aa, Artificial Sequence
  Synthetic
CIYPGNVNTNYNEKFKD

SEQ ID NO: 209 - protein, 11 aa, Artificial Sequence
  Synthetic
SHYGLDWNFDV

SEQ ID NO: 210 - protein, 11 aa, Artificial Sequence
  Synthetic
HASQNIYVWLN

SEQ ID NO: 211 - protein, 7 aa, Artificial Sequence
  Synthetic
KASNLHT

SEQ ID NO: 212 - protein, 9 aa, Artificial Sequence
  Synthetic
QQGQTYPYT

SEQ ID NO: 213 - protein, 120 aa, Artificial Sequence
  Synthetic
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 214 - protein, 108 aa, Artificial Sequence
  Synthetic
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKR

SEQ ID NO: 215 - protein, 17 aa, Artificial Sequence
  Synthetic
SIYPGNVNTNYNEKFKD

SEQ ID NO: 216 - protein, 120 aa, Artificial Sequence
  Synthetic
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSS

SEQ ID NO: 217 - protein, 10 aa, Artificial Sequence
  Synthetic
GFTFSKFGMS

SEQ ID NO: 218 - protein, 17 aa, Artificial Sequence
  Synthetic
SISGSGRDTLYAESVKG

SEQ ID NO: 219 - protein, 6 aa, Artificial Sequence
  Synthetic
GGSLSV

SEQ ID NO: 220 - protein, 115 aa, Artificial Sequence
  Synthetic
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 221 - protein, 514 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAG

SEQ ID NO: 222 - protein, 515 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSDVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSHHHHHH

SEQ ID NO: 223 - protein, 901 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 224 - protein, 407 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGSAWSHPQFEKGGGSGGGSGGSSAWSHPQFEK

SEQ ID NO: 225 - protein, 407 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGSAWSHPQFEKGGGSGGGSGGSSAWSHPQFEK

SEQ ID NO: 226 - protein, 506 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGSGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 227 - protein, 506 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKRGGGSGGGSQVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 228 - protein, 506 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 229 - protein, 506 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSSGGGSGGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKRGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 230 - protein, 506 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 231 - protein, 506 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 232 - protein, 374 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRHHHHHH

SEQ ID NO: 233 - protein, 374 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSHHHHHH

SEQ ID NO: 234 - protein, 893 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 235 - protein, 893 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 236 - protein, 893 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 237 - protein, 893 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 238 - protein, 893 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 239 - protein, 893 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGSSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 240 - protein, 893 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 241 - protein, 893 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 242 - protein, 906 aa, Artificial Sequence
  Synthetic
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGGSGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 243 - protein, 906 aa, Artificial Sequence
  Synthetic
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSEVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 244 - protein, 883 aa, Artificial Sequence
  Synthetic
QVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSQVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 245 - protein, 883 aa, Artificial Sequence
  Synthetic
QVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSQVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 246 - protein, 888 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSQVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 247 - protein, 888 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSQVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 248 - protein, 888 aa, Artificial Sequence
  Synthetic
QVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 249 - protein, 888 aa, Artificial Sequence
  Synthetic
QVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 250 - protein, 914 aa, Artificial Sequence
  Synthetic
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGGSGGGGSGGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 251 - protein, 908 aa, Artificial Sequence
  Synthetic
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSGGPGPAGMKGLPGSEVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGGSGGGGSGGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 252 - protein, 901 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 253 - protein, 901 aa, Artificial Sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGSGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLSGGPGPAGMKGLPGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 254 - protein, 8 aa, Artificial Sequence
  Synthetic
GPAGMKGL

SEQ ID NO: 255 - protein, 15 aa, Artificial Sequence
  Synthetic
SGGPGPAGMKGLPGS

SEQ ID NO: 256 - protein, 17 aa, Artificial Sequence
  Synthetic
SGGGPGPAGMKGLPGGS

SEQ ID NO: 257 - protein, 16 aa, Artificial Sequence
  Synthetic
GGGGKKLADEPEGGGS

SEQ ID NO: 258 - protein, 8 aa, Artificial Sequence
  Synthetic
KKLADEPE

SEQ ID NO: 259 - protein, 13 aa, Artificial Sequence
  Synthetic
GGGKFLADEPEGG

SEQ ID NO: 260 - protein, 18 aa, Artificial Sequence
  Synthetic
SGGGGVYADSLEDGGGGS

SEQ ID NO: 261 - protein, 10 aa, Artificial Sequence
  Synthetic
GVYADSLEDG

SEQ ID NO: 262 - protein, 16 aa, Artificial Sequence
  Synthetic
GGGSLSGRSDNHGGGS

SEQ ID NO: 263 - protein, 10 aa, Artificial Sequence
  Synthetic
GLSGRSDNHG

SEQ ID NO: 264 - protein, 17 aa, Artificial Sequence
  Synthetic
SGGGSFTRQARVVGGGS

SEQ ID NO: 265 - protein, 9 aa, Artificial Sequence
  Synthetic
SFTRQARVV

SEQ ID NO: 266 - protein, 14 aa, Artificial Sequence
  Synthetic
ARLQSAAPAGLKGA

SEQ ID NO: 267 - protein, 13 aa, Artificial Sequence
  Synthetic
GGPGPAGMHGLPG

SEQ ID NO: 268 - protein, 13 aa, Artificial Sequence
  Synthetic
GGPGPAGMEGLPG

SEQ ID NO: 269 - protein, 16 aa, Artificial Sequence
  Synthetic
GGGGLVPRGSLGGGGS

SEQ ID NO: 270 - protein, 15 aa, Artificial Sequence
  Synthetic
SSGGGMPRSFRGGGS

SEQ ID NO: 271 - protein, 16 aa, Artificial Sequence
  Synthetic
GGGGDYKDDDDKGGGS

SEQ ID NO: 272 - protein, 16 aa, Artificial Sequence
  Synthetic
SGGGQNPYSAGRGGGS

SEQ ID NO: 273 - protein, 16 aa, Artificial Sequence
  Synthetic
SGGGQNPYSAGGGSGG

SEQ ID NO: 274 - protein, 16 aa, Artificial Sequence
  Synthetic
SGGGRNVYSAGGGSGG

SEQ ID NO: 275 - protein, 16 aa, Artificial Sequence
  Synthetic
SGGGQNTWSAGKGGGS

SEQ ID NO: 276 - protein, 16 aa, Artificial Sequence
  Synthetic
GGGSHTGRSAYFGGGS

SEQ ID NO: 277 - protein, 15 aa, Artificial Sequence
  Synthetic
SGGPGPAGLKGAPGS

SEQ ID NO: 278 - protein, 16 aa, Artificial Sequence
  Synthetic
SGGGARLQSAAPGGGS

SEQ ID NO: 279 - protein, 8 aa, Artificial Sequence
  Synthetic
ARLQSAAP

SEQ ID NO: 280 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 281 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 282 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 283 - protein, 316 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 284 - protein, 534 aa, Homo sapiens
MLRHRGSPGMGVHVGAALGALWFCLTGALEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 285 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 286 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 287 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 288 - protein, 346 aa, Homo sapiens [sequence omitted]

SEQ ID NO: 289 - protein, 609 aa, Homo sapiens [sequence omitted]

SEQ ID NO: 290 - protein, 557 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYAAPQLRSIRGQPGPNHEEDADSYENMDNPDGPDPAWGGGGRMGTWSTR

SEQ ID NO: 291 - protein, 847 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 292 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 293 - protein, 581 aa, Homo sapiens
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 294 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 295 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 296 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 297 - protein, 519 aa, Artificial Sequence
  Synthetic
MKGLPGSQVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 298 - protein, 513 aa, Artificial Sequence
  Synthetic
MKGLPGSQVQLVESGGGLVQPGRSLRLSCAASGRTFSNERLGWFRQAPGKERELVAAIRWSGVIIGYADSVRGRFTISRDNAKNTLYLQMNSLRAEDTAVYFCAADRGVYGTWDYWGQGTLVTVSSGGGGSGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGSGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 299 - protein, 118 aa, Artificial Sequence
  Synthetic
QVQLQESGGGLVQPGGSLRLTCAASGRAISSYAISWFRQAPGEEREFVAAINWSGTHTNYADSVKGRFTISRDNVKNTVYLQMNSLKPEDTATYFCSRGWSVFDPDYRGQGTQVTVSS

SEQ ID NO: 300 - protein, 115 aa, Artificial Sequence
  Synthetic
QVQLQESGGGLVQAGGSLRLSCVASGNIFGFNVMGWYRQVPGKQRELVAISTGGGSTNYADSAKGRFTVSRNNAKDTVYLQMNSLKPEDTAVYYCKMTMASVDYWGQGTQVTVSS

SEQ ID NO: 301 - protein, 124 aa, Artificial Sequence
  Synthetic
QVQLLESGGGLVQPGGSLRLSCVASRGTFSRYSMGWFRQAPGKEREFVGRISWSGDMTYYADSVKGRFTISRDNSKNTLYLQMNSLRAEDTAVYYCAASTLGYVWNHPNMYGYWGHGTLVTVSS

SEQ ID NO: 302 - protein, 8 aa, Artificial Sequence
  Synthetic
GGGSGGGS

SEQ ID NO: 303 - protein, 2 aa, Artificial Sequence
  Synthetic
  MISC_FEATURE (1)..(2): may be repeated 1-10 times
GS

SEQ ID NO: 304 - protein, 3 aa, Artificial Sequence
  Synthetic
  MISC_FEATURE (1)..(3): may be repeated 1-10 times
GGS

SEQ ID NO: 305 - protein, 4 aa, Artificial Sequence
  Synthetic
  MISC_FEATURE (1)..(4): may be repeated 1-10 times
GGGS

SEQ ID NO: 306 - protein, 4 aa, Artificial Sequence
  Synthetic
  MISC_FEATURE (1)..(4): may be repeated 1-10 times
GGSG

SEQ ID NO: 307 - protein, 5 aa, Artificial Sequence
  Synthetic
  MISC_FEATURE (1)..(5): may be repeated 1-10 times
GGSGG

SEQ ID NO: 308 - protein, 5 aa, Artificial Sequence
  Synthetic
  MISC_FEATURE (1)..(5): may be repeated 1-10 times
GGGGS